(19) United States
(12) Patent Application Publication
(10) Pub. No.: US 2025/0120927 A1
(43) Pub. Date: Apr. 17, 2025

(54) CNDP2 MODULATORS AND METHODS FOR THEIR USE

(71) Applicant: Metabolon Inc., Morrisville, NC (US)

(72) Inventors: Rangaprasad Sarangarajan, Boylston, MA (US); Dinesh Chimmanamada, Arlington, MA (US); Janice Jones, Apex, NC (US); Joseph Patrick McGinley, Dublin (IE); Patricia A. Sheridan, Chapel Hill, NC (US); Joshua Bryan Simpson, Durham, NC (US)

(21) Appl. No.: 18/648,984

(22) Filed: Apr. 29, 2024

Related U.S. Application Data
(60) Provisional application No. 63/499,066, filed on Apr. 28, 2023.

(51) Int. Cl. A61K 31/165; A61K 31/41; A61K 31/4406; C07C 237/20; C07D 213/55; C07D 257/06

(52) U.S. Cl. CPC A61K 31/165; A61K 31/41; A61K 31/4406; C07C 237/20; C07D 213/55; C07D 257/06

(57) ABSTRACT

This disclosure features chemical entities (e.g., a compound or a pharmaceutically acceptable salt, and/or hydrate, and/or cocrystal, and/or drug combination and/or stereoisomers (e.g., enantiomers and diastereomers) of the compound) that inhibit (e.g., antagonize) Carnosine Dipeptidase 2 (CNDP2). Said chemical entities are useful, e.g., for treating a condition, disease or disorder in which increased (e.g., excessive) CNDP2 activation contributes to the pathology and/or symptoms and/or progression of the condition, disease or disorder (e.g., cancer) in a subject (e.g., a human). This disclosure also features compositions containing the same as well as methods of using and making the same.

### CROSS REFERENCE TO RELATED APPLICATIONS

This application claims the benefit of U.S. Provisional Application No. 63/499,066, filed on Apr. 28, 2023, which is incorporated herein by reference in its entirety.

### TECHNICAL FIELD

This disclosure features chemical entities (e.g., a compound or a pharmaceutically acceptable salt, and/or hydrate, and/or cocrystal, and/or drug combination of the compound) that inhibit (e.g., antagonize) Carnosine Dipeptidase 2 (CNDP2). The chemical entities are useful, e.g., for treating a condition, disease or disorder in which increased (e.g., excessive) CNDP2 expression contributes to the pathology and/or symptoms and/or progression of the condition, disease or disorder (e.g., cancer) in a subject (e.g., a human). This disclosure also features compositions containing the same as well as methods of using and making the same.

### BACKGROUND

CNDP Dipeptidase 2 (EC 3.4.13.18) (CNDP2), is a cytosolic, non-specific dipeptidase which is a part of the peptidase M20A protein family.

### SUMMARY

This disclosure features chemical entities (e.g., a compound or a pharmaceutically acceptable salt, and/or hydrate, and/or cocrystal, and/or drug combination, and/or stereoisomers (e.g., enantiomers and diastereomers) of the compound)) that inhibit (e.g., antagonize) Carnosine Dipeptidase 2 (CNDP2). The chemical entities are useful, e.g., for treating a condition, disease or disorder in which altered (e.g., excessive) CNDP2 expression or activity contributes to the pathology and/or symptoms and/or progression of the condition, disease or disorder (e.g., cancer) in a subject (e.g., a human). This disclosure also features compositions containing the same and methods of using and making the same.

Without wishing to be bound by any particular theory, inhibition of CNDP2 may interfere with the growth and/or proliferation of cancer cells in at least two different ways.

First, while CNDP2 is primarily seen as a dipeptidase, in the presence of high lactate levels, CNDP2 can act as a transpeptidase to generate N-lactoyl amino acids (NLAAs). Cancer cells have higher levels of lactate due to increased glycolytic flux. The high lactate levels may lead to the generation of N-lactoyl amino acids (NLAAs) by CNDP2. The cancer cells may be able to use NLAAs as a readily bioavailable source of amino acids to support cell growth and proliferation. Thus, inhibiting CNDP2 may reduce production of NLAAs in cancer cells and reduce their growth and/or proliferation.

Glutathione (GSH) is the most abundant intracellular anti-oxidant in eukaryotic cells, and cancer cells maintain high levels of GSH to counteract the redox environment associated with rapid proliferation. Cysteinyl-glycine (Cys-Gly) is an important precursor for GSH biosynthesis. Cys-Gly is a substrate for CNDP2, producing cysteine and glycine. Inhibiting CNDP2 may reduce production of GSH in cancer cells and reduce their ability to fight oxidative stress and this may result in an increase in programmed cell death.

Other substrates for CNDP2 include various dipeptides (e.g., beta-alanine-Histidine, N-methyl beta-alanine-Histidine; Leucine-Histidine; Serine-Histidine; Tyrosine-Histidine; Alanine-Histidine; Valine-Histidine; Isoleucine-Histidine; Methionine-Histidine; Glycine-Histidine; and Histidine-Histidine); Glutathione-derived peptides (e.g., Cysteine-Glycine and Serine-Glutamine); lactoyl amino acids (e.g., N-Lactoyl-phenylalanine; N-Lactoyl-tyrosine; N-lactoyl-tryptophan; N-lactoyl-leucine; N-lactoyl-isoleucine; N-lactoyl-valine; and N-lactoyl-methionine); and various threonine-containing peptides (e.g., Threonine-Threonine; Threonine-Serine and Serine-Threonine).

As noted above, inhibition of CNDP2 may be useful in treatment of metabolic disorder, frailty, muscle loss, mitochondrial dysfunction and obesity. These uses are supported by a variety of studies, including studies showing increased levels of N-lactoyl amino acid(s) (sometimes referred to herein as “NLAA” or “NLAAs”) in subjects suffering from certain metabolic disorders. As noted above, NLAA can be produced by the transpeptidase activity of CNDP2 when lactate levels are sufficiently high.

The Microbiome and Insulin Longitudinal Evaluation Study (MILES) cohort is comprised of African-American and non-Hispanic Caucasian participants (40-80 years) without underlying diabetes. The study was initiated to investigate the role of gut microbiome on insulin sensitivity, secretion, and clearance as a measure of metabolic status. It enrolled 129 individuals of African American descent and 224 non-Hispanic Caucasians. Body Mass Index (BMI), Sex, Age, Insulin, C-peptide, and glucose levels at baseline and 2 hours following an oral glucose tolerance test (OGTT) were among the metadata collected in the study. Insulin sensitivity index (ISI) data was calculated from the glucose and OGTT values.

We used a metabolomics discovery platform generate metabolomic profiles from 554 plasma samples collected from individuals in the MILES cohort. A total of 1,525 compounds were assessed. Of these, 1,253 compounds were of known identity. The remaining 272 compounds assessed had spectral characteristics consistent with novel compounds of unknown structure and function.

Statistical analysis (correlations, partial correlations, ANOVAs and ANCOVAs) of the metabolites and their dependencies on covariates (from the availed metadata) were performed on the baseline metabolomic datasets. Certain of the significant findings from this analysis are summarized in Table 1 below, which presents Spearman Correlation Analysis for Insulin Sensitivity Index (ISI). Table 1 lists certain metabolites that were positively or negatively correlated to ISI. The p-value describes statistical significance, q-value represents adjustments to potential false discovery rates and Spearman Corr represents performance of each of the metabolites with respect to ISI. A positive correlation (positive Spearman values) suggests, increase in metabolite levels is correlated to improved ISI (in essence, improved glucose homeostasis and insulin sensitivity). A negative correlation (negative Spearman values) suggests an inverse relationship i.e. increase in the metabolite levels is associated with poor glucose homeostasis and insulin sensitivity. Within Table 1, the value of “0” indicates very high significance and values exceeding limit of reporting in this specific table.

TABLE 1

| Metabolite | p-value | q-value | Spearman Corr |
|---|---|---|---|
| lactate | 0 | 0 | −0.420 |
| X-14056 | 0 | 0 | −0.438 |
| 1-carboxyethylisoleucine | 1.19E−18 | 3.64E−17 | −0.450 |
| X-12456 | 1.12E−18 | 3.64E−17 | −0.450 |
| 1-carboxyethyltyrosine | 4.52E−23 | 1.92E−21 | −0.498 |
| 1-carboxyethylvaline | 1.31E−23 | 6.05E−22 | −0.504 |
| 1-carboxyethylphenylalanine | 0 | 0 | −0.522 |
| cortolone glucuronide (1) | 4.05E−26 | 2.03E−24 | −0.528 |
| 1-carboxyethylleucine | 5.85E−28 | 3.24E−26 | −0.544 |

The metabolites with negative (inverse) correlation to the insulin sensitivity index within the MILES cohort that were significant include lactate and five N-lactoyl-amino acids (NLAA). Based on MILES data analysis, an increase in circulating levels of NLAAs is associated with the potential incidence of insulin resistance with important implications for insulin sensitivity within the context of metabolic syndrome, insulin resistance, and type 2 diabetes mellitus (T2DM).

Consistent with these findings, an increase in NLAAs has been found in obese diabetic individuals compared to obese non-diabetic individuals (Scott B et al., 2024). In the same study, further analysis of clinical cohorts demonstrated that although T2DM individuals had a trending increase in circulating levels of NLAAs, individuals specifically on metformin therapy (standard of care for T2DM) had significant elevation of circulating NLAAs compared to untreated cohorts (Scott B et al., 2024).

Metformin significantly increases the formation and secretion of N-lactose-phenylalanine in controlled experimental conditions (Xiao S et al., 2024). One of the many mechanisms attributed to metformin is its seeming ability to act as an inhibitor of mitochondrial complex 1 function, shifting glycolytic pathways toward lactic acid generation (DeFronzo et al., 2015).

Mitochondrial dysfunction is common to MELAS, PKU, sepsis, T2DM, and cancer, resulting in a shift in cellular metabolism from oxidative phosphorylation to glycolysis (anaerobic or aerobic) to meet increased energy demands (Warburg Phenotype). The primary consequence of using glycolysis to meet energy demands is the conversion of pyruvate to excess lactate within the cells. The increase in intracellular lactic acid concentrations is important for achieving molar concentration requirements for the transpeptidase activity of CNDP2 enzyme in the formation of NLAA conjugates. Thus, the significant increase in circulating levels of NLAA observed in MELAS, PKU, sepsis, T2DM, and cancer might be due to a mitochondrial dysfunction mediated shift in cellular metabolism to generate excess lactate, a phenomenon similar to that elicited by metformin.

The modulation of the biochemical pathway involved in the formation of NLAAs and their release into the systemic circulation influences health and disease outcomes. This provides the scientific rationale for the development of therapeutic interventions directly targeting the enzymatic activity of CNDP2 to modulate the biosynthesis of NLAAs for the treatment of disease and maintenance of health.

Gait speed and grip strength are validated measures for reliable assessment of physical performance in aging adults and in other clinical conditions. In a population cohort of 1298 participants, N-lactoyl phenylalanine was one of seven metabolites associated with a decline in gait speed, suggesting the involvement of NLAAs in influencing physical performance (Nierenberg J L et al., 2020).

In another population study consisting of 3042 individuals (1290 men and 1752 women) over age 60, a comprehensive metabolomic profile found that an increase in NLAA levels is associated with decreased grip strength. (unpublished data).

In one aspect, compounds of Formula (I), or a pharmaceutically acceptable salt thereof, are featured:

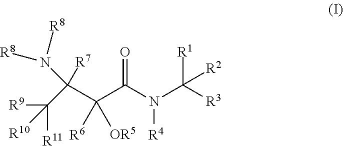

in which R¹, R², R³, R⁴, R⁵, R⁶, R⁷, R⁸, R⁹, R¹⁰, R¹¹, R¹², R¹³, R¹⁴, R¹⁵, R^{a}, R^{b}, R^{c}, R^{d}, R^{e}, R^{f}, R^{g}, R′, R″, L¹, L², L³, and L⁴ can be as defined anywhere herein.

In another aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In a further aspect, this disclosure features methods of inhibiting Carnosine Dipeptidase 2 (CNDP2) in a subject, which include administering to the subject an effective amount of a compound described herein, or a pharmaceutically acceptable salt thereof.

In one aspect, methods of treating a condition, disease or disorder ameliorated by modulating (e.g., inhibiting) CNDP2 are featured, e.g., treating a condition, disease or disorder in which altered (e.g., increased) CNDP2 expression or activity contributes to the pathology and/or symptoms and/or progression of the condition, disease or disorder (e.g., cancer) in a subject (e.g., a human). The methods include administering to a subject in need of such treatment an effective amount of a chemical entity described herein (e.g., a compound described generically or specifically herein or a pharmaceutically acceptable salt thereof or compositions containing the same).

In another aspect, methods of treating cancer are featured that include administering to a subject in need of such treatment an effective amount of a chemical entity described herein (e.g., a compound described generically or specifically herein or a pharmaceutically acceptable salt thereof or compositions containing the same).

In a further aspect, methods of treating frailty or one or more frailty conditions are featured that include administering to a subject in need of such treatment an effective amount of a chemical entity described herein (e.g., a compound described generically or specifically herein or a pharmaceutically acceptable salt thereof or compositions containing the same).

As used herein, “frailty” refers to a syndrome characterized by reduced physical endurance and increased susceptibility to endogenous and exogenous stressors (see, e.g., Song X, et al., (2010) J Am Geriatr Soc. 58(4):681-7). Phenotypic frailty typically includes one or more of the following criteria: unintentional weight loss predominantly due to loss of muscle tissue, slow walking pace, exhaustion, weak grip strength, and physical inactivity (see, e.g., Fried, L., et al., (2001) J. Gerontology: Medical Sciences, 56A:3, M146-M156).

In some embodiments, the frailty condition includes one or more (e.g., 2, 3, or 4) of: weight loss, exhaustion, weakness, and physical inactivity.

In certain embodiments, the frailty condition is an age-related frailty condition (e.g., the subject is 60 years old or older; e.g., 60-69 years old, 70-79 years old, or 80-89 years old, 90-99 years old, or 100 years old or older).

In other embodiments, the frailty condition is not age-related.

In some embodiments, the subject has one or more (e.g., 2, 3, 4, or 5) symptoms of: decreased grip strength, muscle wasting, cachexia, physical injury, impaired locomotion, and decreased physical stability. In certain embodiments, the subject has decreased grip strength.

In some embodiments, the subject is male.

In some embodiments, the subject is female.

In some embodiments, the subject exhibits a BMI that is less than 18.5. In some embodiments, the subject exhibits a BMI that is 18.5 to <25. In some embodiments, the subject exhibits a BMI that is 25.0 to <30. In some embodiments, the subject exhibits a BMI that is 30.0 or higher.

In some embodiments, the subject the subject has an HbA1c level that is equal to or greater than 6.5% of whole blood. In some embodiments, the subject has an HbA1c level ranging from 6.5% to 20% of whole blood. In some embodiments, the subject has an HbA1c level that is equal to or greater than 7.0% of whole blood. In some embodiments, the subject has an HbA1c level ranging from 7.0% to 20% of whole blood. In some embodiments, the subject has an HbA1c level that is equal to or greater than 7.5% of whole blood. In some embodiments, the subject has an HbA1c level ranging from 7.5% to 20% of whole blood.

In another aspect, methods of treating mitochondrial dysfunction that include administering to a subject in need of such treatment an effective amount of a chemical entity described herein (e.g., a compound described generically or specifically herein or a pharmaceutically acceptable salt thereof or compositions containing the same).

In some embodiments, the disease or symptom caused by mitochondrial dysfunction is selected from the group consisting of a mitochondrial disease, e.g., Leigh syndrome, mitochondrial encephalopathy, lactic acidosis and stroke-like episodes (MELAS), sepsis, or Leber's disease; diabetes and cancer.

In another aspect, methods of treating muscle loss (e.g., loss of muscle mass) that include administering to a subject in need of such treatment an effective amount of a chemical entity described herein (e.g., a compound described generically or specifically herein or a pharmaceutically acceptable salt thereof or compositions containing the same).

In another aspect, methods of treating metabolic disorder (e.g., Type-2 diabetes, increased hemoglobin A1c, PKU, metabolic syndrome, or increased serum glucose) that include administering to a subject in need of such treatment an effective amount of a chemical entity described herein (e.g., a compound described generically or specifically herein or a pharmaceutically acceptable salt thereof or compositions containing the same).

In another aspect, methods of treating obesity that include administering to a subject in need of such treatment an effective amount of a chemical entity described herein (e.g., a compound described generically or specifically herein or a pharmaceutically acceptable salt thereof or compositions containing the same).

In another aspect, methods of treating insulin resistance or insulin sensitivity that include administering to a subject in need of such treatment an effective amount of a chemical entity described herein (e.g., a compound described generically or specifically herein or a pharmaceutically acceptable salt thereof or compositions containing the same).

In another aspect, there is provided a compound, or a pharmaceutically acceptable salt or tautomer thereof, as described herein for use in the treatment of a condition, disease or disorder associated with increased (e.g., excessive) CNDP2 expression.

In another aspect, there is provided a compound, or a pharmaceutically acceptable salt or tautomer thereof, described herein for use in the treatment of cancer.

In another aspect, there is provided a compound, or a pharmaceutically acceptable salt or tautomer thereof, as described herein for use in the treatment of cancer selected from the group consisting of: ovarian cancer, colorectal cancer, and esophageal cancer, melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, glioblastoma multiforme, endometrial cancers, and hepatocellular carcinoma.

In another aspect, there is provided a compound, or a pharmaceutically acceptable salt or tautomer thereof, as described herein for use in the treatment of frailty or one or more frailty conditions. Embodiments can include any one of the features delineated herein.

In another aspect, there is provided a compound, or a pharmaceutically acceptable salt or tautomer thereof, as described herein for use in the treatment of mitochondrial dysfunction.

In some embodiments, the disease or symptom caused by mitochondrial dysfunction is selected from the group consisting of a mitochondrial disease, e.g., Leigh syndrome, mitochondrial encephalopathy, lactic acidosis and stroke-like episodes (MELAS), sepsis, or Leber's disease; diabetes and cancer.

In another aspect, there is provided a compound, or a pharmaceutically acceptable salt or tautomer thereof, as described herein for use in the treatment of muscle loss (e.g., loss of muscle mass).

In another aspect, there is provided a compound, or a pharmaceutically acceptable salt or tautomer thereof, as described herein for use in the treatment of a metabolic disorder (e.g., Type-2 diabetes, increased hemoglobin A1c, PKU, metabolic syndrome, or increased serum glucose).

In another aspect, there is provided a compound, or a pharmaceutically acceptable salt or tautomer thereof, as described herein for use in the treatment of obesity.

In another aspect, there is provided a compound, or a pharmaceutically acceptable salt or tautomer thereof, as described herein for use in the treatment of insulin resistance or insulin sensitivity.

In another aspect, there is provided the use of a compound, or a pharmaceutically acceptable salt or tautomer thereof, as described herein in the manufacture of a medicament for the treatment of a condition, disease or disorder associated with increased (e.g., excessive) CNDP2 expression.

In another aspect, there is provided the use of a compound, or a pharmaceutically acceptable salt or tautomer thereof, as described herein in the manufacture of a medicament for the treatment of cancer.

In another aspect, there is provided the use of a compound, or a pharmaceutically acceptable salt or tautomer thereof, as described herein in the manufacture of a medicament for the treatment of cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, glioblastoma multiforme, endometrial cancers, or hepatocellular carcinoma.

In another aspect, there is provided the use of a compound, or a pharmaceutically acceptable salt or tautomer thereof, as described herein in the manufacture of a medicament for the treatment of frailty or one or more frailty conditions. Embodiments can include any one of the features delineated herein.

In another aspect, there is provided the use of a compound, or a pharmaceutically acceptable salt or tautomer thereof, as described herein in the manufacture of a medicament for the treatment of mitochondrial dysfunction.

In another aspect, there is provided the use of a compound, or a pharmaceutically acceptable salt or tautomer thereof, as described herein in the manufacture of a medicament for the treatment of mitochondrial disease, e.g., Leigh syndrome, mitochondrial encephalopathy, lactic acidosis and stroke-like episodes (MELAS), sepsis, or Leber's disease; diabetes and cancer.

In another aspect, there is provided the use of a compound, or a pharmaceutically acceptable salt or tautomer thereof, as described herein in the manufacture of a medicament for the treatment of muscle loss (e.g., loss of muscle mass).

In another aspect, there is provided the use of a compound, or a pharmaceutically acceptable salt or tautomer thereof, as described herein in the manufacture of a medicament for the treatment of a metabolic disorder (e.g., Type-2 diabetes, increased hemoglobin A1c, PKU, metabolic syndrome, or increased serum glucose).

In another aspect, there is provided the use of a compound, or a pharmaceutically acceptable salt or tautomer thereof, as described herein in the manufacture of a medicament for the treatment of obesity.

In another aspect, there is provided the use of a compound, or a pharmaceutically acceptable salt or tautomer thereof, as described herein in the manufacture of a medicament for the treatment of insulin resistance or insulin sensitivity.

In another aspect, this disclosure features methods for modulating CNDP2 in a mammalian cell, the methods include contacting the mammalian cell with an effective amount of a compound of claim 1, or a pharmaceutically acceptable salt thereof.

In some embodiments, the contacting occurs in vivo.

In some embodiments, the contacting occurs in vitro.

In some embodiments, the cell includes altered (e.g., elevated) levels of lactate.

In some of the foregoing embodiments, the mammalian cell is a mammalian cancer cell.

In some embodiments, the subject is a subject identified or diagnosed as having one or more cancer cells, which have altered (e.g., elevated) levels of lactate.

In some embodiments, the subject is a subject identified or diagnosed as having one or more cancer cells, which have altered (e.g., excessive) levels of CNDP2 expression or activity.

In some embodiments, the subject is a subject identified or diagnosed as having one or more cancer cells, which have altered (e.g., elevated) levels of lactate and altered (e.g., excessive) levels of CNDP2 expression or activity.

In certain embodiments, the altered (e.g., elevated) levels of lactate are 5, 10, 20, 30, 40, 50, 60, 70, 80, 90, 100, 250, 500, or 1000 time greater than normal or baseline levels of lactate.

In certain embodiments, the altered (e.g., excessive) levels of CNDP2 expression or activity are 5, 10, 20, 30, 40, 50, 60, 70, 80, 90, 100, 250, 500, or 1000 time greater than normal or baseline levels of CNDP2 expression or activity.

The chemical entity can be administered in combination with one or more additional therapeutic agents and/or regimens. For examples, methods can further include administering one or more (e.g., two, three, four, five, six, or more) additional agents.

The chemical entity can be administered in combination with one or more additional therapeutic agents and/or regimens that are useful for treating other CNDP2-associated conditions, e.g., cancer.

The subject can have cancer; e.g., the subject has undergone and/or is undergoing and/or will undergo one or more cancer therapies.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “CNDP2” is meant to include, without limitation, nucleic acids, polynucleotides, oligonucleotides, sense and antisense polynucleotide strands, complementary sequences, peptides, polypeptides, proteins, antibodies, homologous and/or orthologous CNDP2 molecules, isoforms, precursors, mutants, variants, derivatives, splice variants, alleles, different species, and active fragments thereof.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates the administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “halo” refers to fluoro (F), chloro (Cl), bromo (Br), or iodo (I).

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “alkoxy” refers to an —O-alkyl radical (e.g., —OCH₃).

The term “alkylene” refers to a divalent alkyl (e.g., —CH₂—).

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, dihydro-1H-indenyl and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butanyl, bicyclo[2.1.0]pentanyl, bicyclo[1.1.1]pentanyl, bicyclo[3.1.0]hexanyl, bicyclo[2.1.1]hexanyl, bicyclo[3.2.0]heptanyl, bicyclo[4.1.0]heptanyl, bicyclo[2.2.1]heptanyl, bicyclo[3.1.1]heptanyl, bicyclo[4.2.0]octanyl, bicyclo[3.2.1]octanyl, bicyclo[2.2.2]octanyl, and the like. Cycloalkyl also includes spirocyclic rings (e.g., spirocyclic bicycle wherein two rings are connected through just one atom). Non-limiting examples of spirocyclic cycloalkyls include spiro[2.2]pentanyl, spiro[2.5]octanyl, spiro[3.5]nonanyl, spiro[3.5]nonanyl, spiro[3.5]nonanyl, spiro[4.4]nonanyl, spiro[2.6]nonanyl, spiro[4.5]decanyl, spiro[3.6]decanyl, spiro[5.5]undecanyl, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic, and at least one ring in the system contains one or more heteroatoms independently selected from the group consisting of N, O, and S (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butanyl, 2-azabicyclo[2.1.0]pentanyl, 2-azabicyclo[1.1.1]pentanyl, 3-azabicyclo[3.1.0]hexanyl, 5-azabicyclo[2.1.1]hexanyl, 3-azabicyclo[3.2.0]heptanyl, octahydrocyclopenta[c]pyrrolyl, 3-azabicyclo[4.1.0]heptanyl, 7-azabicyclo[2.2.1]heptanyl, 6-azabicyclo[3.1.1]heptanyl, 7-azabicyclo[4.2.0]octanyl, 2-azabicyclo[2.2.2]octanyl, 3-azabicyclo[3.2.1]octanyl, 2-oxabicyclo[1.1.0]butanyl, 2-oxabicyclo[2.1.0]pentanyl, 2-oxabicyclo[1.1.1]pentanyl, 3-oxabicyclo[3.1.0]hexanyl, 5-oxabicyclo[2.1.1]hexanyl, 3-oxabicyclo[3.2.0]heptanyl, 3-oxabicyclo[4.1.0]heptanyl, 7-oxabicyclo[2.2.1]heptanyl, 6-oxabicyclo[3.1.1]heptanyl, 7-oxabicyclo[4.2.0]octanyl, 2-oxabicyclo[2.2.2]octanyl, 3-oxabicyclo[3.2.1]octanyl, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentanyl, 4-azaspiro[2.5]octanyl, 1-azaspiro[3.5]nonanyl, 2-azaspiro[3.5]nonanyl, 7-azaspiro[3.5]nonanyl, 2-azaspiro[4.4]nonanyl, 6-azaspiro[2.6]nonanyl, 1,7-diazaspiro[4.5]decanyl, 7-azaspiro[4.5]decanyl 2,5-diazaspiro[3.6]decanyl, 3-azaspiro[5.5]undecanyl, 2-oxaspiro[2.2]pentanyl, 4-oxaspiro[2.5]octanyl, 1-oxaspiro[3.5]nonanyl, 2-oxaspiro[3.5]nonanyl, 7-oxaspiro[3.5]nonanyl, 2-oxaspiro[4.4]nonanyl, 6-oxaspiro[2.6]nonane, 1,7-dioxaspiro[4.5]decanyl, 2,5-dioxaspiro[3.6]decanyl, 1-oxaspiro[5.5]undecanyl, 3-oxaspiro[5.5]undecanyl, 3-oxa-9-azaspiro[5.5]undecanyl and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, when a ring is described as being “aromatic”, it means said ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

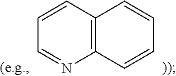

(ii) a single ring atom (spiro-fused ring systems)

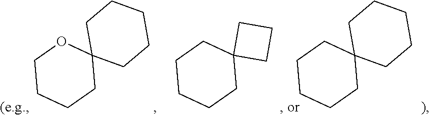

or (iii) a contiguous array of ring atoms (bridged ring systems having all bridge lengths >0)

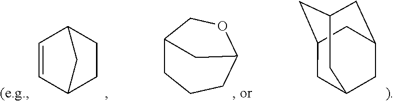 [SMILES: C1=CC2CCC1C2 ; C1CC2COC(C1)C2 ; C1C2CC3CC1CC(C2)C3]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. As used herein, the term “tautomer” is used to designate 2 molecules with the same molecular formula but different connectivity, which can interconvert in a rapid equilibrium. Thus, by way of example, a compound containing the moiety:

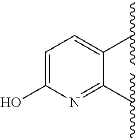 [SMILES: *c1ccc(O)nc1*]

encompasses the tautomeric form containing the moiety:

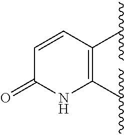 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms. Other heteroaryl group exists in various tautomeric forms. Another representative example is a pyrazolyl group:

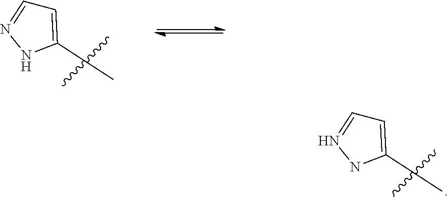 [SMILES: *c1ccn[nH]1 ; *c1ccn[nH]1]

In addition, any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

More particularly, the compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound. Further, unless otherwise indicated, when structures are presented having the solid and dashed wedges, the structure is intended to depict absolute chemistry. For example, a structure drawn with a center with high to low priority substituents (as determined by Cahn-Ingold-Prelog rules) arranged in clockwise fashion, means that the center has the “R” configuration. Likewise, a structure drawn with a center with high to low priority substituents (as determined by Cahn-Ingold-Prelog rules) arranged in counter-clockwise fashion, means that the center has the “S” configuration

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the phrase “optionally substituted” when used in conjunction with a structural moiety (e.g., alkyl) is intended to encompass both the unsubstituted structural moiety (i.e., none of the substitutable hydrogen atoms are replaced with one or more non-hydrogen substituents) and substituted structural moieties substituted with the indicated range of non-hydrogen substituents. For example, “C₁-C₄ alkyl optionally substituted with 1-4 R^{a}” is intended to encompass both unsubstituted C₁-C₄ alkyl and C₁-C₄ alkyl substituted with 1-4 R^{a}.

As used herein, the term “carboxylic acid isostere” refers to a a moiety having a similar size, same number of atoms, and same number of valence electrons as the carboxylic acid group (—COOH). The term “carboxylic acid bioisostere” refers to a moiety having similar physical and/or chemical properties as a carboxylic acid, and when incorporated into a chemical compound, said chemical compound exhibits similar properties to the corresponding carboxylic acid-containing chemical compound. Additional non-limiting examples of carboxylic acid isosteres or bioisosteres can be selected from those delineated in J. Med. Chem. 2016, 59, 3183-3203, which is incorporated herein by reference in its entirety.

In certain embodiments, the carboxylic acid isostere or bioisostere is selected from the group consisting of tetrazole, oxazole, isoxazole, isothiazole, —SO₃H, —SO₂NHR, —PO₂(R)₂, —CN, —PO₃(R)₂, —OR, —SR, —N(R)₂, —NHC(O)R, —NN(R)₂, —C(O)N(R)₂, —RC(O)N(CN)H, —N(CN)C(O)(R), —C(O)NHOR, —C(O)NHNHSO₂R, —C(O)NHSO₂R, —C(O)ONRCN, boronic acid, benzoxaborole, acyl sulfonamide, cyclobutenedione, cyclopentenedione, wherein R is hydrogen or a carbon chain or ring or a carbon-linked group, such as an alkyl, alkenyl, alkynyl, cycloalkyl, or aryl group, or a heterocycloalkyl or heteroaryl group where the bond is to a carbon, any of which may be optionally substituted.

Non-limiting examples of the foregoing include —CH₂CO₂H; —CF₂CO₂H; —C(CH₃)₂CO₂H; —CO(NH)—OH; —CO(NCH₃)—OH; —CO(NH)—OCH₃; —CO(NCH₃)—OCH₃; —N(OH)(C(O)Me); —PO₃H (i.e., phosphonic acid, or salt thereof); —PO₂H (i.e., phosphinic acid, or salt thereof); —SO₃H (i.e., sulfonic acid, or salt thereof); —SO₂H (i.e., sulfinic acid, or salt thereof); —SO₂NH₂; —NH—SO₂CH₃; —C(O)NH—SO₂CH₃; —C(O)NH—SO₂NH₂; —C(O)NH—SO₂N(CH₃)₂; —NHC(O)NH—SO₂CH₃; —NHC(O)NH—C(O)CH₃; —CO—NH—CN; —CO—N(CH₃)—CN; —CO—NH—CH₂—CF₃; —CO—N(CH₃)—CH₂—CF₃; oxazolyl, isoxazolyl, isothiazolyl, tetrazolyl, thiazolidinediones, oxazolidinediones, oxadiazol-5(4H)-one, thiadiazol-5(4H)-one, oxathiadiazole-2-oxide, isoxazolyl, tetramic acid, cyclopentane-1,3-diones, boronic acid, benzoxaborole, cyclobutene-1,2-dione, and:

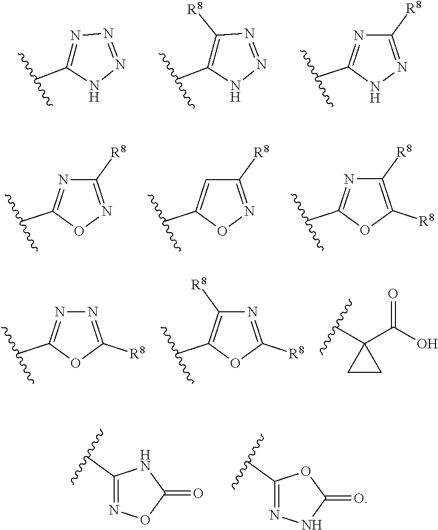

The details of one or more embodiments of the invention are set forth in the accompanying drawings and the description below. Other features and advantages of the invention will be apparent from the description and drawings, and from the claims.

### DETAILED DESCRIPTION

This disclosure features chemical entities (e.g., a compound or a pharmaceutically acceptable salt, and/or hydrate, and/or cocrystal, and/or drug combination of the compound) that inhibit (e.g., antagonize) Carnosine Dipeptidase 2 (CNDP2). Said chemical entities are useful, e.g., for treating a condition, disease or disorder in which increased (e.g., excessive) CNDP2 expression contributes to the pathology and/or symptoms and/or progression of the condition, disease or disorder (e.g., cancer) in a subject (e.g., a human). This disclosure also features compositions containing the same as well as methods of using and making the same.

### Formula I Compounds

In one aspect, the disclosure features compounds having Formula I:

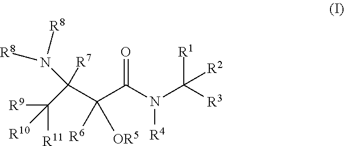

or a pharmaceutically acceptable thereof, wherein:
R¹, R², and R³ are defined according to (A) and (B) below:
   (A)
R¹ is selected from the group consisting of —CO₂H, —CO—NR¹²R¹³, and R^{1A};
wherein:
R^{1A} is a carboxylic acid isostere or bioisostere;
each occurrence of R¹² and R¹³ is independently selected from the group consisting of: H and C₁₋₃ alkyl;
R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(Rd), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
      (B)
R¹, R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
R⁴, R⁵, R⁶, R⁷, R⁸, R⁹, R¹⁰, and R¹¹ are defined according to (C) and D) below: each occurrence of R⁴, R⁵, and R⁸ is independently selected from the group consisting of: H and C₁₋₃ alkyl;
   (C)
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R^{g}) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R^{g} is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a}; and
each occurrence of R′ and R″ is independently selected from the group consisting of: H; —OH; and C₁₋₄ alkyl.
A) In some embodiments, it is provided when:
R¹ is CO₂H, CO₂Me, or CO₂Et,
each of R⁴, R⁵, R⁶, R⁷, R⁸, R⁹, and R¹⁰ is H,
R¹¹ is phenyl that is optionally substituted with one or more of NO₂, F, Cl, —OCH₃, —CH₃, phenyl, 2-benzothiazolyloxy, benzyloxy, iodo, —SCH₃, —SO₂CH₃, iso-propyl, iso-propoxy, iso-butyl, —OH, N₃, and NH₂; and
one of R² and R³ is H,
then the other of R² and R³ cannot be:
(i) the amino acid sidechain present in leucine, iso-leucine, threonine, tyrosine, O-benzyl-protected tyrosine, tryptophan, allothreonine, histidine, valine, alanine, arginine, glutamine, glutamic acid, aspartic acid, serine, lysine, N-benzyloxy-protected lysine, or methionine; or
(ii) n-hexyl, tert-butyl, n-butyl, or n-propyl;
B) In some embodiments, it is further provided when:
R¹ is CO₂H, CO₂Me, or CO₂Et,
each of R⁴, R⁵, R⁶, R⁷, R⁸, R⁹, and R¹⁰ is H,
R² and R³ together with the carbon atom to which each is attached form a 3, 4, 5, or 6 member ring; and
R¹ is phenyl
then the phenyl ring cannot be substituted at the para position with —SO₂CH₃; and
C) In some embodiments, it is further provided the compound is not

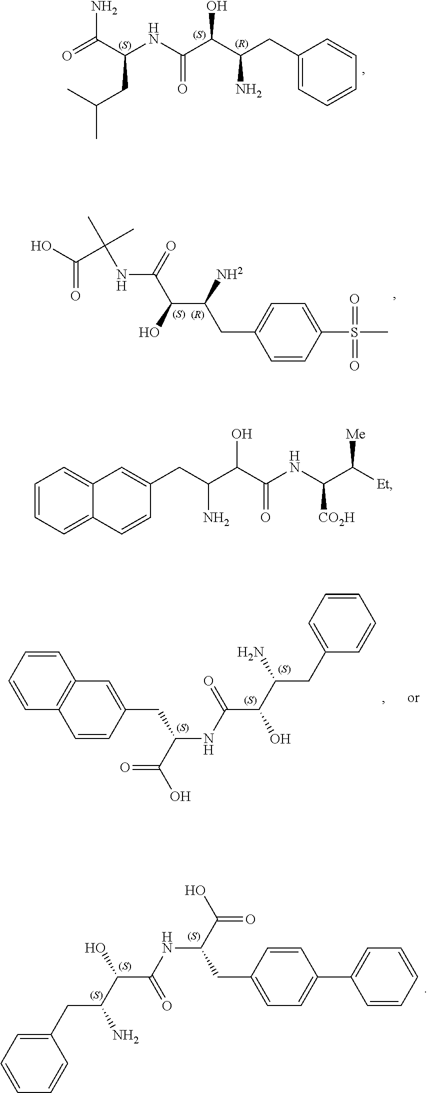

In one aspect, this disclosure features compounds having formula I:

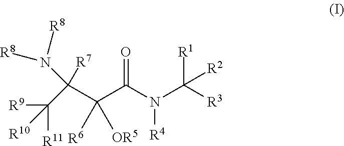

or a pharmaceutically acceptable thereof, wherein:
R¹, R², and R³ are defined according to (A) and (B) below:
R¹ is selected from the group consisting of —CO₂H, —CR^{12a}R^{13a}CO₂H—, —CO(NR¹²)—OH, —CO—NR¹²R¹³, —CO—NR¹²—SO₂R¹⁴, —CO—NR¹²—CN, —CO—NR¹²—CH₂—CF₃,

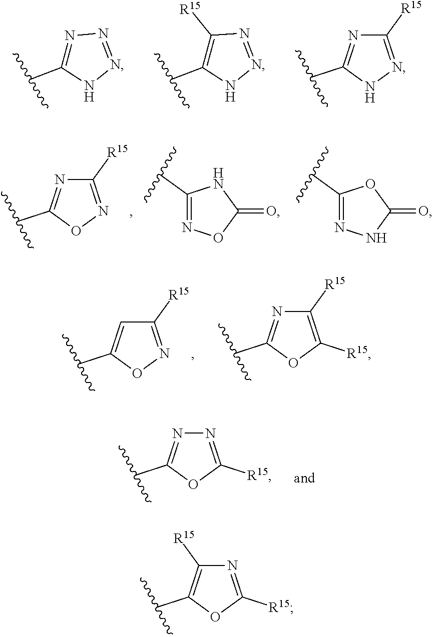

wherein:
each occurrence of R¹² and R¹³ is independently selected from the group consisting of: H and C₁₋₃ alkyl; R^{12a} and R^{13a} are each independently selected from the group consisting of H, halogen, and C₁₋₃ alkyl; or R¹² and R¹³, together with the carbon atom to which each is attached, form a C₃-C₆ cycloalkyl;
R⁴ is:
   H;
   C₁₋₆ alkyl optionally substituted with 1-3 independently selected R^{a}; or
   C₃₋₆ cycloalkyl which is optionally substituted with 1-6 independently selected R^{b};
each occurrence of R¹⁵ is independently selected from the group consisting of:
   H;
   halo;
   C₁₋₆ alkyl optionally substituted with 1-3 independently selected R^{a}; and
   C₃₋₆ cycloalkyl which is optionally substituted with 1-6 independently selected R^{b};
R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
      (B)
R¹, R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
R⁴, R⁵, R⁶, R⁷, R⁸, R⁹, R¹⁰, and R¹¹ are defined according to (C) and (D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
C₃₋₆ cycloalkyl or C₃₋₆₀ cycloalkenyl, each of which is optionally substituted with 1-6 substituents independently selected from the group consisting of oxo and R^{b}; and
heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R⁸ is selected from the group consisting of: H and C₁₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a}; and
each occurrence of R′ and R″ is independently selected from the group consisting of: H; —OH; and C₁₋₄ alkyl.
A) In some embodiments, it is provided when:
R¹ is CO₂H,
each of R⁴, R⁵, R⁶, R⁷, R⁸, R⁹, and R¹⁰ is H,
R¹¹ is phenyl that is optionally substituted with one or more of NO₂, F, Cl, —OCH₃, —CH₃, phenyl, 2-benzothiazolyloxy, benzyloxy, iodo, —SCH₃, —SO₂CH₃, iso-propyl, iso-propoxy, iso-butyl, —OH, N₃, and NH₂; and
one of R² and R³ is H,
then the other of R² and R³ cannot be:
(i) the amino acid sidechain present in leucine, iso-leucine, threonine, tyrosine, O-benzyl-protected tyrosine, tryptophan, allothreonine, histidine, valine, alanine, arginine, glutamine, glutamic acid, aspartic acid, serine, lysine, N-benzyloxy-protected lysine, or methionine; or
(ii) n-hexyl, tert-butyl, n-butyl, or n-propyl;
B) In some embodiments, it is further provided when:
R¹ is CO₂H,
each of R⁴, R⁵, R⁶, R⁷, R⁸, R⁹, and R¹⁰ is H,
R² and R³ together with the carbon atom to which each is attached form a 3, 4, 5, or 6 member ring; and
R¹ is phenyl
then the phenyl ring cannot be substituted at the para position with —SO₂CH₃; and
C) In some embodiments, it is further provided the compound is not

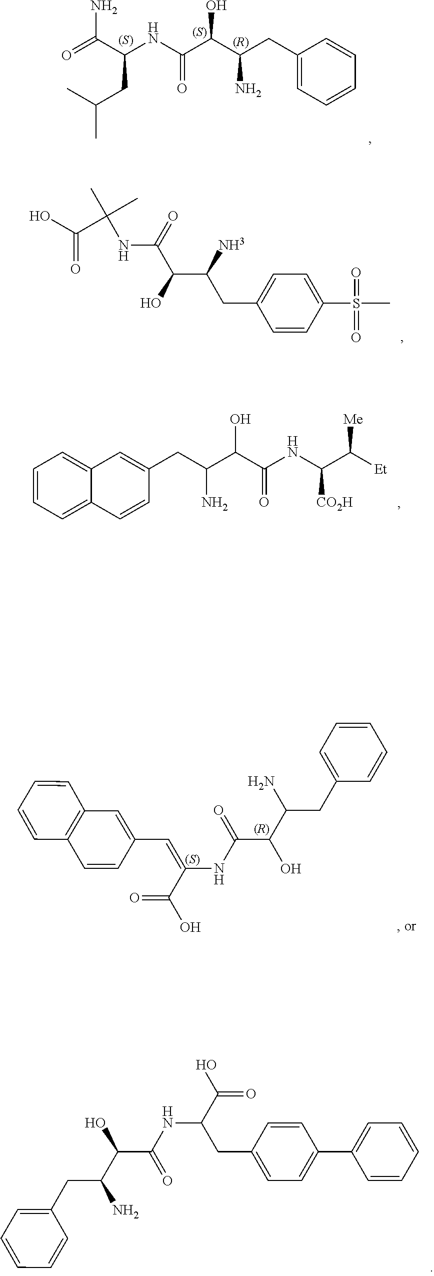

Variable R¹, R², and R³

In some embodiments, R¹, R², and R³ are defined according to (A).

In some embodiments, R¹ is —CO₂H.

In some embodiments, R¹ is —CO—NR¹²R¹³.

In some embodiments, R¹ is R^{1A}, wherein, R^{1A} is a carboxylic acid isostere or bioisostere.

In certain embodiments, the carboxylic acid isostere or bioisostere is selected from the group consisting of tetrazole, oxazole, isoxazole, isothiazole, —SO₃H, —SO₂NHR, —PO₂(R)₂, —CN, —PO₃(R)₂, —OR, —SR, —N(R)₂, —NHC(O)R, —NN(R)₂, —C(O)N(R)₂, —RC(O)N(CN)H, —N(CN)C(O)(R), —C(O)NHOR, —C(O)NHNHSO₂R, —C(O)NHSO₂R, —C(O)ONRCN, boronic acid, benzoxaborole, acyl sulfonamide, cyclobutenedione, cyclopentenedione, wherein R is hydrogen or a carbon chain or ring or a carbon-linked group, such as an alkyl, alkenyl, alkynyl, cycloalkyl, or aryl group, or a heterocycloalkyl or heteroaryl group where the bond is to a carbon, any of which may be optionally substituted as described anywhere herein.

Non-limiting examples of the foregoing include —CH₂CO₂H; —CF₂CO₂H; —C(CH₃)₂CO₂H; —CO(NH)—OH; —CO(NCH₃)—OH; —CO(NH)—OCH₃; —CO(NCH₃)—OCH₃; —N(OH)(C(O)Me); —PO₃H (i.e., phosphonic acid, or salt thereof); —PO₂H (i.e., phosphinic acid, or salt thereof); —SO₃H (i.e., sulfonic acid, or salt thereof); —SO₂H (i.e., sulfinic acid, or salt thereof); —SO₂NH—₂; —NH—SO₂CH₃; —C(O)NH—SO₂CH₃; —C(O)NH—SO₂NH₂; —C(O)NH—SO₂N(CH₃)₂; —NHC(O)NH—SO₂CH₃; —NHC(O)NH—C(O)CH₃; —CO—NH—CN; —CO—N(CH₃)—CN; —CO—NH—CH₂—CF₃; —CO—N(CH₃)—CH₂—CF₃; oxazolyl, isoxazolyl, isothiazolyl, tetrazolyl, thiazolidinediones, oxazolidinediones, oxadiazol-5(4H)-one, thiadiazol-5(4H)-one, oxathiadiazole-2-oxide, isoxazolyl, tetramic acid, cyclopentane-1,3-diones, boronic acid, benzoxaborole, cyclobutene-1,2-dione, and:

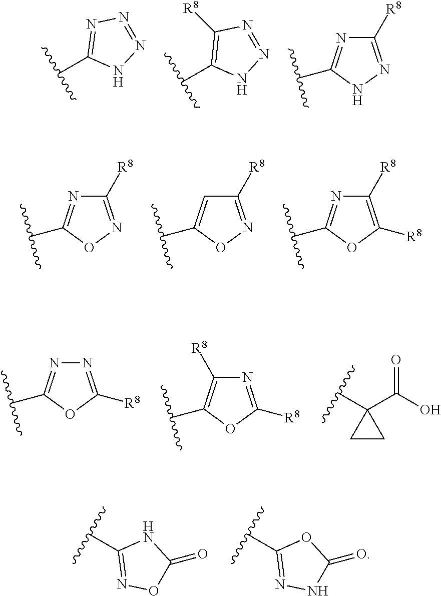

In some embodiments, R¹ is selected from the group consisting of —CO₂H, —CR¹²R¹³CO₂H, —CO(NR¹²)—OH, —CO—NR¹²R¹³, —CO—NR¹²—SO₂R′, —CO—NR¹²—CN, and —CO—NR¹²—CH₂—CF₃.

In some embodiments, R^{1A} is selected from the group consisting of —CR^{12a}R^{13a}CO₂H, —CO(NR¹²)—OH, —CO—NR¹²—SO₂R⁴, —CO—NR¹²—CN, and —CO—NR¹²—CH₂—CF₃.

In some embodiments, R^{1A} is selected from the group consisting of:

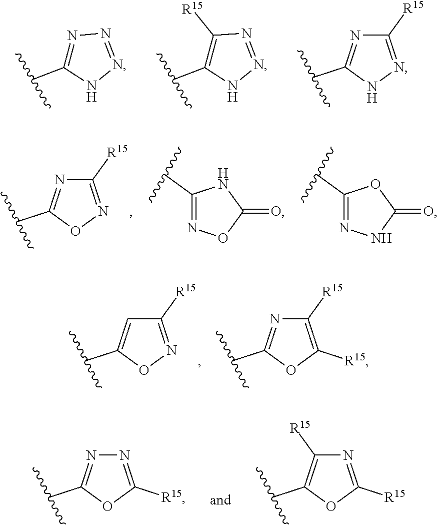

In some embodiments, R^{1A} is

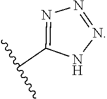 [SMILES: *c1nnn[nH]1]

In some embodiments, R¹ is selected from the group consisting of —CO(NR¹²)—OH and —CO—NR¹²R¹³. In certain embodiments, R¹ is —CO(NR¹²)—OH.

In some embodiments, R¹ is —CO—NR¹²—CN.

In some embodiments, R¹ is —CO—NR¹²—SO₂R¹⁴.

In some embodiments, R¹ is —CO—NR¹²—CH₂—CF₃.

In some embodiments, R¹ is selected from the group consisting of:

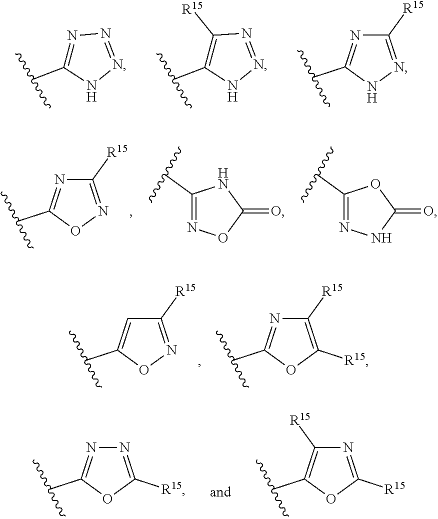

In some embodiments, R¹ is

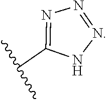 [SMILES: *c1nnn[nH]1]

In some embodiments, R¹ is selected from the group consisting of —CO₂H, —CR¹²R¹³CO₂H, —CO(NR¹²)—OH, —CO—NR¹²R¹³, —CO—NR¹²—SO₂R¹⁴, —CO—NR¹²—CN, and —CO—NR¹²—CH₂—CF₃.

In some embodiments, R¹ is CR^{12a}R^{13a}CO₂H.

In some embodiments, R^{12a} is a halogen, e.g., R^{12a} is F, Cl, or Br. In certain embodiments, R^{12a} is F.

In some embodiments, R^{13a} is a halogen, e.g., R^{13a} is F, Cl, or Br. In certain embodiments, R^{13a} is F.

In some embodiments, R^{12a} and R^{13a}, together with the carbon atom to which each is attached, form a C₃-C₆ cycloalkyl. In some embodiments, R^{12a} and R^{13a}, together with the carbon atom to which each is attached, form cyclopropyl. In some embodiments, R^{12a} and R^{13a}, together with the carbon atom to which each is attached, form cyclobutyl. In some embodiments, R^{12a} and R^{13a}, together with the carbon atom to which each is attached, form cyclopentyl. In some embodiments, R^{12a} and R^{13a}, together with the carbon atom to which each is attached, form cyclohexyl.

In certain embodiments, R¹ is selected from the group consisting of

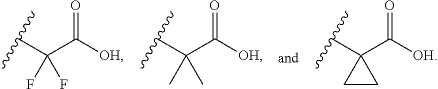

In some embodiments, R¹ is

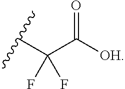 [SMILES: *C(F)(F)C(=O)O]

In some embodiments, R¹ is

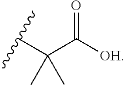 [SMILES: *C(C)(C)C(=O)O]

In some embodiments, R¹ is

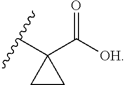 [SMILES: *C1(C(=O)O)CC1]

In some embodiments, R¹² is H. In some embodiments, R¹² is C₁₋₃ alkyl. In some embodiments, R¹² is CH₃. In some embodiments, R¹² is —CH₂CH₃. In some embodiments, R¹² is —CH₂CH₂CH₃. In some embodiments, R¹² is —CH(CH₃)₂.

In some embodiments, R¹³ is H. In some embodiments, R¹³ is C₁₋₃ alkyl. In some embodiments, R¹³ is CH₃. In some embodiments, R¹³ is —CH₂CH₃. In some embodiments, R¹³ is —CH₂CH₂CH₃. In some embodiments, R¹³ is —CH(CH₃)₂.

In some embodiments, R¹ is H. In some embodiments, R¹⁴ is C₃₋₆ cycloalkyl which is optionally substituted with 1-6 independently selected R^{b}. In some embodiments, R⁴ is cyclobutyl optionally substituted with 1-6 independently selected R^{b}. In some embodiments, R¹⁴ is cyclopentyl optionally substituted with 1-6 independently selected R^{b}. In some embodiments, R¹⁴ is cyclohexyl optionally substituted with 1-6 independently selected R^{b}. In some embodiments, R¹⁴ is C₁₋₆ alkyl optionally substituted with 1-3 independently selected R^{a}. In some embodiments, R¹⁴ is C₁₋₃ alkyl optionally substituted with 1-3 independently selected R^{a}. In some embodiments, R⁴ is CH₃. In some embodiments, R⁴ is —CH₂CH₃. In some embodiments, R⁴ is —CH₂CH₂CH₃. In some embodiments, R⁴ is —CH(CH₃)₂.

In some embodiments, R⁵ is H. In some embodiments, R⁵ is halo. In some embodiments, R¹⁵ is F, Cl, or Br. In some embodiments, R¹⁵ is F. In some embodiments, R¹⁵ is C₃₋₆ cycloalkyl which is optionally substituted with 1-6 independently selected R^{b}. In some embodiments, R¹⁵ is cyclobutyl optionally substituted with 1-6 independently selected R^{b}. In some embodiments, R¹⁵ is cyclopentyl optionally substituted with 1-6 independently selected R^{b}. In some embodiments, R⁵ is cyclohexyl optionally substituted with 1-6 independently selected R^{b}. In some embodiments, R⁵ is C₁₋₆ alkyl optionally substituted with 1-3 independently selected R^{a}. In some embodiments, R⁵ is C₁₋₃ alkyl optionally substituted with 1-3 independently selected R^{a}. In some embodiments, R¹⁵ is CH₃. In some embodiments, R¹⁵ is CH₂CH₃. In some embodiments, R¹⁵ is —CH₂CH₂CH₃. In some embodiments, R¹⁵ is CH(CH₃)₂.

In some embodiments, R² and R³ are each independently selected from the group consisting of:
H;
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H.

In some embodiments, one of R² and R³ is selected from the group consisting of:
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; and
the other of R² and R³ is H.

In some embodiments, one of R² and R³ is selected from the group consisting of:
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; and
the other of R² and R³ is H.

In some embodiments, one of R² and R³ is selected from the group consisting of:
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene; and
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene; and
the other of R² and R³ is H.

In some embodiments, one of R² and R³ is selected from the group consisting of:
L¹-C₃₋₁₀ cycloalkyl optionally substituted with 1-6 substituents independently selected from the group consisting of oxo and R^{b}, wherein L¹ is a bond or unsubstituted C₁₋₆ alkylene; and
L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and
the other of R² and R³ is H.

In some embodiments, one of R² and R³ is selected from the group consisting of:
L¹-C₃₋₇ cycloalkyl optionally substituted with 1-6 substituents independently selected from the group consisting of oxo and R^{b}, wherein L¹ is a bond or unsubstituted C₁₋₆ alkylene; and
L²-heterocyclyl of 3-7 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and
the other of R² and R³ is H.

In some embodiments, one of R² and R³ is L-C₃₋₁₀ cycloalkyl or L-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene; and the other of R² and R³ is H.

In some embodiments, one of R² and R³ is L¹-C₃₁₀ cycloalkyl, which is optionally substituted with 1-6 substituents independently selected from the group consisting of oxo and R^{b}, wherein L¹ is a bond or unsubstituted C₁₋₆ alkylene; and the other of R² and R³ is H.

In some embodiments, one of R² and R³ is L¹-C₃₋₇ cycloalkyl, which is optionally substituted with 1-6 substituents independently selected from the group consisting of oxo and R^{b}, wherein L¹ is a bond or unsubstituted C₁₋₆ alkylene; and the other of R² and R³ is H.

In some embodiments, one of R² and R³ is L¹-cyclopropyl and the other of R² and R³ is H. In some embodiments, one of R² and R³ is L-cyclobutyl and the other of R² and R³ is H. In some embodiments, one of R² and R³ is L¹-cyclopentyl and the other of R² and R³ is H. In some embodiments, one of R² and R³ is L-cyclohexyl and the other of R² and R³ is H. In some embodiments, one of R² and R³ is L-cycloheptyl and the other of R² and R³ is H.

In some of the foregoing embodiments, L¹ is a bond. In some embodiments, L¹ is C₁₋₆ alkylene. In some embodiments, L¹ is C₁₋₃ alkylene. In some embodiments, L¹ is CH₂.

In certain embodiments, one of R² and R³ is L¹-cyclopentyl and the other of R² and R³ is H, and L¹ is a bond.

In certain embodiments, one of R² and R³ is L¹-cyclohexyl and the other of R² and R³ is H, and L¹ is a bond.

In certain embodiments, one of R² and R³ is L¹-cyclohexyl optionally substituted with 1-6 (e.g., 2) substituents independently selected from the group consisting of oxo and R^{b} (e.g., F). and the other of R² and R³ is H, and L¹ is a bond. For example one of R² and R³ is 4,4-difluorocyclohexyl.

In certain embodiments, one of R² and R³ is L¹-cycloheptyl and the other of R² and R³ is H, and L¹ is a bond.

In certain embodiments, one of R² and R³ is L¹-cyclopentyl and the other of R² and R³ is H, and L¹ is a CH₂.

In some embodiments, one of R² and R³ is L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene; and the other of R² and R³ is H.

In some embodiments, one of R² and R³ is L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

In some embodiments, one of R² and R³ is L²-heterocyclyl of 3-7 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

In some embodiments, one of R² and R³ is L²-heterocyclyl of 4-6 ring atoms, wherein 1-3 ring atoms are heteroatoms, each independently selected from the group consisting of N, N(H), and N(R^{d}), and wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

In certain embodiments, one of R² and R³ is L²-heterocyclyl of 4-6 ring atoms, wherein 1-3 ring atoms are heteroatoms, each independently selected from the group consisting of O and S(O)₀₋₂ wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

In certain embodiments, one of R² and R³ is L²-heterocyclyl of 4-6 ring atoms, wherein 1-2 (e.g., 1) ring atoms is/are O, wherein the heterocyclyl is optionally substituted with 1-4 substituents independently selected from the group consisting of oxo and R^{b}, wherein L² is a bond or unsubstituted C₁₋₆ alkylene; and the other of R² and R³ is H. For example, the heterocyclyl can be

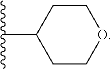 [SMILES: *C1CCOCC1]

For example, one of R² and R³ is

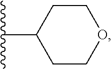 [SMILES: *C1CCOCC1]

the other of R² and R³ is H, and L² is CH₂.

In some of the foregoing embodiments, L² is a bond. In some embodiments, L² is C₁₋₆ alkylene. In some embodiments, L² is C₁₋₃ alkylene. In some embodiments, L² is CH₂.

In some embodiments, one of R² and R³ is selected from the group consisting of:
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently selected from the group consisting of oxo and R^{c}, wherein L⁴ is a bond or unsubstituted C₁₋₆ alkylene; and
the other of R² and R³ is H.

In some embodiments, one of R² and R³ is L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and the other of R² and R³ is H.

In some embodiments, one of R² and R³ is L³-heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and the other of R² and R³ is H.

In some embodiments, one of R² and R³ is L³-heteroaryl of 6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and the other of R² and R³ is H.

In certain embodiments, one of R² and R³ is L³-heteroaryl of 6 ring atoms, wherein 1-2 ring atoms are ring nitrogen atoms, and wherein the heteroaryl is optionally substituted with 1-3 R^{c}; and the other of R² and R³ is H. In certain of these embodiments, one of R² and R³ is L³-pyridyl, which is optionally substituted with 1-3 R^{c}; and the other of R² and R³ is H. In certain of the foregoing embodiments, one of R² and R³ is L³-pyridyl, wherein the pyridyl is selected from the group consisting of:

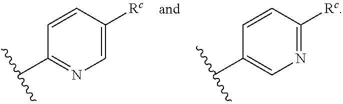

In some embodiments, L³ is a bond. In some embodiments, L³ is C₁₋₆ alkylene. In some embodiments, L³ is C₁₋₃ alkylene. In some embodiments, L³ is CH₂.

In some embodiments, one of R² and R³ is L⁴-C₆₋₁₀ aryl optionally substituted with 1-4 substituents independently selected from the group consisting of oxo and R^{c}, wherein L⁴ is a bond or unsubstituted C₁₋₆ alkylene; and the other of R² and R³ is H.

In some embodiments, one of R² and R³ is L⁴-phenyl optionally substituted with 1-4 substituents independently selected from the group consisting of oxo and R^{c}, wherein L⁴ is a bond or unsubstituted C₁₋₆ alkylene; and the other of R² and R³ is H.

In some embodiments, one of R² and R³ is L⁴-phenyl optionally substituted with 1-3 substituents R^{c}, wherein L⁴ is a bond or unsubstituted C₁₋₆ alkylene; and the other of R² and R³ is H. In some embodiments, one of R² and R³ is L⁴-phenyl substituted with 1-2 substituents R^{c}, wherein the phenyl is selected from the group consisting of:

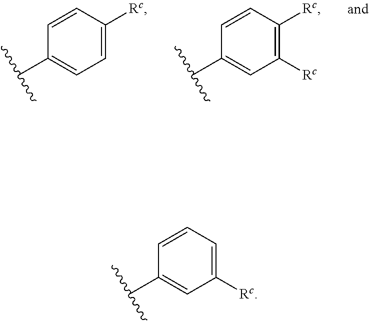

In some of the foregoing embodiments, L⁴ is a bond. In some embodiments, L⁴ is C₁₋₆ alkylene. In some embodiments, L⁴ is C₁₋₃ alkylene. In some embodiments, L⁴ is CH₂.

In certain embodiments, one of R² and R³ is L⁴-phenyl, optionally substituted with 1-3 R^{c} (e.g., substituted with 1-2 R^{c} or 1 R^{c} (e.g., OCF₃ or CF₃)), and the other of R² and R³ is H, and L⁴ is a bond. For example, one of R² and R³ is meta-CF₃-phenyl, and L⁴ is a bond, and the other of R² and R³ is H. As another example, one of R² and R³ is meta-OCF₃-phenyl, and L⁴ is a bond, and the other of R² and R³ is H.

In certain embodiments, one of R² and R³ is L⁴-phenyl, optionally substituted with 1-3 R^{c} (e.g., substituted with 1-2 R^{c} or 1 R^{c} (e.g., Br or CF₃)), and the other of R² and R³ is H, and L⁴ is a CH₂. For example, one of R² and R³ is para-CF₃-phenyl, the other of R² and R³ is H, and L⁴ is a CH₂. As another example, one of R² and R³ is para-Br-phenyl, the other of R² and R³ is H, and L⁴ is a CH₂.

In some embodiments, R² and R³, together with the carbon atom to which each is attached, form a ring selected from the group consisting of:
C₃₋₁₀ cycloalkyl or C₃₋₁₀ cycloalkenyl, each of which is optionally substituted with 1-6 substituents independently selected from the group consisting of oxo and R^{b}; and
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}.

In some embodiments, R² and R³, together with the carbon atom to which each is attached, form a C₃₋₁₀ cycloalkyl optionally substituted with 1-6 substituents independently selected from the group consisting of oxo and R^{b}.

In some embodiments, R² and R³, together with the carbon atom to which each is attached, form a C₃₋₆ cycloalkyl optionally substituted with 1-4 substituents independently selected from the group consisting of oxo and R^{b}.

In some embodiments, R² and R³, together with the carbon atom to which each is attached, form a cyclopropyl optionally substituted with R^{b}. In some embodiments, R² and R³, together with the carbon atom to which each is attached, form a cyclobutyl optionally substituted with 1-2 R^{b}. In some embodiments, R² and R³, together with the carbon atom to which each is attached, form a cyclopentyl optionally substituted with 1-3 R^{b}. In some embodiments, R² and R³, together with the carbon atom to which each is attached, form a cyclohexyl optionally substituted with 1-3 R^{b}.

In some embodiments, R² and R³, together with the carbon atom to which each is attached, form a heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R.

In some embodiments, R² and R³, together with the carbon atom to which each is attached, form a heterocyclyl of 4-7 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R.

In some embodiments, R¹, R², and R³ are defined according to B).

In some embodiments, R¹, R² and R³, together with the carbon atom to which each is attached, form C₆₋₁₀ aryl optionally substituted with 1-4 substituents independently selected from the group consisting of oxo and R^{c}.

In some embodiments, R¹, R² and R³, together with the carbon atom to which each is attached, form phenyl optionally substituted with 1-4 R^{c}.

In some embodiments, R¹, R² and R³, together with the carbon atom to which each is attached, form phenyl optionally substituted with 1-3 substituents R^{c}. In some embodiments, R¹, R² and R³, together with the carbon atom to which each is attached, form phenyl substituted with 1-2 substituents R^{c}, wherein the phenyl is selected from the group consisting of:

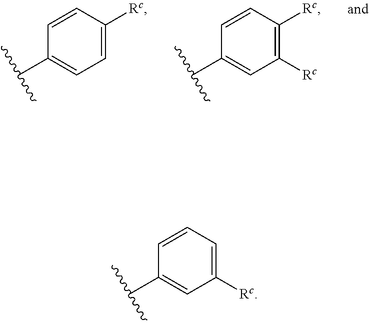

Variables R⁴, R⁵, R⁶, R⁷, R⁸, R⁹, R¹⁰, and R¹¹

In some embodiments, R⁴, R⁵, R⁶, R⁷, R⁸, R⁹, R¹⁰, and R¹¹ are defined according to (C).

In some embodiments, each occurrence of R⁴, R⁵, and R⁸ is independently selected from the group consisting of: H and C₁₋₃ alkyl. In some embodiments, at least one of R⁴, R⁵, and R⁸ is H. In some embodiments, each occurrence of R⁴, R⁵, and R⁸ is H.

In some embodiments, at least one of R⁴, R⁵, and R⁸ is C₁₋₃ alkyl. In some embodiments, each occurrence of R⁴, R⁵, and R⁸ is C₁₋₃ alkyl. In some embodiments, at least one of R⁴, R⁵, and R⁸ is CH₃. In some embodiments, each occurrence of R⁴, R⁵, and R⁸ is CH₃.

In some embodiments, each occurrence of R⁶ and R⁷ is independently selected from the group consisting of: H and C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}.

In some embodiments, each occurrence of R⁶ and R⁷ is independently selected from the group consisting of: H; C₁₋₆ alkyl optionally substituted with 1-4 independently selected R^{a}; and C₃₋₆ cycloalkyl optionally substituted with 1-6 substituents independently selected from the group consisting of oxo and R^{b}.

In some embodiments, one of R⁶ and R⁷ is H. In some embodiments, R⁶ and R⁷ are H.

In some embodiments, one of R⁶ and R⁷ is C₁₋₆ alkyl optionally substituted with 1-4 independently selected R^{a}. In some embodiments, each occurrence of R⁶ and R⁷ is C₁₋₆ alkyl optionally substituted with 1-4 independently selected R^{a}. In some embodiments, at least one of R⁶ and R⁷ is CH₃. In some embodiments, each occurrence of R⁶ and R⁷ is CH₃.

In some embodiments, one of R⁶ and R⁷ is C₃₋₆ cycloalkyl optionally substituted with 1-6 substituents independently selected from the group consisting of oxo and R^{b}. In some embodiments, one of R⁶ and R⁷ is cyclopropyl optionally substituted with 1-6 substituents independently selected from the group consisting of oxo and R^{b}. In some embodiments, one of R⁶ and R⁷ is cyclobutyl optionally substituted with 1-6 substituents independently selected from the group consisting of oxo and R^{b}. In some embodiments, one of R⁶ and R⁷ is cyclopentyl optionally substituted with 1-6 substituents independently selected from the group consisting of oxo and R^{b}. In some embodiments, one of R⁶ and R⁷ is cyclohexyl optionally substituted with 1-6 substituents independently selected from the group consisting of oxo and R^{b}.

In some embodiments, one of R⁶ and R⁷ is C₃₋₆ cycloalkyl (e.g., cyclopropyl) optionally substituted with 1-6 substituents independently selected from the group consisting of oxo and R^{b} or C₁₋₆ alkyl (e.g., CH₃) optionally substituted with 1-4 independently selected R^{a}; and the other of R⁶ and R⁷ is H.

In some embodiments, each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}.

In some embodiments, one of R⁹ and R¹⁰ is H. In some embodiments, R⁹ and R¹⁰ are H.

In some embodiments, one of R⁹ and R¹⁰ is F. In some embodiments, R⁹ and R¹⁰ are F.

In some embodiments, one of R⁹ and R¹⁰ is C₁₋₆ alkyl optionally substituted with 1-4 independently selected R^{a}. In some embodiments, each occurrence of R⁹ and R¹⁰ is C₁₋₆ alkyl optionally substituted with 1-4 independently selected R^{a}. In some embodiments, one of R⁹ and R¹⁰ is CH₃. In some embodiments, each occurrence of R⁹ and R¹⁰ is CH₃.

In some embodiments, one of R⁹ and R¹⁰ is C₃₋₆ cycloalkyl optionally substituted with 1-6 substituents independently selected from the group consisting of oxo and R^{b}. In some embodiments, each occurrence of R⁹ and R¹⁰ is C₃₋₆ cycloalkyl optionally substituted with 1-6 substituents independently selected from the group consisting of oxo and R^{b}. In some embodiments, one of R⁹ and R¹⁰ is cyclopropyl optionally substituted with 1-6 substituents independently selected from the group consisting of oxo and R^{b}. In some embodiments, one of R⁹ and R¹⁰ is cyclobutyl optionally substituted with 1-6 substituents independently selected from the group consisting of oxo and R^{b}. In some embodiments, one of R⁹ and R¹⁰ is cyclopentyl optionally substituted with 1-6 substituents independently selected from the group consisting of oxo and R^{b}. In some embodiments, one of R⁹ and R¹⁰ is cyclohexyl optionally substituted with 1-6 substituents independently selected from the group consisting of oxo and R^{b}.

In some embodiments, R⁹ and R¹⁰, together with the carbon atom to which each is attached, form a ring selected from the group consisting of:
C₃₋₆ cycloalkyl or C₃₋₆₀ cycloalkenyl, each of which is optionally substituted with 1-6 substituents independently selected from the group consisting of oxo and R^{b}; and
heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}.

In some embodiments, R⁹ and R¹⁰, together with the carbon atom to which each is attached, form C₃₋₆ cycloalkyl or C₃₋₆₀ cycloalkenyl, each of which is optionally substituted with 1-6 substituents independently selected from the group consisting of oxo and R^{b}.

In some embodiments, R⁹ and R¹⁰, together with the carbon atom to which each is attached, form C₃₋₆ cycloalkyl optionally substituted with 1-6 substituents independently selected from the group consisting of oxo and R^{b}. In some embodiments, R⁹ and R¹⁰, together with the carbon atom to which each is attached, form cyclopropyl optionally substituted with 1-6 substituents independently selected from the group consisting of oxo and R^{b}. In some embodiments, R⁹ and R¹⁰, together with the carbon atom to which each is attached, form cyclobutyl optionally substituted with 1-6 substituents independently selected from the group consisting of oxo and R^{b}. In some embodiments, R⁹ and R¹⁰, together with the carbon atom to which each is attached, form cyclopentyl optionally substituted with 1-6 substituents independently selected from the group consisting of oxo and R^{b}. In some embodiments, R⁹ and R¹⁰, together with the carbon atom to which each is attached, form cyclohexyl optionally substituted with 1-6 substituents independently selected from the group consisting of oxo and R^{b}.

In some embodiments, R⁹ and R¹⁰, together with the carbon atom to which each is attached, form heterocyclyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}.

In some embodiments, R⁹ and R¹⁰, together with the carbon atom to which each is attached, form heterocyclyl of 5-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R.

In some embodiments, R⁴, R⁵, R⁶, R⁷, R⁸, R⁹, and R¹⁰ are H.

In some embodiments, R¹¹ is selected from the group consisting of:
C₃₋₁₀ cycloalkyl or C₃₋₁₀ cycloalkenyl, each of which is optionally substituted with 1-6 substituents independently selected from the group consisting of oxo and R^{b};
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
C₆₋₁₀ aryl optionally substituted with 1-4 substituents independently independently selected from the group consisting of oxo and R^{c}.

In some embodiments, R¹¹ is selected from the group consisting of:
C₃₋₁₀ cycloalkyl or C₃₋₁₀ cycloalkenyl, each of which is optionally substituted with 1-6 substituents independently selected from the group consisting of oxo and R^{b}; and
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and

In some embodiments, R¹¹ is selected from the group consisting of:
C₃₋₁₀ cycloalkyl optionally substituted with 1-6 substituents independently selected from the group consisting of oxo and R^{b}; and
heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}.

In some embodiments, R¹¹ is selected from the group consisting of:
C₃₋₇ cycloalkyl optionally substituted with 1-6 substituents independently selected from the group consisting of oxo and R^{b}; and
heterocyclyl of 3-7 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}.

In some embodiments, R¹¹ is C₃₋₁₀ cycloalkyl or C₃₋₁₀ cycloalkenyl, each of which is optionally substituted with 1-6 substituents independently selected from the group consisting of oxo and R^{b}.

In some embodiments, R¹¹ is C₃₋₁₀ cycloalkyl, which is optionally substituted with 1-6 substituents independently selected from the group consisting of oxo and R^{b}.

In some embodiments, R¹¹ is C₃₋₇ cycloalkyl, which is optionally substituted with 1-6 substituents independently selected from the group consisting of oxo and R^{b}.

In some embodiments, R¹¹ is cyclopropyl. In some embodiments, R¹¹ is cyclobutyl. In some embodiments, R¹¹ is cyclopentyl. In some embodiments, R¹¹ is cyclohexyl, each of which is optionally substituted with 1-6 substituents independently selected from the group consisting of oxo and R^{b}.

In some embodiments, R¹¹ is heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}.

In some embodiments, R¹¹ is heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}.

In some embodiments, R¹¹ is heterocyclyl of 3-7 ring atoms, wherein 1-3 ring atoms are heteroatoms, each independently selected from the group consisting of N, N(H), N(R), O, and S(O)₀₋₂, and wherein the heterocyclyl is optionally substituted with 1-4 substituents independently selected from the group consisting of oxo and R^{b}.

In some embodiments, R¹¹ is heterocyclyl of 4-6 ring atoms, wherein 1-3 ring atoms are heteroatoms, each independently selected from the group consisting of N, N(H), and N(R^{d}), and wherein the heterocyclyl is optionally substituted with 1-4 substituents independently selected from the group consisting of oxo and R^{b}. In some embodiments, R¹¹ is heterocyclyl of 4-6 ring atoms, wherein 1-3 ring atoms are heteroatoms, each independently selected from the group consisting of O and S(O)₀₋₂ wherein the heterocyclyl is optionally substituted with 1-4 substituents independently selected from the group consisting of oxo and R^{b}.

In some embodiments, R¹¹ is selected from the group consisting of:
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
C₆₋₁₀ aryl optionally substituted with 1-4 substituents independently selected from the group consisting of oxo and R^{c}.

In some embodiments, R¹¹ is heteroaryl of 5-10 ring atoms, wherein 1-4 ring atoms are heteroatoms, each independently selected from the group consisting of N, N(H), N(R), O, and S(O)₀₋₂, and wherein the heteroaryl is optionally substituted with 1-4 substituents independently selected from the group consisting of oxo and R^{c}.

In some embodiments, R¹¹ is heteroaryl of 5-6 ring atoms, wherein 1-4 ring atoms are heteroatoms, each independently selected from the group consisting of N, N(H), N(R), O, and S(O)₀₋₂, and wherein the heteroaryl is optionally substituted with 1-4 substituents independently selected from the group consisting of oxo and R^{c}.

In some embodiments, R¹¹ is heteroaryl of 6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}.

In certain embodiments, R¹¹ is heteroaryl of 6 ring atoms, wherein 1-2 ring atoms are ring nitrogen atoms. In certain of these embodiments, R¹¹ is pyridyl, which is optionally substituted with 1-3 R^{c}. In certain of the foregoing embodiments, R¹¹ is pyridyl, wherein the pyridyl is selected from the group consisting of:

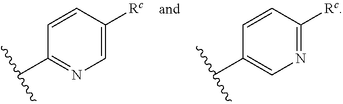

In some embodiments, R¹¹ is C₆₋₁₀ aryl optionally substituted with 1-4 substituents independently selected from the group consisting of oxo and R^{c}.

In some embodiments, R¹¹ is phenyl optionally substituted with 1-4 substituents independently selected from the group consisting of oxo and R^{c}.

In some embodiments, R¹¹ is phenyl optionally substituted with 1-3 substituents R^{c}.

In some embodiments, R¹¹ is phenyl substituted with 1-2 substituents R^{c}, wherein the phenyl is selected from the group consisting of:

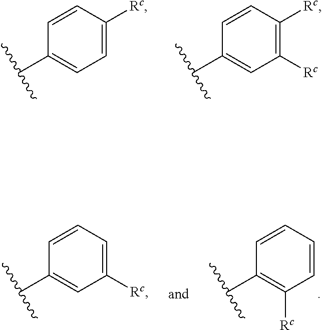

In some embodiments, R¹¹ is unsubstituted phenyl.

In some embodiments, R⁴, R⁵, R⁶, R⁷, R⁸, R⁹, R¹⁰, and R¹¹ are defined according to (D).

In some embodiments, one of R⁸ and R¹¹, together with the atoms to which each is attached, form a ring selected from the group consisting of:
heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R⁸ is selected from the group consisting of: H and C₁₃ alkyl.

In some embodiments, one of R⁸ and R¹¹, together with the atoms to which each is attached, form a heterocyclyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and the other of R⁸ is selected from the group consisting of: H and C₁₋₃ alkyl.

In some embodiments, one of R⁸ and R¹¹, together with the atoms to which each is attached, form a heterocyclyl of 5-6 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and the other of R⁸ is selected from the group consisting of: H and C₁₋₃ alkyl.

In some embodiments, one of R⁸ and R¹¹, together with the atoms to which each is attached, form a heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and the other of R⁸ is selected from the group consisting of: H and C₁₋₃ alkyl.

In some embodiments, one of R⁸ and R¹¹, together with the atoms to which each is attached, form a heteroaryl of 5-6 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and the other of R⁸ is selected from the group consisting of: H and C₁₋₃ alkyl.

In some embodiments, each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}.

In some embodiments, R⁴, R⁵, R⁶, R⁷, R⁹, and R¹⁰ are H.

Variables R^{a}, R^{b}, R^{c}, R^{d}, R^{e}, R^{f}, R⁹, R′, and R¹¹

In some embodiments, each occurrence of R^{b} and R^{c} is independently selected from the group consisting of: halo; cyano; —OH; C₁₋₄ alkoxy; C₁₋₄ haloalkoxy; and C₁₋₁₀ alkyl which is optionally substituted with 1-6 independently selected R^{a} e.g., C₁₋₆ alkyl, C₁₋₃ alkyl; e.g., each occurrence of R^{a} is F.

In some embodiments, each occurrence of R^{b} and R^{c} is independently selected from the group consisting of: C₂₋₆ alkenyl; C₂₋₆ alkynyl; C₁₋₄ alkoxy; C₁₋₄ haloalkoxy; —S(O)₁₋₂(C₁₋₄ alkyl); and —S(O)(=NH)(C₁₋₄ alkyl).

In some embodiments, each occurrence of R^{d} is C₁₋₆ alkyl optionally substituted with 1-3 independently selected R^{a}.

### Stereochemistry of the Compounds of Formula (I)

In some embodiments, the carbon atom attached to R¹, R², and R³ has the S-configuration.

In some embodiments, the carbon atom attached to R¹, R², and R³ has the R-configuration.

In some embodiments, the carbon atom attached to OR⁵ and R⁶ has the S-configuration.

In some embodiments, the carbon atom attached to OR⁵ and R⁶ has the R-configuration.

In some embodiments, the carbon atom attached to N(R⁸)₂ and R⁷ has the S-configuration.

In some embodiments, the carbon atom attached to N(R⁸)₂ and R⁷ has the R-configuration.

In some embodiments, the carbon atom attached to OR⁵ and R⁶ has the S-configuration, and the carbon atom attached to N(R⁸)₂ and R⁷ has the R-configuration.

In some embodiments, the carbon atom attached to R¹, R², and R³ has the S-configuration, the carbon atom attached to OR⁵ and R⁶ has the S-configuration, and the carbon atom attached to N(R⁸)₂ and R⁷ has the R-configuration.

In some embodiments, the carbon atom attached to R¹, R², and R³ has the S-configuration, the carbon atom attached to OR⁵ and R⁶ has the R-configuration, and the carbon atom attached to N(R⁸)₂ and R⁷ has the R-configuration.

In some embodiments, the carbon atom attached to R¹, R², and R³ has the S-configuration, the carbon atom attached to OR⁵ and R⁶ has the R-configuration, and the carbon atom attached to N(R⁸)₂ and R⁷ has the S-configuration.

In some embodiments, the carbon atom attached to R¹, R², and R³ has the R-configuration, the carbon atom attached to OR⁵ and R⁶ has the S-configuration, and the carbon atom attached to N(R⁸)₂ and R⁷ has the R-configuration.

In some embodiments, the carbon atom attached to R¹, R², and R³ has the R-configuration, the carbon atom attached to OR⁵ and R⁶ has the R-configuration, and the carbon atom attached to N(R⁸)₂ and R⁷ has the R-configuration.

In some embodiments, the carbon atom attached to R¹, R², and R³ has the R-configuration, the carbon atom attached to OR⁵ and R⁶ has the R-configuration, and the carbon atom attached to N(R⁸)₂ and R⁷ has the S-configuration.

### Non-Limiting Combinations

In some embodiments, the compound is a compound of Formula (I-i), (I-j), (I-k), or (I-1):

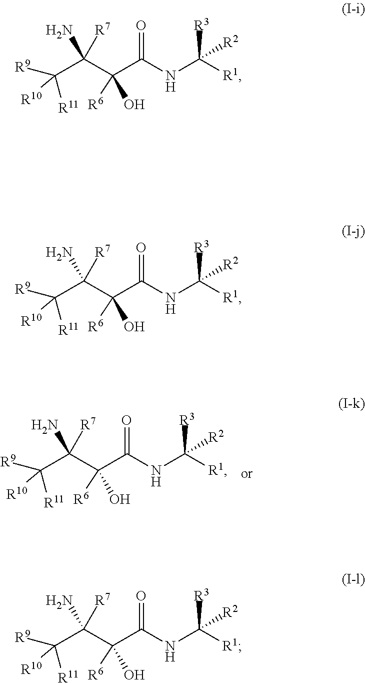

wherein:
R¹ is CO₂H, —CO—NR¹²R¹³, and R^{1A};
wherein:
R^{1A} is a carboxylic acid isostere or bioisostere optionally substituted with 1-3 independently selected R¹⁵;
R² is H or CH₃;
R³ is selected from the group consisting of:
   H
   C₁₋₆ alkyl optionally substituted with 1-6 independently selected R^{a};
   L¹-C₃₋₁₀ cycloalkyl optionally substituted with 1-6 R^{b}, wherein L¹ is a bond or unsubstituted C₁₋₆ alkylene;
   L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
   L⁴-C₆₋₁₀ aryl optionally substituted with 1-4 R^{c}, wherein L⁴ is a bond or unsubstituted C₁₋₆ alkylene; provided that one or both of R² and R³ is other than H.

In certain embodiments, the compound is a compound of Formula (I-i):

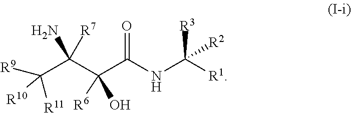

In certain embodiments, the compound is a compound of Formula (I-j):

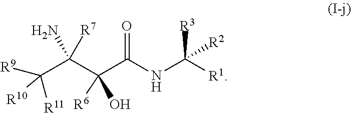

In certain embodiments, compound is a compound of Formula (I-k):

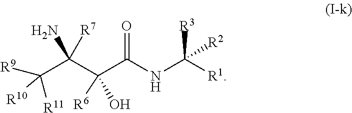

In certain embodiments, the compound is a compound of Formula (I-l):

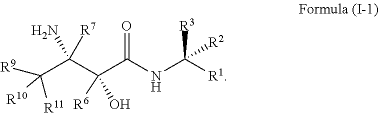

In some embodiments, the compound is a compound of Formula (I-ia), (I-ja), (I-ka), or (I-la):

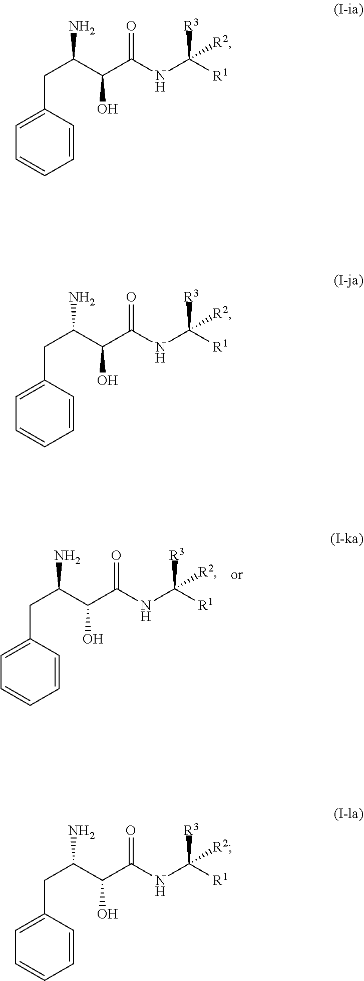

wherein:
R¹ is CO₂H, —CO—NR¹²R¹³, and R^{1A},
wherein:
R^{1A} is a carboxylic acid isostere or bioisostere optionally substituted with 1-3 independently selected R¹⁵;
R² is H or CH₃;
R³ is selected from the group consisting of:
   H
   C₁₋₆ alkyl optionally substituted with 1-6 independently selected R^{a};
   L¹-C₃₋₁₀ cycloalkyl optionally substituted with 1-6 R^{b}, wherein L¹ is a bond or unsubstituted C₁₋₆ alkylene;
   L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
   L⁴-C₆₋₁₀ aryl optionally substituted with 1-4 R^{c}, wherein L⁴ is a bond or unsubstituted C₁₋₆ alkylene; provided that one or both of R² and R³ is other than H.

In certain embodiments, the compound is a compound of Formula (I-ia):

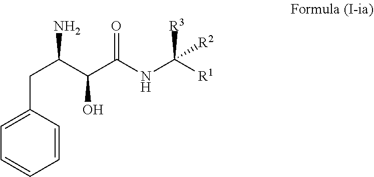

In certain embodiments, the compound is a compound of Formula (I-ja):

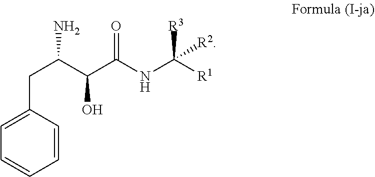

In certain embodiments, compound is a compound of Formula (I-ka):

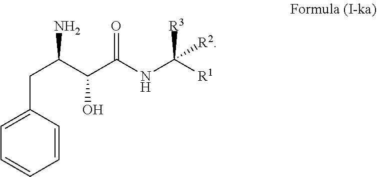

In certain embodiments, the compound is a compound of Formula (I-la):

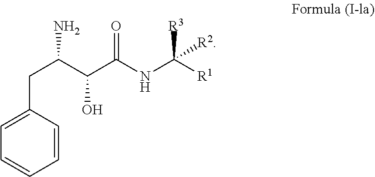

In some embodiments of Formula (I-i), (I-j), (I-k), (I-l), (I-ia), (I-ja), (I-ka), or (I-la), R¹ is CO₂H.

In some embodiments of Formula (I-i), (I-j), (I-k), (I-l), (I-ia), (I-ja), (I-ka), or (I-la), R¹ is R^{1A}, wherein R^{1A} is a carboxylic acid isostere or bioisostere.

In some embodiments of Formula (I-i), (I-j), (I-k), (I-l), (I-ia), (I-ja), (I-ka), or (I-la), R¹ is R^{1A}, wherein R^{1A} is

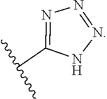 [SMILES: *c1nnn[nH]1]

### OTHER EMBODIMENTS

In one aspect, provided herein are compounds having formula I:

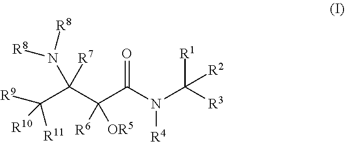

or a pharmaceutically acceptable thereof, wherein:
R¹ is —CO₂H;
R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
R⁴, R⁵, R⁶, R⁷, R⁸, R⁹, R¹⁰, and R¹¹ are defined according to (Q) and (D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R^{b}; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R;
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R⁸ is selected from the group consisting of: H and C₁₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a};
each occurrence of R′ and R″ is independently selected from the group consisting of: H; —OH; and C₁₋₄ alkyl;
A) provided when:
R¹ is CO₂H,
each of R⁴, R⁵, R⁶, R⁷, R⁸, R⁹, and R¹⁰ is H,
R¹¹ is phenyl that is optionally substituted with one or more of NO₂, F, Cl, —OCH₃, —CH₃, phenyl, 2-benzothiazolyloxy, benzyloxy, iodo, —SCH₃, —SO₂CH₃, iso-propyl, iso-propoxy, iso-butyl, —OH, N₃, and NH₂; and
one of R² and R³ is H,
then the other of R² and R³ cannot be:
(i) the amino acid sidechain present in leucine, iso-leucine, threonine, tyrosine, O-benzyl-protected tyrosine, tryptophan, allothreonine, histidine, valine, alanine, arginine, glutamine, glutamic acid, aspartic acid, serine, lysine, N-benzyloxy-protected lysine, or methionine; or
(ii) n-hexyl, tert-butyl, n-butyl, or n-propyl;
B) further provided when:
R is CO₂H,
each of R⁴, R⁵, R⁶, R⁷, R⁸, R⁹, and R¹⁰ is H,
R² and R³ together with the carbon atom to which each is attached form a 3, 4, 5, or 6 member ring; and
R¹ is phenyl
then the phenyl ring cannot be substituted at the para position with —SO₂CH₃; and
C) further provided the compound is not

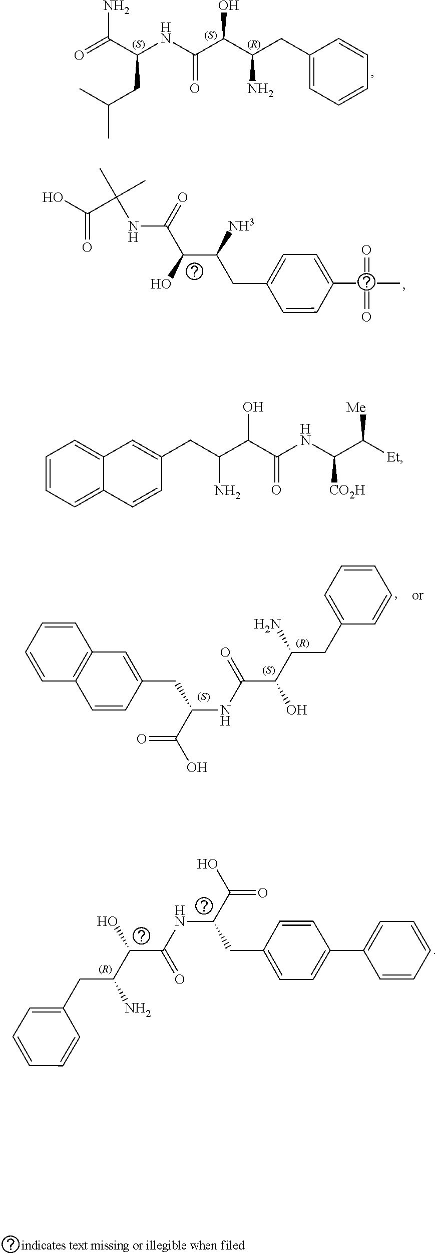

In one aspect, provided herein are compounds of Formula (I):

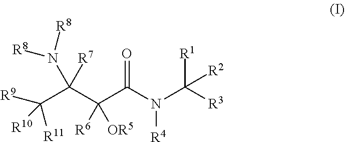

or a pharmaceutically acceptable thereof, wherein:
R¹ is R^{1A};
wherein:
R^{1A} is a carboxylic acid isostere or bioisostere optionally substituted with 1-3 independently selected R′⁵;
each occurrence of R¹⁵ is independently selected from the group consisting of:
H;
halo;
C₁₋₆ alkyl optionally substituted with 1-3 independently selected R^{a}; and
C₃₋₆ cycloalkyl which is optionally substituted with 1-6 independently selected R^{b}
R² and R³ are each independently selected from the group consisting of:
H;
halo;
CN;
C₁₋₂₀ alkyl optionally substituted with 1-6 independently selected R^{a};
L-C₃₋₁₀ cycloalkyl or L-C₃₋₁₀ cycloalkenyl, each of which is optionally substituted with 1-6 substituents independently selected from the group consisting of oxo and R^{b}, wherein L is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
C₃₋₁₀ cycloalkyl or C₃₋₁₀ cycloalkenyl, each of which is optionally substituted with 1-6 substituents independently selected from the group consisting of oxo and R^{b}; and
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
R⁴, R⁵, R⁶, R⁷, R⁸, R⁹, R¹⁰, and R¹¹ are defined according to (C) and (D) below:
(C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
C₃₋₆ cycloalkyl or C₃₋₆₀ cycloalkenyl, each of which is optionally substituted with 1-6 substituents independently selected from the group consisting of oxo and R^{b}; and
heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
C₃₋₁₀ cycloalkyl or C₃₋₁₀ cycloalkenyl, each of which is optionally substituted with 1-6 substituents independently selected from the group consisting of oxo and R^{b};
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
C₆₋₁₀ aryl optionally substituted with 1-4 substituents independently selected from the group consisting of oxo and R^{c};
(D)
one of R⁸ and R¹¹, together with the atoms to which each is attached, form a ring selected from the group consisting of:
heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R⁸ is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently from the group consisting of:
C₃₋₈ cycloalkyl or C₃₋₈ cycloalkenyl, each of which is optionally substituted with 1-6 substituents independently selected from the group consisting of C₁₋₆ alkyl and R^{a};
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
C₆₋₁₀ aryl optionally substituted with 1-4 substituents independently selected from the group consisting of C₁₋₆ alkyl and R^{a};
each occurrence of R′ and R″ is independently selected from the group consisting of: H; —OH; and C₁₋₄ alkyl.

In one aspect, provided herein are compounds Formula (I):

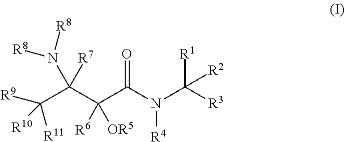

or a pharmaceutically acceptable thereof, wherein:
R¹ is

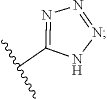 [SMILES: *c1nnn[nH]1]

R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
R⁴, R⁵, R⁶, R⁷, R⁸, R⁹, R¹⁰, and R¹¹ are defined according to (C) and (D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R^{b}; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R^{g}) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R^{g}) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R^{g} is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a};
each occurrence of R′ and R″ is independently selected from the group consisting of: H; —OH; and C₁₋₄ alkyl.

### Non-Limiting Exemplary Compounds

In some embodiments, the compound is selected from the group consisting of the compounds delineated in Table C1 and Table C2 or a pharmaceutically acceptable salt thereof.

TABLE C1

| Compound | |
|---|---|
| Compound 1 | 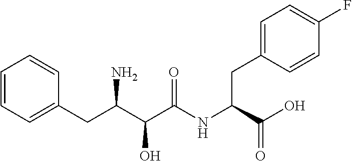 [SMILES: N[C@H](Cc1ccccc1)[C@H](O)C(=O)N[C@@H](Cc1ccc(F)cc1)C(=O)O] |
| Compound 2 | 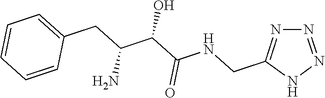 [SMILES: N[C@H](Cc1ccccc1)[C@H](O)C(=O)NCc1nnn[nH]1] |
| Compound 3 | 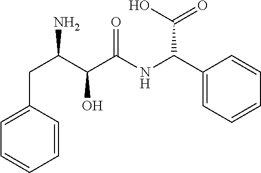 |
| Compound 4 | 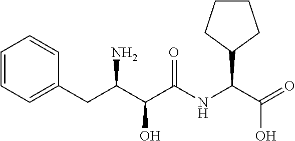 [SMILES: N[C@H](Cc1ccccc1)[C@H](O)C(=O)N[C@H](C(=O)O)C1CCCC1] |
| Compound 5 | 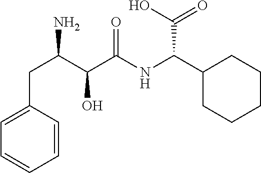 [SMILES: N[C@H](Cc1ccccc1)[C@H](O)C(=O)N[C@H](C(=O)O)C1CCCCC1] |
| Compound 6 | 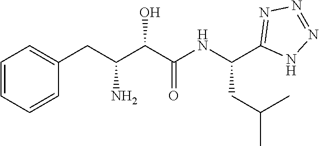 [SMILES: CC(C)C[C@H](NC(=O)[C@@H](O)[C@H](N)Cc1ccccc1)c1nnn[nH]1] |
| Compound 7 | 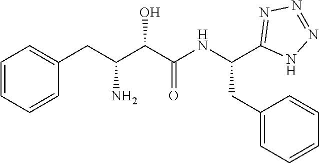 [SMILES: N[C@H](Cc1ccccc1)[C@H](O)C(=O)N[C@@H](Cc1ccccc1)c1nnn[nH]1] |
| Compound 8 | 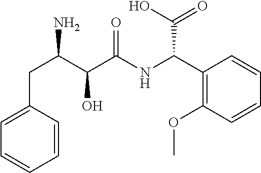 [SMILES: COc1ccccc1[C@H](NC(=O)[C@@H](O)[C@H](N)Cc1ccccc1)C(=O)O] |
| Compound 9 | 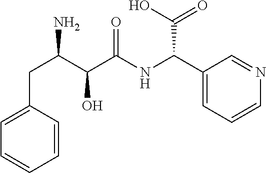 [SMILES: N[C@H](Cc1ccccc1)[C@H](O)C(=O)N[C@H](C(=O)O)c1cccnc1] |
| Compound 10 | 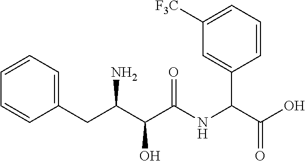 [SMILES: N[C@H](Cc1ccccc1)[C@H](O)C(=O)NC(C(=O)O)c1cccc(C(F)(F)F)c1] |
| Compound 11 | 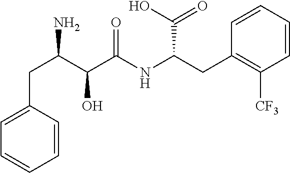 [SMILES: N[C@H](Cc1ccccc1)[C@H](O)C(=O)N[C@@H](Cc1ccccc1C(F)(F)F)C(=O)O] |
| Compound 12 | 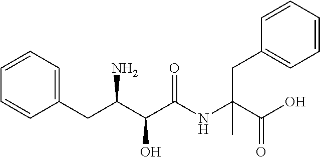 [SMILES: CC(Cc1ccccc1)(NC(=O)[C@@H](O)[C@H](N)Cc1ccccc1)C(=O)O] |
| Compound 13 | 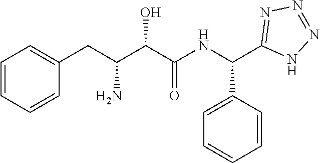 [SMILES: N[C@H](Cc1ccccc1)[C@H](O)C(=O)N[C@@H](c1ccccc1)c1nnn[nH]1] |
| Compound 14 | 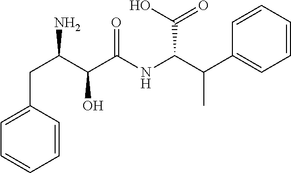 [SMILES: CC(c1ccccc1)[C@H](NC(=O)[C@@H](O)[C@H](N)Cc1ccccc1)C(=O)O] |
| Compound 15 | 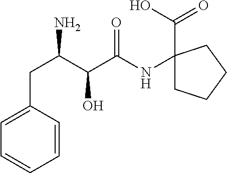 [SMILES: N[C@H](Cc1ccccc1)[C@H](O)C(=O)NC1(C(=O)O)CCCC1] |
| Compound 16 | 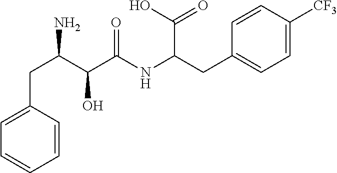 [SMILES: N[C@H](Cc1ccccc1)[C@H](O)C(=O)NC(Cc1ccc(C(F)(F)F)cc1)C(=O)O] |
| Compound 17 | 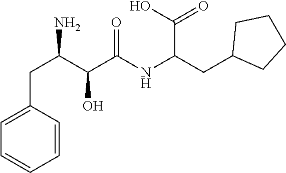 [SMILES: N[C@H](Cc1ccccc1)[C@H](O)C(=O)NC(CC1CCCC1)C(=O)O] |
| Compound 18 | 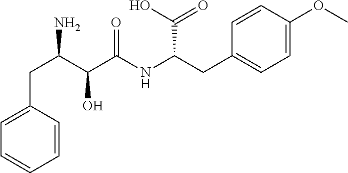 [SMILES: COc1ccc(C[C@H](NC(=O)[C@@H](O)[C@H](N)Cc2ccccc2)C(=O)O)cc1] |
| Compound 19 | 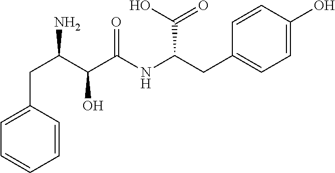 [SMILES: N[C@H](Cc1ccccc1)[C@H](O)C(=O)N[C@@H](Cc1ccc(O)cc1)C(=O)O] |
| Compound 20 | 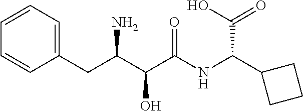 [SMILES: N[C@H](Cc1ccccc1)[C@H](O)C(=O)N[C@H](C(=O)O)C1CCC1] |
| Compound 21 | 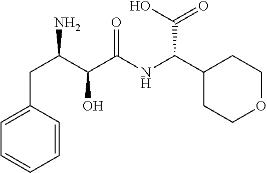 [SMILES: N[C@H](Cc1ccccc1)[C@H](O)C(=O)N[C@H](C(=O)O)C1CCOCC1] |
| Compound 22 | 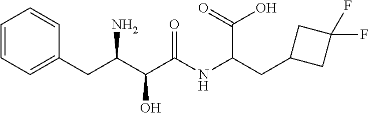 [SMILES: N[C@H](Cc1ccccc1)[C@H](O)C(=O)NC(CC1CC(F)(F)C1)C(=O)O] |
| Compound 23 | 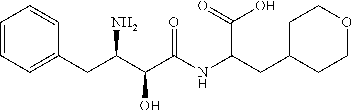 [SMILES: N[C@H](Cc1ccccc1)[C@H](O)C(=O)NC(CC1CCOCC1)C(=O)O] |
| Compound 24 | 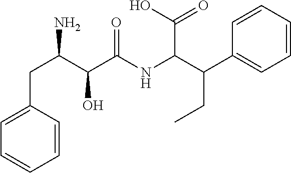 [SMILES: CCC(c1ccccc1)C(NC(=O)[C@@H](O)[C@H](N)Cc1ccccc1)C(=O)O] |
| Compound 25 | 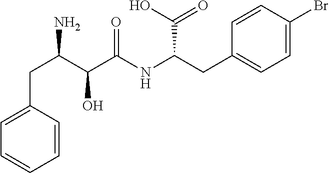 [SMILES: N[C@H](Cc1ccccc1)[C@H](O)C(=O)N[C@@H](Cc1ccc(Br)cc1)C(=O)O] |
| Compound 26 | 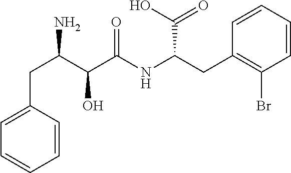 [SMILES: N[C@H](Cc1ccccc1)[C@H](O)C(=O)N[C@@H](Cc1ccccc1Br)C(=O)O] |
| Compound 27 | 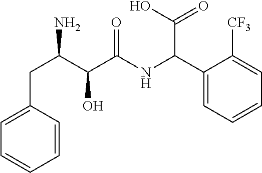 [SMILES: N[C@H](Cc1ccccc1)[C@H](O)C(=O)NC(C(=O)O)c1ccccc1C(F)(F)F] |
| Compound 28 | 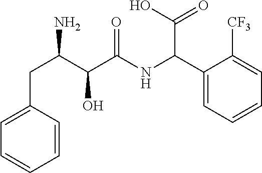 [SMILES: N[C@H](Cc1ccccc1)[C@H](O)C(=O)NC(C(=O)O)c1ccccc1C(F)(F)F] |
| Compound 29 | 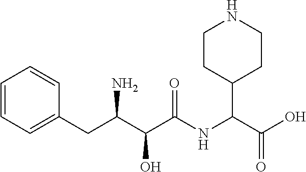 [SMILES: N[C@H](Cc1ccccc1)[C@H](O)C(=O)NC(C(=O)O)C1CCNCC1] |
| Compound 30 | 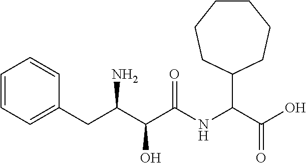 [SMILES: N[C@H](Cc1ccccc1)[C@H](O)C(=O)NC(C(=O)O)C1CCCCCC1] |
| Compound 31 | 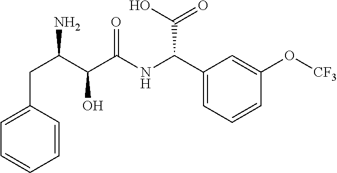 [SMILES: N[C@H](Cc1ccccc1)[C@H](O)C(=O)N[C@H](C(=O)O)c1cccc(OC(F)(F)F)c1] |
| Compound 32 | 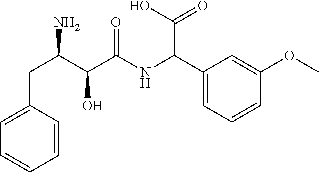 [SMILES: COc1cccc(C(NC(=O)[C@@H](O)[C@H](N)Cc2ccccc2)C(=O)O)c1] |
| Compound 33 | 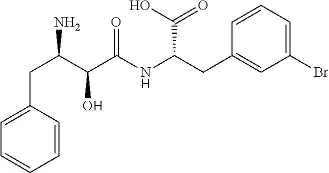 [SMILES: N[C@H](Cc1ccccc1)[C@H](O)C(=O)N[C@@H](Cc1cccc(Br)c1)C(=O)O] |
| Compound 34 | 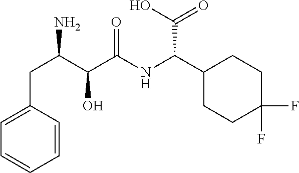 [SMILES: N[C@H](Cc1ccccc1)[C@H](O)C(=O)N[C@H](C(=O)O)C1CCC(F)(F)CC1] |
| Compound 35 | 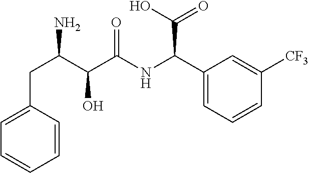 [SMILES: N[C@H](Cc1ccccc1)[C@H](O)C(=O)N[C@@H](C(=O)O)c1cccc(C(F)(F)F)c1] |
| Compound 36 | 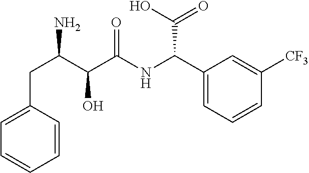 [SMILES: N[C@H](Cc1ccccc1)[C@H](O)C(=O)N[C@H](C(=O)O)c1cccc(C(F)(F)F)c1] |
| Compound 37 | 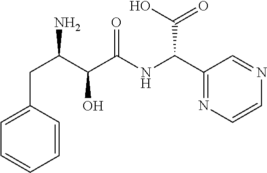 [SMILES: N[C@H](Cc1ccccc1)[C@H](O)C(=O)N[C@H](C(=O)O)c1cnccn1] |
| Compound 38 | 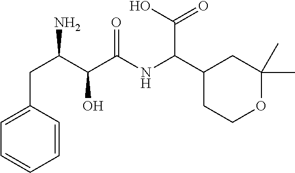 [SMILES: CC1(C)CC(C(NC(=O)[C@@H](O)[C@H](N)Cc2ccccc2)C(=O)O)CCO1] |
| Compound 39 | 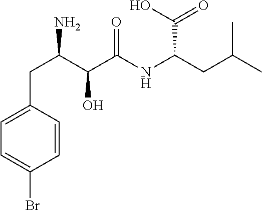 [SMILES: CC(C)C[C@H](NC(=O)[C@@H](O)[C@H](N)Cc1ccc(Br)cc1)C(=O)O] |
| Compound 40 | 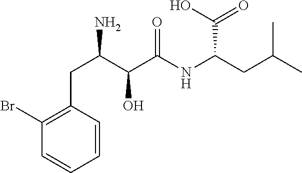 [SMILES: CC(C)C[C@H](NC(=O)[C@@H](O)[C@H](N)Cc1ccccc1Br)C(=O)O] |
| Compound 41 | 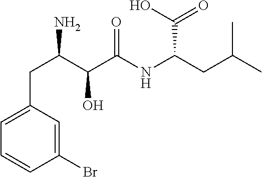 [SMILES: CC(C)C[C@H](NC(=O)[C@@H](O)[C@H](N)Cc1cccc(Br)c1)C(=O)O] |
| Compound 42 | 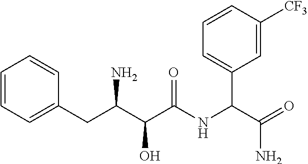 [SMILES: NC(=O)C(NC(=O)[C@@H](O)[C@H](N)Cc1ccccc1)c1cccc(C(F)(F)F)c1] |
| Compound 43 | 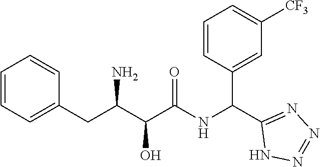 [SMILES: N[C@H](Cc1ccccc1)[C@H](O)C(=O)NC(c1cccc(C(F)(F)F)c1)c1nnn[nH]1] |
| Compound 44 | 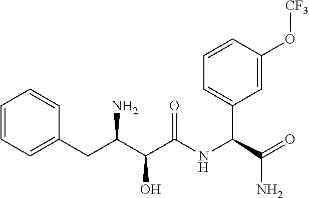 [SMILES: NC(=O)[C@@H](NC(=O)[C@@H](O)[C@H](N)Cc1ccccc1)c1cccc(OC(F)(F)F)c1] |
| Compound 45 | 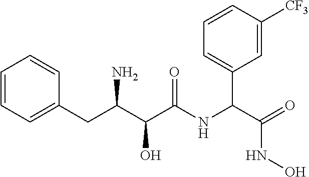 [SMILES: N[C@H](Cc1ccccc1)[C@H](O)C(=O)NC(C(=O)NO)c1cccc(C(F)(F)F)c1] |
| Compound 46 | 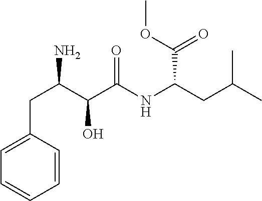 [SMILES: COC(=O)[C@H](CC(C)C)NC(=O)[C@@H](O)[C@H](N)Cc1ccccc1] |
| Compound 47 | 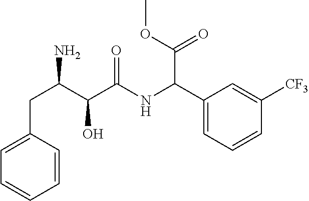 [SMILES: COC(=O)C(NC(=O)[C@@H](O)[C@H](N)Cc1ccccc1)c1cccc(C(F)(F)F)c1] |
| Compound 48 | 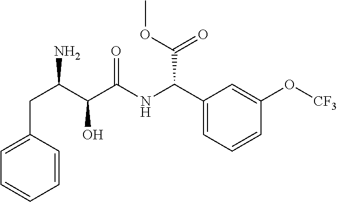 [SMILES: COC(=O)[C@@H](NC(=O)[C@@H](O)[C@H](N)Cc1ccccc1)c1cccc(OC(F)(F)F)c1] |
| Compound 49 | 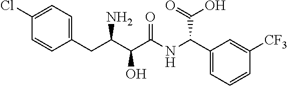 [SMILES: N[C@H](Cc1ccc(Cl)cc1)[C@H](O)C(=O)N[C@H](C(=O)O)c1cccc(C(F)(F)F)c1] |
| Compound 50 | 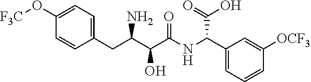 [SMILES: N[C@H](Cc1ccc(OC(F)(F)F)cc1)[C@H](O)C(=O)N[C@H](C(=O)O)c1cccc(OC(F)(F)F)c1] |
| Compound 51 | 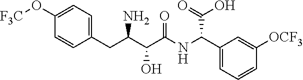 [SMILES: N[C@H](Cc1ccc(OC(F)(F)F)cc1)[C@@H](O)C(=O)N[C@H](C(=O)O)c1cccc(OC(F)(F)F)c1] |
| Compound 52 | 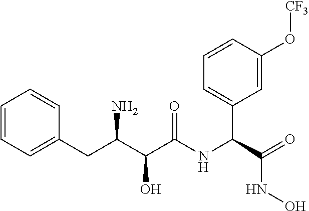 [SMILES: N[C@H](Cc1ccccc1)[C@H](O)C(=O)N[C@H](C(=O)NO)c1cccc(OC(F)(F)F)c1] |
| Compound 53 | 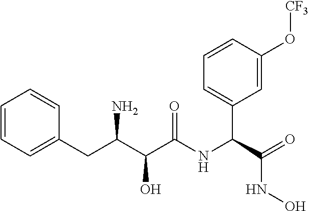 [SMILES: N[C@H](Cc1ccccc1)[C@H](O)C(=O)N[C@H](C(=O)NO)c1cccc(OC(F)(F)F)c1] |
| Compound 54 | 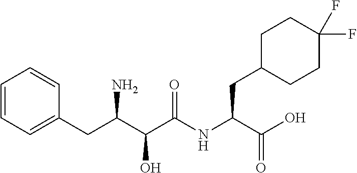 [SMILES: N[C@H](Cc1ccccc1)[C@H](O)C(=O)N[C@@H](CC1CCC(F)(F)CC1)C(=O)O] |
| Compound 55 | 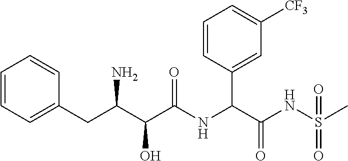 [SMILES: CS(=O)(=O)NC(=O)C(NC(=O)[C@@H](O)[C@H](N)Cc1ccccc1)c1cccc(C(F)(F)F)c1] |
| Compound 56 | 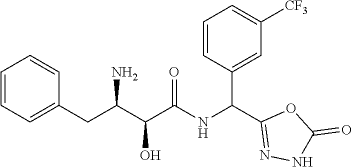 [SMILES: N[C@H](Cc1ccccc1)[C@H](O)C(=O)NC(c1cccc(C(F)(F)F)c1)c1n[nH]c(=O)o1] |
| Compound 57 | 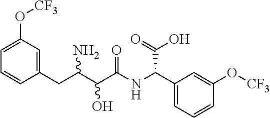 [SMILES: NC(Cc1cccc(OC(F)(F)F)c1)C(O)C(=O)N[C@H](C(=O)O)c1cccc(OC(F)(F)F)c1] |
| Compound 58 | 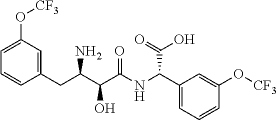 [SMILES: N[C@H](Cc1cccc(OC(F)(F)F)c1)[C@H](O)C(=O)N[C@H](C(=O)O)c1cccc(OC(F)(F)F)c1] |
| Compound 59 | 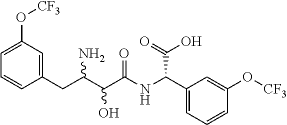 [SMILES: NC(Cc1cccc(OC(F)(F)F)c1)C(O)C(=O)N[C@H](C(=O)O)c1cccc(OC(F)(F)F)c1] |
| Compound 60 | 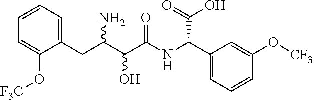 [SMILES: NC(Cc1ccccc1OC(F)(F)F)C(O)C(=O)N[C@H](C(=O)O)c1cccc(OC(F)(F)F)c1] |
| Compound 61 | 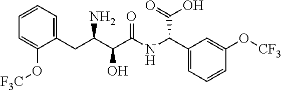 [SMILES: N[C@H](Cc1ccccc1OC(F)(F)F)[C@H](O)C(=O)N[C@H](C(=O)O)c1cccc(OC(F)(F)F)c1] |
| Compound 62 | 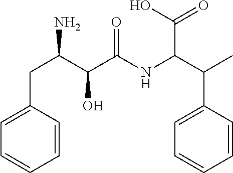 [SMILES: CC(c1ccccc1)C(NC(=O)[C@@H](O)[C@H](N)Cc1ccccc1)C(=O)O] |
| Compound 63 | 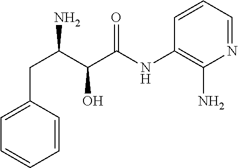 [SMILES: Nc1ncccc1NC(=O)[C@@H](O)[C@H](N)Cc1ccccc1] |

TABLE C2

| Compound | |
|---|---|
| Compound 64 | 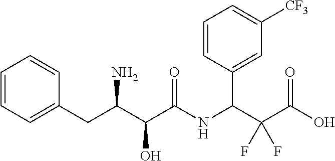 [SMILES: N[C@H](Cc1ccccc1)[C@H](O)C(=O)NC(c1cccc(C(F)(F)F)c1)C(F)(F)C(=O)O] |
| Compound 65 | 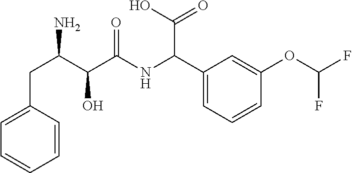 [SMILES: N[C@H](Cc1ccccc1)[C@H](O)C(=O)NC(C(=O)O)c1cccc(OC(F)F)c1] |
| Compound 66 | 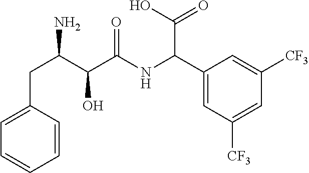 [SMILES: N[C@H](Cc1ccccc1)[C@H](O)C(=O)NC(C(=O)O)c1cc(C(F)(F)F)cc(C(F)(F)F)c1] |
| Compound 67 | 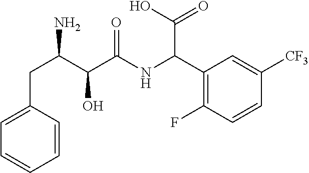 [SMILES: N[C@H](Cc1ccccc1)[C@H](O)C(=O)NC(C(=O)O)c1cc(C(F)(F)F)ccc1F] |
| Compound 68 | 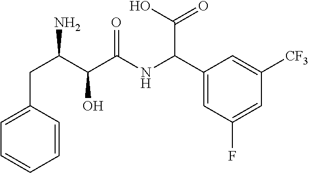 [SMILES: N[C@H](Cc1ccccc1)[C@H](O)C(=O)NC(C(=O)O)c1cc(F)cc(C(F)(F)F)c1] |
| Compound 69 | 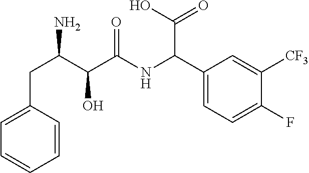 [SMILES: N[C@H](Cc1ccccc1)[C@H](O)C(=O)NC(C(=O)O)c1ccc(F)c(C(F)(F)F)c1] |
| Compound 70 | 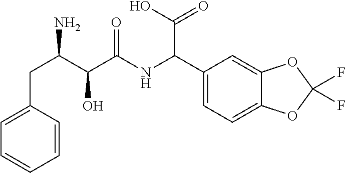 [SMILES: N[C@H](Cc1ccccc1)[C@H](O)C(=O)NC(C(=O)O)c1ccc2c(c1)OC(F)(F)O2] |
| Compound 71 | 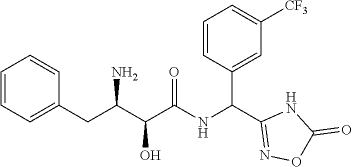 [SMILES: N[C@H](Cc1ccccc1)[C@H](O)C(=O)NC(c1cccc(C(F)(F)F)c1)c1noc(=O)[nH]1] |
| Compound 72 | 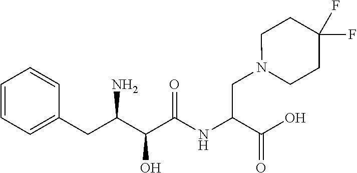 [SMILES: N[C@H](Cc1ccccc1)[C@H](O)C(=O)NC(CN1CCC(F)(F)CC1)C(=O)O] |
| Compound 73 | 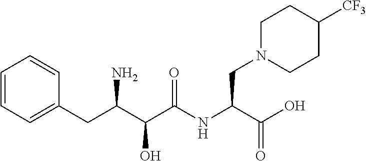 [SMILES: N[C@H](Cc1ccccc1)[C@H](O)C(=O)N[C@@H](CN1CCC(C(F)(F)F)CC1)C(=O)O] |
| Compound 74 | 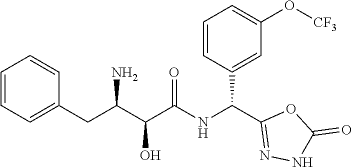 [SMILES: N[C@H](Cc1ccccc1)[C@H](O)C(=O)N[C@H](c1cccc(OC(F)(F)F)c1)c1n[nH]c(=O)o1] |
| Compound 75 | 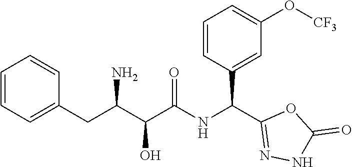 [SMILES: N[C@H](Cc1ccccc1)[C@H](O)C(=O)N[C@@H](c1cccc(OC(F)(F)F)c1)c1n[nH]c(=O)o1] |
| Compound 76 | 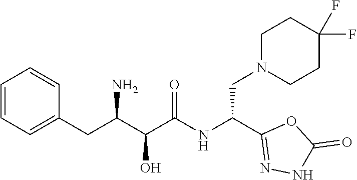 [SMILES: N[C@H](Cc1ccccc1)[C@H](O)C(=O)N[C@H](CN1CCC(F)(F)CC1)c1n[nH]c(=O)o1] |
| Compound 77 | 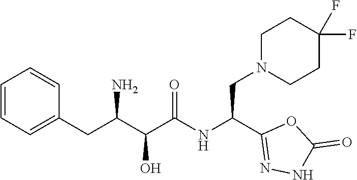 [SMILES: N[C@H](Cc1ccccc1)[C@H](O)C(=O)N[C@@H](CN1CCC(F)(F)CC1)c1n[nH]c(=O)o1] |
| Compound 78 | 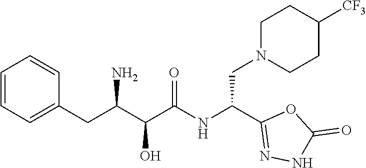 [SMILES: N[C@H](Cc1ccccc1)[C@H](O)C(=O)N[C@H](CN1CCC(C(F)(F)F)CC1)c1n[nH]c(=O)o1] |
| Compound 79 | 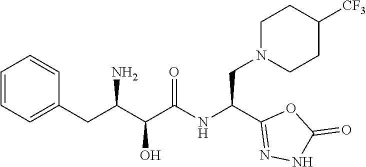 [SMILES: N[C@H](Cc1ccccc1)[C@H](O)C(=O)N[C@@H](CN1CCC(C(F)(F)F)CC1)c1n[nH]c(=O)o1] |
| Compound 80 | 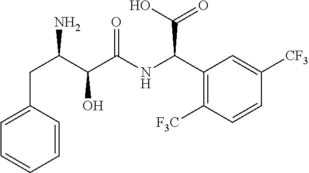 [SMILES: N[C@H](Cc1ccccc1)[C@H](O)C(=O)N[C@@H](C(=O)O)c1cc(C(F)(F)F)ccc1C(F)(F)F] |
| Compound 81 | 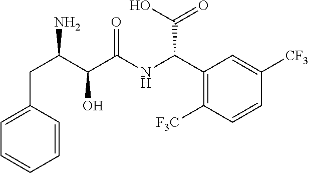 [SMILES: N[C@H](Cc1ccccc1)[C@H](O)C(=O)N[C@H](C(=O)O)c1cc(C(F)(F)F)ccc1C(F)(F)F] |
| Compound 82 | 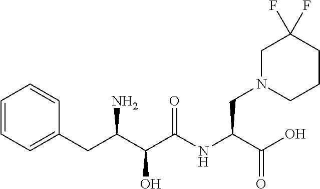 [SMILES: N[C@H](Cc1ccccc1)[C@H](O)C(=O)N[C@@H](CN1CCCC(F)(F)C1)C(=O)O] |
| Compound 83 | 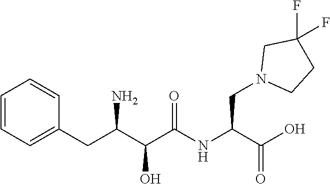 [SMILES: N[C@H](Cc1ccccc1)[C@H](O)C(=O)N[C@@H](CN1CCC(F)(F)C1)C(=O)O] |
| Compound 84 | 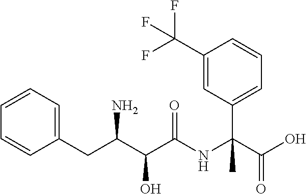 [SMILES: C[C@](NC(=O)[C@@H](O)[C@H](N)Cc1ccccc1)(C(=O)O)c1cccc(C(F)(F)F)c1] |
| Compound 85 | 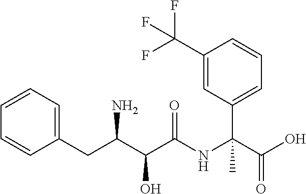 [SMILES: C[C@@](NC(=O)[C@@H](O)[C@H](N)Cc1ccccc1)(C(=O)O)c1cccc(C(F)(F)F)c1] |
| Compound 86 | 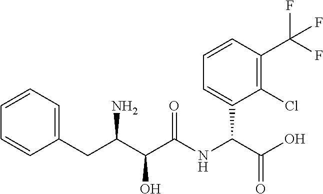 [SMILES: N[C@H](Cc1ccccc1)[C@H](O)C(=O)N[C@@H](C(=O)O)c1cccc(C(F)(F)F)c1Cl] |
| Compound 87 | 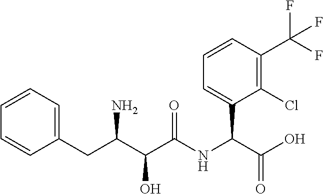 [SMILES: N[C@H](Cc1ccccc1)[C@H](O)C(=O)N[C@H](C(=O)O)c1cccc(C(F)(F)F)c1Cl] |
| Compound 88 | 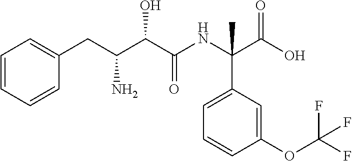 [SMILES: C[C@@](NC(=O)[C@@H](O)[C@H](N)Cc1ccccc1)(C(=O)O)c1cccc(OC(F)(F)F)c1] |
| Compound 89 | 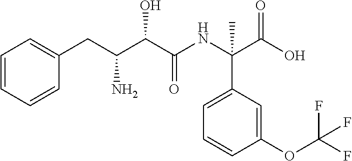 [SMILES: C[C@](NC(=O)[C@@H](O)[C@H](N)Cc1ccccc1)(C(=O)O)c1cccc(OC(F)(F)F)c1] |
| Compound 90 | 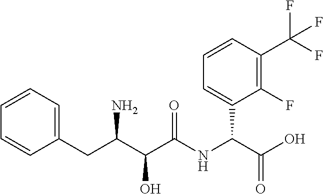 [SMILES: N[C@H](Cc1ccccc1)[C@H](O)C(=O)N[C@@H](C(=O)O)c1cccc(C(F)(F)F)c1F] |
| Compound 91 | 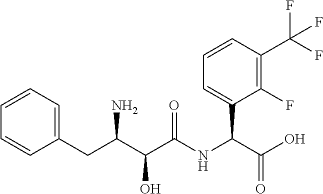 [SMILES: N[C@H](Cc1ccccc1)[C@H](O)C(=O)N[C@H](C(=O)O)c1cccc(C(F)(F)F)c1F] |
| Compound 92 | 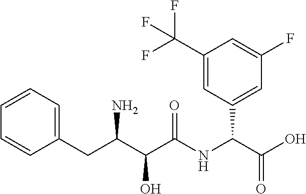 [SMILES: N[C@H](Cc1ccccc1)[C@H](O)C(=O)N[C@@H](C(=O)O)c1cc(F)cc(C(F)(F)F)c1] |
| Compound 93 | 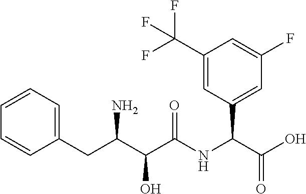 [SMILES: N[C@H](Cc1ccccc1)[C@H](O)C(=O)N[C@H](C(=O)O)c1cc(F)cc(C(F)(F)F)c1] |
| Compound 94 | 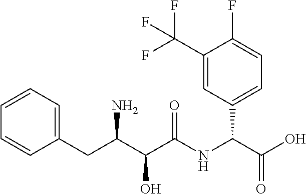 [SMILES: N[C@H](Cc1ccccc1)[C@H](O)C(=O)N[C@@H](C(=O)O)c1ccc(F)c(C(F)(F)F)c1] |
| Compound 95 | 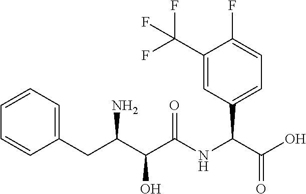 [SMILES: N[C@H](Cc1ccccc1)[C@H](O)C(=O)N[C@H](C(=O)O)c1ccc(F)c(C(F)(F)F)c1] |
| Compound 96 | 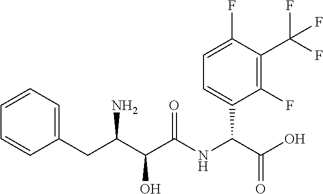 [SMILES: N[C@H](Cc1ccccc1)[C@H](O)C(=O)N[C@@H](C(=O)O)c1ccc(F)c(C(F)(F)F)c1F] |
| Compound 97 | 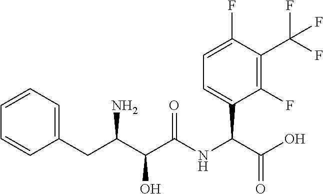 [SMILES: N[C@H](Cc1ccccc1)[C@H](O)C(=O)N[C@H](C(=O)O)c1ccc(F)c(C(F)(F)F)c1F] |
| Compound 98 | 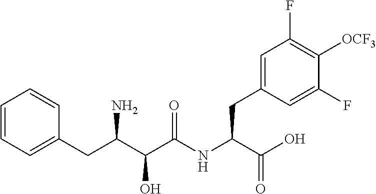 [SMILES: N[C@H](Cc1ccccc1)[C@H](O)C(=O)N[C@@H](Cc1cc(F)c(OC(F)(F)F)c(F)c1)C(=O)O] |
| Compound 99 | 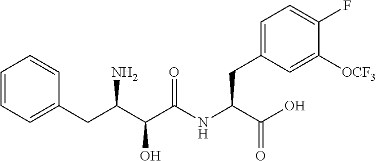 [SMILES: N[C@H](Cc1ccccc1)[C@H](O)C(=O)N[C@@H](Cc1ccc(F)c(OC(F)(F)F)c1)C(=O)O] |
| Compound 100 | 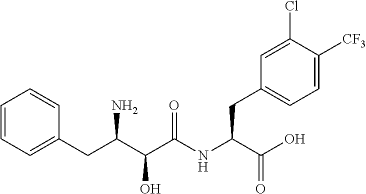 [SMILES: N[C@H](Cc1ccccc1)[C@H](O)C(=O)N[C@@H](Cc1ccc(C(F)(F)F)c(Cl)c1)C(=O)O] |
| Compound 101 | 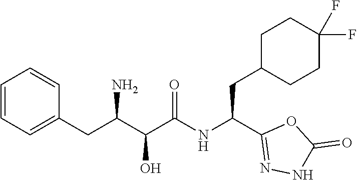 [SMILES: N[C@H](Cc1ccccc1)[C@H](O)C(=O)N[C@@H](CC1CCC(F)(F)CC1)c1n[nH]c(=O)o1] |
| Compound 102 | 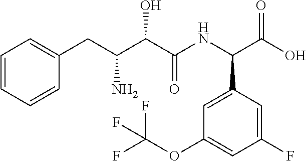 [SMILES: N[C@H](Cc1ccccc1)[C@H](O)C(=O)N[C@@H](C(=O)O)c1cc(F)cc(OC(F)(F)F)c1] |
| Compound 103 | 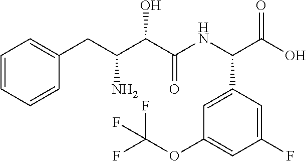 [SMILES: N[C@H](Cc1ccccc1)[C@H](O)C(=O)N[C@H](C(=O)O)c1cc(F)cc(OC(F)(F)F)c1] |
| Compound 104 | 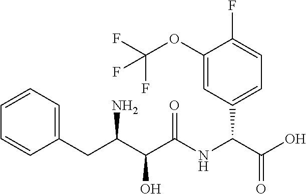 [SMILES: N[C@H](Cc1ccccc1)[C@H](O)C(=O)N[C@@H](C(=O)O)c1ccc(F)c(OC(F)(F)F)c1] |
| Compound 105 | 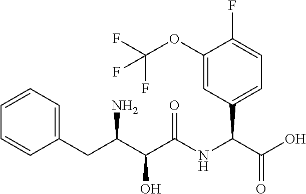 [SMILES: N[C@H](Cc1ccccc1)[C@H](O)C(=O)N[C@H](C(=O)O)c1ccc(F)c(OC(F)(F)F)c1] |
| Compound 106 | 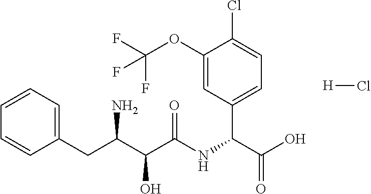 [SMILES: N[C@H](Cc1ccccc1)[C@H](O)C(=O)N[C@@H](C(=O)O)c1ccc(Cl)c(OC(F)(F)F)c1 ; Cl] |
| Compound 107 | 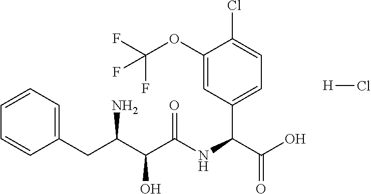 [SMILES: N[C@H](Cc1ccccc1)[C@H](O)C(=O)N[C@H](C(=O)O)c1ccc(Cl)c(OC(F)(F)F)c1 ; Cl] |
| Compound 108 | 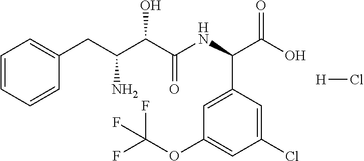 |
| Compound 109 | 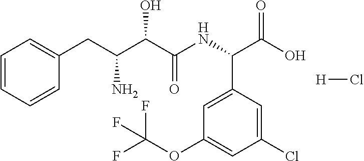 |
| Compound 110 | 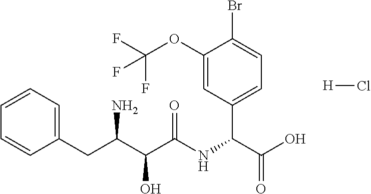 [SMILES: N[C@H](Cc1ccccc1)[C@H](O)C(=O)N[C@@H](C(=O)O)c1ccc(Br)c(OC(F)(F)F)c1 ; Cl] |
| Compound 111 | 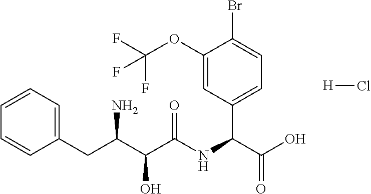 [SMILES: N[C@H](Cc1ccccc1)[C@H](O)C(=O)N[C@H](C(=O)O)c1ccc(Br)c(OC(F)(F)F)c1 ; Cl] |
| Compound 112 | 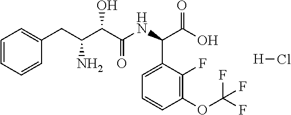 |
| Compound 113 | 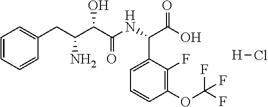 |
| Compound 114 | 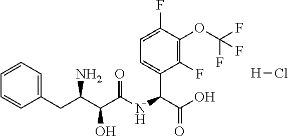 |
| Compound 115 | 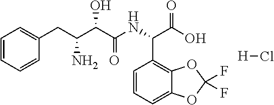 |
| Compound 116 | 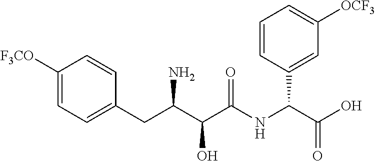 [SMILES: N[C@H](Cc1ccc(OC(F)(F)F)cc1)[C@H](O)C(=O)N[C@@H](C(=O)O)c1cccc(OC(F)(F)F)c1] |
| Compound 117 | 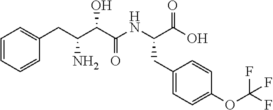 [SMILES: N[C@H](Cc1ccccc1)[C@H](O)C(=O)N[C@@H](Cc1ccc(OC(F)(F)F)cc1)C(=O)O] |
| Compound 118 | 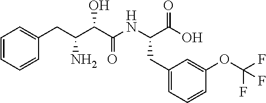 [SMILES: N[C@H](Cc1ccccc1)[C@H](O)C(=O)N[C@@H](Cc1cccc(OC(F)(F)F)c1)C(=O)O] |
| Compound 119 | 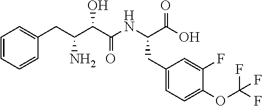 [SMILES: N[C@H](Cc1ccccc1)[C@H](O)C(=O)N[C@@H](Cc1ccc(OC(F)(F)F)c(F)c1)C(=O)O] |
| Compound 120 | 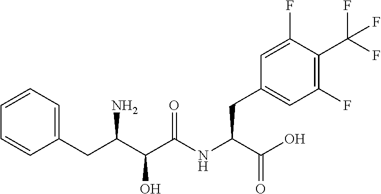 [SMILES: N[C@H](Cc1ccccc1)[C@H](O)C(=O)N[C@@H](Cc1cc(F)c(C(F)(F)F)c(F)c1)C(=O)O] |
| Compound 121 | 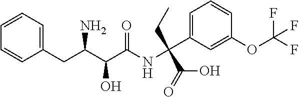 [SMILES: CC[C@](NC(=O)[C@@H](O)[C@H](N)Cc1ccccc1)(C(=O)O)c1cccc(OC(F)(F)F)c1] |
| Compound 122 | 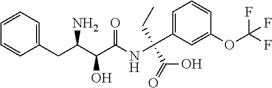 [SMILES: CC[C@@](NC(=O)[C@@H](O)[C@H](N)Cc1ccccc1)(C(=O)O)c1cccc(OC(F)(F)F)c1] |
| Compound 123 | 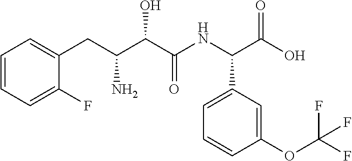 [SMILES: N[C@H](Cc1ccccc1F)[C@H](O)C(=O)N[C@H](C(=O)O)c1cccc(OC(F)(F)F)c1] |
| Compound 124 | 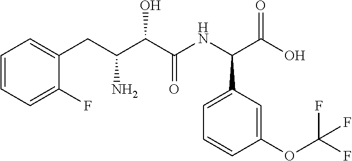 [SMILES: N[C@H](Cc1ccccc1F)[C@H](O)C(=O)N[C@@H](C(=O)O)c1cccc(OC(F)(F)F)c1] |
| Compound 125 | 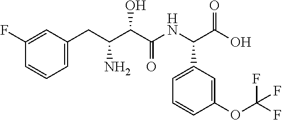 [SMILES: N[C@H](Cc1cccc(F)c1)[C@H](O)C(=O)N[C@H](C(=O)O)c1cccc(OC(F)(F)F)c1] |
| Compound 126 | 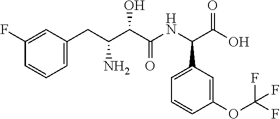 [SMILES: N[C@H](Cc1cccc(F)c1)[C@H](O)C(=O)N[C@@H](C(=O)O)c1cccc(OC(F)(F)F)c1] |
| Compound 127 | 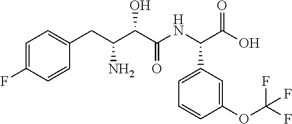 [SMILES: N[C@H](Cc1ccc(F)cc1)[C@H](O)C(=O)N[C@H](C(=O)O)c1cccc(OC(F)(F)F)c1] |
| Compound 128 | 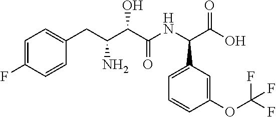 [SMILES: N[C@H](Cc1ccc(F)cc1)[C@H](O)C(=O)N[C@@H](C(=O)O)c1cccc(OC(F)(F)F)c1] |
| Compound 129 | 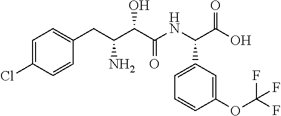 [SMILES: N[C@H](Cc1ccc(Cl)cc1)[C@H](O)C(=O)N[C@H](C(=O)O)c1cccc(OC(F)(F)F)c1] |
| Compound 130 | 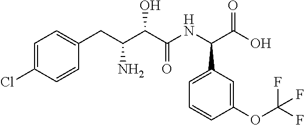 [SMILES: N[C@H](Cc1ccc(Cl)cc1)[C@H](O)C(=O)N[C@@H](C(=O)O)c1cccc(OC(F)(F)F)c1] |
| Compound 131 | 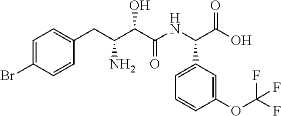 [SMILES: N[C@H](Cc1ccc(Br)cc1)[C@H](O)C(=O)N[C@H](C(=O)O)c1cccc(OC(F)(F)F)c1] |
| Compound 132 | 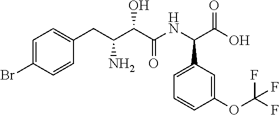 [SMILES: N[C@H](Cc1ccc(Br)cc1)[C@H](O)C(=O)N[C@@H](C(=O)O)c1cccc(OC(F)(F)F)c1] |
| Compound 133 | 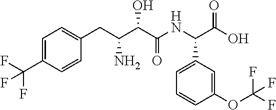 [SMILES: N[C@H](Cc1ccc(C(F)(F)F)cc1)[C@H](O)C(=O)N[C@H](C(=O)O)c1cccc(OC(F)(F)F)c1] |
| Compound 134 | 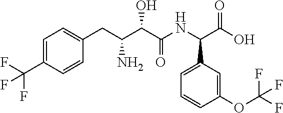 [SMILES: N[C@H](Cc1ccc(C(F)(F)F)cc1)[C@H](O)C(=O)N[C@@H](C(=O)O)c1cccc(OC(F)(F)F)c1] |
| Compound 135 | 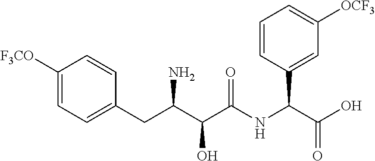 [SMILES: N[C@H](Cc1ccc(OC(F)(F)F)cc1)[C@H](O)C(=O)N[C@H](C(=O)O)c1cccc(OC(F)(F)F)c1] |

### Pharmaceutical Compositions and Administration

### General

In some embodiments, a chemical entity (e.g., a compound that inhibits (e.g., antagonizes) CNDP2, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-o-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.01 mg/Kg to about 100 mg/Kg; from about 0.01 mg/Kg to about 10 mg/Kg; from about 0.01 mg/Kg to about 1 mg/Kg; from about 0.01 mg/Kg to about 0.1 mg/Kg; from about 0.1 mg/Kg to about 100 mg/Kg; from about 0.1 mg/Kg to about 10 mg/Kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

In some embodiments, methods for treating a subject having condition, disease or disorder in which increased (e.g., excessive) CNDP2 activity contributes to the pathology and/or symptoms and/or progression of the condition, disease or disorder (e.g., immune disorders, cancer) are provided.

### Indications

In some embodiments, the condition, disease or disorder is cancer. Non-limiting examples of cancer include melanoma, carcinoma, lymphoma, blastoma, sarcoma, and leukemia or lymphoid malignancies. More particular examples of such cancers include breast cancer, colon cancer, rectal cancer, colorectal cancer, kidney or renal cancer, clear cell cancer lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, prostatic neoplasms, liver cancer, bladder cancer, cancer of the peritoneum, hepatocellular cancer, gastric or stomach cancer including gastrointestinal cancer, gastrointestinal stromal tumor, pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma, myelodysplasia disorders, myeloproliferative disorders, chronic myelogenous leukemia, and acute hematologic malignancies, endometrial or uterine carcinoma, endometriosis, endometrial stromal sarcoma, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, mast cell sarcoma, ovarian sarcoma, uterine sarcoma, melanoma, malignant mesothelioma, skin carcinomas, Schwannoma, oligodendroglioma, neuroblastomas, neuroectodermal tumor, rhabdomyosarcoma, osteogenic sarcoma, leiomyosarcomas, Ewing Sarcoma, peripheral primitive neuroectodermal tumor, urinary tract carcinomas, thyroid carcinomas, Wilm's tumor, as well as abnormal vascular proliferation associated with phakomatoses, edema (such as that associated with brain tumors), and Meigs' syndrome. In some cases, the cancer is melanoma.

In some embodiments, the cancer is selected from the group consisting of: ovarian cancer, colorectal cancer, and esophageal cancer, melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, glioblastoma multiforme, endometrial cancers, and hepatocellular carcinoma.

Still other examples can include those indications discussed herein and below in contemplated combination therapy regimens.

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the methods described herein can further include administering one or more additional cancer therapies.

The one or more additional cancer therapies can include, without limitation, surgery, radiotherapy, chemotherapy, toxin therapy, immunotherapy, cryotherapy, cancer vaccines (e.g., HPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the one or more additional cancer therapies is chemotherapy, which can include administering one or more additional chemotherapeutic agents.

In certain embodiments, the additional chemotherapeutic agent is an alkylating agent. Alkylating agents are so named because of their ability to alkylate many nucleophilic functional groups under conditions present in cells, including, but not limited to cancer cells. In a further embodiment, an alkylating agent includes, but is not limited to, Cisplatin, carboplatin, mechlorethamine, cyclophosphamide, chlorambucil, ifosfamide and/or oxaliplatin. In an embodiment, alkylating agents can function by impairing cell function by forming covalent bonds with the amino, carboxyl, sulfhydryl, and phosphate groups in biologically important molecules or they can work by modifying a cell's DNA. In a further embodiment an alkylating agent is a synthetic, semisynthetic or derivative.

In certain embodiments, the additional chemotherapeutic agent is an antimetabolite. Anti-metabolites masquerade as purines or pyrimidines, the building-blocks of DNA and in general, prevent these substances from becoming incorporated in to DNA during the “S” phase (of the cell cycle), stopping normal development and division. Anti-metabolites can also affect RNA synthesis. In an embodiment, an antimetabolite includes, but is not limited to azathioprine and/or mercaptopurine. In a further embodiment an antimetabolite is a synthetic, semisynthetic or derivative.

In certain embodiments, the additional chemotherapeutic agent is a plant alkaloid and/or terpenoid. These alkaloids are derived from plants and block cell division by, in general, preventing microtubule function. In an embodiment, a plant alkaloid and/or terpenoid is a vinca alkaloid, a podophyllotoxin and/or a taxane. Vinca alkaloids, in general, bind to specific sites on tubulin, inhibiting the assembly of tubulin into microtubules, generally during the M phase of the cell cycle. In an embodiment, a vinca alkaloid is derived, without limitation, from the Madagascar periwinkle, Catharanthus roseus (formerly known as Vinca rosea). In an embodiment, a vinca alkaloid includes, without limitation, Vincristine, Vinblastine, Vinorelbine and/or Vindesine. In an embodiment, a taxane includes, but is not limited, to Taxol, Paclitaxel and/or Docetaxel. In a further embodiment a plant alkaloid or terpernoid is a synthetic, semisynthetic or derivative. In a further embodiment, a podophyllotoxin is, without limitation, an etoposide and/or teniposide. In an embodiment, a taxane is, without limitation, docetaxel and/or ortataxel. [021] In an embodiment, a cancer therapeutic is a topoisomerase. Topoisomerases are essential enzymes that maintain the topology of DNA. Inhibition of type I or type II topoisomerases interferes with both transcription and replication of DNA by upsetting proper DNA supercoiling. In a further embodiment, a topoisomerase is, without limitation, a type I topoisomerase inhibitor or a type II topoisomerase inhibitor. In an embodiment a type I topoisomerase inhibitor is, without limitation, a camptothecin. In another embodiment, a camptothecin is, without limitation, exatecan, irinotecan, lurtotecan, topotecan, BNP 1350, CKD 602, DB 67 (AR67) and/or ST 1481. In an embodiment, a type II topoisomerase inhibitor is, without limitation, epipodophyllotoxin. In a further embodiment an epipodophyllotoxin is, without limitation, an amsacrine, etoposid, etoposide phosphate and/or teniposide. In a further embodiment a topoisomerase is a synthetic, semisynthetic or derivative, including those found in nature such as, without limitation, epipodophyllotoxins, substances naturally occurring in the root of American Mayapple (Podophyllum peltatum).

In certain embodiments, the additional chemotherapeutic agent is a stilbenoid. In a further embodiment, a stilbenoid includes, but is not limited to, Resveratrol, Piceatannol, Pinosylvin, Pterostilbene, Alpha-Viniferin, Ampelopsin A, Ampelopsin E, Diptoindonesin C, Diptoindonesin F, Epsilon- Vinferin, Flexuosol A, Gnetin H, Hemsleyanol D, Hopeaphenol, Trans-Diptoindonesin B, Astringin, Piceid and Diptoindonesin A. In a further embodiment a stilbenoid is a synthetic, semisynthetic or derivative.

In certain embodiments, the additional chemotherapeutic agent is a cytotoxic antibiotic. In an embodiment, a cytotoxic antibiotic is, without limitation, an actinomycin, an anthracenedione, an anthracycline, thalidomide, dichloroacetic acid, nicotinic acid, 2-deoxyglucose and/or chlofazimine. In an embodiment, an actinomycin is, without limitation, actinomycin D, bacitracin, colistin (polymyxin E) and/or polymyxin B. In another embodiment, an antracenedione is, without limitation, mitoxantrone and/or pixantrone. In a further embodiment, an anthracycline is, without limitation, bleomycin, doxorubicin (Adriamycin), daunorubicin (daunomycin), epirubicin, idarubicin, mitomycin, plicamycin and/or valrubicin. In a further embodiment a cytotoxic antibiotic is a synthetic, semisynthetic or derivative.

In certain embodiments, the additional chemotherapeutic agent is selected from endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, signal transduction inhibitors, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-3), vasculostatin, vasostatin (calreticulin fragment) and the like.

In certain embodiments, the additional chemotherapeutic agent is selected from abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In certain embodiments, the additional chemotherapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the additional chemotherapeutic agent can be selected from those delineated in U.S. Pat. No. 7,927,613, which is incorporated herein by reference in its entirety.

In some embodiments, an additional therapeutic agent is metformin, insulin, sulfonylureas (such as glyburide, glipizide, and glimepiride), meglitinides (such as repaglinide and nateglinide), thiazolidinediones (such as rosiglitazone and pioglitazone), DPP-4 inhibitors (such as sitagliptin, saxagliptin, and linagliptin), GLP-1 receptor agonists (such as exenatide, liraglutide, and semaglutide), and SGLT2 inhibitors (such as canagliflozin, dapagliflozin, and empagliflozin). In some embodiments, the therapeutic agent is metformin, insulin, glyburide, glipizide, glimepiride, repaglinide, nateglinide, rosiglitazone, pioglitazone, sitagliptin, saxagliptin, linagliptin, exenatide, liraglutide, semaglutide, canagliflozin, dapagliflozin, or empagliflozin. In some embodiments, the therapeutic agent is metformin. In some embodiments, the therapeutic agent is insulin. In some embodiments, the therapeutic agent is glyburide. In some embodiments, the therapeutic agent is glipizide. In some embodiments, the therapeutic agent is glimepiride. In some embodiments, the therapeutic agent is repaglinide. In some embodiments, the therapeutic agent is nateglinide. In some embodiments, the therapeutic agent is rosiglitazone. In some embodiments, the therapeutic agent is pioglitazone. In some embodiments, the therapeutic agent is sitagliptin. In some embodiments, the therapeutic agent is saxagliptin. In some embodiments, the therapeutic agent is linagliptin. In some embodiments, the therapeutic agent is exenatide. In some embodiments, the therapeutic agent is liraglutide. In some embodiments, the therapeutic agent is semaglutide. In some embodiments, the therapeutic agent is canagliflozin. In some embodiments, the therapeutic agent is dapagliflozin. In some embodiments, the therapeutic agent is empagliflozin.

In certain embodiments, the therapeutic agent is GLUCOPHAGE® or GLUMETZA® (metformin), a sulfonylurea (DIABETA® or GLYNASE® (glyburide), GLUCOTROL® (glipizide), and AMARYL® (glimepiride)), a meglitinide (PRANDIN® (repaglinide) and STARLIX® (nateglinide)), a thiazolidinediones (AVANDIA® (rosiglitazone) and ACTOS® (pioglitazone)), a dipeptidyl peptidase-4 (DPP-4) inhibitor (JANUVIA® (sitagliptin), ONGLYZA® (saxagliptin) and TRADJENTA® (linagliptin)), a glucagon-like peptide-1 (GLP-1) receptor agonist (BYETTA® (exenatide) and VICTOZA® (liraglutide)), an SGLT2 inhibitor (INVOKANA® (canagliflozin) and FARXIGA® (dapagliflozin)), or APIDRA® (insulin glulisine), HUMALOG® (insulin lispro), NOVOLOG® (insulin aspart), LANTUS® (insulin glargine), LEVEMIR® (insulin detemir), or HUMULIN® N or NOVOLIN® N (insulin isophane), PRALUENT® (alirocumab), or any combination thereof. In some embodiments, the therapeutic agent is PRALUENT® (alirocumab).

In certain embodiments, the therapeutic agent is metformin, a sulfonylurea (glyburide, glipizide, or glimepiride), a meglitinide (repaglinide or nateglinide), a thiazolidinediones (rosiglitazone or pioglitazone), a dipeptidyl peptidase-4 (DPP-4) inhibitor (sitagliptin, saxagliptin, or linagliptin), a glucagon-like peptide-1 (GLP-1) receptor agonist (exenatide or liraglutide), an SGLT2 inhibitor (canagliflozin or dapagliflozin), or an insulin (glulisine, insulin lispro, insulin aspart, insulin glargine, insulin detemir, or insulin isophane), or alirocumab, or any combination thereof. In some embodiments, the therapeutic agent is alirocumab.

In some embodiments, the therapeutic agent is an miRNAs, e.g., miR-30c, miR-126, miR-155-5p. In some embodiments, the therapeutic agent is fibroblast growth factor 21 (FGF21) or growth differentiation factor-15 (GDF-15).

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

### Compound Preparation

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and R G M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

### EXAMPLES

The following abbreviations have the indicated meanings:

| | |
|---|---|
| Boc.anhydride | Di-tert-butyl dicarbonate |
| DCC | N,N′-Dicyclohexylcarbodiimide |
| DCM | Dichloromethane |
| DIPEA | Diisopropylethylamine |
| DMF | Dimethylformamide |
| DMSO | Dimethylsulfoxide |
| h | Hour(s) |
| HPLC | High-Performance Liquid Chromatography |
| LC-MS | Liquid chromatography-mass spectrometry |
| NMR | Nuclear Magnetic Resonance |
| NMR (splitting) | s - singlet, m- multiplet, d-doublet, dd-doublet- |
| | of-doublet, br s - broad singlet, br d- - broad |
| | doublet |
| ppm | Parts Per Million |
| TEA | Triethylamine |
| TLC | Thin Layer Chromatography |

### Materials and Methods

### PREPARATIVE EXAMPLES

### Example 1: Synthesis of (S)-2-((2S,3R)-3-amino-2-hydroxy-4-phenylbutanamido)-3-(4-fluorophenyl)propanoic acid (Compound 1)

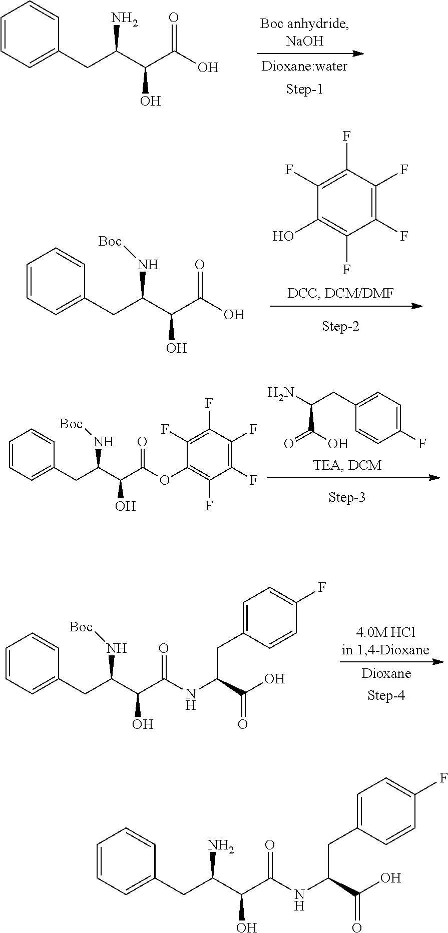

### Step 1: Synthesis of (2R,3R)-3-((tert-butoxycarbonyl)amino)-2-hydroxy-4-phenylbutanoic acid

To a stirred solution (2S,3R)-3-amino-2-hydroxy-4-phenylbutanoic acid (10 g, 51.28 mmol) in 1,4 dioxane: water (1:1) (100 mL) was added NaOH (6 g, 153.84 mmol), followed by the addition of di-tert-butyl dicarbonate (18 mL, 76.92 mmol). The reaction mixture was stirred at room temperature for 16 h and the progress of the reaction was monitored by TLC. After completion of the reaction, the solvent was removed under reduced pressure and treated with 1M solution of KHSO₄ (30 mL). The resultant mixture was then extracted with EtOAc (2×200 mL). The combined organic layer was washed with water and concentrated under reduced pressure to afford (2R,3R)-3-((tert-butoxycarbonyl)amino)-2-hydroxy-4-phenylbutanoic acid (15 g, crude). LCMS: calculated for C₁₅H₂₁NO₅ is 295.14; found 294.1 (M−1).

### Step 2: Synthesis of (2,3,4,5,6-pentafluorophenyl) (2S,3R)-3-(tert-butoxycarbonylamino)-2-hydroxy-4-phenyl-butanoate

To a stirred solution of (2S,3R)-3-((tert-butoxycarbonyl)amino)-2-hydroxy-4-phenylbutanoic acid (0.5 g, 1.69 mmol) in DMF (5 mL) were added a solution of pentafluorophenol (374.0 mg, 2.03 mmol) in DCM (2 mL) and a solution of DCC (696.0 mg, 3.38 mmol) in DCM (3.0 mL). The resultant reaction mixture was stirred at room temperature for 2 h. The progress of the reaction was monitored by TLC. After completion, the reaction mixture was filtered, and the residue was washed with pentane and dried under reduced pressure to afford (2,3,4,5,6-pentafluorophenyl) (2S,3R)-3-(tert-butoxycarbonylamino)-2-hydroxy-4-phenyl-butanoate (1.4 g, crude). LCMS: calculated for C₂₁H₂₀F₅NO₅ is 461.13; found 361.2 (M−100).

### Step 3: Synthesis of (S)-2-((2S,3R)-3-((tert-butoxycarbonyl)amino)-2-hydroxy-4-phenylbutanamido)-3-(4-fluorophenyl)propanoic acid

To a stirred solution of (2,3,4,5,6-pentafluorophenyl) (2S,3R)-3-(tert-butoxycarbonylamino)-2-hydroxy-4-phenyl-butanoate (1.0 g, 2.16 mmol) in DCM (20 mL) was added triethylamine (1.0 mL, 6.48 mmol). To this was added (S)-2-amino-3-(4-fluorophenyl)propanoic acid (0.6 g, 3.25 mmol) and the reaction mixture was stirred at room temperature for 16 h. The progress of the reaction was monitored by TLC. After completion, the reaction mixture was concentrated under reduced pressure. The reaction mixture was quenched with KHSO₄ solution (30.0 mL) and extracted with EtOAc (2×200 mL). The combined organic layer was washed with water and concentrated under vacuum pressure to get crude product. The crude product was purified by prep-HPLC [(Column: xbridge phenyl (C18, 20 mm×150 mm)), mobile phase: A=0.1% HCOOH in water, Phase B=MeCN, Flow: 12 ml/min, Gradient Programme: Time-0,2,10% B-40,50,60), followed by lyophilization of the fractions] to afford (S)-2-((2S,3R)-3-((tert-butoxycarbonyl)amino)-2-hydroxy-4-phenylbutanamido)-3-(4-fluorophenyl)propanoic acid (150 mg). LCMS: calculated for C₂₄H₂₉FN₂O₆ is 460.20; found 459.05 (M−1).

### Step 4: Synthesis of (S)-2-((2S,3R)-3-amino-2-hydroxy-4-phenylbutanamido)-3-(4-fluorophenyl)propanoic acid (Compound 1)

To a stirred solution of (S)-2-((2S,3R)-3-((tert-butoxycarbonyl)amino)-2-hydroxy-4-phenylbutanamido)-3-(4-fluorophenyl)propanoic acid (100 mg, 0.217 mmol) was added HCl in 1,4-Dioxane (5 mL) at 0° C. and the reaction mixture was stirred at room temperature for 1 h. The progress of the reaction was monitored by TLC. After completion the reaction mixture was evaporated to dryness under vacuum to get the crude. The crude was washed with diethyl ether to afford (S)-2-((2S,3R)-3-amino-2-hydroxy-4-phenylbutanamido)-3-(4-fluorophenyl)propanoic acid as off-white solid (30 mg, 78%). ¹H NMR (400 MHz, DMSO-d₆) δ 2.54-2.62 (m, 1H) 2.81 (dd, J=13.86, 7.26 Hz, 1H) 2.97-3.16 (m, 2H) 3.42 (br d, J=3.37 Hz, 1H) 3.88-4.02 (m, 1H) 4.39-4.55 (m, 1H) 6.54-6.73 (m, 1H) 6.99-7.11 (m, 2H) 7.12-7.31 (m, 5H) 7.32-7.42 (m, 2H) 8.07-8.21 (m, 1H) ppm; LCMS: calculated for C₁₉H₂₁FN₂O₄ is 360.15, found: 359 (M−1).

### Example 2: Synthesis of (2S,3R)—N-((1H-tetrazol-5-yl)methyl)-3-amino-2-hydroxy-4-phenylbutanamide (Compound 2)

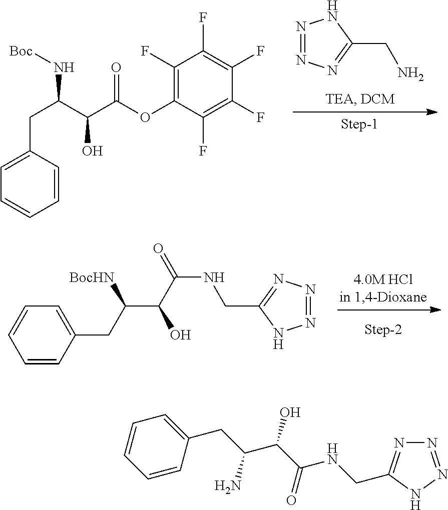

### Step 1: Synthesis of tert-butyl ((2R,3S)-4-(((1H-tetrazol-5-yl)methyl)amino)-3-hydroxy-4-oxo-1-phenylbutan-2-yl)carbamate

The solution of (2,3,4,5,6-pentafluorophenyl) (2S,3R)-3-(tert-butoxycarbonylamino)-2-hydroxy-4-phenyl-butanoate (750 mg, 1.62 mmol) in DCM (5 mL) were added a solution of (1H-tetrazol-5-yl)methanamine (193 mg, 1.95 mmol) in DCM (5 mL) and triethylamine (0.68 mL, 4.86 mmol) at room temperature and the resultant mixture was stirred for 16 h. The progress of the reaction was monitored by TLC. After completion the reaction mixture was quenched with ice-water and extracted with EtOAc. The organic layer was washed with water and concentrated under vacuum to afford tert-butyl ((2R,3S)-4-(((1H-tetrazol-5-yl)methyl)amino)-3-hydroxy-4-oxo-1-phenylbutan-2-yl)carbamate (500 mg, crude). LCMS: calculated for C₁₇H₂₄N₆O₄ is 376.19; found 375.2 (M−1).

### Step 2: Synthesis of (2S,3R)—N-((1H-tetrazol-5-yl)methyl)-3-amino-2-hydroxy-4-phenylbutanamide

To a stirred solution of tert-butyl ((2R,3S)-4-(((1H-tetrazol-5-yl)methyl)amino)-3-hydroxy-4-oxo-1-phenylbutan-2-yl)carbamate (40 mg, 1.32 mmol) in 1,4-dioxane (1 mL) was added HCl in 1,4-dioxane (1 mL, 20 vol) at 0° C. and the reaction mixture was stirred at room temperature for 16 h. The progress of the reaction was monitored by TLC. After completion, the reaction mixture was evaporated to dryness under vacuum to get the crude product. The crude was washed with diethyl ether to afford (2S,3R)—N-((1H-tetrazol-5-yl)methyl)-3-amino-2-hydroxy-4-phenylbutanamide.HCl as a white solid (50 mg, 13.6%). ¹H NMR (400 MHz, DMSO-d₆) δ 2.85-2.95 (m, 2H) 3.64-3.72 (m, 2H) 4.00 (br s, 1H) 4.46-4.55 (m, 1H) 4.60-4.70 (m, 1H) 6.78-6.91 (m, 1H) 7.25-7.41 (m, 5H) 7.97 (br s, 3H) 8.73-8.87 (m, 1H) ppm; LCMS: calculated for C₁₂H₁₆N₆O₂ is 276.13, found 277.2 (M+1).

### Example 3: Synthesis of 2-[[(2S,3R)-3-amino-2-hydroxy-4-phenyl-butanoyl]amino]-2-phenyl-acetic acid (Compound 3)

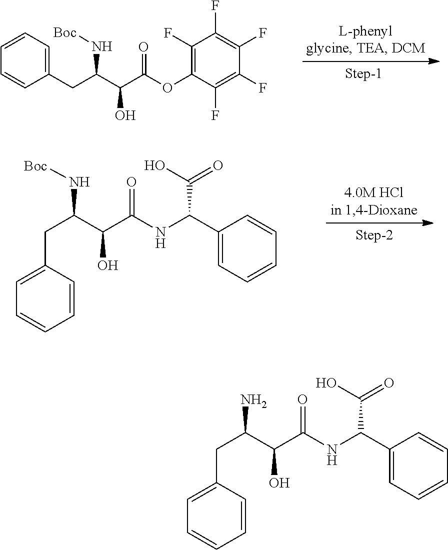

### Step 1: Synthesis of (2S)-2-[[(2S,3R)-3-(tert-butoxycarbonylamino)-2-hydroxy-4-phenyl-butanoyl]amino]-2-phenyl-acetic acid

Following the protocol described for compound 1, (2S)-2-[[(2S,3R)-3-(tert-butoxycarbonylamino)-2-hydroxy-4-phenyl-butanoyl]amino]-2-phenyl-acetic acid (280 mg, crude) was obtained as an off-white solid. LCMS: calculated for C₂₃H₂₈N₂O₆ is 428.19; found 427.05 (M−1).

### Step 2: Synthesis of 2-[[(2S,3R)-3-amino-2-hydroxy-4-phenyl-butanoyl]amino]-2-phenyl-acetic acid

Following the protocol described for compound 1, the product 2-[[(2S,3R)-3-amino-2-hydroxy-4-phenyl-butanoyl]amino]-2-phenyl-acetic acid was isolated in crude form. This crude product was purified by prep-HPLC [HPLC (Column: X-Bridge (C18, 19 mm×150 mm), mobile phase: A=0.1% HCOOH in water, Phase B=MeCN, Flow: 15 ml/min, Gradient Programme: Time-0, 2, 10%, B-10, 20, 60), followed by lyophilization of the fractions] to afford pure 2-[[(2S,3R)-3-amino-2-hydroxy-4-phenyl-butanoyl]amino]-2-phenyl-acetic acid as white solid (20 mg, 50%). ¹H NMR (400 MHz, DMSO-d₆) δ 2.71 (br dd, J=13.06, 6.31 Hz, 1H) 2.93 (br dd, J=13.20, 8.22 Hz, 1H) 3.59 (br s, 1H) 3.95 (d, J=2.93 Hz, 1H) 4.87-4.93 (m, 1H) 7.19-7.28 (m, 6H) 7.29-7.36 (m, 5H) 8.31-8.53 (m, 1H) ppm; LCMS: calculated for C₁₈H₂₀N₂O₄ is 328.14 and found 329.21 (M+1).

### Example 4: Synthesis of (2S)-2-[[(2S,3R)-3-amino-2-hydroxy-4-phenyl-butanoyl]amino]-2-cyclopentyl-acetic acid (Compound 4)

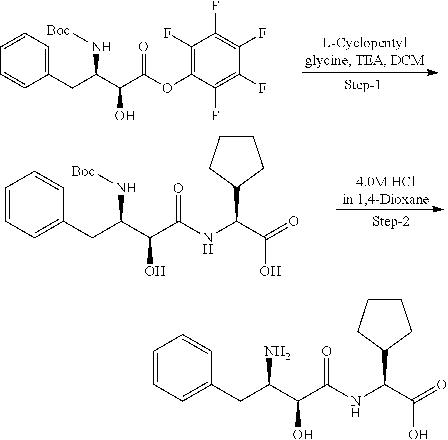

### Step 1: Synthesis of (2S)-2-[[(2S,3R)-3-(tert-butoxycarbonylamino)-2-hydroxy-4-phenyl-butanoyl]amino]-2-cyclopentyl-acetic acid

Following the protocol described for compound 1, the compound (2S)-2-[[(2S,3R)-3-(tert-butoxycarbonylamino)-2-hydroxy-4-phenyl-butanoyl]amino]-2-cyclopentyl-acetic acid was isolated in crude form. The crude product was then purified by prep-HPLC (Column: X-Bridge (C18, 19 mm×150 mm), mobile phase: A=0.1% HCOOH in water, Phase B=MeCN, Flow: 15 ml/min, Gradient Programme: Time-0,2,10% B-10,20,60), followed by lyophilization of the fractions to afford pure compound (40 mg). LCMS: calculated for C₂₂H₃₂N₂O₆ is 420.23; found 419.15 (M−1).

### Step 2: Synthesis of (2S)-2-[[(2S,3R)-3-amino-2-hydroxy-4-phenyl-butanoyl]amino]-2-cyclopentyl-acetic acid

Following the protocol described for compound 1, the product (2S)-2-[[(2S,3R)-3-amino-2-hydroxy-4-phenyl-butanoyl]amino]-2-cyclopentyl-acetic acid was obtained in crude form. The crude was then purified by prep-HPLC (Column: X-Bridge (C18, 19 mm×150 mm), mobile phase: A=0.1% HCOOH in water, Phase B=MeCN, Flow: 15 ml/min, Gradient Programme: Time-0,2,10% B-10,20,60), followed by lyophilization of the fractions to afford pure product (2 mg, 6%) as white solid. ¹H NMR (400 MHz, Methanol-d₄) δ 1.28-1.49 (m, 2H) 1.51-1.69 (m, 4H) 1.69-1.82 (m, 2H) 2.28-2.36 (m, 1H) 2.91 (dd, J=13.64, 7.34 Hz, 1H) 3.08-3.17 (m, 1H) 3.77 (td, J=7.48, 3.23 Hz, 1H) 4.12 (d, J=3.08 Hz, 1H) 4.17 (d, J=7.19 Hz, 1H) 7.21-7.42 (m, 5H) ppm; LCMS: Calculated for C₁₇H₂₄N₂O₄ is 320.17; found 321.2 (M+1).

### Example 5: Synthesis of (2S)-2-[[(2S,3R)-3-amino-2-hydroxy-4-phenyl-butanoyl]amino]-2-cyclohexyl-acetic acid (Compound 5)

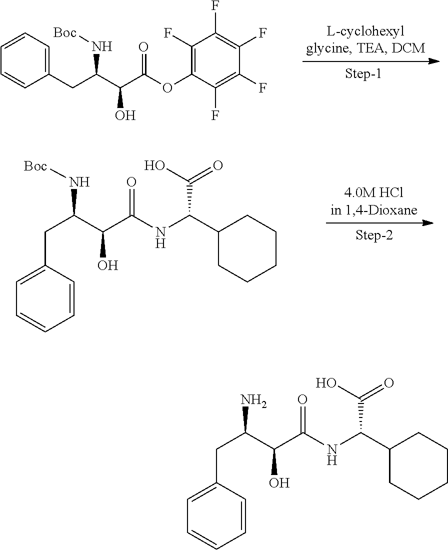 [SMILES: CC(C)(C)OC(=O)N[C@H](Cc1ccccc1)[C@H](O)C(=O)Oc1c(F)c(F)c(F)c(F)c1F ; CC(C)(C)OC(=O)N[C@H](Cc1ccccc1)[C@H](O)C(=O)N[C@H](C(=O)O)C1CCCCC1 ; N[C@H](Cc1ccccc1)[C@H](O)C(=O)N[C@H](C(=O)O)C1CCCCC1]

### Step 1: Synthesis of (2S)-2-[[(2S,3R)-3-(tert-butoxycarbonylamino)-2-hydroxy-4-phenyl-butanoyl]amino]-2-cyclohexyl-acetic acid

Following the protocol described for compound 1, a crude form of (2S)-2-[[(2S,3R)-3-(tert-butoxycarbonylamino)-2-hydroxy-4-phenyl-butanoyl]amino]-2-cyclohexyl-acetic acid was isolated. This crude mixture was purified by prep-HPLC (Column: ZORBAX (C18.20 mm×250 mm), mobile phase: A=0.1% HCOOH in water, Phase B=MeCN, Flow: 15 ml/min, Gradient Programme: Time-0,2,6,10% B-35,45,65,65), followed by lyophilization of the fractions, to afford the pure compound (30 mg, 12%). ¹H NMR (400 MHz, DMSO-d₆) δ 1.02-1.18 (m, 6H) 1.28 (s, 7H) 1.56 (br d, J=11.15 Hz, 3H) 1.66 (br s, 2H) 2.73-2.84 (m, 1H) 3.83 (br s, 1H) 3.88-4.00 (m, 1H) 4.09 (br s, 1H) 6.11 (br d, J=5.14 Hz, 1H) 6.19 (brd, J=9.24 Hz, 1H) 7.19-7.28 (m, 4H) 7.46-7.56 (m, 1H) ppm; LCMS: calculated for C₂₃H₃₄N₂O₆ is 434.24; found 433.10 (M−1).

### Step 2: Synthesis of (2S)-2-[[(2S,3R)-3-amino-2-hydroxy-4-phenyl-butanoyl]amino]-2-cyclohexyl-acetic acid

Following the protocol described for compound 1, the compound (2S)-2-[[(2S,3R)-3-amino-2-hydroxy-4-phenyl-butanoyl]amino]-2-cyclohexyl-acetic acid (10 mg, 33.3%) was isolated in pure form. ¹H NMR (300 MHz, DMSO-d₆) δ 0.94-1.30 (m, 4H) 1.48-1.80 (m, 4H) 2.33 (s, 1H) 2.68-2.85 (m, 1H) 2.88-3.00 (m, 1H) 3.45-3.60 (m, 1H) 3.95-4.06 (m, 1H) 4.11 (br dd, J=7.58, 5.89 Hz, 1H) 6.52-6.79 (m, 1H) 7.17-7.50 (m, 3H) 7.90 (br s, 2H) 8.02-8.17 (m, 1H) 12.31-13.03 (m, 2H) ppm; LCMS: calculated for C₁₈H₂₆N₂O₄ is 334.19 and found 335.20 (M+1).

### Example 6: Synthesis of (2S,3R)-3-amino-2-hydroxy-N-[(1S)-3-methyl-1-(1H-tetrazol-5-yl)butyl]-4-phenyl-butanamide (Compound 6)

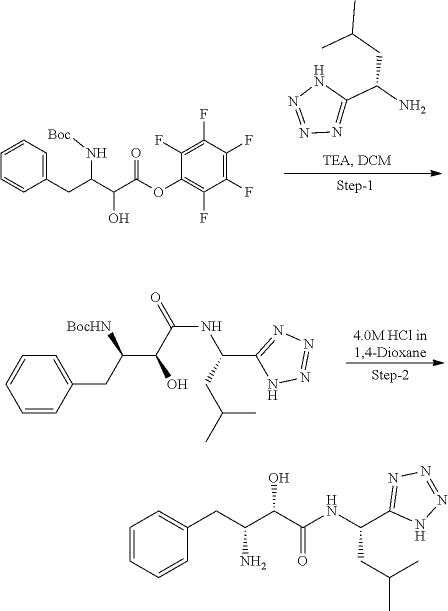 [SMILES: CC(C)(C)OC(=O)NC(Cc1ccccc1)C(O)C(=O)Oc1c(F)c(F)c(F)c(F)c1F ; CC(C)C[C@H](N)c1nnn[nH]1 ; CC(C)C[C@H](NC(=O)[C@@H](O)[C@@H](Cc1ccccc1)NC(=O)OC(C)(C)C)c1nnn[nH]1 ; CC(C)C[C@H](NC(=O)[C@@H](O)[C@H](N)Cc1ccccc1)c1nnn[nH]1]

### Step 1: Synthesis of tert-butyl N-[(1R,2S)-1-benzyl-2-hydroxy-3-[[(1S)-3-methyl-1-(1H-tetrazol-5-yl)butyl]amino]-3-oxo-propyl]carbamate

Following the protocol described for the compound 2, the compound tert-butyl N-[(1R,2S)-1-benzyl-2-hydroxy-3-[[(1S)-3-methyl-1-(1H-tetrazol-5-yl)butyl]amino]-3-oxo-propyl]carbamate (700 mg, crude) was obtained. LCMS: calculated for C₂₁H₃₂N₆O₄ is 432.25; found 431.30 (M−1). It was taken to the next step without purification.

### Step 2: Synthesis of (2S,3R)-3-amino-2-hydroxy-N-[(1S)-3-methyl-1-(1H-tetrazol-5-yl)butyl]-4-phenyl-butanamide

Following the protocol described for the synthesis of compound 2, the compound (2S,3R)-3-amino-2-hydroxy-N-[(1S)-3-methyl-1-(1H-tetrazol-5-yl)butyl]-4-phenyl-butanamide was obtained as white solid (50 mg, 8.7%). ¹H NMR (400 MHz, DMSO-d₆) δ 0.89-0.91 (m, 6H) 1.53-1.61 (m, 1H) 1.79-1.84 (m, 2H) 2.82-2.93 (m, 2H) 3.53-3.61 (m, 2H) 4.01 (dd, J=5.36, 3.01 Hz, 1H) 5.16-5.24 (m, 1H) 6.61 (br d, J=5.72 Hz, 1H) 7.29 (br d, J=6.46 Hz, 5H) 7.75-8.02 (m, 2H) 8.67 (br d, J=7.92 Hz, 1H) 15.87-16.54 (m, 1H) ppm; LCMS: calculated for C₁₆H₂₄N₆O₂ is 332.20 and found 333.20 (M+1).

### Example 7: Synthesis of (2S,3R)-3-amino-2-hydroxy-4-phenyl-N-[(1S)-2-phenyl-1-(1H-tetrazol-5-yl)ethyl]butanamide (Compound 7)

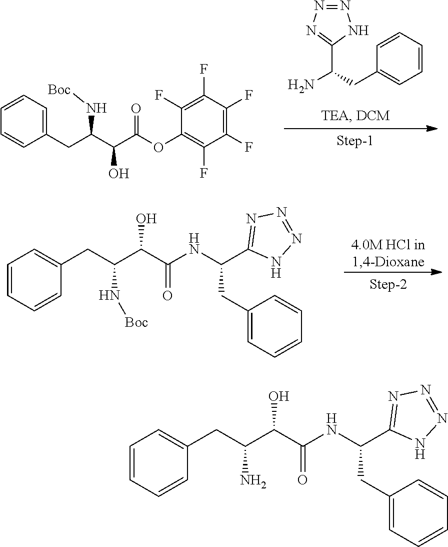

### Step 1: Synthesis of tert-butyl N-[(1R,2S)-1-benzyl-2-hydroxy-3-oxo-3-[[(1S)-2-phenyl-1-(1H-tetrazol-5-yl)ethyl]amino]propyl]carbamate

Following the protocol described for the synthesis of the compound 2, the crude product tert-butyl N-[(1R,2S)-1-benzyl-2-hydroxy-3-oxo-3-[[(1S)-2-phenyl-1-(1H-tetrazol-5-yl)ethyl]amino]propyl]carbamate was obtained. It was purified by prep-HPLC (Column: Gemini (250 mm×21.2 mm), 5.0p), mobile phase: A=0.1% HCOOH in water, Phase B=MeCN, Flow: 18 ml/min, Gradient Programme: Time-0,2,5% B-40,45,75), followed by lyophilization of the fractions) to afford tert-butyl N-[(1R,2S)-1-benzyl-2-hydroxy-3-oxo-3-[[(1S)-2-phenyl-1-(1H-tetrazol-5-yl)ethyl]amino]propyl]carbamate (20 mg, 33%). LCMS: calculated for C24H30N6O4 is 466, found: 465-1 (M−1).

### Step 2: Synthesis of (2S,3R)-3-amino-2-hydroxy-4-phenyl-N-[(1S)-2-phenyl-1-(1H-tetrazol-5-yl)ethyl]butanamide

Following the protocol described for the synthesis of the compound 2, a pure form of the product (2S,3R)-3-amino-2-hydroxy-4-phenyl-N-[(1S)-2-phenyl-1-(1H-tetrazol-5-yl)ethyl]butanamide was obtained as off-white gummy solid (10 mg, 66%). ¹H NMR (400 MHz, DMSO-d₆) δ 1.56-1.79 (m, 2H) 2.71-2.85 (m, 1H) 3.44-3.50 (m, 2H) 3.52-3.62 (m, 1H) 4.01 (br s, 1H) 4.07-4.20 (m, 1H) 5.33-5.60 (m, 1H) 6.50-6.65 (m, 1H) 7.19-7.35 (m, 8H) 7.66-7.75 (m, 1H) 8.37-8.79 (m, 1H) ppm; LCMS: calculated for C₁₉H₂₂N₆O₂ is 366.18, found: 365 (M−1).

### Example 8: Synthesis of (2S)-2-[[(2S,3R)-3-amino-2-hydroxy-4-phenyl-butanoyl]amino]-2-(2-methoxyphenyl)acetic acid (Compound 8)

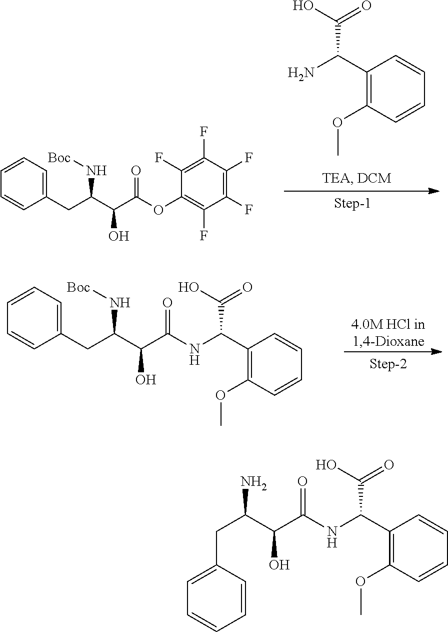 [SMILES: CC(C)(C)OC(=O)N[C@H](Cc1ccccc1)[C@H](O)C(=O)Oc1c(F)c(F)c(F)c(F)c1F ; COc1ccccc1[C@H](N)C(=O)O ; COc1ccccc1[C@H](NC(=O)[C@@H](O)[C@@H](Cc1ccccc1)NC(=O)OC(C)(C)C)C(=O)O ; COc1ccccc1[C@H](NC(=O)[C@@H](O)[C@H](N)Cc1ccccc1)C(=O)O]

### Step 1: Synthesis of (2S)-2-[[(2S,3R)-3-(tert-butoxycarbonylamino)-2-hydroxy-4-phenyl-butanoyl]amino]-2-(2-methoxyphenyl)acetic acid

Following the protocol described for the synthesis of the compound 1, the crude product (2S)-2-[[(2S,3R)-3-(tert-butoxycarbonylamino)-2-hydroxy-4-phenyl-butanoyl]amino]-2-(2-methoxyphenyl)acetic acid was obtained. It was then purified by prep-HPLC [(KINETEX (C18, 20 mm×150 mm), Phase A: 0.1% HCOOH in water, Phase B: MeCN, Gradient Elusion), followed by lyophilization] to afford (2S)-2-[[(2S,3R)-3-(tert-butoxycarbonylamino)-2-hydroxy-4-phenyl-butanoyl]amino]-2-(2-methoxyphenyl)acetic acid (30 mg, 15%). LCMS: calculated for C₂₄H₃₀N₂O₇ is 458.21 and found 458.95 (M+1).

### Step 2: Synthesis of (2S)-2-[[(2S,3R)-3-amino-2-hydroxy-4-phenyl-butanoyl]amino]-2-(2-methoxyphenyl)acetic acid

Following the protocol described for the synthesis of the compound 1, the compound (2S)-2-[[(2S,3R)-3-amino-2-hydroxy-4-phenyl-butanoyl]amino]-2-(2-methoxyphenyl)acetic acid was obtained as off-white solid (10 mg, 43.5%). ¹H NMR (400 MHz, DMSO-d₆) δ 1.00-1.06 (m, 1H) 1.09 (t, J=6.97 Hz, 1H) 1.25 (br d, J=11.30 Hz, 1H) 1.56-1.76 (m, 2H) 2.72-2.84 (m, 1H) 2.85-2.97 (m, 1H) 3.38 (br d, J=7.04 Hz, 2H) 3.53 (br s, 1H) 3.80 (s, 3H) 4.05 (dd, J=5.65, 3.45 Hz, 1H) 5.55-5.63 (m, 1H) 6.76 (d, J=6.02 Hz, 1H) 6.89-7.01 (m, 1H) 7.06 (d, J=8.07 Hz, 1H) 7.27-7.37 (m, 7H) 7.87 (br s, 3H) 8.41 (d, J=8.07 Hz, 1H) 12.46-13.03 (m, 1H) ppm; LCMS: calculated for C₁₉H₂₂N₂O₅ is 358.15 and found 359.05 (M+1).

### Example 9: Synthesis of (2S)-2-[[(2S,3R)-3-amino-2-hydroxy-4-phenyl-butanoyl]amino]-2-(3-pyridyl)acetic acid (Compound 9)

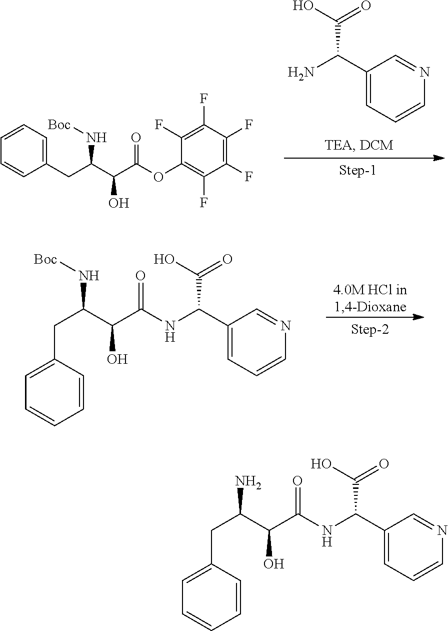 [SMILES: CC(C)(C)OC(=O)N[C@H](Cc1ccccc1)[C@H](O)C(=O)Oc1c(F)c(F)c(F)c(F)c1F ; N[C@H](C(=O)O)c1cccnc1 ; CC(C)(C)OC(=O)N[C@H](Cc1ccccc1)[C@H](O)C(=O)N[C@H](C(=O)O)c1cccnc1 ; N[C@H](Cc1ccccc1)[C@H](O)C(=O)N[C@H](C(=O)O)c1cccnc1]

### Step 1: Synthesis of (2S)-2-[[(2S,3R)-3-(tert-butoxycarbonylamino)-2-hydroxy-4-phenyl-butanoyl]amino]-2-(3-pyridyl)acetic acid

Following the protocol described for the synthesis of the compound 1, the crude compound was obtained. It was purified by prep-HPLC [(Column: Xbridge phenyl (C18, 20 mm×150 mm)5.0μ), Phase A: 0.1% HCOOH in water, Phase B: MeCN, Gradient Elusion), followed by lyophilization of the fractions] to afford (2S)-2-[[(2S,3R)-3-(tert-butoxycarbonylamino)-2-hydroxy-4-phenyl-butanoyl]amino]-2-(3-pyridyl)acetic acid (20 mg, 10%). ¹H NMR (400 MHz, DMSO-d₆) δ 0.99-1.11 (m, 2H) 1.22 (s, 9H) 1.41-1.54 (m, 1H) 1.56-1.77 (m, 3H) 2.75 (br dd, J=13.13, 6.24 Hz, 1H) 3.79 (s, 3H) 3.89 (br d, J=5.14 Hz, 2H) 5.51-5.61 (m, 2H) 6.03-6.15 (m, 2H) 6.91 (t, J=7.26 Hz, 1H) 7.02 (br d, J=8.07 Hz, 1H) 7.14-7.30 (m, 7H) 8.00-8.09 (m, 1H) 12.59-12.85 (m, 1H) ppm; LCMS: calculated for C₂₂H₂₇N₃O₆ is 429.19; found 429.95 (M+1).

### Step 2: (2S)-2-[[(2S,3R)-3-amino-2-hydroxy-4-phenyl-butanoyl]amino]-2-(3-pyridyl)acetic acid

The compound (2S)-2-[[(2S,3R)-3-amino-2-hydroxy-4-phenyl-butanoyl]amino]-2-(3-pyridyl)acetic acid was synthesized following the protocol described for the synthesis of compound 1 (yield: 10 mg, 65.3%). ¹H NMR (400 MHz, DMSO-d₆) δ 2.74-2.84 (m, 1H) 2.85-2.99 (m, 2H) 3.04-3.13 (m, 1H) 3.93-4.08 (m, 1H) 5.37-5.55 (m, 1H) 7.14-7.40 (m, 6H) 7.44-7.60 (m, 1H) 7.81-8.03 (m, 4H) 8.54-8.63 (m, 1H) 8.68 (br d, J=15.11 Hz, 1H) 8.83-8.90 (m, 1H) ppm; LCMS: calculated for C₁₇H₁₉N₃O₄ is 329.14; found 329.90 (M+1).

### Example 10: Synthesis of 2-[[(2S,3R)-3-amino-2-hydroxy-4-phenyl-butanoyl]amino]-2-[3-(trifluoro methyl)phenyl]acetic acid (Compound 10)

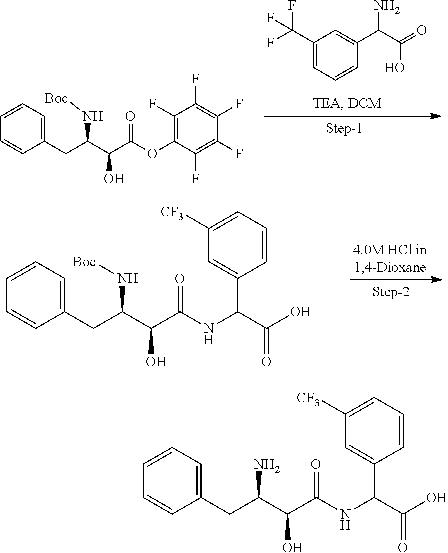

### Step 1: Synthesis of 2-[[(2S,3R)-3-(tert-butoxycarbonylamino)-2-hydroxy-4-phenyl-butanoyl]amino]-2-[3-(trifluoromethyl)phenyl]acetic acid

To a stirred solution of 2-amino-2-(3-(trifluoromethyl)phenyl)acetic acid (49.2 mg, 0.224 mmol) in DCM (1 mL) was added triethylamine (0.05 mL, 0.34 mmol) at 0° C., followed by (2,3,4,5,6-pentafluorophenyl) (2S,3R)-3-(tert-butoxycarbonylamino)-2-hydroxy-4-phenyl-butanoate (50 mg, 0.112 mmol) dissolved in DCM (1 mL). The resultant mixture was stirred at room temperature for 2 h. The progress of the reaction was monitored by TLC. After completion, the reaction mixture was concentrated under reduced pressure to afford 2-[[(2S,3R)-3-(tert-butoxycarbonylamino)-2-hydroxy-4-phenyl-butanoyl]amino]-2-[3-(trifluoromethyl)phenyl]acetic acid (114 mg, crude). LCMS: Calculated for C₂₄H₂₇F₃N₂O₆ is 496.18; found:495 (M−1).

### Step 2: Synthesis of 2-[[(2S,3R)-3-amino-2-hydroxy-4-phenyl-butanoyl]amino]-2-[3-(trifluoro methyl)phenyl]acetic acid

To a stirred solution of 2-[[(2S,3R)-3-(tert-butoxycarbonylamino)-2-hydroxy-4-phenyl-butanoyl]amino]-2-[3-(trifluoromethyl)phenyl]acetic acid (114 mg, 0.23 mmol) in 1, 4-dioxane (0.6 mL) cooled to 0° C. and was added 4 M HCl in 1,4-dioxane (0.86 mL, 3.44 mmol) and the reaction mixture was stirred at room temperature for 4 h. The progress of the reaction was monitored by TLC. After completion, the reaction mixture was concentrated under reduced pressure to get the crude product. It was then purified by prep-HPLC [method: HPLC (Column: Gemini (21.2 mm×250 mm), mobile phase: A=0.1% HCOOH in water, Phase B=MeCN, Flow: 20 ml/min, Gradient Programme: Time-0,2,8% B-10,20,45), followed by lyophilization of the fractions] to afford 2-[[(2S,3R)-3-amino-2-hydroxy-4-phenyl-butanoyl]amino]-2-[3-(trifluoro methyl)phenyl]acetic acid (2 mg, 2%) as white solid. ¹H NMR (400 MHz, DMSO-d₆) δ 7.12-7.23 (m, 2H) 7.32 (s, 1H) 7.39-7.57 (m, 1H) 7.63-7.71 (m, 1H) 8.36-8.48 (m, 1H) ppm; LCMS: Calculated for C₁₉H₁₉F₃N₂O₄ is 396.37; found 397.37 (M+1).

### Example 11: Synthesis of 2-[[(2S,3R)-3-amino-2-hydroxy-4-phenyl-butanoyl]amino]-3-[2-(trifluoro methyl)phenyl]propanoic acid (Compound 11)

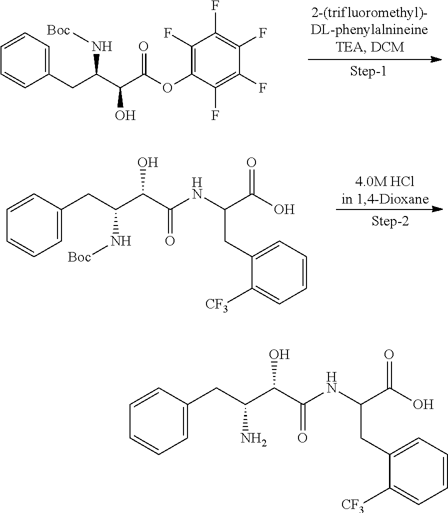 [SMILES: CC(C)(C)OC(=O)N[C@H](Cc1ccccc1)[C@H](O)C(=O)Oc1c(F)c(F)c(F)c(F)c1F ; CC(C)(C)OC(=O)N[C@H](Cc1ccccc1)[C@H](O)C(=O)NC(Cc1ccccc1C(F)(F)F)C(=O)O ; N[C@H](Cc1ccccc1)[C@H](O)C(=O)NC(Cc1ccccc1C(F)(F)F)C(=O)O]

### Step 1: Synthesis of 2-[[(2S,3R)-3-(tert-butoxycarbonylamino)-2-hydroxy-4-phenyl-butanoyl]amino]-3-[2-(trifluoromethyl)phenyl]propanoic acid

Following the protocol described for the synthesis of the compound 1, the crude 2-[[(2S,3R)-3-(tert-butoxycarbonylamino)-2-hydroxy-4-phenyl-butanoyl]amino]-3-[2-(trifluoromethyl)phenyl]propanoic acid was obtained. It was further purified by prep-HPLC [(Column: LUNA (C18, 19 mm×150 mm), mobile phase: A=0.1% HCOOH in water, Phase B=MeCN, Flow: 15 ml/min, Gradient Programme: Time-0,2,10% B-10,20,40), followed by lyophilization of the fractions] to afford the pure compound (50.0 mg, 16.66%). LCMS: calculated for C₂₅H₂₉F₃N₂O₆ is 510.20 and found 411.1 (M−99).

### Step 2: Synthesis of 2-[[(2S,3R)-3-amino-2-hydroxy-4-phenyl-butanoyl]amino]-3-[2-(trifluoro methyl)phenyl]propanoic acid

This compound was synthesized following the protocol described for the synthesis of compound 1. The crude product was washed with diethyl ether to afford pure 2-[[(2S,3R)-3-amino-2-hydroxy-4-phenyl-butanoyl]amino]-3-[2-(trifluoro methyl)phenyl]propanoic acid (10 mg, 20%) as white solid. ¹H NMR (400 MHz, DMSO-d₆) δ 2.77-2.90 (m, 1H) 3.12-3.24 (m, 2H) 3.43-3.56 (m, 1H) 4.05 (br t, J=4.25 Hz, 1H) 4.38-4.64 (m, 1H) 6.70 (br d, J=5.43 Hz, 1H) 7.21-7.31 (m, 3H) 7.33-7.44 (m, 3H) 7.54 (d, J=3.96 Hz, 2H) 7.68 (d, J=7.78 Hz, 1H) 7.78-7.97 (m, 2H) 8.51 (d, J=8.36 Hz, 1H) 11.81-13.34 (m, 1H) ppm; LCMS: calculated for C₂₀H₂₁F₃N₂O₄ is 410.15, found 411.55 (M+1).

### Example 12: Synthesis of 2-[[(2S,3R)-3-(tert-butoxycarbonylamino)-2-hydroxy-4-phenyl-butanoyl]amino]-2-methyl-3-phenyl-propanoic acid (Compound 12)

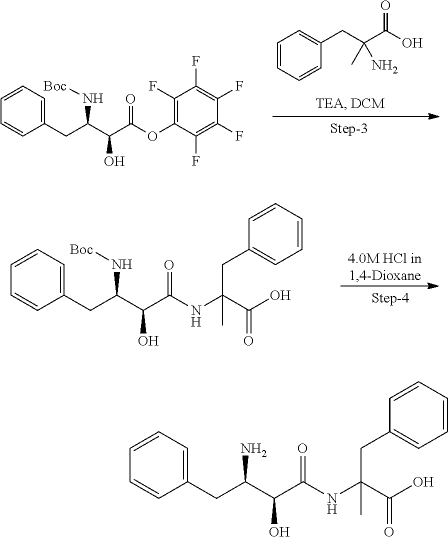

### Step 1: Synthesis of 2-[[(2S,3R)-3-(tert-butoxycarbonylamino)-2-hydroxy-4-phenyl-butanoyl]amino]-2-methyl-3-phenyl-propanoic acid

To a stirred solution of 2-amino-2-methyl-3-phenylpropanoic acid (80.47 mg, 0.449 mmol) in DCM (1 mL) was added triethylamine (0.09 mL, 0.673 mmol) at 0° C. followed by (2,3,4,5,6-pentafluorophenyl) (2S,3R)-3-(tert-butoxycarbonylamino)-2-hydroxy-4-phenyl-butanoate (100 mg, 0.22 mmol) dissolved in DCM (1 mL) and the resultant mixture was stirred at room temperature for 2 h. The progress of the reaction was monitored by TLC. Reaction mixture was concentrated under reduced pressure to obtain 2-[[(2S,3R)-3-(tert-butoxycarbonylamino)-2-hydroxy-4-phenyl-butanoyl]amino]-2-methyl-3-phenyl-propanoic acid (120 mg, crude). LCMS: Calculated for C₂₅H₃₂N₂O₆ is 456.23 and found:455.15 (M−1).

### Step 2: Synthesis of 2-[[(2S,3R)-3-(tert-butoxycarbonylamino)-2-hydroxy-4-phenyl-butanoyl]amino]-2-methyl-3-phenyl-propanoic acid

To a stirred solution of 2-[[(2S,3R)-3-(tert-butoxycarbonylamino)-2-hydroxy-4-phenyl-butanoyl]amino]-2-methyl-3-phenyl-propanoic acid (120 mg, 0.26 mmol) in dioxane (0.6 mL) was added 4 M HCl in 1,4-dioxane (0.98 mL, 3.94 mmol) dropwise at 0° C. and the reaction mixture was stirred at room temperature for 4 h. The progress of the reaction was monitored by TLC. After completion, the reaction mixture was concentrated under reduced pressure to obtain crude product. The crude product was purified by prep-HPLC [(Column: LUNA (C18, 21.2 mm×250 mm), mobile phase: A=0.1% HCOOH in water, Phase B=MeCN, Flow: 15 ml/min, Gradient Programme: Time-0,2,10% B-10,20,40), followed by lyophilization of the fractions] to afford 2-[[(2S,3R)-3-(tert-butoxycarbonylamino)-2-hydroxy-4-phenyl-butanoyl]amino]-2-methyl-3-phenyl-propanoic acid (2 mg, 2%) as white fluffy solid. ¹H NMR (400 MHz, Methanol-d₄) δ 1.39 (s, 3H) 1.60-1.68 (m, 2H) 2.89-2.97 (m, 2H) 2.99-3.08 (m, 2H) 3.09-3.24 (m, 3H) 3.62-3.78 (m, 4H) 3.87-3.90 (m, 1H) 3.95 (d, J=2.35 Hz, 1H) 7.08-7.25 (m, 10H) 7.27-7.42 (m, 11H) ppm; LCMS: Calculated for C₂₀H₂₄N₂O₄ is 356.17 and found 357.2 (M+1).

### Example 13: Synthesis of (2S,3R)-3-amino-2-hydroxy-4-phenyl-N-[phenyl(1H-tetrazol-5-yl)methyl]butanamide (Compound 13)

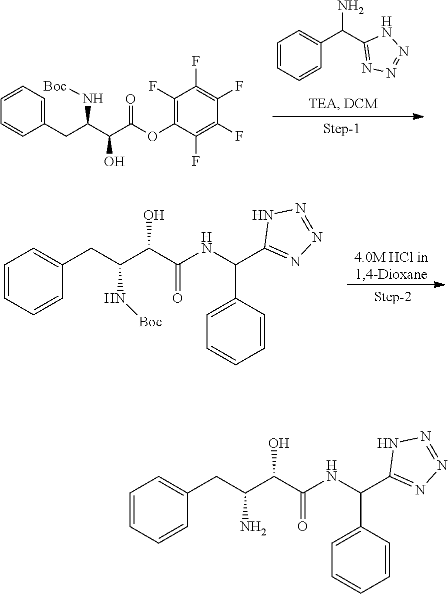

### Step 1: Synthesis of tert-butyl N-[(1R,2S)-1-benzyl-2-hydroxy-3-oxo-3-[[phenyl(1H-tetrazol-5-yl)methyl]amino]propyl]carbamate

A solution of (2,3,4,5,6-pentafluorophenyl) (2S,3R)-3-(tert-butoxycarbonylamino)-2-hydroxy-4-phenyl-butanoate (157 mg, 0.34 mmol) in DCM (1 mL) was cooled to 0° C. To this were added triethylamine (0.12 mL, 0.85 mmol) and phenyl(1H-tetrazol-5-yl)methanamine (50 mg, 0.28 mmol) in DCM (1 mL). The reaction mixture was stirred at room temperature for 16 h. The progress of the reaction was monitored by TLC. After completion, the reaction mixture was quenched with ice cold water and extracted with DCM. The organic layer was washed with water, dried over sodium sulfate and concentrated under vacuum pressure to get crude product. It was purified by prep-HPLC [(Column: Xbridge phenyl (C18, 20 mm×150 mm)5.0μ), Phase A: 10 mm ammonium bicarbonate, Phase B: MeCN, Gradient Elusion), followed by lyophilization of the fractions] to afford tert-butyl N-[(1R,2S)-1-benzyl-2-hydroxy-3-oxo-3-[[phenyl(1H-tetrazol-5-yl)methyl]amino]propyl]carbamate (20 mg, 15%). ¹H NMR (400 MHz, DMSO-d₆) δ 1.25 (d, J=13.35 Hz, 4H) 2.63-2.68 (m, 1H) 2.73-2.86 (m, 1H) 3.85-4.04 (m, 1H) 5.71-6.06 (m, 1H) 6.18-6.42 (m, 1H) 7.16-7.33 (m, 5H) 8.52-8.75 (m, 1H) ppm; LCMS: calculated for C₂₃H₂₈N6O₄ is 452.22; found 451.2 (M−1).

### Step 2: Synthesis of (2S,3R)-3-amino-2-hydroxy-4-phenyl-N-[phenyl(1H-tetrazol-5-yl)methyl]butanamide

To a stirred solution of tert-butyl N-[(1R,2S)-1-benzyl-2-hydroxy-3-oxo-3-[[phenyl(1H-tetrazol-5-yl)methyl]amino]propyl]carbamate (20 mg, 0.044 mmol) in 1,4-dioxane (1 mL) was added 4 M HCl in 1,4-dioxane (0.4 mL, 20 Vol) at 0° C. and the reaction mixture was stirred at room temperature for 16 h. The progress of the reaction was monitored by TLC. After completion, the reaction mixture was evaporated to dryness under vacuum to get the crude product. The crude product was washed with diethyl ether to afford 2S,3R)-3-amino-2-hydroxy-4-phenyl-N-[phenyl(1H-tetrazol-5-yl)methyl]butanamide as pale-brown solid (10 mg, 64.5%). ¹H NMR (400 MHz, DMSO-d₆) δ 2.82-2.96 (m, 2H) 3.59-3.70 (m, 2H) 3.99-4.13 (m, 1H) 6.43 (dd, J=11.08, 7.85 Hz, 1H) 6.65-6.72 (m, 1H) 6.86 (br d, J=5.43 Hz, 1H) 7.27-7.41 (m, 11H) 7.94 (br d, J=1.47 Hz, 3H) 9.01-9.12 (m, 1H) 16.25-16.67 (m, 1H) ppm; LCMS: calculated for C₁₈H₂₀N6O₂ is 352.16 and found 352.95 (M+1).

### Example 14: Synthesis of 2-[[(2S,3R)-3-amino-2-hydroxy-4-phenyl-butanoyl]amino]-3-phenyl-butanoic acid (Compound 14)

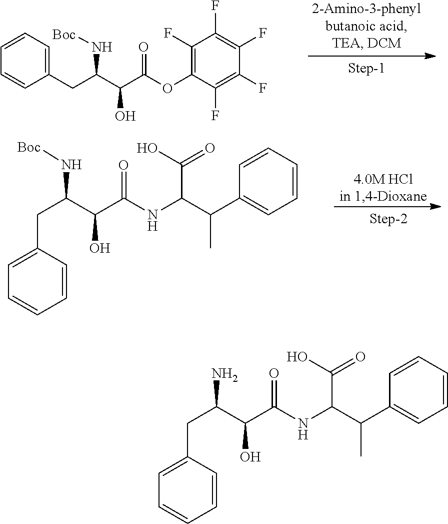

### Step 1: Synthesis of 2-[[(2S,3R)-3-(tert-butoxycarbonylamino)-2-hydroxy-4-phenyl-butanoyl]amino]-3-phenyl-butanoic acid

Following the protocol described for the synthesis of the compound 1, the compound 2-[[(2S,3R)-3-(tert-butoxycarbonylamino)-2-hydroxy-4-phenyl-butanoyl]amino]-3-phenyl-butanoic acid was isolated in crude form. It was purified by prep-HPLC [(Column: Gemini (21.2 mm×250 mm), mobile phase: A=0.02% NH₃ in water, Phase B=MeCN, Flow: 15 ml/min, Gradient Programme: Time-0,2,8% B-15,20,40), followed by lyophilization of the fractions] to afford 2-[[(2S,3R)-3-(tert-butoxycarbonylamino)-2-hydroxy-4-phenyl-butanoyl]amino]-3-phenyl-butanoic acid (50 mg, 33.3%). LCMS: calculated for C₂₅H₃₂N₂O₆ is 456.23; found 455.05 (M−1).

### Step 2: Synthesis of 2-[[(2S,3R)-3-amino-2-hydroxy-4-phenyl-butanoyl]amino]-3-phenyl-butanoic acid

Following the protocol described for the synthesis of the compound 1, the compound 2-[[(2S,3R)-3-amino-2-hydroxy-4-phenyl-butanoyl]amino]-3-phenyl-butanoic acid was isolated as off-white solid (6.0 mg, 20%). ¹H NMR (400 MHz, Methanol-d₄) δ 1.18-1.43 (m, 9H) 2.80-3.08 (m, 2H) 3.18-3.26 (m, 1H) 3.38-3.51 (m, 1H) 3.58-3.77 (m, 2H) 3.96-4.08 (m, 1H) 4.63-4.75 (m, 1H) 7.05-7.43 (m, 11H) ppm; LCMS: calculated for C₂₀H₂₄N₂O₄ is 356.17 and found 357.15 (M+1).

### Example 15: Synthesis of 1-[[(2S,3R)-3-amino-2-hydroxy-4-phenyl-butanoyl]amino]cyclopentane carboxylic acid (Compound 15)

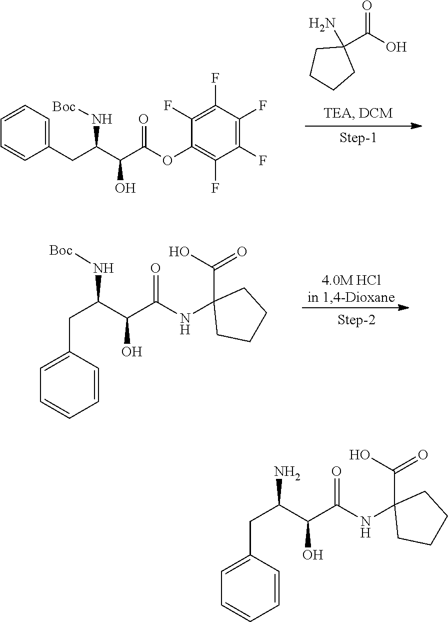 [SMILES: CC(C)(C)OC(=O)N[C@H](Cc1ccccc1)[C@H](O)C(=O)Oc1c(F)c(F)c(F)c(F)c1F ; NC1(C(=O)O)CCCC1 ; CC(C)(C)OC(=O)N[C@H](Cc1ccccc1)[C@H](O)C(=O)NC1(C(=O)O)CCCC1 ; N[C@H](Cc1ccccc1)[C@H](O)C(=O)NC1(C(=O)O)CCCC1]

### Step 1: Synthesis of 1-[[(2S,3R)-3-(tert-butoxycarbonylamino)-2-hydroxy-4-phenyl-butanoyl]amino]cyclopentanecarboxylic acid

Following the protocol described for the synthesis of compound 1 and using prep-HPLC (conditions similar to described above) the compound 1-[[(2S,3R)-3-(tert-butoxycarbonylamino)-2-hydroxy-4-phenyl-butanoyl]amino]cyclopentanecarboxylic acid was isolated in pure form (90 mg, 17%). LCMS: calculated for C₂₁H₃₀N₂O₆ is 406.21, found: 405.1 (M−1).

### Step 2: Synthesis of 1-[[(2S,3R)-3-amino-2-hydroxy-4-phenyl-butanoyl]amino]cyclopentane carboxylic acid

Reaction conditions are similar to what is described for the synthesis of compound 1. The crude was purified by prep-HPLC [(Column: X SELECT (250 mm×20.0 mm), 5.0p), mobile phase: A=0.02% NH₄₀H in water, Phase B=MeCN, Flow: 15 ml/min, Gradient Programme: Time-0,2,8% B-5,10,25), followed by lyophilization of the fractions] to afford 1-[[(2S,3R)-3-amino-2-hydroxy-4-phenyl-butanoyl]amino]cyclopentane carboxylic acid as an off-white solid (5 mg, 16%). ¹H NMR (400 MHz, DMSO-d₆) δ 1.58-1.66 (m, 4H) 1.73-1.82 (m, 1H) 1.89-2.03 (m, 2H) 2.16 (br d, J=5.72 Hz, 1H) 2.84 (br d, J=6.90 Hz, 2H) 3.65 (br s, 2H) 6.05-6.20 (m, 1H) 7.26-7.34 (m, 4H) 7.39-7.47 (m, 1H) ppm. LCMS: calculated for C₁₆H₂₂N₂O₄ is 306.16, found: 306.95 (M+1).

### Example 16: Synthesis of 2-[[(2S,3R)-3-amino-2-hydroxy-4-phenyl-butanoyl]amino]-3-[4-(trifluoromethyl)phenyl]propanoic acid (Compound 16)

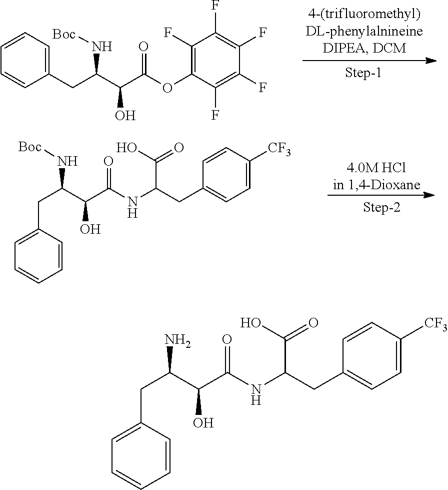

### Step 1: Synthesis of 2-[[(2S,3R)-3-(tert-butoxycarbonylamino)-2-hydroxy-4-phenyl-butanoyl]amino]-3-[4-(trifluoromethyl)phenyl]propanoic acid

The crude product that was obtained following the protocol that is described for the synthesis of the compound 1, was purified by prep-HPLC [(Column: Gemini (21.2 mm×250 mm), mobile phase: A=0.02% NH₃ in water, Phase B=MeCN, Flow: 15 ml/min, Gradient Programme: Time-0,2,8% B-10,20,35), followed by lyophilization of the fractions] to afford 2-[[(2S,3R)-3-(tert-butoxycarbonylamino)-2-hydroxy-4-phenyl-butanoyl]amino]-3-[4-(trifluoromethyl)phenyl]propanoic acid (10 mg, 10%) as white solid. ¹H NMR (300 MHz, DMSO-d₆) δ 1.15 (s, 3H) 1.25 (s, 9H) 2.57 (br s, 1H) 2.73 (br d, J=2.02 Hz, 1H) 3.09 (br s, 2H) 6.06 (d, J=6.74 Hz, 1H) 6.17 (d, J=9.77 Hz, 1H) 7.11-7.20 (m, 3H) 7.22-7.31 (m, 2H) 7.32-7.42 (m, 2H) 7.52-7.60 (m, 2H) 7.69-7.87 (m, 1H) ppm; LCMS: calculated for C₂₅H₂₉F₃N₂O₆ is 510.2; found 509.15 (M−1).

### Step 2: Synthesis of 2-[[(2S,3R)-3-amino-2-hydroxy-4-phenyl-butanoyl]amino]-3-[4-(trifluoromethyl)phenyl]propanoic acid

Following the protocol described for the synthesis of the compound 1, the compound 2-[[(2S,3R)-3-amino-2-hydroxy-4-phenyl-butanoyl]amino]-3-[4-(trifluoromethyl)phenyl]propanoic acid was obtained as white solid (3.0 mg, 30%). ¹H NMR (400 MHz, DMSO-d₆) δ 1.21-1.33 (m, 2H) 2.60-2.72 (m, 1H) 2.79 (br dd, J=13.86, 6.82 Hz, 1H) 3.10-3.22 (m, 2H) 3.49-3.62 (m, 1H) 3.98-4.09 (m, 1H) 4.53-4.67 (m, 1H) 6.70 (br d, J=5.87 Hz, 1H) 7.21 (br d, J=7.04 Hz, 2H) 7.26 (s, 1H) 7.30-7.38 (m, 2H) 7.48 (br d, J=7.92 Hz, 2H) 7.61 (br d, J=8.07 Hz, 2H) 7.77-7.96 (m, 2H) 8.22-8.37 (m, 1H) 12.10-13.62 (m, 1H) ppm; LCMS: calculated for C₂₀H₂₁F₃N₂O₄ is 410.15; found 411.15 (M+1).

### Example 17: Synthesis of 2-[[(2S,3R)-3-amino-2-hydroxy-4-phenyl-butanoyl]amino]-3-cyclopentyl-propanoic acid (Compound 17)

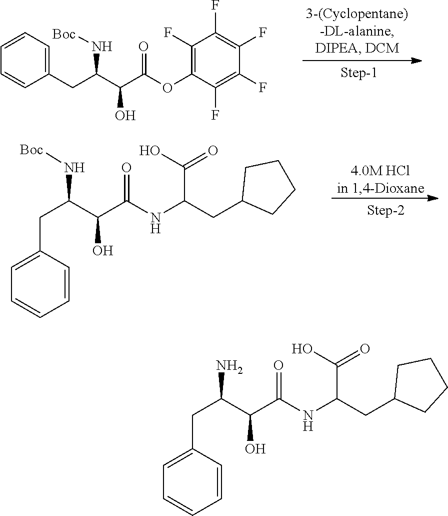

### Step 1: Synthesis of 2-[[(2S,3R)-3-(tert-butoxycarbonylamino)-2-hydroxy-4-phenyl-butanoyl]amino]-3-cyclopentyl-propanoic acid

Using the protocol that is described for the synthesis of compound 1, the compound 2-[[(2S,3R)-3-(tert-butoxycarbonylamino)-2-hydroxy-4-phenyl-butanoyl]amino]-3-cyclopentyl-propanoic acid was obtained in crude form. It was further purified by prep-HPLC [(Column: Gemini (21.2 mm×250 mm), mobile phase: A=0.02% NH₃ in water, Phase B=MeCN, Flow: 20 ml/min, Gradient Programme: Time-0,3,8% B-10,20,30), followed by lyophilization of the fractions] to obtain pure product (120 mg, 24%). ¹H NMR (300 MHz, DMSO-d₆) δ 0.95-1.13 (m, 6H) 1.15 (s, 2H) 1.27 (s, 9H) 1.43 (br d, J=4.72 Hz, 2H) 1.50-1.57 (m, 2H) 1.62-1.76 (m, 4H) 1.78-1.87 (m, 1H) 2.62 (br dd, J=13.14, 8.08 Hz, 1H) 2.70 (s, 1H) 3.80 (br s, 1H) 3.87-3.98 (m, 1H) 4.05 (s, 1H) 6.05 (br s, 1H) 6.11-6.16 (m, 1H) 7.13-7.22 (m, 3H) 7.23-7.31 (m, 2H) 7.67-7.83 (m, 1H) ppm; LCMS: calculated for C₂₃H₃₄N₂O₆ is 434.24; found 433.10 (M−1).

### Step 2: Synthesis of 2-[[(2S,3R)-3-amino-2-hydroxy-4-phenyl-butanoyl]amino]-3-cyclopentyl-propanoic acid

Using the protocol that is described for the synthesis of the compound 1, the compound 2-[[(2S,3R)-3-(tert-butoxycarbonylamino)-2-hydroxy-4-phenyl-butanoyl]amino]-3-cyclopentyl-propanoic acid (9.0 mg, 18%) was obtained in pure form. ¹H NMR (300 MHz, DMSO-d₆) δ 1.10 (br dd, J=11.62, 7.24 Hz, 1H) 1.22-1.31 (m, 1H) 1.41-1.62 (m, 3H) 1.66-1.86 (m, 4H) 2.69-2.86 (m, 1H) 2.87-3.06 (m, 1H) 3.45-3.59 (m, 1H) 3.92-4.05 (m, 1H) 4.13-4.26 (m, 1H) 6.56-6.73 (m, 1H) 7.21-7.39 (m, 4H) 7.91 (br s, 2H) 8.17-8.28 (m, 1H) 12.44-12.89 (m, 1H) ppm; LCMS: calculated for C₁₈H₂₆N₂O₄ is 334.19; found 335.20 (M+1).

### Example 18: Synthesis of (2S)-2-[[(2S,3R)-3-amino-2-hydroxy-4-phenyl-butanoyl]amino]-3-(4-methoxyphenyl)propanoic acid (Compound 18)

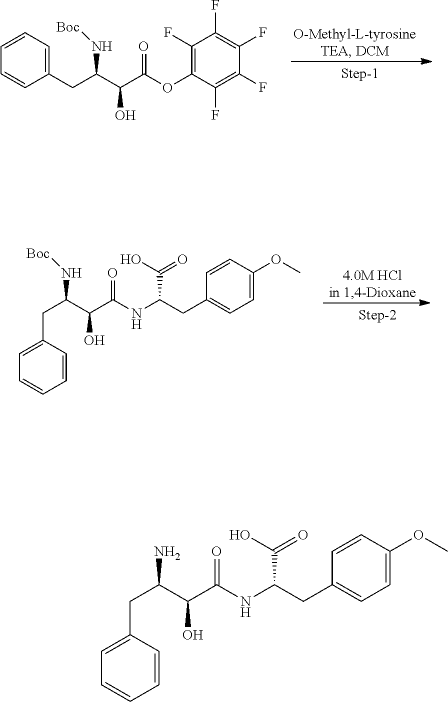 [SMILES: CC(C)(C)OC(=O)N[C@H](Cc1ccccc1)[C@H](O)C(=O)Oc1c(F)c(F)c(F)c(F)c1F ; COc1ccc(C[C@H](NC(=O)[C@@H](O)[C@@H](Cc2ccccc2)NC(=O)OC(C)(C)C)C(=O)O)cc1 ; COc1ccc(C[C@H](NC(=O)[C@@H](O)[C@H](N)Cc2ccccc2)C(=O)O)cc1]

### Step 1: Synthesis of (2S)-2-[[(2S,3R)-3-(tert-butoxycarbonylamino)-2-hydroxy-4-phenyl-butanoyl]amino]-3-(4-methoxyphenyl)propanoic acid

Following the protocol that is described for the synthesis of compound 1 and purification using prep-HPLC, the compound (2S)-2-[[(2S,3R)-3-(tert-butoxycarbonylamino)-2-hydroxy-4-phenyl-butanoyl]amino]-3-(4-methoxyphenyl)propanoic acid was isolated in pure form. (100 mg, 28.57%). ¹H NMR (300 MHz, DMSO-d₆) δ 1.27 (s, 9H) 2.70 (br dd, J=13.31, 5.89 Hz, 1H) 2.85-3.02 (m, 2H) 3.63 (s, 3H) 3.81 (br s, 1H) 3.94 (br d, J=6.74 Hz, 1H) 4.29 (br d, J=5.39 Hz, 1H) 6.08 (br d, J=9.43 Hz, 1H) 6.76 (br d, J=8.42 Hz, 2H) 7.07 (br d, J=8.42 Hz, 2H) 7.13-7.20 (m, 3H) 7.24 (br d, J=6.40 Hz, 2H) 7.54-7.75 (m, 1H) ppm; LCMS: calculated for C₂₅H₃₂N₂O₇ is 472.22; found 471.40 (M−1).

### Step 2: Synthesis of (2S)-2-[[(2S,3R)-3-amino-2-hydroxy-4-phenyl-butanoyl]amino]-3-(4-methoxyphenyl)propanoic acid

The compound (2S)-2-[[(2S,3R)-3-amino-2-hydroxy-4-phenyl-butanoyl]amino]-3-(4-methoxyphenyl)propanoic acid was synthesized and isolated as white solid (12 mg, 8.0%), using the protocol that is described for the synthesis of the compound 1. ¹H NMR (300 MHz, DMSO-d₆) δ 2.72-2.84 (m, 1H) 2.97-3.06 (m, 2H) 3.61 (s, 3H) 3.89-4.05 (m, 1H) 4.39-4.54 (m, 1H) 6.73 (d, J=5.73 Hz, 1H) 6.80 (br d, J=8.42 Hz, 2H) 7.11-7.17 (m, 2H) 7.21 (br d, J=6.74 Hz, 2H) 7.26-7.38 (m, 3H) 7.77-7.92 (m, 2H) 8.12 (s, 1H) 12.68-12.99 (m, 1H) ppm; LCMS: calculated for C₂₀H₂₄N₂O₅ is 372.17; found 373.15 (M+1).

### Example 19: Synthesis of 2-[[(2S,3R)-3-amino-2-hydroxy-4-phenyl-butanoyl]amino]-3-(4-hydroxyphenyl)propanoic acid (Compound 19)

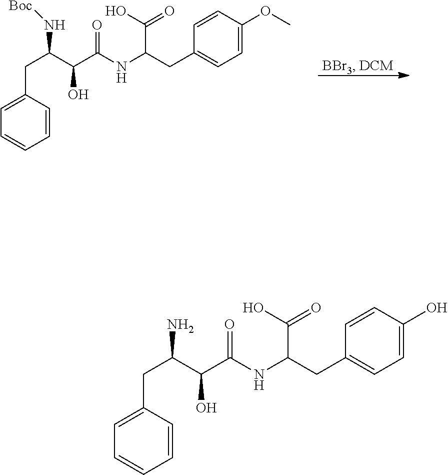 [SMILES: COc1ccc(CC(NC(=O)[C@@H](O)[C@@H](Cc2ccccc2)NC(=O)OC(C)(C)C)C(=O)O)cc1 ; N[C@H](Cc1ccccc1)[C@H](O)C(=O)NC(Cc1ccc(O)cc1)C(=O)O]

To a stirred solution of 2-[[(2S,3R)-3-(tert-butoxycarbonylamino)-2-hydroxy-4-phenyl-butanoyl]amino]-3-(4-methoxyphenyl)propanoic acid (30 mg, 0.06 mmol) in DCM (0.5 mL) cooled to 0° C. was added BBr₃ (15 mg, 0.06 mmol) and the reaction mixture was stirred at room temperature for 4 h. The progress of the reaction was monitored by TLC. After completion, the reaction mass was quenched with water and extracted with ethyl acetate (2×50 mL). The organic layer was collected and concentrated under reduced pressure to get crude product. It was then purified by prep-HPLC [(Column: Gemini (21.2 mm×250 mm), mobile phase: A=0.02% NH₃ in water, Phase B=MeCN, Flow: 20 ml/min, Gradient Programme: Time-0,3,8% B-10,20,30), followed by lyophilization of the fractions] to afford 2-[[(2S,3R)-3-amino-2-hydroxy-4-phenyl-butanoyl]amino]-3-(4-hydroxyphenyl)propanoic acid as white solid (1.6 mg, 5.33%). ¹H NMR (400 MHz, DMSO-d₆) δ 2.71 (br d, J=6.16 Hz, 1H) 2.82-2.91 (m, 2H) 3.09 (br d, J=10.42 Hz, 1H) 3.51 (br s, 1H) 3.77 (br s, 1H) 4.00-4.13 (m, 1H) 5.49-5.76 (m, 1H) 6.80 (d, J=8.22 Hz, 1H) 6.98 (dd, J=8.36, 1.76 Hz, 1H) 7.20-7.38 (m, 6H) 7.76-7.92 (m, 1H) 9.71-10.04 (m, 1H) ppm; LCMS: calculated for C₁₉H₂₂N₂O₅ is 358.15 and found 359.10 (M+1).

### Example 20: Synthesis of (2S)-2-[[(2S,3R)-3-amino-2-hydroxy-4-phenyl-butanoyl]amino]-2-cyclobutyl-acetic acid (Compound 20)

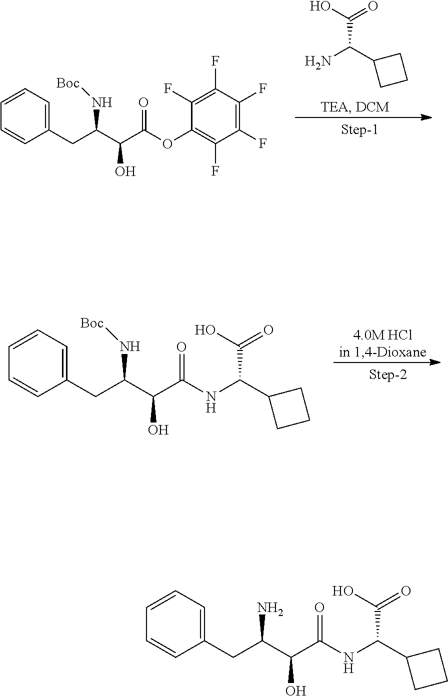 [SMILES: CC(C)(C)OC(=O)N[C@H](Cc1ccccc1)[C@H](O)C(=O)Oc1c(F)c(F)c(F)c(F)c1F ; N[C@H](C(=O)O)C1CCC1 ; CC(C)(C)OC(=O)N[C@H](Cc1ccccc1)[C@H](O)C(=O)N[C@H](C(=O)O)C1CCC1 ; N[C@H](Cc1ccccc1)[C@H](O)C(=O)N[C@H](C(=O)O)C1CCC1]

### Step 1: Synthesis of (2S)-2-[[(2S,3R)-3-(tert-butoxycarbonylamino)-2-hydroxy-4-phenyl-butanoyl]amino]-2-cyclobutyl-acetic acid

The compound (2S)-2-[[(2S,3R)-3-(tert-butoxycarbonylamino)-2-hydroxy-4-phenyl-butanoyl]amino]-2-cyclobutyl-acetic acid was synthesized using the protocol described for compound 1. The crude product was purified by prep-HPLC (Column: Xbridge (C18, 20 mm×150 mm, 5.0p), Phase A: Ammonium bicarbonate, Phase B: MeCN, Gradient Elusion), followed by lyophilization of the fractions] to afford the pure product (20 mg, 22.7%). ¹H NMR (400 MHz, DMSO-d₆) δ 1.26 (s, 9H) 1.66-1.89 (m, 6H) 2.62 (br s, 1H) 2.74-2.85 (m, 1H) 3.80-3.89 (m, 1H) 3.93-4.02 (m, 1H) 4.12-4.24 (m, 1H) 6.08 (br d, J=6.46 Hz, 1H) 6.15 (br d, J=9.54 Hz, 1H) 7.14-7.30 (m, 6H) 7.64 (br d, J=8.66 Hz, 1H) 12.22-13.06 (m, 1H) ppm; LCMS: calculated for C₂₁H₃₀N₂O₆ is 406.21; found 405.05 (M−1).

### Step 2: Synthesis of (2S)-2-[[(2S,3R)-3-amino-2-hydroxy-4-phenyl-butanoyl]amino]-2-cyclobutyl-acetic acid

Using the protocol described for the synthesis of the compound 1, the product (2S)-2-[[(2S,3R)-3-amino-2-hydroxy-4-phenyl-butanoyl]amino]-2-cyclobutyl-acetic acid was isolated as pale-brown solid (10 mg, 66.6%). ¹H NMR (400 MHz, DMSO-d₆) δ 1.70-1.77 (m, 1H) 1.79-1.86 (m, 2H) 1.86-2.00 (m, 3H) 2.74-2.87 (m, 1H) 2.89-2.98 (m, 1H) 3.45-3.55 (m, 1H) 3.98-4.05 (m, 1H) 4.12-4.20 (m, 1H) 6.69 (d, J=6.02 Hz, 1H) 7.29 (br d, J=6.90 Hz, 3H) 7.32-7.40 (m, 2H) 7.91 (br s, 2H) 8.07-8.15 (m, 1H) 12.57-12.73 (m, 1H) ppm; LCMS: calculated for C₁₆H₂₂N₂O₄ is 306.16; found 306.90 (M+1).

### Example 21: Synthesis of 2-[[(2S,3R)-3-amino-2-hydroxy-4-phenyl-butanoyl]amino]-2-tetrahydropyran-4-yl-acetic acid (Compound 21)

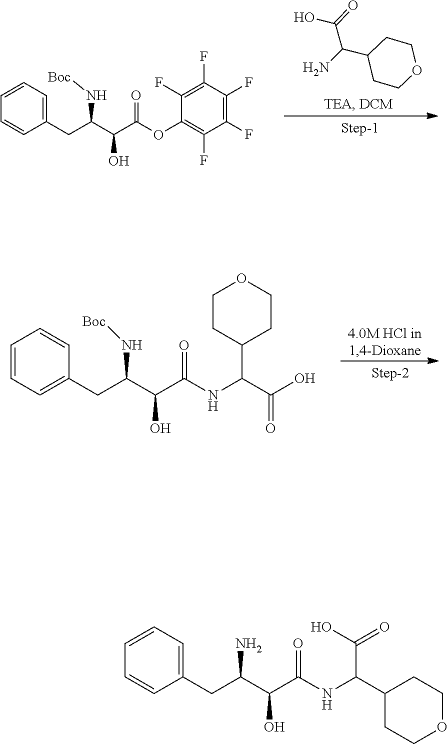 [SMILES: CC(C)(C)OC(=O)N[C@H](Cc1ccccc1)[C@H](O)C(=O)Oc1c(F)c(F)c(F)c(F)c1F ; NC(C(=O)O)C1CCOCC1 ; CC(C)(C)OC(=O)N[C@H](Cc1ccccc1)[C@H](O)C(=O)NC(C(=O)O)C1CCOCC1 ; N[C@H](Cc1ccccc1)[C@H](O)C(=O)NC(C(=O)O)C1CCOCC1]

### Step 1: Synthesis of 2-[[(2S,3R)-3-(tert-butoxycarbonylamino)-2-hydroxy-4-phenyl-butanoyl]amino]-2-tetrahydropyran-4-yl-acetic acid

Using the protocol described for compound 1, the compound 2-[[(2S,3R)-3-(tert-butoxycarbonylamino)-2-hydroxy-4-phenyl-butanoyl]amino]-2-tetrahydropyran-4-yl-acetic acid was synthesized in pure form (23.0 mg, 16.0%). ¹H NMR (400 MHz, DMSO-d₆) δ 1.27 (s, 9H) 1.35-1.48 (m, 2H) 1.86-1.99 (m, 1H) 2.63-2.69 (m, 1H) 2.80 (dd, J=13.20, 6.60 Hz, 1H) 3.21 (br s, 2H) 3.74-3.99 (m, 5H) 6.08 (br d, J=5.43 Hz, 1H) 6.17 (br d, J=9.54 Hz, 1H) 7.12-7.29 (m, 5H) 7.56-7.70 (m, 1H) ppm; LCMS: calculated for C₂₂H₃₂N₂O₇ is 436.2; found 435.2 (M−1).

### Step 2: Synthesis of 2-[[(2S,3R)-3-amino-2-hydroxy-4-phenyl-butanoyl]amino]-2-tetrahydropyran-4-yl-acetic acid

To a stirred solution of 2-[[(2S,3R)-3-(tert-butoxycarbonylamino)-2-hydroxy-4-phenyl-butanoyl]amino]-2-tetrahydropyran-4-yl-acetic acid (23.0 mg, 0.052 mmol) in 1,4-dioxane (0.5 mL) was added HCl in 1,4-dioxane (0.46 mL, 20 vol) at 0° C. and the reaction mixture was stirred at room temperature for 16 h. The progress of the reaction was monitored by TLC. The reaction mixture was evaporated to dryness under vacuum to get the crude. The crude was washed with diethyl ether to afford 2-[[(2S,3R)-3-amino-2-hydroxy-4-phenyl-butanoyl]amino]-2-tetrahydropyran-4-yl-acetic acid as off-white solid (10.0 mg, 58.8%). ¹H NMR (400 MHz, DMSO-d₆) δ 1.09 (t, J=7.04 Hz, 2H) 1.29-1.40 (m, 2H) 1.45-1.54 (m, 2H) 1.96-2.08 (m, 1H) 2.82-2.98 (m, 2H) 3.22-3.29 (m, 3H) 3.36 (br s, 2H) 3.82-3.88 (m, 2H) 4.04 (br d, J=3.23 Hz, 1H) 4.08-4.17 (m, 1H) 6.62-6.77 (m, 1H) 7.06 (s, 1H) 7.19 (s, 1H) 7.27-7.37 (m, 5H) 7.95 (br s, 3H) 8.10-8.19 (m, 1H) ppm; LCMS: calculated for C₁₇H₂₄N₂O₅ is 336.17; found 337.2 (M+1).

### Example 22: Synthesis of 2-[[(2S,3R)-3-amino-2-hydroxy-4-phenyl-butanoyl]amino]-3-(3,3-difluorocyclobutyl)propanoic acid (Compound 22)

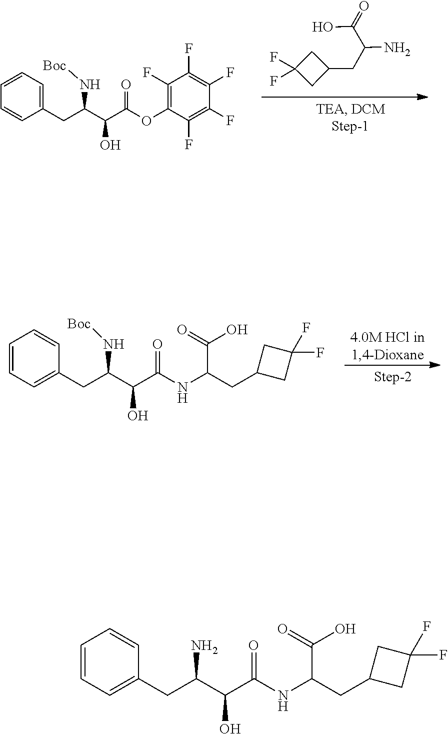 [SMILES: CC(C)(C)OC(=O)N[C@H](Cc1ccccc1)[C@H](O)C(=O)Oc1c(F)c(F)c(F)c(F)c1F ; NC(CC1CC(F)(F)C1)C(=O)O ; CC(C)(C)OC(=O)N[C@H](Cc1ccccc1)[C@H](O)C(=O)NC(CC1CC(F)(F)C1)C(=O)O ; N[C@H](Cc1ccccc1)[C@H](O)C(=O)NC(CC1CC(F)(F)C1)C(=O)O]

### Step 1: Synthesis of 2-[[(2S,3R)-3-(tert-butoxycarbonylamino)-2-hydroxy-4-phenyl-butanoyl]amino]-3-(3,3-difluorocyclobutyl)propanoic acid

Using protocol similar to the synthesis of the compound 1, the compound 2-[[(2S,3R)-3-(tert-butoxycarbonylamino)-2-hydroxy-4-phenyl-butanoyl]amino]-3-(3,3-difluorocyclobutyl)propanoic acid was synthesized in crude form. It was purified further using prep-HPLC [(Column: X-Bridge (150 mm×21.2 mm), 5.0p), Phase A: 10 mM Ammonium bicarbonate in water, Phase B: MeCN, Gradient Elusion) and sample was lyophilized] to afford pure compound (36.0 mg, 20%). LCMS: calculated for C₂₂H₃₀F₂N₂O₆ is 456.21; found 455.2 (M−1).

### Step 2: Synthesis of 2-[[(2S,3R)-3-amino-2-hydroxy-4-phenyl-butanoyl]amino]-3-(3,3-difluorocyclobutyl)propanoic acid

The compound 2-[[(2S,3R)-3-amino-2-hydroxy-4-phenyl-butanoyl]amino]-3-(3,3-difluorocyclobutyl)propanoic acid was obtained as off-white solid (28.0 mg, 89.0%) using the protocol described earlier. ¹H NMR (400 MHz, DMSO-d₆) δ 1.79-1.99 (m, 2H) 2.03-2.28 (m, 3H) 2.67 (br s, 1H) 2.92 (br d, J=7.48 Hz, 1H) 3.46 (br d, J=6.75 Hz, 1H) 3.53-3.59 (m, 1H) 3.90-4.03 (m, 1H) 4.15-4.28 (m, 1H) 6.78-6.93 (m, 1H) 6.99-7.47 (m, 18H) 7.88-8.22 (m, 3H) ppm; LCMS: calculated for C₁₇H₂₂F₂N₂O₄ is 356.15 and found 357.2 (M+1).

### Example 23: Synthesis of 2-[[(2S,3R)-3-amino-2-hydroxy-4-phenyl-butanoyl]amino]-3-tetrahydropyran-4-yl-propanoic acid (Compound 23)

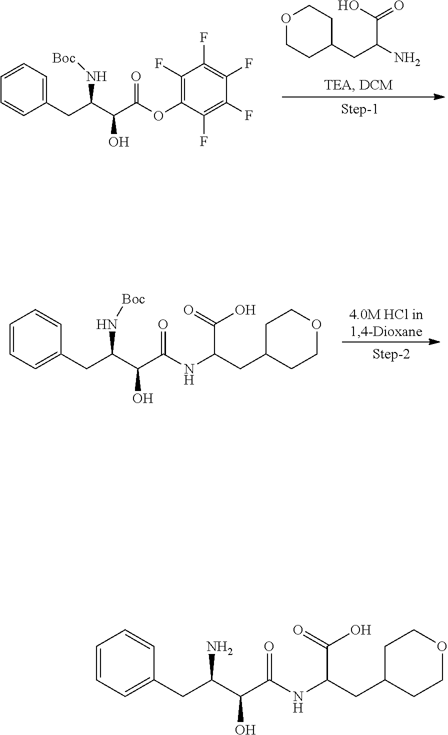 [SMILES: CC(C)(C)OC(=O)N[C@H](Cc1ccccc1)[C@H](O)C(=O)Oc1c(F)c(F)c(F)c(F)c1F ; NC(CC1CCOCC1)C(=O)O ; CC(C)(C)OC(=O)N[C@H](Cc1ccccc1)[C@H](O)C(=O)NC(CC1CCOCC1)C(=O)O ; N[C@H](Cc1ccccc1)[C@H](O)C(=O)NC(CC1CCOCC1)C(=O)O]

### Step 1: Synthesis of 2-[[(2S,3R)-3-(tert-butoxycarbonylamino)-2-hydroxy-4-phenyl-butanoyl]amino]-3-tetrahydropyran-4-yl-propanoic acid

The compound 2-[[(2S,3R)-3-(tert-butoxycarbonylamino)-2-hydroxy-4-phenyl-butanoyl]amino]-3-tetrahydropyran-4-yl-propanoic acid (20.0 mg, 25.6%) was isolated in pure form by using the protocol described earlier. LCMS: calculated for C₂₃H₃₄N₂O₇ is 450.18; found 449.3 (M−1).

### Step 2: Synthesis of 2-[[(2S,3R)-3-amino-2-hydroxy-4-phenyl-butanoyl]amino]-3-tetrahydropyran-4-yl-propanoic acid

Using the protocol that is described earlier, the compound 2-[[(2S,3R)-3-amino-2-hydroxy-4-phenyl-butanoyl]amino]-3-tetrahydropyran-4-yl-propanoic acid was isolated (15.0 mg, 89%). ¹H NMR (400 MHz, DMSO-d₆) δ 1.13-1.26 (m, 2H) 1.50-1.69 (m, 6H) 2.78 (br dd, J=13.79, 6.31 Hz, 1H) 2.93-3.00 (m, 1H) 3.19 (br s, 2H) 3.78-3.86 (m, 2H) 3.92-4.05 (m, 1H) 4.19-4.33 (m, 1H) 6.71 (s, 1H) 7.29 (br d, J=6.60 Hz, 3H) 7.32-7.40 (m, 2H) 7.80-7.94 (m, 3H) 8.18-8.35 (m, 1H) ppm; LCMS: calculated for C₁₈H₂₆N₂O₅ is 350.18; found 350.95 (M+1).

### Example 24: Synthesis of 2-[[(2S,3R)-3-amino-2-hydroxy-4-phenyl-butanoyl]amino]-3-phenyl-pentanoic acid (Compound 24)

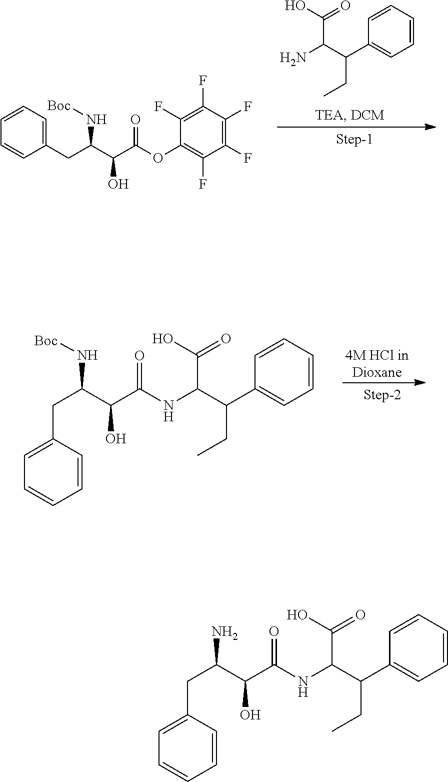 [SMILES: CC(C)(C)OC(=O)N[C@H](Cc1ccccc1)[C@H](O)C(=O)Oc1c(F)c(F)c(F)c(F)c1F ; CCC(c1ccccc1)C(N)C(=O)O ; CCC(c1ccccc1)C(NC(=O)[C@@H](O)[C@@H](Cc1ccccc1)NC(=O)OC(C)(C)C)C(=O)O ; CCC(c1ccccc1)C(NC(=O)[C@@H](O)[C@H](N)Cc1ccccc1)C(=O)O]

### Step 1: Synthesis of 2-[[(2S,3R)-3-(tert-butoxycarbonylamino)-2-hydroxy-4-phenyl-butanoyl]amino]-3-phenyl-pentanoic acid

Using the protocol that is described earlier, the compound 2-[[(2S,3R)-3-(tert-butoxycarbonylamino)-2-hydroxy-4-phenyl-butanoyl]amino]-3-phenyl-pentanoic acid was obtained as a white solid (80.0 mg). LCMS: calculated for C₂₆H₃₄N₂O₆ is 470.24; found 469.15 (M−1).

### Step 2: Synthesis of 2-[[(2S,3R)-3-amino-2-hydroxy-4-phenyl-butanoyl]amino]-3-phenyl-pentanoic acid

Using the protocol that is described for the synthesis of the compound 1, the compound 2-[[(2S,3R)-3-amino-2-hydroxy-4-phenyl-butanoyl]amino]-3-phenyl-pentanoic acid was synthesized (10.0 mg). ¹H NMR (400 MHz, DMSO-d₆) δ 0.64-0.77 (m, 6H) 1.21-1.36 (m, 1H) 1.56-1.88 (m, 4H) 2.60-2.67 (m, 1H) 2.70-2.97 (m, 5H) 3.01-3.15 (m, 2H) 3.88-4.08 (m, 2H) 4.42-4.69 (m, 2H) 6.80 (br dd, J=18.34, 6.16 Hz, 1H) 6.89-6.99 (m, 1H) 7.08-7.42 (m, 21H) 7.56 (t, J=8.58 Hz, 1H) 7.70-8.00 (m, 6H) 8.06 (br d, J=8.36 Hz, 1H) 12.02-13.29 (m, 1H) ppm; LCMS: calculated for C₂₁H₂₆N₂O₄ is 370.19; found 371.55 (M+1).

### Example 25: Synthesis of 2-[[(2S,3R)-3-amino-2-hydroxy-4-phenyl-butanoyl]amino]-3-(4-bromophenyl)propanoic acid (Compound 25)

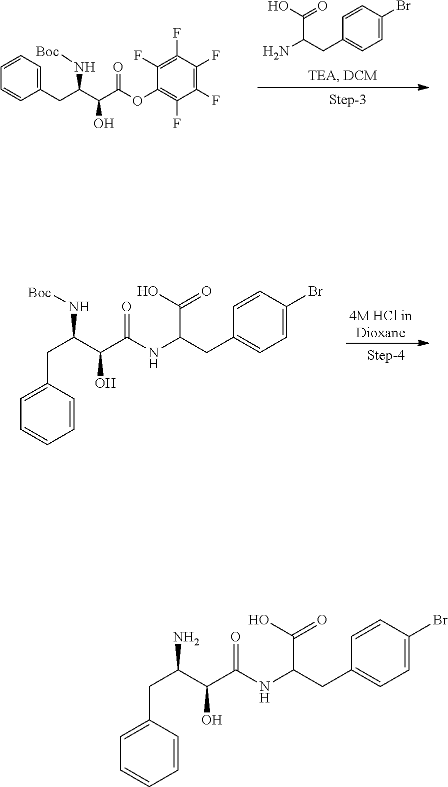 [SMILES: CC(C)(C)OC(=O)N[C@H](Cc1ccccc1)[C@H](O)C(=O)Oc1c(F)c(F)c(F)c(F)c1F ; NC(Cc1ccc(Br)cc1)C(=O)O ; CC(C)(C)OC(=O)N[C@H](Cc1ccccc1)[C@H](O)C(=O)NC(Cc1ccc(Br)cc1)C(=O)O ; N[C@H](Cc1ccccc1)[C@H](O)C(=O)NC(Cc1ccc(Br)cc1)C(=O)O]

### Step 1: Synthesis of 3-(4-bromophenyl)-2-[[(2S,3R)-3-(tert-butoxycarbonylamino)-2-hydroxy-4-phenyl-butanoyl]amino]propanoic acid

Using the protocol described earlier in this document, the compound 3-(4-bromophenyl)-2-[[(2S,3R)-3-(tert-butoxycarbonylamino)-2-hydroxy-4-phenyl-butanoyl]amino]propanoic acid was obtained in crude form. It was further purified using prep-HPLC [Mobile Phase: Mobile Phase: A=10 mM, B=MeCN Column: YMC (150 mm×21.2 mm, 5.0p) Flow: 18 ml/min; Gradient Programmer: Time A, % B: (0, 20), (2, 25), (7, 45). The collected samples were lyophilized] to afford the pure compound (30.0 mg). LCMS: calculated for C₂₄H₂₉BrN₂O₆ is 520.12; found 421.10 (M−100).

### Step 2: Synthesis of 2-[[(2S,3R)-3-amino-2-hydroxy-4-phenyl-butanoyl]amino]-3-(4-bromophenyl)propanoic acid

Using the protocol that is described for the synthesis of the compound 1, the compound 2-[[(2S,3R)-3-amino-2-hydroxy-4-phenyl-butanoyl]amino]-3-(4-bromophenyl)propanoic acid was obtained as white solid (3.0 mg, 10%). ¹H NMR (400 MHz, DMSO-d₆) δ 2.68-2.74 (m, 1H) 2.86-2.96 (m, 2H) 3.23-3.24 (m, 1H) 3.47-3.48 (m, 1H) 3.75 (br s, 1H) 4.07 (br s, 1H) 5.54-5.78 (m, 1H) 7.15 (br d, J=8.36 Hz, 2H) 7.22-7.28 (m, 3H) 7.30-7.35 (m, 2H) 7.39 (d, J=8.22 Hz, 2H) 7.86-7.93 (m, 1H) ppm; LCMS: calculated for C₁₉H₂₁BrN₂O₄ is 420.07; found 423.15 (M+2).

### Example 26: Synthesis of 2-[[(2S,3R)-3-amino-2-hydroxy-4-phenyl-butanoyl]amino]-3-(2-bromophenyl)propanoic acid (Compound 26)

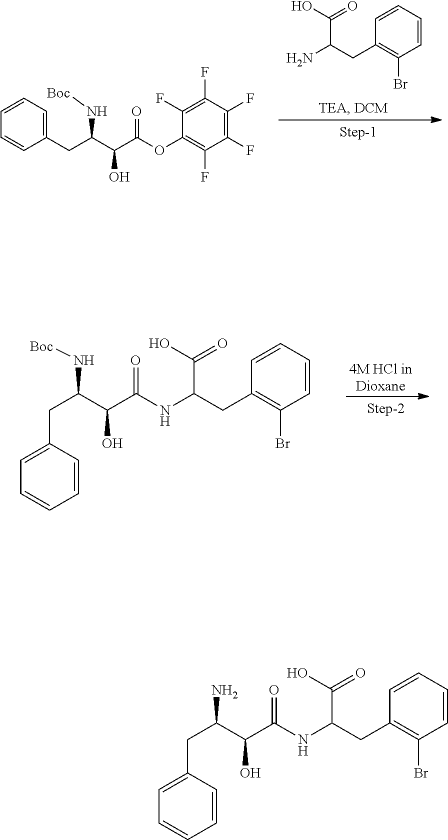 [SMILES: CC(C)(C)OC(=O)N[C@H](Cc1ccccc1)[C@H](O)C(=O)Oc1c(F)c(F)c(F)c(F)c1F ; NC(Cc1ccccc1Br)C(=O)O ; CC(C)(C)OC(=O)N[C@H](Cc1ccccc1)[C@H](O)C(=O)NC(Cc1ccccc1Br)C(=O)O ; N[C@H](Cc1ccccc1)[C@H](O)C(=O)NC(Cc1ccccc1Br)C(=O)O]

### Step 1: Synthesis of 3-(2-bromophenyl)-2-[[(2S,3R)-3-(tert-butoxycarbonylamino)-2-hydroxy-4-phenyl-butanoyl]amino]propanoic acid

Using the protocol that is described for the synthesis of compound 1, the compound 3-(2-bromophenyl)-2-[[(2S,3R)-3-(tert-butoxycarbonylamino)-2-hydroxy-4-phenyl-butanoyl]amino]propanoic acid was synthesized (30.0 mg). LCMS: calculated for C₂₄H₂₉BrN₂O₆ is 520.12; found 521.95 (M+1).

### Step 2: Synthesis of 2-[[(2S,3R)-3-amino-2-hydroxy-4-phenyl-butanoyl]amino]-3-(2-bromophenyl)propanoic acid

The compound 2-[[(2S,3R)-3-amino-2-hydroxy-4-phenyl-butanoyl]amino]-3-(2-bromophenyl)propanoic acid (3.0 mg, 6.0%) was synthesized according to the protocol described for compound 1. ¹H NMR (400 MHz, DMSO-d₆) δ 2.40 (br d, J=7.48 Hz, 1H) 2.72-2.84 (m, 1H) 3.54-3.68 (m, 2H) 3.95-4.14 (m, 1H) 4.56-4.69 (m, 1H) 6.64 (br d, J=2.79 Hz, 1H) 6.94-7.11 (m, 1H) 7.13-7.29 (m, 5H) 7.31-7.41 (m, 3H) 7.58 (d, J=7.48 Hz, 1H) 7.61-8.00 (m, 3H) 8.32-8.62 (m, 2H) 11.59-13.39 (m, 1H)ppm; LCMS: calculated for C₁₉H₂₁BrN₂O₄ is 420.07 and found 420.85 (M+1).

### Example 27: Synthesis of (S)-2-((2S,3R)-3-amino-2-hydroxy-4-phenylbutanamido)-2-(3-(trifluoromethoxy)phenyl)acetic acid (Compound 31)

### Scheme

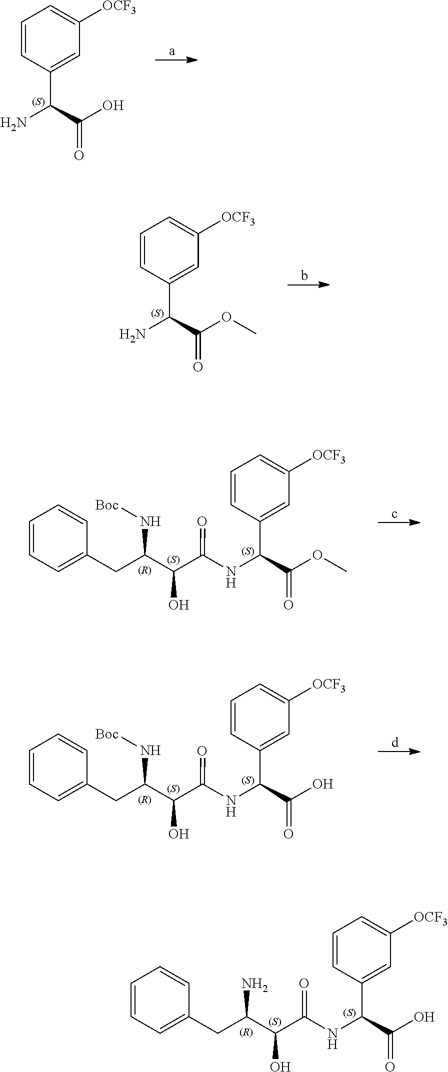

Reagents and conditions: a) SOCl₂, MeOH, 60° C., 16 h; b) Methyl (S)-2-amino-2-(3-(trifluoromethoxy)phenyl)acetate, EDC, HOBt, DMF, rt, 3 h; c) Trimethyltin hydroxide, DCE, 70° C., 2 h; d) 4 M HCl in 1,4-dioxane, 0° C., 3 h.

Synthesis of methyl (S)-2-amino-2-(3-(trifluoromethoxy)phenyl)acetate: To a stirred solution of (S)-2-amino-2-(3-(trifluoromethoxy)phenyl)acetic acid (10.0 g, 42.5 mmol) in Methanol (200 mL) was added thionyl chloride (9.31 ml, 128 mmol) at 0° C. and the reaction mixture was allowed to stir at 60° C. for 16 h. The progress of the reaction was monitored by TLC. After completion, the reaction mixture was cooled to room temperature, evaporated to dryness and the residue was treated with saturated sodium bicarbonate solution (200 mL). This was then extracted with ethyl acetate (2×100 mL) and organic layer was separated. The combined organic fractions were dried over anhydrous Na₂SO₄ and concentrated under reduced pressure to afford methyl (S)-2-amino-2-(3-(trifluoromethoxy)phenyl)acetate (10 g, 39.1 mmol, 92% yield) as a colorless oil. LC-MS: 249.95 (M+H).

Synthesis of methyl (S)-2-((2S,3R)-3-((tert-butoxycarbonyl)amino)-2-hydroxy-4-phenylbutanamido)-2-(3-(trifluoromethoxy)phenyl)acetate: To a stirred solution of (2S,3R)-3-((tert-butoxycarbonyl)amino)-2-hydroxy-4-phenylbutanoic acid (10 g, 33.9 mmol) in DMF (100 mL) at 0° C. was added HOBt (6.22 g, 40.6 mmol)followed by EDC (3.25 g, 16.93 mmol) and stirred for 20 minutes at 0° C. Then, methyl (S)-2-amino-2-(3-(trifluoromethoxy)phenyl)acetate (8.44 g, 33.9 mmol) was added under inert atmosphere. The reaction mixture was kept stirring at room temperature for 3 h. The progress of the reaction was monitored by TLC. After completion, the reaction mixture was treated with cold water (200 mL), extracted with ethyl acetate (2×200 mL) and organic layer was separated. The combined organic fractions were dried over anhydrous Na₂SO₄ and concentrated under reduced pressure to afford the crude mass. The crude compound was purified by silica gel (60-120 mesh silica gel) column chromatography using RediSep® Bronze, 80 g silica gel snap, eluting with 0-60% ethyl acetate in hexane. The desired fractions were collected and evaporated to dryness to afford methyl (S)-2-((2S,3R)-3-((tert-butoxycarbonyl)amino)-2-hydroxy-4-phenylbutanamido)-2-(3-(trifluoromethoxy)phenyl)acetate (11.0 g, 20.43 mmol, 60.3% yield) as a white solid. LC-MS: 527.05 (M+H).

Synthesis of (S)-2-((2S,3R)-3-((tert-butoxycarbonyl)amino)-2-hydroxy-4-phenylbutanamido)-2-(3-(trifluoromethoxy)phenyl)acetic acid: To a stirred solution of methyl (S)-2-((2S,3R)-3-((tert-butoxycarbonyl)amino)-2-hydroxy-4-phenylbutanamido)-2-(3-(trifluoromethoxy)phenyl)acetate (9.5 g, 18.04 mmol) in DCE (100 mL) was added trimethylstannanol (9.79 g, 54.1 mmol) under inert atmosphere. The reaction mixture was stirred at 80° C. for 8 h. The progress of the reaction was monitored by TLC. After completion, the reaction mixture was cooled to room temperature and evaporated to dryness. The residue was dissolved in ethyl acetate (200 mL) and washed with 0.1 N potassium bisulphate solution. The organic layer was separated and dried over anhydrous sodium sulphate and evaporated to dryness to afford (S)-2-((2S,3R)-3-((tert-butoxycarbonyl)amino)-2-hydroxy-4-phenylbutanamido)-2-(3-(trifluoromethoxy)phenyl)acetic acid (5.3 g, 10.22 mmol, 56.7% yield) as white solid. LC-MS: 513.00 (M+H).

This compound was subjected to SFC conditions, as below, to separate the other minor undesired isomer.
Column: Regis (S,S) Whelk-01, 250 mm×21.1 mm, 5 μm
Mobile Phase: CO₂(A) and 10Mm Ammonia in MeOH:EtOH 1:1(B)
Flow: 50 ml
Isocratic: 85(A):15(B)
Injection: Volume-0.50 ml
Run Time: 11.5 min
Instrument Make and Model: SFC-PICLAB PREP 175

### Synthesis of (S)-2-((2S,3R)-3-amino-2-hydroxy-4-phenylbutanamido)-2-(3-(trifluoromethoxy)phenyl)acetic acid (Compound 31)

To a stirred solution of (S)-2-((2S,3R)-3-((tert-butoxycarbonyl)amino)-2-hydroxy-4-phenylbutanamido)-2-(3-(trifluoromethoxy)phenyl)acetic acid (5.1 g, 9.95 mmol) in DCM (50 mL) cooled to 0° C. was added 4M HCl in 1,4-Dioxane (7.46 mL, 29.9 mmol) under inert atmosphere. The reaction mixture was stirred at room temperature for 3 h. The progress of the reaction was monitored by TLC. After completion, the reaction mixture was evaporated to dryness. The residue was washed with diethyl ether (300 mL) and dried to afford (S)-2-((2S,3R)-3-amino-2-hydroxy-4-phenylbutanamido)-2-(3-(trifluoromethoxy)phenyl)acetic acid (3.4 g, 8.15 mmol, 82% yield) as off-white solid. ¹H NMR (DMSO-d₆, 400 MHz) δ 13.3 (brs, 1H), 8.73 (d, J=7.0 Hz, 1H), 8.0-8.1 (m, 3H), 7.4-7.6 (m, 3H), 7.3-7.4 (m, 7H), 6.8-6.9 (m, 1H), 5.4-5.5 (m, 1H), 4.1-4.1 (m, 1H), 3.5-3.6 (m, 1H), 2.9-3.0 (m, 2H). ¹⁹F NMR (DMSO-d₆, 282 MHz) δ −56.68 (s, 3 F); LC-MS: 413.05 (M+H).

### Example 28: Synthesis of (S)-2-((2S,3R)-3-amino-2-hydroxy-4-phenylbutanamido)-2-(3-(trifluoromethoxy)phenyl)propanoic acid and (R)-2-((2S,3R)-3-amino-2-hydroxy-4-phenylbutanamido)-2-(3-(trifluoromethoxy)phenyl)propanoic acid (Compound 88 and Compound 89); Synthesis of (R)-2-((2S,3R)-3-amino-2-hydroxy-4-phenylbutanamido)-2-(3-(trifluoromethoxy)phenyl)butanoic acid and (S)-2-((2S,3R)-3-amino-2-hydroxy-4-phenylbutanamido)-2-(3-(trifluoromethoxy)phenyl)butanoic acid (Compound 121 and Compound 122)

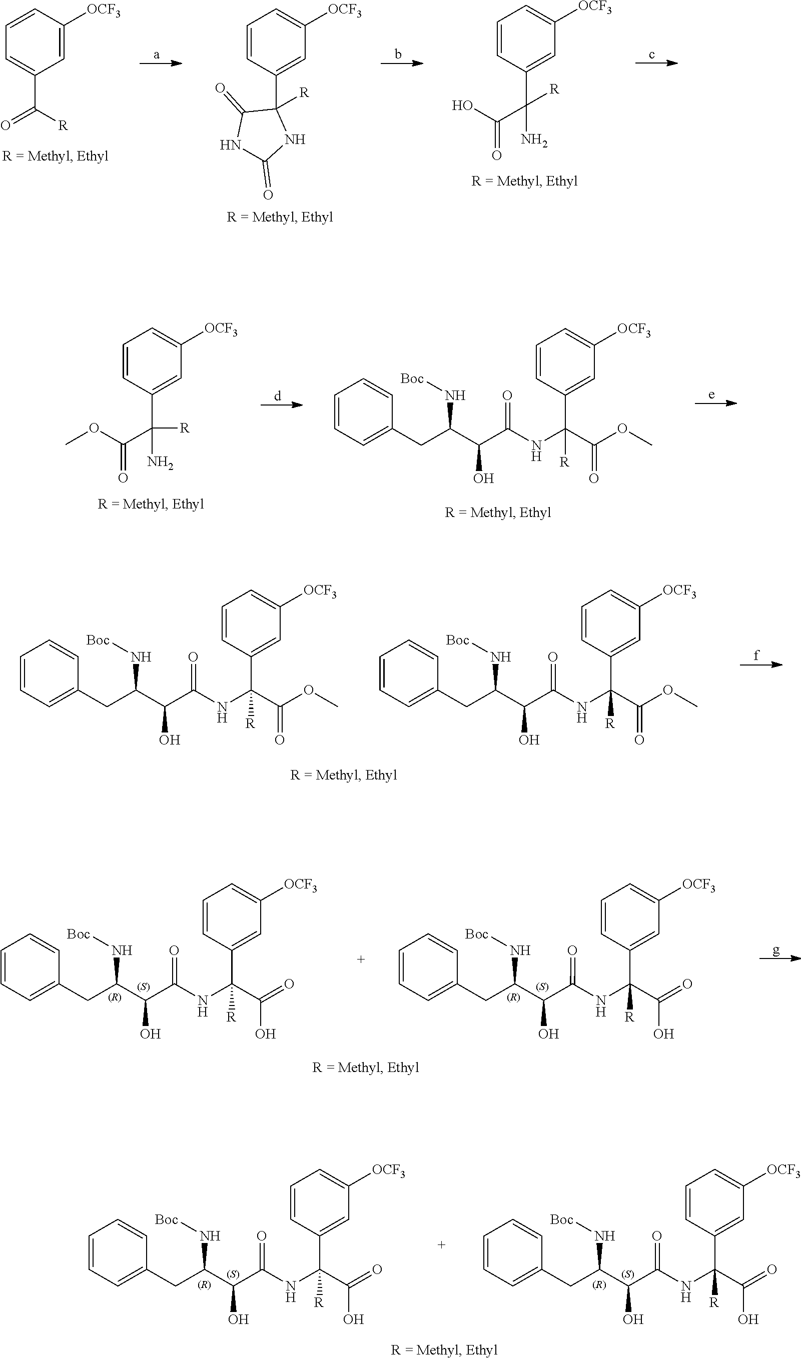

Reagents and conditions: a) KCN, NH₄CO₃, NH₄OH, H₂O, 60° C., 16 h; b) 2N NaOH, reflux, 12 h; c) SOCl₂ in MeOH, 60° C., 16 h; d) Methyl (S)-2-amino-2-(3-(trifluoromethoxy)phenyl)acetate, DCC, HOBt, DMF, rt, 3 h; e) SFC; f) LiOH, THF: H₂O, rt, 8 h; g) 4M HCl in 1,4-dioxane, 0° C., 3 h.

Synthesis of 5-Methyl-5-(3-(trifluoromethoxy)phenyl)imidazolidine-2,4-dione: To a stirred solution of 1-(3-(trifluoromethoxy)phenyl)ethan-1-one (2.5 g, 12.25 mmol) in ethanol (30 mL), and water (30 mL) cooled to 0° C. was added ammonium carbonate (1.400 g, 14.57 mmol) followed by potassium cyanide (3.91 g, 60.0 mmol) under inert atmosphere. The reaction mixture was stirred at 60° C. for 16 h. The progress of the reaction was monitored by TLC. After completion, the reaction mixture was evaporated to dryness to afford the crude mass. The crude compound was purified by silica gel (60-120 mesh silica gel) column chromatography eluting with 70-80% ethyl acetate in hexane. The desired fractions were collected and evaporated to dryness to afford 5-methyl-5-(3-(trifluoromethoxy)phenyl)imidazolidine-2,4-dione (2.5 g, 8.99 mmol, 99% yield) as white solid. LC-MS: 272.9 (M−H).

Synthesis of 2-amino-2-(3-(trifluoromethoxy)phenyl)propanoic acid: To a stirred solution of 5-methyl-5-(3-(trifluoromethoxy)phenyl)imidazolidine-2,4-dione (2.5 g, 9.12 mmol) was added 2N NaOH (75 ml, 9.12 mmol) and the reaction mixture was allowed to stir at 100° C. for 16 h. The progress of the reaction was monitored by TLC. After completion, the reaction mixture was cooled to room temperature, the residue was acidified with 6N HCl (30.0 mL) solution. The resultant precipitated solid was collected by filtration, which was further dissolved in MeOH (150 mL) and filtered. The filtrate was concentrated under reduced pressure to get the product 2-amino-2-(3-(trifluoromethoxy)phenyl)propanoic acid (1.8 g, 5.35 mmol, 58.7% yield) as a white solid. LC-MS: 250.1 (M+H).

Synthesis of methyl 2-amino-2-(3-(trifluoromethoxy)phenyl)propanoate: To a stirred solution of 2-amino-2-(3-(trifluoromethoxy)phenyl)propanoic acid (1.8 g, 7.22 mmol) in MeOH (20 mL) at 0° C. was added thionyl chloride (5.27 ml, 72.2 mmol) under inert atmosphere. The reaction mixture was stirred at 100° C. for 16 h. The progress of the reaction was monitored by TLC. After completion, the reaction mixture was evaporated under reduced pressure to afford the crude mass. The crude compound was purified by silica gel (60-120 mesh silica gel) column chromatography using eluting with 0-6% DCM in Methanol. The desired fractions were collected and evaporated to dryness to afford methyl 2-amino-2-(3-(trifluoromethoxy)phenyl)propanoate (0.45 g, 1.710 mmol, 23.67% yield) as a yellow liquid. LC-MS: 263.95 (M+H).

Synthesis of methyl 2-((2S,3R)-3-((tert-butoxycarbonyl)amino)-2-hydroxy-4-phenylbutanamido)-2-(3-(trifluoromethoxy)phenyl)propanoate: To a stirred solution of (2S,3R)-3-((tert-butoxycarbonyl)amino)-2-hydroxy-4-phenylbutanoic acid (1.5 g, 5.08 mmol) in DMF (15 mL) at 0° C. was added DIPEA (1.774 ml, 10.16 mmol) followed by HATU (2.317 g, 6.09 mmol) and stirred for 20 minutes at 0° C. Then, added methyl 2-amino-2-(3-(trifluoromethoxy)phenyl)propanoate (1.069 g, 4.06 mmol) under inert atmosphere. The reaction mixture was stirred at room temperature for 16 h. The progress of the reaction was monitored by TLC. After completion, the reaction mixture was treated with cold water. This was then extracted with ethyl acetate (2×100 mL) and organic layer was separated. The combined organic fractions were dried over anhydrous Na₂SO₄ and concentrated under reduced pressure to afford the crude mass. The crude compound was purified by silica gel column chromatography eluting with 20-30% ethyl acetate in hexane. The desired fractions were collected and evaporated to dryness to afford methyl 2-((2S,3R)-3-((tert-butoxycarbonyl)amino)-2-hydroxy-4-phenylbutanamido)-2-(3-(trifluoromethoxy)phenyl)propanoate (1.0 g, 0.278 mmol, 5.46% yield) as a white solid. LC-MS: 541.25 (M+H).

This compound was subjected to chiral SFC purification to separate the diastereomers, that resulted in methyl (S)-2-((2S,3R)-3-((tert-butoxycarbonyl)amino)-2-hydroxy-4-phenylbutanamido)-2-(3-(trifluoromethoxy)phenyl)propanoate and methyl (R)-2-((2S,3R)-3-((tert-butoxycarbonyl)amino)-2-hydroxy-4-phenylbutanamido)-2-(3-(trifluoromethoxy)phenyl)propanoate in almost equal proportions.

### Purification Method:

Column: Regis(S,S) Whelk-01, 250 mm×21.1 mm, 5 μm, Mobile Phase: CO₂(A) and 0.1% DEA in EtOH:MeOH(1:1)(B), Flow: 50 ml, Isocratic: 85(A):15(B).

Synthesis of (S)-2-((2S,3R)-3-((tert-butoxycarbonyl)amino)-2-hydroxy-4-phenylbutanamido)-2-(3-(trifluoromethoxy)phenyl)propanoic acid: To a stirred solution of methyl (S)-2-((2S,3R)-3-((tert-butoxycarbonyl)amino)-2-hydroxy-4-phenylbutanamido)-2-(3-(trifluoromethoxy)phenyl)propanoate (100 mg, 0.185 mmol) in THF (2.0 mL) was added LiOH·H₂O (11.66 mg, 0.278 mmol) in water (0.5 mL). The reaction mixture was stirred at room temperature for 8 h. The progress of the reaction was monitored by TLC. After completion, the reaction mixture was evaporated to dryness and the residue was acidified with saturated citric acid solution and adjusted the pH=2. This was then extracted with DCM (2×20 mL) and organic layer was separated. The combined organic fractions were washed with brine solution (5.0 mL), dried over anhydrous Na₂SO₄ and concentrated under reduced pressure to afford (S)-2-((2S,3R)-3-((tert-butoxycarbonyl)amino)-2-hydroxy-4-phenylbutanamido)-2-(3-(trifluoromethoxy)phenyl)propanoic acid (0.06 g, 0.101 mmol, 54.8% yield) as white solid. LC-MS: 527.0 (M+H).

Synthesis of (R)-2-((2S,3R)-3-((tert-butoxycarbonyl)amino)-2-hydroxy-4-phenylbutanamido)-2-(3-(trifluoromethoxy)phenyl)propanoic acid: To a stirred solution of methyl (R)-2-((2S,3R)-3-((tert-butoxycarbonyl)amino)-2-hydroxy-4-phenylbutanamido)-2-(3-(trifluoromethoxy)phenyl)propanoate (120 mg, 0.22 mmol) in THF (2.0 mL) was added LiOH·H₂O (13.99 mg, 0.33 mmol) in water (0.5 mL). The reaction mixture was kept for stirring at room temperature for 8 h. The progress of the reaction was monitored by TLC. After completion, the reaction mixture was evaporated to dryness and the residue was acidified with saturated citric acid solution and adjusted the pH=2. This was then extracted with DCM (2×20 mL) and organic layer was separated. The combined organic fractions were washed with brine solution (5.0 mL), dried over anhydrous Na₂SO₄ and concentrated under reduced pressure to afford (R)-2-((2S,3R)-3-((tert-butoxycarbonyl)amino)-2-hydroxy-4-phenylbutanamido)-2-(3-(trifluoromethoxy)phenyl)propanoic acid (0.1 g, 0.126 mmol, 56.6% yield) as white solid. LC-MS: 527.0 (M+H).

Synthesis of (S)-2-((2S,3R)-3-amino-2-hydroxy-4-phenylbutanamido)-2-(3-(trifluoromethoxy)phenyl)propanoic acid (Compound 88): To a stirred solution of (S)-2-((2S,3R)-3-((tert-butoxycarbonyl)amino)-2-hydroxy-4-phenylbutanamido)-2-(3-(trifluoromethoxy)phenyl)propanoic acid (60 mg, 0.114 mmol) in DCM (0.6 mL) cooled to 0° C. was added 4M HCl in 1,4-Dioxane (0.285 mL, 1.14 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-2-hydroxy-4-phenylbutanamido)-2-(3-(trifluoromethoxy)phenyl)propanoic acid (0.017 g, 0.114 mmol, 34.7% yield) as off-white solid. LC-MS: 427.00 (M+H); HPLC=97.11%; ¹H NMR (DMSO-d₆, 400 MHz) δ 13.2 (brs, 1H), 8.7-8.8 (m, 1H), 7.9-8.4 (m, 3H), 7.2-7.5 (m, 7H), 7.1-7.0 (m, 1H), 6.7-6.8 (m, 1H), 3.9-4.0 (m, 1H), 3.4-3.5 (m, 2H), 2.9-3.0 (m, 1H), 2.7-2.8 (m, 1H), 1.7 (s, 3H). ¹⁹F NMR (DMSO-d₆, 282 MHz) δ −56.49 (s, 3 F).

Synthesis of (R)-2-((2S,3R)-3-amino-2-hydroxy-4-phenylbutanamido)-2-(3-(trifluoromethoxy)phenyl)propanoic acid (Compound 89): To a stirred solution of (R)-2-((2S,3R)-3-((tert-butoxycarbonyl)amino)-2-hydroxy-4-phenylbutanamido)-2-(3-(trifluoromethoxy)phenyl)propanoic acid (100 mg, 0.190 mmol) in DCM (0.6 mL) cooled to 0° C. was added 4M HCl in 1,4-Dioxane (0.475 mL, 1.90 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-2-hydroxy-4-phenylbutanamido)-2-(3-(trifluoromethoxy)phenyl)propanoic acid (0.016 g, 0.19 mmol, 19.59% yield) as off-white solid. LC-MS: 427.00 (M+H); HPLC=98.5%; ¹H NMR (DMSO-d₆, 400 MHz) δ 13.2 (brs, 1H), 8.5-8.7 (m, 1H), 7.9-8.4 (m, 3H), 7.3-7.4 (m, 7H), 7.1-7.2 (m, 1H), 6.9-7.0 (m, 1H), 3.7-3.9 (m, 1H), 3.5-3.7 (m, 1H), 2.8-3.0 (m, 2H), 2.7-2.8 (m, 1H), 1.8 (s, 3H). ¹⁹F NMR (DMSO-d₆, 282 MHz) δ −56.49 (s, 3 F).

Synthesis of (S)-2-((2S,3R)-3-amino-2-hydroxy-4-phenylbutanamido)-2-(3-(trifluoromethoxy)phenyl)butanoic acid (Compound 120): To a stirred solution of (S)-2-((2S,3R)-3-((tert-butoxycarbonyl)amino)-2-hydroxy-4-phenylbutanamido)-2-(3-(trifluoromethoxy)phenyl)butanoic acid (80 mg, 0.148 mmol) in DCM (1 mL) cooled to 0° C. was added 4M HCl in 1,4-Dioxane (0.8 mL, 26.3 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-2-hydroxy-4-phenylbutanamido)-2-(3-(trifluoromethoxy)phenyl)butanoic acid (0.06 mg, 0.136 mmol, 92% yield) as off-white solid. LC-MS: 441.35 (M+H); HPLC=95.5%; ¹H NMR (DMSO-d₆, 400 MHz) δ 13.9 (brs, 1H), 8.5-8.6 (m, 1H), 7.8-8.0 (m, 3H), 7.4-7.5 (m, 2H), 7.3-7.4 (m, 2H), 7.28 (br d, J=7.0 Hz, 4H), 7.10 (d, J=5.9 Hz, 1H), 4.0-4.1 (m, 1H), 3.5-3.6 (m, 2H), 2.9-3.0 (m, 1H), 2.7-2.8 (m, 1H), 2.6-2.7 (m, 1H), 2.44 (br d, J=6.5 Hz, 1H), 0.8-0.9 (m, 3H). ¹⁹F NMR (DMSO-d₆, 282 MHz) δ −56.64 (s, 3 F).

Synthesis of (R)-2-((2S,3R)-3-amino-2-hydroxy-4-phenylbutanamido)-2-(3-(trifluoromethoxy)phenyl)butanoic acid (Compound 119): To a stirred solution of (R)-2-((2S,3R)-3-((tert-butoxycarbonyl)amino)-2-hydroxy-4-phenylbutanamido)-2-(3-(trifluoromethoxy)phenyl)butanoic acid (50 mg, 0.093 mmol) in DCM (1 mL) cooled to 0° C. was added 4M HCl in 1,4-Dioxane (0.5 mL, 16.46 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-2-hydroxy-4-phenylbutanamido)-2-(3-(trifluoromethoxy)phenyl)butanoic acid (0.04 g, 0.091 mmol, 98% yield) as off-white solid. LC-MS: 441.35 (M+H); HPLC=95.2%; ¹H NMR (DMSO-d₆, 400 MHz) δ 13.9 (brs, 1H), 8.6-8.7 (m, 1H), 7.8-8.0 (m, 3H), 7.4-7.5 (m, 2H), 7.3-7.4 (m, 2H), 7.28 (br d, J=7.0 Hz, 4H), 7.10 (d, J=5.9 Hz, 1H), 4.0-4.1 (m, 1H), 3.5-3.6 (m, 2H), 2.9-3.0 (m, 1H), 2.7-2.8 (m, 1H), 2.6-2.7 (m, 1H), 2.44 (br d, J=6.5 Hz, 1H), 0.8-0.9 (m, 3H). ¹⁹F NMR (DMSO-d₆, 282 MHz) δ −56.64 (s, 3 F).

Synthesis of (S)-2-((2S,3R)-3-amino-2-hydroxy-4-phenylbutanamido)-2-(3-(trifluoromethyl)phenyl)propanoic acid (Compound 85): To a stirred solution of (S)-2-((2S,3R)-3-((tert-butoxycarbonyl)amino)-2-hydroxy-4-phenylbutanamido)-2-(3-(trifluoromethyl)phenyl)propanoic acid (20 mg, 0.039 mmol) in DCM (0.2 mL) cooled to 0° C. was added 4M HCl in 1,4-Dioxane (0.098 mL, 0.392 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-2-hydroxy-4-phenylbutanamido)-2-(3-(trifluoromethyl)phenyl)propanoic acid (0.01 g, 0.024 mmol, 61.5% yield) as off white solid. LC-MS: 411.00 (M+H); HPLC=98.9%; ¹H NMR (DMSO-d₆, 400 MHz) δ 13.7 (brs, 1H), 8.8-8.9 (m, 1H), 7.7-7.8 (m, 3H), 7.4-7.5 (m, 2H), 7.2-7.3 (m, 2H), 7.0-7.2 (m, 4H), 6.8-6.9 (m, 1H), 4.0-4.1 (m, 1H), 3.4-3.5 (m, 2H), 2.9-3.0 (m, 1H), 2.6-2.7 (m, 1H), 1.8 (s, 3H). ¹⁹F NMR (DMSO-d₆, 282 MHz) δ −60.93 (s, 3 F).

Synthesis of (R)-2-((2S,3R)-3-amino-2-hydroxy-4-phenylbutanamido)-2-(3-(trifluoromethyl)phenyl)propanoic acid (Compound 84): To a stirred solution of (R)-2-((2S,3R)-3-((tert-butoxycarbonyl)amino)-2-hydroxy-4-phenylbutanamido)-2-(3-(trifluoromethyl)phenyl)propanoic acid (20 mg, 0.039 mmol) in DCM (0.2 mL) cooled to 0° C. was added 4M HCl in 1,4-Dioxane (0.098 mL, 0.392 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-2-hydroxy-4-phenylbutanamido)-2-(3-(trifluoromethyl)phenyl)propanoic acid (0.01 g, 0.024 mmol, 61.5% yield) as off white solid. LC-MS: 411.00 (M+H); HPLC=98.9%; ¹H NMR (DMSO-d₆, 400 MHz) δ 13.7 (brs, 1H), 8.8-8.9 (m, 1H), 7.7-7.8 (m, 3H), 7.4-7.5 (m, 2H), 7.2-7.3 (m, 2H), 7.0-7.2 (m, 4H), 6.8-6.9 (m, 1H), 3.9-4.0 (m, 1H), 3.4-3.5 (m, 2H), 2.9-3.0 (m, 1H), 2.6-2.7 (m, 1H), 1.8 (s, 3H). ¹⁹F NMR (DMSO-d₆, 282 MHz) δ −60.93 (s, 3 F).

### Example 29: Synthesis of (R)-2-((2S,3R)-3-amino-2-hydroxy-4-phenylbutanamido)-2-(2,4-difluoro-3-(trifluoromethyl)phenyl)acetic acid and (S)-2-((2S,3R)-3-amino-2-hydroxy-4-phenylbutanamido)-2-(2,4-difluoro-3-(trifluoromethyl)phenyl)acetic acid (Compound 96 and Compound 97)

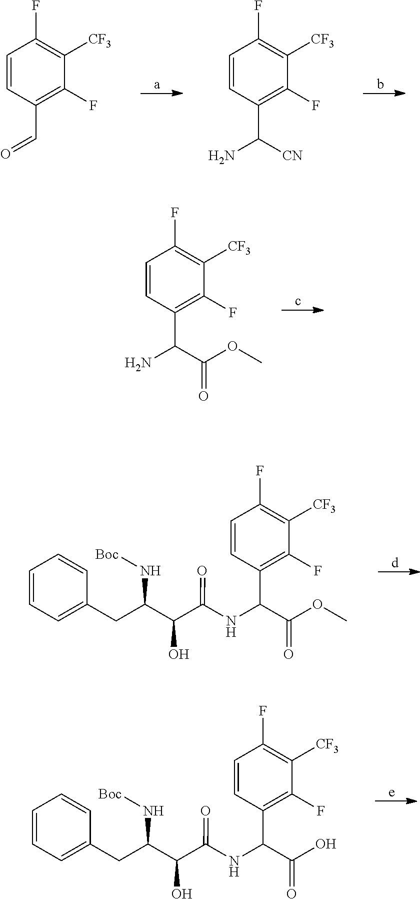 [SMILES: O=Cc1ccc(F)c(C(F)(F)F)c1F ; N#CC(N)c1ccc(F)c(C(F)(F)F)c1F ; COC(=O)C(N)c1ccc(F)c(C(F)(F)F)c1F ; COC(=O)C(NC(=O)[C@@H](O)[C@@H](Cc1ccccc1)NC(=O)OC(C)(C)C)c1ccc(F)c(C(F)(F)F)c1F ; CC(C)(C)OC(=O)N[C@H](Cc1ccccc1)[C@H](O)C(=O)NC(C(=O)O)c1ccc(F)c(C(F)(F)F)c1F]

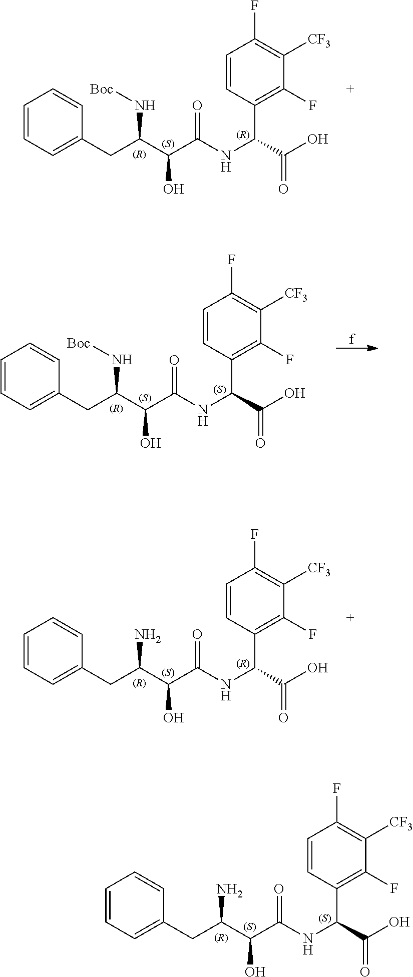

Reagents and conditions: a) ZnI₂, TMSCN, NH₃ in MeOH, 60° C., 3 h; b) HCl in MeOH, 60° C., 16 h; c) Methyl (S)-2-amino-2-(3-(trifluoromethoxy)phenyl)acetate, DCC, HOBt, DMF, rt, 3 h; d) Trimethyltin hydroxide, DCE, 70° C., 2 h; e) SFC f) 4 M HCl in 1,4-dioxane, 0° C., 3 h.

Synthesis of 2-Amino-2-(2,4-difluoro-3-(trifluoromethyl)phenyl)acetonitrile: To a stirred solution of 2,4-difluoro-3-(trifluoromethyl)benzaldehyde (3.0 g, 14.28 mmol) in DCM (20 mL), cooled to 0° C. was added TMSCN (2.87 mL, 21.42 mmol) followed by ZnI₂ (0.45 g, 1.42 mmol) under inert atmosphere. The reaction mixture was stirred for 30 min at 0° C. After 30 min was added NH₃ in MeOH (40 ml, 80.0 mmol). The reaction mixture was stirred at 60° C. for 3 h. The progress of the reaction was monitored by TLC. After completion, the reaction mixture was evaporated to dryness to afford the crude mass. The crude compound was purified by silica gel (60-120 mesh silica gel) column chromatography using RediSep® Bronze, 40 g silica gel eluting with 0-30% ethyl acetate in hexane. The desired fractions were collected and evaporated to dryness to afford 2-amino-2-(2,4-difluoro-3-(trifluoromethyl)phenyl)acetonitrile (2.8 g, 11.86 mmol, 83% yield) as a brown gummy liquid. LCMS: 211.1 (M+H).

Synthesis of methyl 2-amino-2-(2,4-difluoro-3-(trifluoromethyl)phenyl)acetate: To a stirred solution of 2-amino-2-(2,4-difluoro-3-(trifluoromethyl)phenyl)acetonitrile (2.8 g, 11.86 mmol) in methanol (30 mL) was added 4M HCl in 1,4-dioxane (15 mL, 60.0 mmol) and the reaction mixture was allowed to stir at 60° C. for 16 h. The progress of the reaction was monitored by TLC. After completion, the reaction mixture was cooled to room temperature, evaporated to dryness and the residue was treated with saturated sodium bicarbonate solution (15 mL). This was then extracted with ethyl acetate (2×50 mL) and organic layer was separated. The combined organic fractions were dried over anhydrous Na₂SO₄ and concentrated under reduced pressure to afford methyl 2-amino-2-(2,4-difluoro-3-(trifluoromethyl)phenyl)acetate (2.0 g, 7.43 mmol, 62.7% yield) as a white solid. LC-MS: 269.95 (M+H).

Synthesis of methyl 2-((2S,3R)-3-((tert-butoxycarbonyl)amino)-2-hydroxy-4-phenylbutanamido)-2-(2,4-difluoro-3-(trifluoromethyl)phenyl)acetate: To a stirred solution of (2S,3R)-3-((tert-butoxycarbonyl)amino)-2-hydroxy-4-phenylbutanoic acid (1.2 g, 4.06 mmol) in DMF (20 mL) at 0° C. was added HOBt (0.622 g, 4.06 mmol) followed by DCC (0.838 g, 4.06 mmol) and stirred for 20 minutes at 0° C. Then, added methyl 2-amino-2-(2,4-difluoro-3-(trifluoromethyl)phenyl)acetate (0.984 g, 3.66 mmol) under inert atmosphere. The reaction mixture was stirred at room temperature for 16 h. The progress of the reaction was monitored by TLC. After completion, the reaction mixture treated with cold water (50 mL) and then extracted with ethyl acetate (2×50 mL) and organic layer was separated. The combined organic fractions were dried over anhydrous Na₂SO₄ and concentrated under reduced pressure to afford the crude mass. The crude compound was purified by silica gel (60-120 mesh silica gel) column chromatography eluting with 0-50% ethyl acetate in hexane. The desired fractions were collected and evaporated to dryness to afford methyl 2-((2S,3R)-3-((tert-butoxycarbonyl)amino)-2-hydroxy-4-phenylbutanamido)-2-(2,4-difluoro-3-(trifluoromethyl)phenyl)acetate (1.4 g, 2.477 mmol, 61.0% yield) as a white solid. LC-MS: 547.30 (M+H).

Synthesis of 2-((2S,3R)-3-((tert-butoxycarbonyl)amino)-2-hydroxy-4-phenylbutanamido)-2-(2,4-difluoro-3-(trifluoromethyl)phenyl)acetic acid: To a stirred solution of methyl (S)-2-((2S,3R)-3-((tert-butoxycarbonyl)amino)-2-hydroxy-4-phenylbutanamido)-2-(2,4-difluoro-3-(trifluoromethyl)phenyl)acetate (1.4 g, 2.56 mmol) in DCE (20 mL) was trimethyltin hydroxide (1.390 g, 7.69 mmol) under inert atmosphere. The reaction mixture was kept for stirring at 80° C. for 8 h. The progress of the reaction was monitored by TLC. After completion, the reaction mixture was cooled to room temperature and evaporated to dryness. The residue was dissolved in ethyl acetate (100 mL) and washed with 0.1 N potassium bisulphate solution, organic layer was separated and dried over anhydrous sodium sulphate and evaporated to dryness to afford 2-((2S,3R)-3-((tert-butoxycarbonyl)amino)-2-hydroxy-4-phenylbutanamido)-2-(2,4-difluoro-3-(trifluoromethyl)phenyl)acetic acid as white solid.

The compound was purified by SFC to separate the two isomers.
Column: Regis (S,S) Whelk-01, 250 mm×21.1 mm, 5 μm
Mobile Phase: CO₂(A) and 0.1% DEA in EtOH:MeOH(1:1)(B)
Flow:50 mL
Isocratic: 85(A):15(B)
Injection: Volume-0.50 ml
Run Time: 11.5 min
Instrument Make and Model: SFC-PICLAB PREP 175

(S)-2-((2S,3R)-3-((tert-butoxycarbonyl)amino)-2-hydroxy-4-phenylbutanamido)-2-(2,4-difluoro-3-(trifluoromethyl)phenyl)acetic acid (0.22 g, 0.413 mmol, 16.13% yield). LC-MS: 533.05 (M+H).

(R)-2-((2S,3R)-3-((tert-butoxycarbonyl)amino)-2-hydroxy-4-phenylbutanamido)-2-(2,4-difluoro-3-(trifluoromethyl)phenyl)acetic acid (0.35 g, 0.657 mmol, 25.7% yield). LC-MS: 533.05 (M+H).

Synthesis of (S)-2-((2S,3R)-3-amino-2-hydroxy-4-phenylbutanamido)-2-(2,4-difluoro-3-(trifluoromethyl)phenyl)acetic acid (Compound 97): To a stirred solution of (S)-2-((2S,3R)-3-((tert-butoxycarbonyl)amino)-2-hydroxy-4-phenylbutanamido)-2-(2,4-difluoro-3-(trifluoromethyl)phenyl)acetic acid (220 mg, 0.413 mmol) in DCM (10 mL) cooled to 0° C. was added 4M HCl in 1,4-dioxane (3.0 mL, 12.00 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-2-hydroxy-4-phenylbutanamido)-2-(2,4-difluoro-3-(trifluoromethyl)phenyl)acetic acid (0.175 g, 0.394 mmol, 95% yield) as off white-solid. LC-MS: 433.2 (M+H); HPLC=97.4%; ¹H NMR (DMSO-d₆, 400 MHz) δ 13.92 (brs, 1H), 8.6-8.6 (m, 1H), 7.9-8.0 (m, 3H), 7.8-7.9 (m, 1H), 7.3-7.4 (m, 1H), 7.32 (d, J=7.2 Hz, 2H), 7.0-7.3 (m, 4H), 5.6-5.7 (m, 1H), 3.9-4.0 (m, 1H), 3.6-3.6 (m, 1H), 2.89 (br d, J=7.6 Hz, 2H). ¹⁹F NMR (DMSO-d₆, 282 MHz) δ −55.07 (s, 3 F), −115.40 (s, 1 F), −116.13 (s, 1 F).

Synthesis of (R)-2-((2S,3R)-3-amino-2-hydroxy-4-phenylbutanamido)-2-(2,4-difluoro-3-(trifluoromethyl)phenyl)acetic acid (Compound 96): To a stirred solution of (R)-2-((2S,3R)-3-((tert-butoxycarbonyl)amino)-2-hydroxy-4-phenylbutanamido)-2-(2,4-difluoro-3-(trifluoromethyl)phenyl)acetic acid (336 mg, 0.631 mmol) in DCM (10 mL) cooled to 0° C. was added 4M HCl in 1,4-dioxane (3.0 mL, 12.00 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-2-hydroxy-4-phenylbutanamido)-2-(2,4-difluoro-3-(trifluoromethyl)phenyl)acetic acid (0.27 g, 0.603 mmol, 96% yield) as off white-solid. LC-MS: 433.2 (M+H); HPLC=96.6%; ¹H NMR (DMSO-d₆, 400 MHz) δ 13.55 (brs, 1H), 8.9-9.0 (m, 1H), 7.8-7.9 (m, 3H), 7.4-7.5 (m, 2H), 7.3-7.4 (m, 2H), 7.2-7.3 (m, 3H), 6.7-6.8 (m, 1H), 5.7-5.8 (m, 1H), 4.0-4.1 (m, 1H), 3.55 (br d, J=11.7 Hz, 1H), 2.9-3.0 (m, 1H), 2.7-2.8 (m, 1H). ¹⁹F NMR (DMSO-d₆, 282 MHz) δ −55.07 (s, 3 F), −115.40 (s, 1 F), −116.13 (s, 1 F).

### Example 30: Synthesis of (R)-2-((2S,3R)-3-amino-2-hydroxy-4-phenylbutanamido)-2-(4-fluoro-3-(trifluoromethyl)phenyl)acetic acid and (S)-2-((2S,3R)-3-amino-2-hydroxy-4-phenylbutanamido)-2-(4-fluoro-3-(trifluoromethyl)phenyl)acetic acid (Compound 94 and Compound 95)

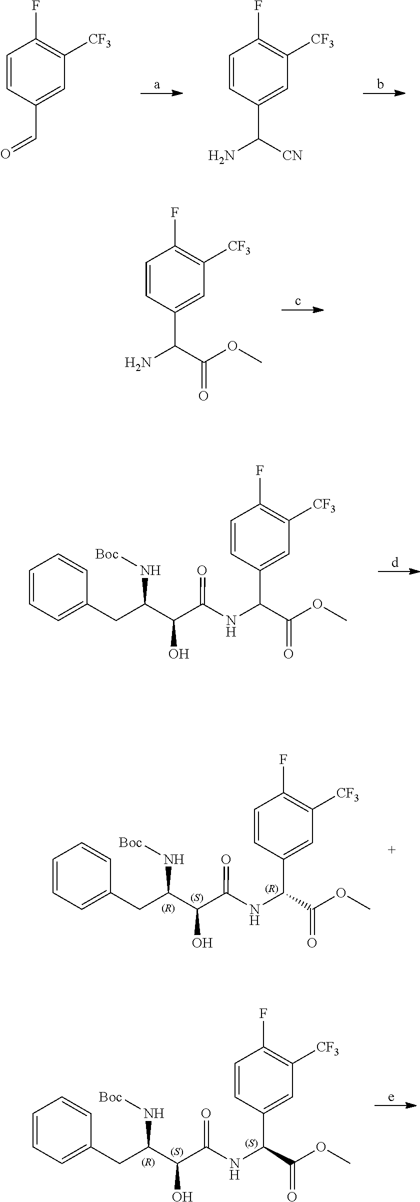

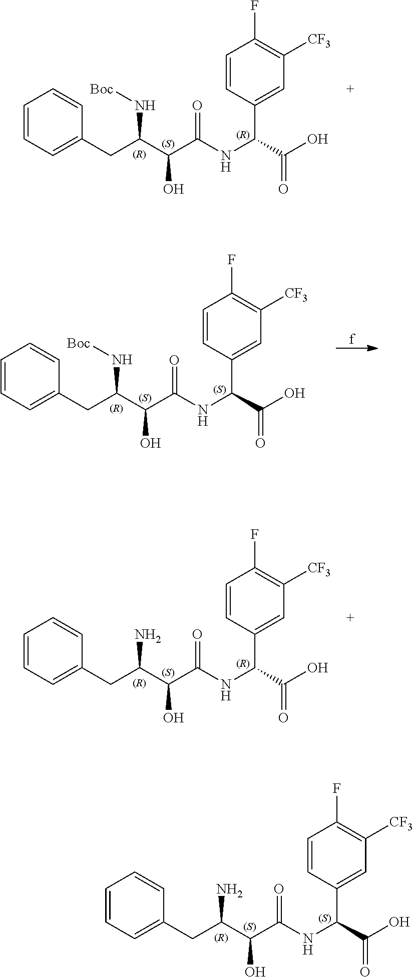

Reagents and conditions: a) ZnI₂, TMSCN, NH₃ in MeOH, 60° C., 3 h; b) HCl in MeOH, 60° C., 16 h; c) Methyl (S)-2-amino-2-(3-(trifluoromethoxy)phenyl)acetate, EDC, HOBt, DMF, rt, 3 h; d) SFC; e) Trimethyltin hydroxide, DCE, 70° C., 2 h; f) 4 M HCl in 1,4-dioxane, 0° C., 3 h.

Synthesis of 2-amino-2-(4-fluoro-3-(trifluoromethyl)phenyl)acetonitrile: To a stirred solution of 4-fluoro-3-(trifluoromethyl)benzaldehyde (10 g, 52.1 mmol) in DCM (100 mL) cooled to 0° C. was added TMSCN (6.98 ml, 52.1 mmol) followed by ZnI₂ (1.661 g, 5.21 mmol) under inert atmosphere. The reaction mixture was stirred for 30 min at 0° C. After 30 min added NH₃ in MeOH (50 mL). The reaction mixture was stirred for 3 h at 60° C. The progress of the reaction was monitored by TLC. After completion, the reaction mixture was evaporated to dryness to afford the crude mass. The crude compound was purified by silica gel (60-120 mesh silica gel) column chromatography using 0-30% o ethyl acetate in hexane as eluent. The desired fractions were collected and evaporated to dryness to afford 2-amino-2-(4-fluoro-3-(trifluoromethyl)phenyl)acetonitrile (3.7 g, 16.96 mmol, 32.6% yield) as a pale yellow solid. LC-MS: 219.06 (M+H).

Synthesis of methyl 2-amino-2-(4-fluoro-3-(trifluoromethyl)phenyl)acetate: To a stirred solution of 2-amino-2-(4-fluoro-3-(trifluoromethyl)phenyl)acetonitrile (3.7 g, 16.96 mmol) in Methanol (40 mL) was added 4M HCl in 1,4-dioxane (35 mL, 140.0 mmol) and the reaction mixture was allowed to stir at 60° C. for 16 h. The progress of the reaction was monitored by TLC. After completion, the reaction mixture was cooled to room temperature, evaporated to dryness and the residue was treated with saturated sodium bicarbonate solution (15 mL). This was then extracted with ethyl acetate (2×50 mL) and organic layer was separated. The combined organic fractions were dried over anhydrous Na₂SO₄ and concentrated under reduced pressure to afford methyl 2-amino-2-(4-fluoro-3-(trifluoromethyl)phenyl)acetate (3 g, 11.94 mmol, 70.4% yield) as an orange-coloured liquid. LC-MS: 251.95 (M+H).

Synthesis of methyl-2-((2S,3R)-3-((tert-butoxycarbonyl)amino)-2-hydroxy-4-phenylbutanamido)-2-(4-fluoro-3-(trifluoromethyl)phenyl)acetate: To a stirred solution of ((2S,3R)-3-((tert-butoxycarbonyl)amino)-2-hydroxy-4-phenylbutanoic acid (2 g, 6.77 mmol) in DMF (20 mL) at 0° C. was added HOBt (2.59 g, 16.93 mmol) followed by EDC (1.947 g, 10.16 mmol) and stirred for 20 minutes at 0° C. Then, added methyl 2-amino-2-(4-fluoro-3-(trifluoromethyl)phenyl)acetate (1.871 g, 7.45 mmol) under inert atmosphere. The reaction mixture was kept for stirring at room temperature for 16 h. The progress of the reaction was monitored by TLC. After completion, the reaction mixture treated with cold water (50 mL) and then extracted with ethyl acetate (2×50 mL) and organic layer was separated. The combined organic fractions were dried over anhydrous Na₂SO₄ and concentrated under reduced pressure to afford the crude mass. The crude compound was purified by silica gel (60-120 mesh silica gel) column chromatography using 0-50% ethyl acetate in hexane as eluent. The desired fractions were collected and evaporated to dryness to afford Methyl-2-((2S,3R)-3-((tert-butoxycarbonyl)amino)-2-hydroxy-4-phenylbutanamido)-2-(4-fluoro-3-(trifluoromethyl)phenyl)acetate as a white solid (2.0 g, 20.43 mmol, 58.3% yield). LC-MS: 529.10 (M+H). The compound was purified by SFC to separate the two isomers.
Column: Regis (S,S) Whelk-01, 250 mm×21.1 mm, 5 μm
Mobile Phase: CO₂(A) and 0.1% DEA in EtOH:MeOH(1:1)(B)
Flow:50 mL
Isocratic:85(A):15(B)
Injection: Volume-0.50 ml
Run Time: 11.5 min
Instrument Make and Model: SFC-PICLAB PREP 175

(Methyl (S)-2-((2S,3R)-3-((tert-butoxycarbonyl)amino)-2-hydroxy-4-phenylbutanamido)-2-(4-fluoro-3-(trifluoromethyl)phenyl)acetate (1 g, 1.816 mmol, 26.8% yield); LC-MS: 529.10 (M+H).

(Methyl (R)-2-((2S,3R)-3-((tert-butoxycarbonyl)amino)-2-hydroxy-4-phenylbutanamido)-2-(4-fluoro-3-(trifluoromethyl)phenyl)acetate (1 g, 1.816 mmol, 26.8% yield); LC-MS: 529.10 (M+H).

Synthesis of (S)-2-((2S,3R)-3-((tert-butoxycarbonyl)amino)-2-hydroxy-4-phenylbutanamido)-2-(4-fluoro-3-(trifluoromethyl)phenyl)acetic acid: To a stirred solution of intermediate methyl (S)-2-((2S,3R)-3-((tert-butoxycarbonyl)amino)-2-hydroxy-4-phenylbutanamido)-2-(4-fluoro-3-(trifluoromethyl)phenyl)acetate (700 mg, 1.325 mmol) DCE (10 mL) was added trimethyltin hydroxide (718 mg, 3.97 mmol) under inert atmosphere. The reaction mixture was kept for stirring at 80° C. for 8 h. The progress of the reaction was monitored by TLC. After completion, the reaction mixture was cooled to room temperature and evaporated to dryness. The residue was dissolved in ethyl acetate (100 mL) and washed with 0.1 N potassium bisulphate solution, organic layer was separated and dried over anhydrous sodium sulphate and evaporated to dryness to afford (S)-2-((2S,3R)-3-((tert-butoxycarbonyl)amino)-2-hydroxy-4-phenylbutanamido)-2-(4-fluoro-3-(trifluoromethyl)phenyl)acetic acid (0.33 g, 0.639 mmol, 48.2% yield) as white solid. LC-MS: 515.05 (M+H).

Synthesis of (R)-2-((2S,3R)-3-((tert-butoxycarbonyl)amino)-2-hydroxy-4-phenylbutanamido)-2-(4-fluoro-3-(trifluoromethyl)phenyl)acetic acid: To a stirred solution of intermediate methyl (R)-2-((2S,3R)-3-((tert-butoxycarbonyl)amino)-2-hydroxy-4-phenylbutanamido)-2-(4-fluoro-3-(trifluoromethyl)phenyl)acetate (800 mg, 1.514 mmol) DCE (10 mL) was added trimethyltin hydroxide (821 mg, 4.54 mmol) under inert atmosphere. The reaction mixture was kept for stirring at 80° C. for 8 h. The progress of the reaction was monitored by TLC. After completion, the reaction mixture was cooled to room temperature and evaporated to dryness. The residue was dissolved in ethyl acetate (100 mL) and washed with 0.1 N potassium bisulphate solution, organic layer was separated and dried over anhydrous sodium sulphate and evaporated to dryness to afford (R)-2-((2S,3R)-3-((tert-butoxycarbonyl)amino)-2-hydroxy-4-phenylbutanamido)-2-(4-fluoro-3-(trifluoromethyl)phenyl)acetic acid (0.32 g, 0.618 mmol, 40.8% yield) as white solid. LC-MS: 515.05 (M+H).

Synthesis of (S)-2-((2S,3R)-3-amino-2-hydroxy-4-phenylbutanamido)-2-(4-fluoro-3-(trifluoromethyl)phenyl)acetic acid (Compound 95): To a stirred solution of (S)-2-((2S,3R)-3-((tert-butoxycarbonyl)amino)-2-hydroxy-4-phenylbutanamido)-2-(4-fluoro-3-(trifluoromethyl)phenyl)acetic acid (270 mg, 0.525 mmol) in DCM (3 mL) cooled to 0^{°} C was added 4M HCl in 1,4-dioxane (3.0 mL, 12.00 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (30 mL) and dried to afford (S)-2-((2S,3R)-3-amino-2-hydroxy-4-phenylbutanamido)-2-(4-fluoro-3-(trifluoromethyl)phenyl)acetic acid (0.085 g, 0.201 mmol, 38.3% yield) as off-white solid. LC-MS: 415.10 (M+H); HPLC=98.7%; ¹H NMR (DMSO-d₆, 400 MHz) δ 13.3 (brs, 1H), 8.0-8.4 (m, 3H), 7.6-7.7 (m, 3H), 7.3-7.4 (m, 6H), 6.9-7.0 (m, 1H), 4.9-5.0 (m, 1H), 3.8-3.9 (m, 1H), 3.5-3.6 (m, 1H), 2.8-2.9 (m, 2H). ¹⁹F NMR (DMSO-d₆, 282 MHz) δ −59.94 (s, 3 F), −119.73 (s, 1 F).

Synthesis of (R)-2-((2S,3R)-3-amino-2-hydroxy-4-phenylbutanamido)-2-(4-fluoro-3-(trifluoromethyl)phenyl)acetic acid (Compound 94): To a stirred solution of (R)-2-((2S,3R)-3-((tert-butoxycarbonyl)amino)-2-hydroxy-4-phenylbutanamido)-2-(4-fluoro-3-(trifluoromethyl)phenyl)acetic acid (270 mg, 0.525 mmol) in DCM (3 mL) cooled to 0° C. was added 4M HCl in 1,4-dioxane (3.0 mL, 12.00 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (30 mL) and dried to afford (R)-2-((2S,3R)-3-amino-2-hydroxy-4-phenylbutanamido)-2-(4-fluoro-3-(trifluoromethyl)phenyl)acetic acid (0.090 g, 0.211 mmol, 40.1% yield) as off-white solid. LC-MS: 415.10 (M+H); HPLC=98.7%; ¹H NMR (DMSO-d₆, 400 MHz) δ 13.3 (brs, 1H), 8.0-8.4 (m, 3H), 7.6-7.7 (m, 3H), 7.3-7.4 (m, 6H), 6.2-6.3 (m, 1H), 4.8-4.9 (m, 1H), 3.9-4.0 (m, 1H), 3.5-3.6 (m, 1H), 2.9-3.0 (m, 1H), 2.7-2.8 (m, 1H). ¹⁹F NMR (DMSO-d₆, 282 MHz) δ −59.94 (s, 3 F), −119.73 (s, 1 F).

### Example 31: Synthesis of (R)-2-((2S,3R)-3-amino-2-hydroxy-4-phenylbutanamido)-2-(2-chloro-3-(trifluoromethyl)phenyl)acetic acid, (S)-2-((2S,3R)-3-amino-2-hydroxy-4-phenylbutanamido)-2-(2-chloro-3-(trifluoromethyl)phenyl)acetic acid, (R)-2-((2S,3R)-3-amino-2-hydroxy-4-phenylbutanamido)-2-(2-fluoro-3-(trifluoromethyl)phenyl)acetic acid, and (S)-2-((2S,3R)-3-amino-2-hydroxy-4-phenylbutanamido)-2-(2-fluoro-3-(trifluoromethyl)phenyl)acetic acid (Compound 86, Compound 87, Compound 90, and Compound 91)

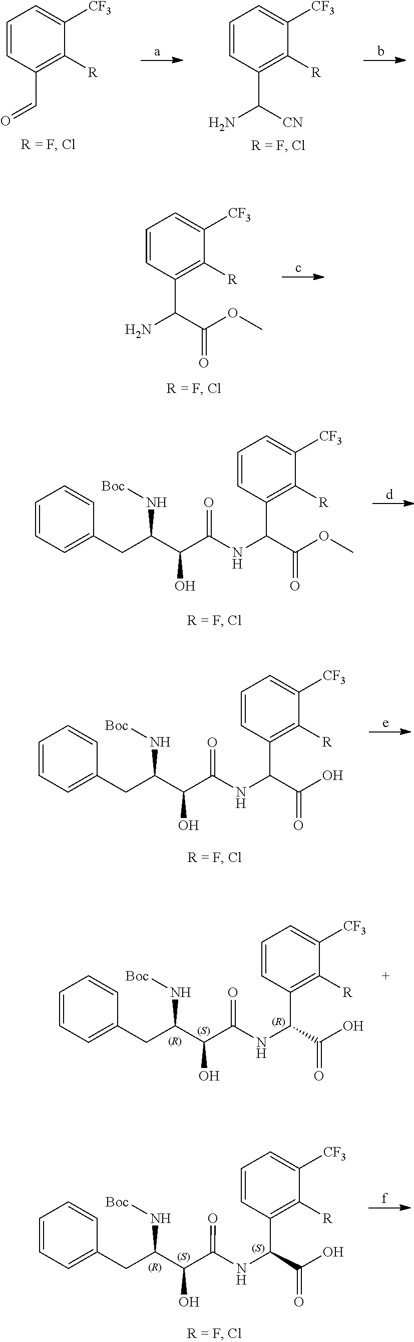

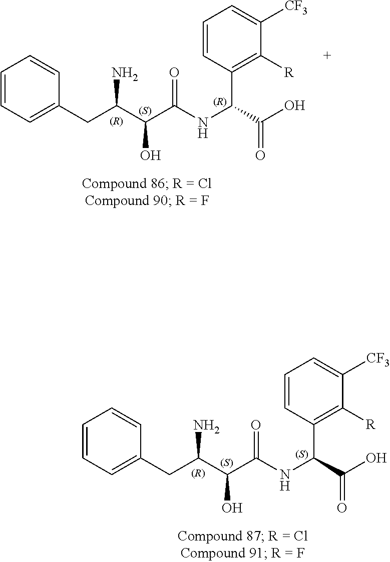

Reagents and conditions: a) ZnI₂, TMSCN, NH₃ in MeOH, 60° C., 3 h; b) HCl in MeOH, 60° C., 16 h; c) Methyl (S)-2-amino-2-(3-(trifluoromethoxy)phenyl)acetate, DCC, HOBt, DMF, rt, 8 h; d) Trimethyltin hydroxide, DCE, 70° C., 2 h; e) SFC f) 4 M HCl in dioxane, 0° C., 3 h.

Synthesis of 2-amino-2-(2-chloro-3-(trifluoromethyl)phenyl)acetonitrile: To a stirred solution of 2-chloro-3-(trifluoromethyl)benzaldehyde (500 mg, 2.397 mmol) in DCM (5 mL), cooled to 0° C. was added TMS-CN (0.321 mL, 2.397 mmol) followed by ZnI₂ (77 mg, 0.240 mmol) under inert atmosphere. The reaction mixture was stirred for 30 min at 0° C. Then, added NH₃ in MeOH (2.5 mL, 17.50 mmol). The reaction mixture was stirred for 3 h at 60° C. The progress of the reaction was monitored by TLC. After completion, the reaction mixture was evaporated to dryness to afford the crude mass. The crude compound was purified by silica gel (60-120 mesh silica gel) column chromatography eluting with 0-30% ethyl acetate in hexane. The desired fractions were collected and evaporated to dryness to afford 2-amino-2-(2-chloro-3-(trifluoromethyl)phenyl)acetonitrile (0.5 g, 2.131 mmol, 89% yield) was isolated as a pale-yellow liquid. LCMS: 235.1 (M+H).

Synthesis of methyl 2-amino-2-(2-chloro-3-(trifluoromethyl)phenyl)acetate: To a stirred solution of 2-amino-2-(2-chloro-3-(trifluoromethyl)phenyl)acetonitrile (1.2 g, 5.11 mmol) in Methanol (20 mL) was added 4M HCl in 1,4-dioxane (12 mL, 48.0 mmol) and the reaction mixture was allowed to stir at 60° C. for 16 h. The progress of the reaction was monitored by TLC. After completion, the reaction mixture was cooled to room temperature, evaporated to dryness and the residue was treated with saturated sodium bicarbonate solution (15 mL). This was then extracted with ethyl acetate (2×50 mL) and organic layer was separated. The combined organic fractions were dried over anhydrous Na₂SO₄ and concentrated under reduced pressure to afford methyl 2-amino-2-(2-chloro-3-(trifluoromethyl)phenyl)acetate (1.2 g, 4.48 mmol, 88% yield) as an brown liquid. LC-MS: 267.95 (M+H).

Synthesis of methyl 2-((2S,3R)-3-((tert-butoxycarbonyl)amino)-2-hydroxy-4-phenylbutanamido)-2-(2-chloro-3-(trifluoromethyl)phenyl)acetate: To a stirred solution of (2S,3R)-3-((tert-butoxycarbonyl)amino)-2-hydroxy-4-phenylbutanoic acid (1.0 g, 3.39 mmol) in DMF (10 mL) at 0° C. was added HOBt (0.519 g, 3.39 mmol) followed by DCC (0.699 g, 3.39 mmol) and the resultant mixture was stirred for 20 minutes at 0° C. Then, added methyl 2-amino-2-(2-chloro-3-(trifluoromethyl)phenyl)acetate (0.725 g, 2.71 mmol) under inert atmosphere. The reaction mixture was stirred at room temperature for 3 h. The progress of the reaction was monitored by TLC. After completion, the reaction mixture was treated with cold water (50 mL). This was then extracted with ethyl acetate (2×50 mL) and organic layer was separated. The combined organic fractions were dried over anhydrous Na₂SO₄ and concentrated under reduced pressure to afford the crude mass. The crude compound was purified by silica gel (60-120 mesh silica gel) column chromatography using 0-60% ethyl acetate in hexane as eluent. The desired fractions were collected and evaporated to dryness to afford methyl 2-((2S,3R)-3-((tert-butoxycarbonyl)amino)-2-hydroxy-4-phenylbutanamido)-2-(2-chloro-3-(trifluoromethyl)phenyl)acetate (0.8 g, 1.468 mmol, 43.4% yield) as a white solid. LC-MS: 545.05 (M+H).

Synthesis of 2-((2S,3R)-3-((tert-butoxycarbonyl)amino)-2-hydroxy-4-phenylbutanamido)-2-(2-chloro-3-(trifluoromethyl)phenyl)acetic acid: To a stirred solution of methyl 2-((2S,3R)-3-((tert-butoxycarbonyl)amino)-2-hydroxy-4-phenylbutanamido)-2-(2-chloro-3-(trifluoromethyl)phenyl)acetate (400 mg, 0.734 mmol) in DCE (8 mL) was added trimethyltin hydroxide (398 mg, 2.202 mmol) under inert atmosphere. The reaction mixture was stirred at 80° C. for 8 h. The progress of the reaction was monitored by TLC. After completion, the reaction mixture was cooled to room temperature and evaporated to dryness. The residue was dissolved in ethyl acetate (20 mL) and washed with 0.1 N potassium bisulphate solution, organic layer was separated and dried over anhydrous sodium sulphate and evaporated to dryness to afford 2-((2S,3R)-3-((tert-butoxycarbonyl)amino)-2-hydroxy-4-phenylbutanamido)-2-(2-chloro-3-(trifluoromethyl)phenyl)acetic acid as white solid. The compound was purified by SFC to separate the two isomers.

The compound was purified by SFC to separate the two isomers.
Column: Regis (S,S) Whelk-01, 250 mm×21.1 mm, 5 μm
Mobile Phase: CO₂(A) and 0.1% DEA in EtOH:MeOH(1:1)(B)
Flow: 50 mL
Isocratic: 85(A):15(B)
Injection: Volume-0.50 ml
Run Time: 11.5 min
Instrument Make and Model: SFC-PICLAB PREP 175

(S)-2-((2S,3R)-3-((tert-butoxycarbonyl)amino)-2-hydroxy-4-phenylbutanamido)-2-(2-chloro-3-(trifluoromethyl)phenyl)acetic acid (0.1 g, 0.188 mmol, 25.7% yield). LC-MS: 531.05 (M+H).

(R)-2-((2S,3R)-3-((tert-butoxycarbonyl)amino)-2-hydroxy-4-phenylbutanamido)-2-(2-chloro-3-(trifluoromethyl)phenyl)acetic acid (0.1 g, 0.188 mmol, 25.7% yield). LC-MS: 531.05 (M+H).

Synthesis of (S)-2-((2S,3R)-3-amino-2-hydroxy-4-phenylbutanamido)-2-(2-chloro-3-(trifluoromethyl)phenyl)acetic acid (Compound 87): To a stirred solution of (S)-2-((2S,3R)-3-((tert-butoxycarbonyl)amino)-2-hydroxy-4-phenylbutanamido)-2-(2-chloro-3-(trifluoromethyl)phenyl)acetic acid (100 mg, 0.188 mmol) in DCM (3 mL) cooled to 0° C. was added 4M HCl in 1,4-dioxane (0.3 mL, 9.87 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (50 mL) and dried to afford (S)-2-((2S,3R)-3-amino-2-hydroxy-4-phenylbutanamido)-2-(2-chloro-3-(trifluoromethyl)phenyl)acetic acid (0.04 g, 0.093 mmol, 94% yield) as off-white solid. LC-MS: 431.10 (M+H); HPLC=98.6%; ¹H NMR (DMSO-d₆, 400 MHz) δ 13.9 (brs, 1H), 8.6-8.7 (m, 1H), 7.94 (br s, 3H), 7.8-7.9 (m, 2H), 7.2-7.4 (m, 6H), 6.9-7.0 (m, 1H), 5.60 (d, J=6.3 Hz, 1H), 3.9-4.0 (m, 1H), 3.5-3.6 (m, 1H), 2.8-2.9 (m, 2H). ¹⁹F NMR (DMSO-d₆, 282 MHz) δ −60.95 (s, 3 F).

Synthesis of (R)-2-((2S,3R)-3-amino-2-hydroxy-4-phenylbutanamido)-2-(2-chloro-3-(trifluoromethyl)phenyl)acetic acid (Compound 86): To a stirred solution of (R)-2-((2S,3R)-3-((tert-butoxycarbonyl)amino)-2-hydroxy-4-phenylbutanamido)-2-(2-chloro-3-(trifluoromethyl)phenyl)acetic acid (100 mg, 0.188 mmol) in DCM (3 mL) cooled to 0° C. was added 4M HCl in 1,4-Dioxane (0.3 mL, 9.87 mmol) (Symax Laboratories) under inert atmosphere. The reaction mixture was stirred at room temperature for 3 h. The progress of the reaction was monitored by TLC. After completion, the reaction mixture was evaporated to dryness. The residue was washed with diethyl ether (50 mL) and dried to afford (R)-2-((2S,3R)-3-amino-2-hydroxy-4-phenylbutanamido)-2-(2-chloro-3-(trifluoromethyl)phenyl)acetic acid (0.03 g, 0.093 mmol, 85% yield) as off-white solid. LC-MS: 431.10 (M+H); HPLC=97.0%; ¹H NMR (DMSO-d₆, 400 MHz) δ 13.9 (brs, 1H), 8.2-8.6 (m, 3H), 8.0-8.1 (m, 1H), 7.73 (br d, J=7.2 Hz, 1H), 7.6-7.7 (m, 1H), 7.5-7.5 (m, 1H), 7.3-7.4 (m, 2H), 7.2-7.3 (m, 3H), 6.88 (br d, J=3.5 Hz, 1H), 5.4-5.5 (m, 1H), 3.78 (br d, J=2.6 Hz, 1H), 3.53 (br t, J=7.2 Hz, 1H), 2.91 (br d, J=7.8 Hz, 2H). ¹⁹F NMR (DMSO-d₆, 282 MHz) δ −60.95 (s, 3 F).

Synthesis of (S)-2-((2S,3R)-3-amino-2-hydroxy-4-phenylbutanamido)-2-(2-fluoro-3-(trifluoromethyl)phenyl)acetic acid (Compound 91): To a stirred solution of (S)-2-((2S,3R)-3-((tert-butoxycarbonyl)amino)-2-hydroxy-4-phenylbutanamido)-2-(2-fluoro-3-(trifluoromethyl)phenyl)acetic acid (100 mg, 0.194 mmol) in DCM (3 mL) cooled to 0° C. was added 4M HCl in 1,4-dioxane (0.3 mL, 9.96 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (50 mL) and dried to afford (S)-2-((2S,3R)-3-amino-2-hydroxy-4-phenylbutanamido)-2-(2-fluoro-3-(trifluoromethyl)phenyl)acetic acid (0.015 g, 0.0033 mmol, 16.8% yield) as off-white solid. LC-MS: 414.95 (M+H); HPLC=91.6%; ¹H NMR (DMSO-d₆, 400 MHz) δ 13.7 (brs, 1H), 8.3-8.5 (m, 1H), 7.9-8.3 (m, 3H), 7.7-7.8 (m, 2H), 7.2-7.4 (m, 6H), 6.9-7.0 (m, 1H), 5.4-5.6 (m, 1H), 3.9-4.0 (m, 1H), 3.6-3.7 (m, 1H), 2.8-2.9 (m, 2H). ¹⁹F NMR (DMSO-d₆, 282 MHz) δ −59.88 (s, 3 F), −121.14 (s, 1 F).

Synthesis of (R)-2-((2S,3R)-3-amino-2-hydroxy-4-phenylbutanamido)-2-(2-fluoro-3-(trifluoromethyl)phenyl)acetic acid (Compound 90): To a stirred solution of (R)-2-((2S,3R)-3-((tert-butoxycarbonyl)amino)-2-hydroxy-4-phenylbutanamido)-2-(2-fluoro-3-(trifluoromethyl)phenyl)acetic acid (60 mg, 0.117 mmol) in DCM (3 mL) cooled to 0° C. was added 4M HCl in 1,4-dioxane (0.3 mL, 9.2 mmol) under inert atmosphere. The reaction mixture was stirred at room temperature for 3 h. The progress of the reaction was monitored by TLC. After completion, the reaction mixture was evaporated to dryness. The residue was washed with diethyl ether (50 mL) and dried to afford (R)-2-((2S,3R)-3-amino-2-hydroxy-4-phenylbutanamido)-2-(2-fluoro-3-(trifluoromethyl)phenyl)acetic acid (0.015 g, 0.036 mmol, 31% yield) as off-white solid. LC-MS: 414.95 (M+H); HPLC=94.3%; ¹H NMR (DMSO-d₆, 400 MHz) δ 13.7 (brs, 1H), 8.9-9.0 (m, 1H), 7.8-7.9 (m, 3H), 7.7-7.8 (m, 2H), 7.4-7.5 (m, 1H), 7.3-7.4 (m, 5H), 6.7-6.8 (m, 1H), 5.7-5.8 (m, 1H), 4.0-4.1 (m, 1H), 3.5-3.6 (m, 1H), 2.9-3.0 (m, 1H), 2.7-2.8 (m, 1H). ¹⁹F NMR (DMSO-d₆, 282 MHz) δ −59.88 (s, 3 F), −121.14 (s, 1 F).

Synthesis of (S)-2-((2S,3R)-3-amino-2-hydroxy-4-phenylbutanamido)-2-(5-fluoro-3-(trifluoromethyl)phenyl)acetic acid (Compound 93): To a stirred solution of (S)-2-((2S,3R)-3-((tert-butoxycarbonyl)amino)-2-hydroxy-4-phenylbutanamido)-2-(5-fluoro-3-(trifluoromethyl)phenyl)acetic acid (280 mg, 0.544 mmol) in DCM (3 mL) cooled to 0° C. was added 4M HCl in 1,4-dioxane (1 mL, 4.0 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (50 mL) and dried to afford (S)-2-((2S,3R)-3-amino-2-hydroxy-4-phenylbutanamido)-2-(5-fluoro-3-(trifluoromethyl)phenyl)acetic acid (0.14 g, 0.33 mmol, 61.3% yield) as off-white solid. LC-MS: 413.15 (M+H); HPLC=98.8%; ¹H NMR (DMSO-d₆, 400 MHz) δ 13.8 (brs, 1H), 8.1-8.3 (m, 3H), 7.5-7.5 (m, 1H), 7.4-7.5 (m, 2H), 7.3-7.4 (m, 2H), 7.2-7.3 (m, 4H), 6.9-7.0 (m, 1H), 4.9-5.0 (m, 1H), 3.8-3.9 (m, 1H), 3.5-3.6 (m, 1H), 2.9-3.0 (m, 2H). ¹⁹F NMR (DMSO-d₆, 282 MHz) δ −61.03 (s, 3 F), −111.85 (s, 1 F).

Synthesis of (R)-2-((2S,3R)-3-amino-2-hydroxy-4-phenylbutanamido)-2-(5-fluoro-3-(trifluoromethyl)phenyl)acetic acid (Compound 92): To a stirred solution of (R)-2-((2S,3R)-3-((tert-butoxycarbonyl)amino)-2-hydroxy-4-phenylbutanamido)-2-(5-fluoro-3-(trifluoromethyl)phenyl)acetic acid (280 mg, 0.544 mmol) in DCM (3 mL) cooled to 0° C. was added 4M HCl in 1,4-dioxane (1 mL, 4.0 mmol) under inert atmosphere. The reaction mixture was stirred at room temperature for 3 h. The progress of the reaction was monitored by TLC. After completion, the reaction mixture was evaporated to dryness. The residue was washed with diethyl ether (50 mL) and dried to afford (S)-2-((2S,3R)-3-amino-2-hydroxy-4-phenylbutanamido)-2-(5-fluoro-3-(trifluoromethyl)phenyl)acetic acid (0.12 g, 0.28 mmol, 51.5% yield) as off-white solid. LC-MS: 413.15 (M+H); HPLC=96.7%; ¹H NMR (DMSO-d₆, 400 MHz) δ 13.8 (brs, 1H), 8.6-8.7 (m, 1H), 7.8-8.3 (m, 3H), 7.6-7.6 (m, 1H), 7.5-7.5 (m, 2H), 7.3-7.4 (m, 2H), 7.2-7.3 (m, 3H), 6.3-6.5 (m, 1H), 4.9-5.0 (m, 1H), 4.0-4.0 (m, 1H), 3.5-3.6 (m, 1H), 2.9-3.0 (m, 1H), 2.7-2.8 (m, 1H). ¹⁹F NMR (DMSO-d₆, 282 MHz) δ −61.03 (s, 3 F), −111.85 (s, 1 F).

### Example 32: Synthesis of Compound 121 to Compound 132

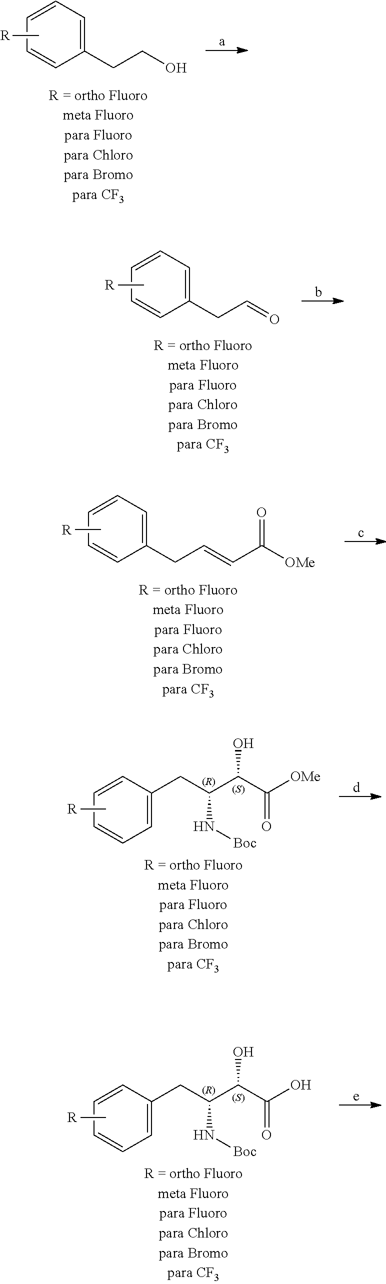

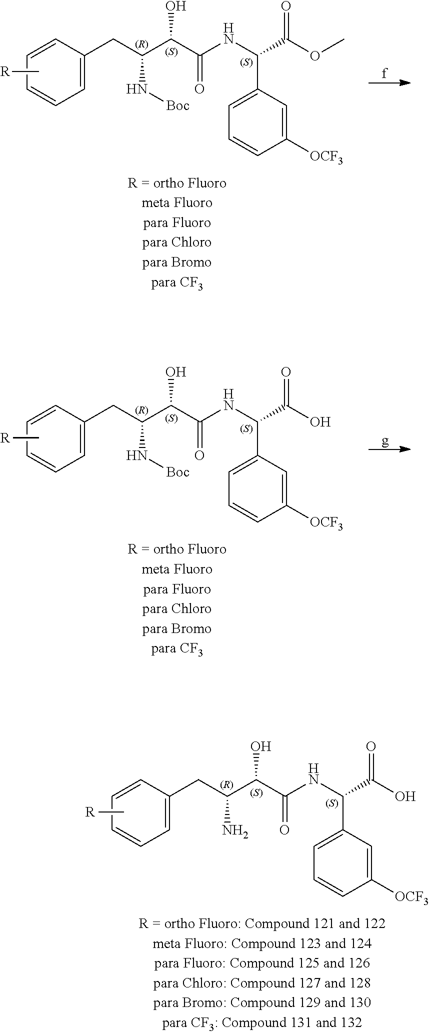

Reagents and conditions: a) DMP, DCM, rt, 12 h; b) Methyl 2-(diethoxyphosphoryl)acetate, NaH, Et₂O, 0° C., 1 h; c) Tert-butyl carbamate, (DHQD)₂PHAL, K(OsO₂(OH) 4), t-BuOCl, NaOH, n-PrOH—H₂O, 0° C., 2 h; d) Trimethyltin hydroxide, DCE, 70° C., 2 h; e) Methyl (S)-2-amino-2-(3-(trifluoromethoxy)phenyl)acetate, DCC, HOBt, DMF, rt, 3 h; f) Trimethyltin hydroxide, DCE, 70° C., 2 h; g) 4 M HCl in 1,4-dioxane, 0° C., 3 h.

Synthesis of 2-(4-chlorophenyl)acetaldehyde: To a stirred solution of 2-(4-chlorophenyl)ethan-1-ol (2.0 g, 12.77 mmol) in DCM (20 mL), cooled to 0° C. was added dess-martinperiodinane (5.42 g, 12.77 mmol) under inert atmosphere. The reaction mixture was stirred room temperature for 8 h. The progress of the reaction was monitored by TLC. After completion, the reaction mixture was evaporated to dryness to afford the crude mass. The crude compound was purified by silica gel (60-120 mesh silica gel) column chromatography using 0-30% ethyl acetate in hexane as eluent. The desired fractions were collected and evaporated to dryness to afford 2-(4-chlorophenyl)acetaldehyde (1.0 g, 6.47 mmol, 50.7% yield) as a pale-yellow liquid LCMS: 155.1 (M+H).

Synthesis of methyl (E)-4-(4-chlorophenyl)but-2-enoate: To a stirred solution of methyl 2-(diethoxyphosphoryl)acetate (1.432 mL, 7.77 mmol) in diethyl ether (20 mL) cooled to 0° C. was added NaH (0.186 g, 7.76 mmol) followed by potassium cyanide (2.059 g, 31.6 mmol) under inert atmosphere. The reaction mixture was stirred for 30 minutes at 0° C. Then, added 2-(4-chlorophenyl)acetaldehyde (1.0 g, 6.47 mmol). The reaction mixture was stirred for 3 h at 0° C. The progress of the reaction was monitored by TLC. After completion, the reaction mixture was evaporated to dryness to afford the crude mass. The crude compound was purified by silica gel (60-120 mesh silica gel) column chromatography using 0-30% ethyl acetate in hexane as eluent. The desired fractions were collected and evaporated to dryness to afford methyl (E)-4-(4-chlorophenyl)but-2-enoate (1.0 g, 4.75 mmol, 73.4% yield) as a colorless liquid. LCMS: 211.1 (M+H).

Synthesis of methyl (2S,3R)-3-((tert-butoxycarbonyl)amino)-4-(4-chlorophenyl)-2-hydroxybutanoate: To a stirred solution of tert-butyl carbamate (834 mg, 7.12 mmol) in 2-propanol (12 mL) at 0° C. was added NaOH (285 mg, 7.12 mmol) followed by tert-butyl hypochlorite (0.807 mL, 7.12 mmol) and stirred for 20 minutes at 0° C. Then, added AD-mix-beta (111 mg, 0.142 mmol) dissolved in 2-propanol (12 mL), methyl (E)-4-(4-chlorophenyl)but-2-enoate (500 mg, 2.374 mmol) dissolved in 2-propanol (12 mL) and potassium osmate dihydrate (35.0 mg, 0.095 mmol) under inert atmosphere. The reaction mixture was stirred at room temperature for 16 h. The progress of the reaction was monitored by TLC. After completion, the reaction mixture treated with cold saturated sodium bicarbonate solution (5 mL). This was then extracted with ethyl acetate (2×20 mL) and organic layer was separated. The combined organic fractions were dried over anhydrous Na₂SO₄ and concentrated under reduced pressure to afford the crude mass. The crude compound was purified by silica gel (60-120 mesh silica gel) column chromatography using 0-20% ethyl acetate in hexane as eluent. The desired fractions were collected and evaporated to dryness to afford white solid. Then the separated the major and minor isomers from SFC.

### Major Peak:

Methyl (2S,3R)-3-((tert-butoxycarbonyl)amino)-4-(4-chlorophenyl)-2-hydroxybutanoate (0.12 g, 0.321 mmol, 13.52% yield).

### Minor Peak:

Methyl (2R,3S)-3-((tert-butoxycarbonyl)amino)-4-(4-chlorophenyl)-2-hydroxybutanoate (0.06 g, 0.119 mmol, 5.00% yield)

Note: The stereochemistry mentioned in the product is relative. The absolute stereochemistry is not known for the chiral centre adjacent to amine and alcohol.

Synthesis of (2S,3R)-3-((tert-butoxycarbonyl)amino)-4-(4-chlorophenyl)-2-hydroxybutanoic acid: To a stirred solution of methyl (2S,3R)-3-((tert-butoxycarbonyl)amino)-4-(4-chlorophenyl)-2-hydroxybutanoate (60 mg, 0.175 mmol) in DCE (1 mL) was added trimethyltin hydroxide (95 mg, 0.524 mmol) under inert atmosphere. The reaction mixture was kept for stirring at 80° C. for 6 h. The progress of the reaction was monitored by TLC. After completion, the reaction mixture was cooled to room temperature and evaporated to dryness. The residue was dissolved in ethyl acetate (20 mL) and washed with 0.1 N potassium bisulphate solution, organic layer was separated and dried over anhydrous sodium sulphate and evaporated to dryness to afford (2S,3R)-3-((tert-butoxycarbonyl)amino)-4-(4-chlorophenyl)-2-hydroxybutanoic acid (0.05 g, 0.152 mmol, 87% yield) as white solid. LCMS: 329.95 (M+H).

Synthesis of methyl (S)-2-((2S,3R)-3-((tert-butoxycarbonyl)amino)-4-(4-chlorophenyl)-2-hydroxybutanamido)-2-(3-(trifluoromethoxy)phenyl)acetate: To a stirred solution of (2S,3R)-3-((tert-butoxycarbonyl)amino)-4-(4-chlorophenyl)-2-hydroxybutanoic acid (50 mg, 0.152 mmol) in DMF (5 mL) at 0° C. was added HOBt (25.5 mg, 0.167 mmol) followed by EDC (32.0 mg, 0.167 mmol) and stirred for 20 minutes at 0° C. Then, added methyl (S)-2-amino-2-(3-(trifluoromethoxy)phenyl)acetate (30.2 mg, 0.121 mmol) under inert atmosphere. The reaction mixture was kept for stirring at room temperature for 16 h. The progress of the reaction was monitored by TLC. After completion, the reaction mixture was treated with cold saturated sodium bicarbonate solution (5 mL). This was then extracted with ethyl acetate (2×20 mL) and organic layer was separated. The combined organic fractions were dried over anhydrous Na₂SO₄ and concentrated under reduced pressure to afford the crude mass. The crude compound was purified by silica gel (60-120 mesh silica gel) column chromatography using 0-50% ethyl acetate in hexane as eluent. The desired fractions were collected and evaporated to dryness to afford methyl (S)-2-((2S,3R)-3-((tert-butoxycarbonyl)amino)-4-(4-chlorophenyl)-2-hydroxybutanamido)-2-(3-(trifluoromethoxy)phenyl)acetate (0.04 g, 0.071 mmol, 47.0% yield) as a white solid. LC-MS: 562.90 (M+H).

Synthesis of 2-((2S,3R)-3-((tert-butoxycarbonyl)amino)-4-(4-chlorophenyl)-2-hydroxybutanamido)-2-(3-(trifluoromethoxy)phenyl)acetic acid: To a stirred solution of methyl (2S,3R)-3-((tert-butoxycarbonyl)amino)-4-(4-chlorophenyl)-2-hydroxybutanoate (60 mg, 0.175 mmol) in DCE (1 mL) was added trimethyltin hydroxide (95 mg, 0.524 mmol) under inert atmosphere. The reaction mixture was kept for stirring at 80° C. for 6 h. The progress of the reaction was monitored by TLC. After completion, the reaction mixture was cooled to room temperature and evaporated to dryness. The residue was dissolved in ethyl acetate (20 mL) and washed with 0.1 N potassium bisulphate solution, organic layer was separated and dried over anhydrous sodium sulphate and evaporated to dryness to afford 2-((2S,3R)-3-((tert-butoxycarbonyl)amino)-4-(4-chlorophenyl)-2-hydroxybutanamido)-2-(3-(trifluoromethoxy)phenyl)acetic acid. The compound was purified by SFC to separate the two isomers.
Column: Regis (S,S) Whelk-01, 250 mm×21.1 mm, 5 μm
Mobile Phase: CO₂(A) and 0.1% DEA in EtOH:MeOH(1:1)(B)
Flow:50 mL
Isocratic: 85(A):15(B)
Injection: Volume-0.50 ml
Run Time: 11.5 min
Instrument Make and Model: SFC-PICLAB PREP 175

(S)-2-((2S,3R)-3-((tert-butoxycarbonyl)amino)-4-(4-chlorophenyl)-2-hydroxybutanamido)-2-(3-(trifluoromethoxy)phenyl)acetic acid (0.012 g, 0.022 mmol, 13.68% yield) as white solid. LCMS: 547.2 (M+H).

(R)-2-((2S,3R)-3-((tert-butoxycarbonyl)amino)-4-(4-chlorophenyl)-2-hydroxybutanamido)-2-(3-(trifluoromethoxy)phenyl)acetic acid (0.011 g, 0.020 mmol, 12.54% yield) as white solid. LCMS: 547.2 (M+H).

Note: The above stereochemistry is assigned and are relative.

Synthesis of (S)-2-((2S,3R)-3-amino-4-(4-chlorophenyl)-2-hydroxybutanamido)-2-(3-(trifluoromethoxy)phenyl)acetic acid (Compound 127): To a stirred solution of (S)-2-((2S,3R)-3-((tert-butoxycarbonyl)amino)-4-(4-chlorophenyl)-2-hydroxybutanamido)-2-(3-(trifluoromethoxy)phenyl)acetic acid (10 mg, 0.018 mmol) in DCM (3 mL) cooled to 0° C. was added 4M HCl in 1,4-Dioxane (0.556 uL, 0.018 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-4-(4-chlorophenyl)-2-hydroxybutanamido)-2-(3-(trifluoromethoxy)phenyl)acetic acid (0.006 g, 0.022 mmol, 80% yield) as off-white solid. LC-MS: 447.00 (M+H); HPLC=97.7%; ¹H NMR (DMSO-d₆, 400 MHz) δ 13.30 (brs, 1H), 9.3-9.4 (m, 1H), 7.9-8.3 (m, 3H), 7.5-7.6 (m, 1H), 7.4-7.5 (m, 1H), 7.3-7.4 (m, 4H), 7.2-7.3 (m, 2H), 5.7-5.8 (m, 1H), 5.4-5.5 (m, 1H), 3.9-4.0 (m, 1H), 3.8-3.9 (m, 1H), 3.0-3.1 (m, 1H), 2.7-2.8 (m, 1H). ¹⁹F NMR (DMSO-d₆, 282 MHz) δ −56.70 (s, 3 F).

Synthesis of (R)-2-((2S,3R)-3-amino-4-(4-chlorophenyl)-2-hydroxybutanamido)-2-(3-(trifluoromethoxy)phenyl)acetic acid (Compound 128): To a stirred solution of (R)-2-((2S,3R)-3-((tert-butoxycarbonyl)amino)-4-(4-chlorophenyl)-2-hydroxybutanamido)-2-(3-(trifluoromethoxy)phenyl)acetic acid (11 mg, 0.02 mmol) in DCM (3 mL) cooled to 0° C. was added 4M HCl in 1,4-Dioxane (0.3 mL, 0.028 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-4-(4-chlorophenyl)-2-hydroxybutanamido)-2-(3-(trifluoromethoxy)phenyl)acetic acid (0.005 g, 0.011 mmol, 55% yield) as off-white solid. LC-MS: 447.00 (M+H); HPLC=98.8%; ¹H NMR (DMSO-d₆, 400 MHz) δ 13.36 (brs, 1H), 8.8-9.0 (m, 1H), 7.9-8.3 (m, 3H), 7.4-7.5 (m, 2H), 7.35 (s, 1H), 7.31 (d, J=8.4 Hz, 2H), 7.23 (br d, J=6.6 Hz, 1H), 7.12 (d, J=8.4 Hz, 2H), 5.4-5.8 (m, 1H), 5.2-5.2 (m, 1H), 3.9-3.9 (m, 1H), 3.80 (br d, J=4.8 Hz, 1H), 2.6-2.7 (m, 1H), 2.5-2.6 (m, 1H). ¹⁹F NMR (DMSO-d₆, 282 MHz) δ −56.66 (s, 3 F).

Synthesis of (S)-2-((2S,3R)-3-amino-4-(2-fluorophenyl)-2-hydroxybutanamido)-2-(3-(trifluoromethoxy)phenyl)acetic acid (Compound 121): To a stirred solution of (S)-2-((2S,3R)-3-((tert-butoxycarbonyl)amino)-4-(2-fluorophenyl)-2-hydroxybutanamido)-2-(3-(trifluoromethoxy)phenyl)acetic acid (10 mg, 0.18 mmol) in DCM (3 mL) cooled to 0° C. was added 4M HCl in 1,4-Dioxane (0.3 mL, 0.028 mmol) under inert atmosphere. The reaction mixture was kept for stirring at 0.3 mL, 0.028 mmol for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-4-(2-fluorophenyl)-2-hydroxybutanamido)-2-(3-(trifluoromethoxy)phenyl)acetic acid (0.01 g, 0.018 mmol, 12.5% yield) as off-white solid. LC-MS: 431.15 (M+H); HPLC=93.3%; ¹H NMR (DMSO-d₆, 400 MHz) δ 13.5 (brs, 1H), 8.8-8.9 (m, 1H), 7.9-8.3 (m, 3H), 7.4-7.5 (m, 2H), 7.3-7.4 (m, 2H), 7.2-7.3 (m, 2H), 7.1-7.2 (m, 2H), 5.4-5.6 (m, 1H), 5.0-5.1 (m, 1H), 4.0-4.1 (m, 1H), 3.6-3.7 (m, 1H), 2.9-3.0 (m, 1H), 2.7-2.8 (m, 1H). ¹⁹F NMR (DMSO-d₆, 282 MHz) δ −56.57 (s, 3F), −117.93 (s, 1F).

Synthesis of (R)-2-((2S,3R)-3-amino-4-(2-fluorophenyl)-2-hydroxybutanamido)-2-(3-(trifluoromethoxy)phenyl)acetic acid (Compound 122): To a stirred solution of (R)-2-((2S,3R)-3-((tert-butoxycarbonyl)amino)-4-(2-fluorophenyl)-2-hydroxybutanamido)-2-(3-(trifluoromethoxy)phenyl)acetic acid (100 mg, 0.18 mmol) in DCM (3 mL) cooled to 0° C. was added 4M HCl in 1,4-Dioxane (0.3 mL, 0.1.8 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-4-(2-fluorophenyl)-2-hydroxybutanamido)-2-(3-(trifluoromethoxy)phenyl)acetic acid (0.012 g, 0.020 mmol, 15% yield) as off-white solid. LC-MS: 431.15 (M+H); HPLC=97.3%; ¹H NMR (DMSO-d₆, 400 MHz) δ 13.5 (brs, 1H), 8.8-8.9 (m, 1H), 7.9-8.3 (m, 3H), 7.4-7.5 (m, 2H), 7.2-7.3 (m, 4H), 7.0-7.1 (m, 2H), 5.3-5.6 (m, 1H), 5.0-5.1 (m, 1H), 4.0-4.0 (m, 1H), 3.6-3.7 (m, 1H), 2.7-2.8 (m, 2H). ¹⁹F NMR (DMSO-d₆, 282 MHz) δ −56.68 (s, 3 F), −118.07 (s, 1 F).

Synthesis of (S)-2-((2S,3R)-3-amino-4-(3-fluorophenyl)-2-hydroxybutanamido)-2-(3-(trifluoromethoxy)phenyl)acetic acid (Compound 123): To a stirred solution of (S)-2-((2S,3R)-3-((tert-butoxycarbonyl)amino)-4-(3-fluorophenyl)-2-hydroxybutanamido)-2-(3-(trifluoromethoxy)phenyl)acetic acid (35 mg, 0.066 mmol) in DCM (3 mL) cooled to 0° C. was added 4M HCl in 1,4-Dioxane (1 mL, 4.00 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-4-(3-fluorophenyl)-2-hydroxybutanamido)-2-(3-(trifluoromethoxy)phenyl)acetic acid (0.01 g, 0.021 mmol, 32.4% yield) as off-white solid. LC-MS: 431.15 (M+H); HPLC=92.4%; ¹H NMR (DMSO-d₆, 400 MHz) δ 13.6 (brs, 1H), 8.8-8.9 (m, 1H), 7.9-8.0 (m, 3H), 7.3-7.4 (m, 2H), 7.3-7.3 (m, 1H), 7.1-7.2 (m, 5H), 6.8-6.9 (m, 1H), 4.8-4.9 (m, 1H), 3.8-3.8 (m, 1H), 3.5-3.6 (m, 1H), 2.9-3.0 (m, 2H). ¹⁹F NMR (DMSO-d₆, 282 MHz) δ −56.57 (s, 3 F), −113.23 (s, 1 F).

Synthesis of (R)-2-((2S,3R)-3-amino-4-(3-fluorophenyl)-2-hydroxybutanamido)-2-(3-(trifluoromethoxy)phenyl)acetic acid (Compound 124): To a stirred solution of (R)-2-((2S,3R)-3-((tert-butoxycarbonyl)amino)-4-(3-fluorophenyl)-2-hydroxybutanamido)-2-(3-(trifluoromethoxy)phenyl)acetic acid (35 mg, 0.066 mmol) in DCM (3 mL) cooled to 0° C. was added 4M HCl in 1,4-Dioxane (1 mL, 4.00 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-4-(3-fluorophenyl)-2-hydroxybutanamido)-2-(3-(trifluoromethoxy)phenyl)acetic acid (0.015 g, 0.03 mmol, 46.0% yield) as off-white solid. LC-MS: 431.15 (M+H); HPLC=92.4%; ¹H NMR (DMSO-d₆, 400 MHz) δ 13.6 (brs, 1H), 8.8-8.9 (m, 1H), 7.9-8.0 (m, 3H), 7.3-7.5 (m, 2H), 7.1-7.2 (m, 1H), 7.0-7.1 (m, 5H), 6.8-6.9 (m, 1H), 4.9-5.0 (m, 1H), 3.8-3.9 (m, 1H), 3.5-3.6 (m, 1H), 2.9-3.0 (m, 2H). ¹⁹F NMR (DMSO-d₆, 282 MHz) δ −56.57 (s, 3 F), −113.23 (s, 1 F).

Synthesis of (S)-2-((2S,3R)-3-amino-4-(4-fluorophenyl)-2-hydroxybutanamido)-2-(3-(trifluoromethoxy)phenyl)acetic acid (Compound 125): To a stirred solution of (S)-2-((2S,3R)-3-((tert-butoxycarbonyl)amino)-4-(4-fluorophenyl)-2-hydroxybutanamido)-2-(3-(trifluoromethoxy)phenyl)acetic acid (15 mg, 0.028 mmol) in DCM (3 mL) cooled to 0° C. was added 4M HCl in 1,4-Dioxane (0.35 mL, 1.414 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-4-(4-fluorophenyl)-2-hydroxybutanamido)-2-(3-(trifluoromethoxy)phenyl)acetic acid (0.008 g, 0.018 mmol, 64.5% yield) as off-white solid. LC-MS: 431.00 (M+H); HPLC=98.2%; ¹H NMR (DMSO-d₆, 400 MHz) δ 13.37 (brs, 1H), 9.3-9.4 (m, 1H), 8.2-8.4 (m, 3H), 7.4-7.6 (m, 3H), 7.35 (br d, J=7.6 Hz. 1H), 7.1-7.2 (m, 4H), 5.6-5.7 (m, 1H), 5.5-5.6 (m, 1H), 3.9-4.0 (m, 2H), 2.6-2.7 (m, 2H). ¹⁹F NMR (DMSO-d₆, 282 MHz) δ −56.77 (s, 3 F), −117.15 (s, 1 F).

Synthesis of (R)-2-((2S,3R)-3-amino-4-(4-fluorophenyl)-2-hydroxybutanamido)-2-(3-(trifluoromethoxy)phenyl)acetic acid (Compound 126): To a stirred solution of (R)-2-((2S,3R)-3-((tert-butoxycarbonyl)amino)-4-(4-fluorophenyl)-2-hydroxybutanamido)-2-(3-(trifluoromethoxy)phenyl)acetic acid (18 mg, 0.034 mmol) in DCM (3 mL) cooled to 0° C. was added 4M HCl in 1,4-Dioxane (0.424 mL, 1.7 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-4-(4-fluorophenyl)-2-hydroxybutanamido)-2-(3-(trifluoromethoxy)phenyl)acetic acid (0.008 g, 0.017 mmol, 51.1% yield) as off-white solid. LC-MS: 431.00 (M+H); HPLC=93.4%; ¹H NMR (DMSO-d₆, 400 MHz) δ 13.40 (brs, 1H), 9.3-9.4 (m, 1H), 8.1-8.3 (m, 3H), 7.4-7.6 (m, 2H), 7.3-7.4 (m, 2H), 7.1-7.2 (m, 4H), 5.6-5.7 (m, 1H), 5.5-5.6 (m, 1H), 3.9-4.0 (m, 2H), 2.6-2.7 (m, 2H). ¹⁹F NMR (DMSO-d₆, 282 MHz) δ −56.77 (s, 3 F), −117.15 (s, 1 F).

Synthesis of (S)-2-((2S,3R)-3-amino-4-(4-bromophenyl)-2-hydroxybutanamido)-2-(3-(trifluoromethoxy)phenyl)acetic acid (Compound 129): To a stirred solution of (S)-2-((2S,3R)-3-((tert-butoxycarbonyl)amino)-4-(4-bromophenyl)-2-hydroxybutanamido)-2-(3-(trifluoromethoxy)phenyl)acetic acid (20 mg, 0.034 mmol) in DCM (3 mL) cooled to 0° C. was added 4M HCl in 1,4-Dioxane (0.6 mL, 0.17 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-4-(4-bromophenyl)-2-hydroxybutanamido)-2-(3-(trifluoromethoxy)phenyl)acetic acid (0.004 g, 0.008 mmol, 24.08% yield) as off-white solid. LC-MS: 491.05 (M+H); HPLC=97.4%; ¹H NMR (DMSO-d₆, 400 MHz) δ 13.70 (brs, 1H), 8.5-8.6 (m, 1H), 7.9-8.3 (m, 3H), 7.5-7.6 (m, 2H), 7.3-7.4 (m, 2H), 7.1-7.2 (m, 1H), 6.9-7.0 (m, 3H), 5.9-6.1 (m, 1H), 4.9-5.0 (m, 1H), 4.0-4.1 (m, 1H), 3.5-3.6 (m, 1H), 2.9-3.0 (m, 1H), 2.7-2.8 (m, 1H). ¹⁹F NMR (DMSO-d₆, 282 MHz) δ −56.75 (s, 3 F).

Synthesis of (R)-2-((2S,3R)-3-amino-4-(4-bromophenyl)-2-hydroxybutanamido)-2-(3-(trifluoromethoxy)phenyl)acetic acid (Compound 130): To a stirred solution of (R)-2-((2S,3R)-3-((tert-butoxycarbonyl)amino)-4-(4-bromophenyl)-2-hydroxybutanamido)-2-(3-(trifluoromethoxy)phenyl)acetic acid (20 mg, 0.034 mmol) in DCM (3 mL) cooled to 0° C. was added 4M HCl in 1,4-Dioxane (0.3 mL, 0.17 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-4-(4-bromophenyl)-2-hydroxybutanamido)-2-(3-(trifluoromethoxy)phenyl)acetic acid (0.005 g, 0.01 mmol, 30.1% yield) as off-white solid. LC-MS: 491.05 (M+H); HPLC=97.4%; ¹H NMR (DMSO-d₆, 400 MHz) δ 13.70 (brs, 1H), 8.2-8.3 (m, 1H), 7.9-8.3 (m, 3H), 7.5-7.6 (m, 2H), 7.3-7.4 (m, 2H), 7.1-7.2 (m, 1H), 6.9-7.0 (m, 3H), 5.9-6.1 (m, 1H), 4.7-4.8 (m, 1H), 3.7-3.8 (m, 1H), 3.4-3.5 (m, 1H), 3.2-3.3 (m, 1H), 2.8-2.9 (m, 1H). ¹⁹F NMR (DMSO-d₆, 282 MHz) δ −56.75 (s, 3 F).

Synthesis of (S)-2-((2S,3R)-3-amino-2-hydroxy-4-(4-(trifluoromethyl)phenyl)butanamido)-2-(3-(trifluoromethoxy)phenyl)acetic acid (Compound 131): To a stirred solution of (S)-2-((2S,3R)-3-((tert-butoxycarbonyl)amino)-4-(4-(trifluoromethyl)-2-hydroxybutanamido)-2-(3-(trifluoromethoxy)phenyl)acetic acid (20 mg, 0.034 mmol) in DCM (3 mL) cooled to 0° C. was added 4M HCl in 1,4-Dioxane (0.86 mL, 3.4 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (S)-2-((2S,3R)-3-amino-4-(4-(trifluoromethyl)-2-hydroxybutanamido)-2-(3-(trifluoromethoxy)phenyl)acetic acid (0.004 g, 0.083 mmol, 24.17% yield) as off-white solid. LC-MS: 481.15 (M+H); HPLC=98.6%; ¹H NMR (DMSO-d₆, 400 MHz) δ 13.50 (brs, 1H), 8.8-8.9 (m, 1H), 7.9-8.3 (m, 3H), 7.66 (d, J=8.1 Hz, 2H), 7.4-7.5 (m, 2H), 7.3-7.4 (m, 2H), 7.33 (s, 1H), 7.20 (d, J=5.3 Hz, 1H), 5.5-5.8 (m, 1H), 5.0-5.1 (m, 1H), 4.0-4.1 (m, 1H), 3.66 (br s, 1H), 2.97 (d, J=11.3 Hz, 1H), 2.7-2.8 (m, 1H). ¹⁹F NMR (DMSO-d₆, 282 MHz) δ −56.57 (s, 3 F), −60.74 (s, 3 F).

Synthesis of (R)-2-((2S,3R)-3-amino-2-hydroxy-4-(4-(trifluoromethyl)phenyl)butanamido)-2-(3-(trifluoromethoxy)phenyl)acetic acid (Compound 132): To a stirred solution of (R)-2-((2S,3R)-3-((tert-butoxycarbonyl)amino)-4-(4-(trifluoromethyl)-2-hydroxybutanamido)-2-(3-(trifluoromethoxy)phenyl)acetic acid (20 mg, 0.034 mmol) in DCM (3 mL) cooled to 0° C. was added 4M HCl in 1,4-Dioxane (0.3 mL, 3.4 mmol) under inert atmosphere. The reaction mixture was kept for stirring at room temperature for 3 h. The progress of the reaction was monitored by TLC. After completion, the reaction mixture was evaporated to dryness. The residue was washed with diethyl ether (5.0 mL) and dried to afford (R)-2-((2S,3R)-3-amino-4-(4-(trifluoromethyl)-2-hydroxybutanamido)-2-(3-(trifluoromethoxy)phenyl)acetic acid (0.004 g, 0.083 mmol, 24.17% yield) as off-white solid. LC-MS: 481.15 (M+H); HPLC=96.2%; ¹H NMR (DMSO-d₆, 400 MHz) δ 13.50 (brs, 1H), 8.7-8.9 (m, 1H), 7.9-8.3 (m, 3H), 7.66 (d, J=8.1 Hz, 2H), 7.4-7.5 (m, 2H), 7.3-7.4 (m, 2H), 7.33 (s, 1H), 7.20 (d, J=5.3 Hz, 1H), 5.4-5.7 (m, 1H), 5.0-5.1 (m, 1H), 3.9-4.0 (m, 1H), 3.70 (d, J=2.6 Hz, 1H), 2.7-2.8 (m, 1H), 2.5-2.6 (m, 1H). ¹⁹F NMR (DMSO-d₆, 282 MHz) δ −56.57 (s, 3 F), −60.74 (s, 3 F).

### Example 33

### General Procedure-A: Strecker Synthesis

To a stirred solution of aldehyde (1.0 equiv) in 7 N ammonia in methanol (4.0 equiv) was added titanium isopropoxide (1.07 equiv) in a sealed tube at ambient temperature and the reaction mixture was stirred at same temperature for 15 min. Then trimethylsilylcyanide (1.6 equiv) was added to the reaction mixture and stirred at ambient temperature for 20 h. After that, concentrated the reaction mixture under vacuum and obtained residue was dissolved in ethyl acetate (30 vol). The organic layer was washed with water (10 vol) and dried over anhydrous Na₂SO₄, filtered and concentrated under vacuum. The crude residue was purified by column chromatography over silica gel using 20% ethyl acetate in hexane to obtain nitrile derivative.

### General Procedure-B: Hydrolysis of Nitrile Derivative

To a stirred ice-cold solution of nitrile (1.0 equiv) derivative in methanol (10 vol) was added thionylchloride (3 vol) drop wise at 0° C. Then the resultant reaction mixture was slowly warmed to 80° C. and stirred at this temperature for 18 h under argon. After that, cooled the reaction mixture to rt and concentrated under vacuum. The obtained crude residue was dissolved in ethyl acetate (30 vol) and washed with water (10 vol). The organic layer was dried over anhydrous Na₂SO₄, filtered and concentrated under vacuum. The crude residue was purified by column chromatography over silica gel using 25% ethyl acetate in hexane to obtain ester derivative.

### General Procedure-C: Ester Hydrolysis

To a stirred solution of ester derivative (1.0 equiv) in methanol: tetrahydrofuran: water (1:1:1) (20 vol) was added lithium hydroxide monohydrate (2.0 equiv) at ambient temperature and the reaction mixture stirred at same temperature for 2 h. After that, concentrated the reaction mixture under vacuum to obtain lithium salt of amino acid derivative. Crude Lithium salt was used in next step without further purification.

### General Procedure-D: Boc Protection

To a stirred suspension of lithium salt of amino acid derivative (1.0 equiv) in 1,4-dioxane (10 vol) and sat. aq. NaHCO₃ solution (5 vol) was added Boc anhydride (1.5 equiv) at ambient temperature and the reaction mixture was stirred at room temperature for 18 h. After that, diluted the reaction mixture with water (10 vol) and acidified with saturated aqueous citric acid solution to pH ˜5. The aqueous layer was extracted with ethyl acetate (2×20 vol) and the combined organic layer were dried over anhydrous Na₂SO₄, filtered and concentrated under vacuum to obtain Boc protected amino acid derivative.

### General Procedure-E: Benzylation of Acid Using Benzyl Bromide

To a stirred solution of Boc protected amino acid derivative (1.0 equiv) in N,N-Dimethylformamide (10 vol) was added potassium carbonate (1.5 equiv) followed by benzyl bromide (1.2 equiv) at ambient temperature. Then the reaction mixture was slowly warmed to 50° C. and stirred for 1 h under argon. After that, cooled the reaction mixture to room temperature and diluted with water (10 vol) and extracted with ethyl acetate (30 vol). The organic layer was washed with water (3×30 vol) and brine solution (30 vol). The organic layer was dried over anhydrous Na₂SO₄, filtered and concentrated under vacuum. The crude compound was purified by column chromatography over silica gel (100-200 mesh) using 15% ethyl acetate in hexane to obtain benzylester derivative.

### General Procedure-F: Deprotection of Boc-Group

To a stirred solution of Boc derivative (1.0 equiv) in 2,2,2-Trifluoroethanol (10 vol) was added Trimethylsilyl chloride (3.0 equiv) was added at 0° C. The reaction mixture was slowly warmed to ambient temperature and stirred at this temperature for 8 h. After that, concentrated the reaction mixture under vacuum and Sat. Aq. NaHCO₃ (5 vol) was added to the crude residue. The aqueous layer was extracted with ethyl acetate (2×30 vol). The combined organic layer was dried over anhydrous Na₂SO₄, filtered and concentrated under vacuum to obtain amine derivative.

### General Procedure-G: Amide Coupling

To a stirred solution of acid derivative (1.0 equiv) in dichloromethane (10 vol) and dimethylformamide (0.5 vol) were added N,N′-Dicyclohexylcarbodiimide (DCC) (1.1 equiv) and Hydroxybenzotriazole (HOBt) (1.1 equiv) at ambient temperature and stirred for 15 min under argon. After that cooled the reaction mixture to 0° C. and added a solution of amine (1.0 equiv) in dichloromethane (10 vol) and slowly warmed the reaction mixture to room temperature and stirred at this temperature for 18 h under argon. After that, the resulted solids were filtered and solids were washed with ethyl acetate (30 vol). The filtrate was washed with water (2×10 vol) and brine (10 vol). The organic layer was dried over anhydrous Na₂SO₄, filtered and concentrated under vacuum. The crude compound was purified by column chromatography over silica gel (100-200 mesh) using 25% ethyl acetate in hexanes to obtain amide derivatives.

General Procedure-H: De protection of benzyl ester and Cbz-group

To a stirred solution of protected amino acid derivative (1.0 equiv) was dissolved in tetrahydrofuran (10 vol) was added 10% Pd/C (10% wt/wt) at ambient temperature and the reaction mixture was stirred at this temperature for 3 h under hydrogen atmosphere. After that, filtered the reaction mixture through celite bed and bed was washed with methanol (50 vol). The filtrate was concentrated under vacuum and the obtained solids were washed with methanol (2 vol) dried under vacuum to obtain amino acid derivative.

### General Procedure-I: Fmoc Protection

To a stirred suspension of lithium salt of amino acid derivative (1.0 equiv) in 1,4-dioxane (10 vol) and sat. aq. NaHCO₃ solution (5 vol) was added Fmoc-Cl (1.2 equiv) at ambient temperature and the reaction mixture was stirred at room temperature for 18 h. After that, diluted the reaction mixture with water (10 vol) and acidified with saturated aqueous citric acid solution to pH ˜5. The aqueous layer was extracted with ethyl acetate (2×20 vol) and the combined organic layer were dried over anhydrous Na₂SO₄, filtered and concentrated under vacuum. Crude compound was purified by column chromatography over silica gel (100-200 mesh) using 7% methanol in dichloromethane to obtain Fmoc protected amino acid derivative.

### General Procedure-J: Tert Butyl Ester Synthesis

To a stirred solution of Fmoc protected amino acid derivative (1.0 equiv) in toluene (10 vol) was added tert-Butyl 2,2,2-trichloroacetimidate (2.0 equiv) at ambient temperature. Then the reaction mixture was slowly warmed to 120° C. and stirred for 18 h under argon. After that, cooled the reaction mixture to room temperature and added additional tert-Butyl 2,2,2-trichloroacetimidate (2.0 equiv) at to ambient temperature. The resultant reaction mixture was slowly warmed to 120° C. and stirred for additional 18 h under argon. After that, cooled the reaction mixture to room temperature and diluted with water (10 vol) and extracted with ethyl acetate (30 vol). The organic layer was washed with water (3×30 vol) and brine solution (30 vol). The organic layer was dried over anhydrous Na₂SO₄, filtered and concentrated under vacuum. The crude compound was purified by column chromatography over silica gel (100-200 mesh) using 15% ethyl acetate in hexane to obtain tert butyl ester derivative.

### General Procedure-K: Deprotection of Fmoc-Group

To a stirred solution of Fmoc derivative (1.0 equiv) in N,N-Dimethylformamide (10 vol) was added piperidine (1.0 equiv) at 0° C. The reaction mixture was slowly warmed to ambient temperature and stirred at this temperature for 2 h under argon. After that, diluted the reaction mixture with water (10 vol) and extracted with ethyl acetate (30 vol). The organic layer was washed with water (3×30 vol) and brine solution (30 vol). The organic layer was dried over anhydrous Na₂SO₄, filtered and concentrated under vacuum. The crude compound was purified by column chromatography over silica gel (100-200 mesh) using 35% ethyl acetate in hexane to obtain amine derivative.

### General Procedure-L: De Protection of Tert Butyl Ester and Boc-Group

To a stirred solution of protected amino acid derivative (1.0 equiv) in 2,2,2-Trifluoroethanol (20 vol) was added Trimethylsilyl chloride (5.0 equiv) was added at 0° C. The reaction mixture was slowly warmed to ambient temperature and stirred at this temperature for 8 h. After that, concentrated the reaction mixture under vacuum. The obtained solids were washed with DCM and dried under vacuum to obtain amino acid derivative.

### General Procedure-M: Preparation of Negishi Reagent

To a stirred suspension of pre activated zinc (10.0 equiv) in N,N-Dimethylformamide (10 vol) was added 1,2-dibromethane (0.3 equiv) followed by Trimethylsilyl chloride (0.06 equiv) at ambient temperature under argon. The resultant reaction mixture was stirred at this temperature for 30 mins under argon. After that added a solution of iodo derivative (1.0 equiv) in N,N-Dimethylformamide (1 vol) at ambient temperature and stirred the reaction mixture for 30 mins at this temperature under argon. The supernatant liquid was used in next step.

### General Procedure-N: Negishi Coupling:

To a stirred solution of aryl bromide derivative (1.0 equiv) in N,N-Dimethylformamide (5 vol) were added Tris(dibenzylideneacetone)dipalladium(0) (0.05 equiv) and Tri(o-tolyl)phosphine (0.1 equiv) at ambient temperature and stirred the reaction mixture at this temperature for 15 min under argon. After that added a solution of freshly prepared negishi reagent (2.0 equiv) at ambient temperature and stirred the reaction mixture at this temperature for 4 h under argon. After that, diluted the reaction mixture with water (30 vol) and extracted with EtOAc (2×20 vol). The combined organic layer was washed with water (3×20 vol) followed by brine solution (20 vol). The organic layer was dried over anhydrous Na₂SO₄, filtered and concentrated under vacuum. The crude compound was purified by column chromatography over silica gel (100-200 mesh) using 15% ethyl acetate in hexane.

### Synthesis of (R)-2-((2S,3R)-3-amino-2-hydroxy-4-phenylbutanamido)-2-(3-fluoro-5-(trifluoromethoxy)phenyl)acetic acid (Compound 102) and (S)-2-((2S,3R)-3-amino-2-hydroxy-4-phenylbutanamido)-2-(3-fluoro-5-(trifluoromethoxy)phenyl)acetic acid (Compound 103)

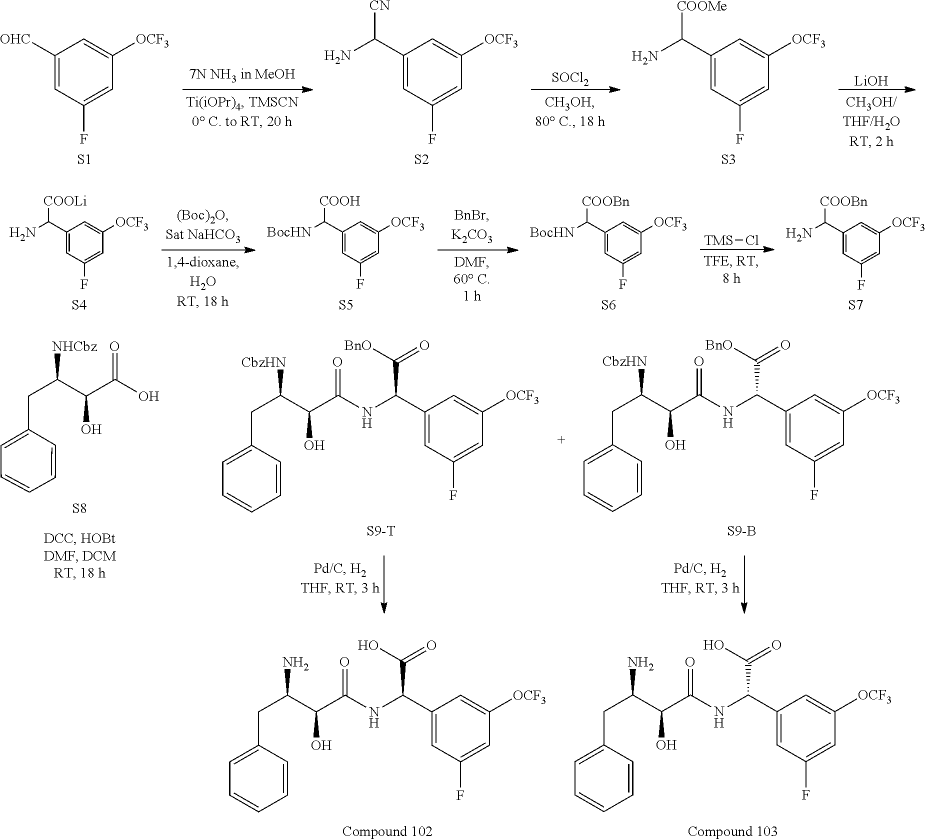

### Synthesis of 2-amino-2-(3-fluoro-5-(trifluoromethoxy)phenyl)acetonitrile

Title compound was synthesized by using general procedure-A and obtained as a gummy solid (2.00 g, 71%). ¹H-NMR (CDCl₃, 400 MHz): δ 7.78-7.59 (m, 1H), 7.43-7.35 (m, 2H), 7.29 (s, 1H), 5.71 (brs, 2H).

### Synthesis of methyl 2-amino-2-(3-fluoro-5-(trifluoromethoxy)phenyl)acetate

Title compound was synthesized by using general procedure-B and obtained as an off white solid (1.70 g, 75%). m/z (LC-MS): Calculated for C₁₀H₉F₄NO₃ is 267.18; found, 268.20 (M+H)+.

### Synthesis of lithium 2-amino-2-(3-fluoro-5-(trifluoromethoxy)phenyl)acetate

Title compound was synthesized by using general procedure-C and obtained as a color less gummy solid (1.60 g, 95%). m/z (LC-MS): Calculated for C₉H₆F₄LiNO₃ is 259.08; found, 254.20 (M-Li+H)⁺.

### Synthesis of 2-((tert-butoxycarbonyl)amino)-2-(3-fluoro-5-(trifluoromethoxy)phenyl)acetic acid

Title compound was synthesized by using general procedure-D and obtained as a color less gummy solid (2.50 g, crude). ¹H-NMR (DMSO-d₆, 400 MHz): δ 7.18-7.11 (m, 3H), 6.57 (d, J=5.2 Hz, 1H), 4.57 (d, J=4.0 Hz, 1H), 1.36 (s, 9H). m/z (LC-MS): Calculated for C₁₄H₁₅F₄NO₅ is 353.27; found, 376.30 (M+Na)⁺.

### Synthesis of benzyl 2-((tert-butoxycarbonyl)amino)-2-(3-fluoro-5-(trifluoromethoxy)phenyl)acetate

Title compound was synthesized by using general procedure-E and obtained as a white solid (1.70 g, 55% over two steps). ¹H-NMR (DMSO-d₆, 400 MHz): δ 8.02 (d, J=8.4 Hz, 1H), 7.39-7.30 (m, 6H), 7.24-7.22 (m, 2H), 5.47 (d, J=8.4 Hz, 1H), 5.19-5.09 (m, 2H), 1.39 (s, 9H). m/z (LC-MS): Calculated for C₂₁H₂₁F₄NO₅ is 443.39; found, 442.4 (M−H)—.

### Synthesis of benzyl 2-amino-2-(3-fluoro-5-(trifluoromethoxy)phenyl)acetate

Title compound was synthesized by using general procedure-F and obtained as a white solid (1.20 g, 91%). ¹H-NMR (DMSO-d₆, 400 MHz): δ 7.37-7.29 (m, 6H), 7.25-7.21 (m, 2H), 5.16-5.08 (m, 2H), 4.73 (s, 1H). m/z (LC-MS): Calculated for C₁₆H₁₃F₄NO₃ is 343.27; found, 344.30 (M+H)⁺.

### Synthesis of (R)-benzyl 2-((2S,3R)-3-(((benzyloxy)carbonyl)amino)-2-hydroxy-4-phenylbutanamido)-2-(3-fluoro-5-(trifluoromethoxy)phenyl)acetate (Top spot and (S)-benzyl 2-((2S,3R)-3-(((benzyloxy)carbonyl)amino)-2-hydroxy-4-phenylbutanamido)-2-(3-fluoro-5-(trifluoromethoxy)phenyl)acetate (Bottom spot)

Title compounds were synthesized by using general procedure-G and obtained top spot as a white solid (150 mg, 26%) and Bottom spot as a white solid (130 mg, 23%).

Top spot: ¹H-NMR (DMSO-d₆, 400 MHz): δ 8.77 (d, J=7.60 Hz, 1H), 7.34-7.18 (m, 18H), 6.89 (d, J=8.0 Hz, 1H), 5.97 (d, J=6.4 Hz, 1H), 5.63 (d, J=7.2 Hz, 1H), 5.18-5.09 (m, 2H), 4.93 (d, J=12.8 Hz, 1H), 4.83 (d, J=12.8 Hz, 1H), 4.10-4.03 (m, 1H), 3.97-3.95 (m, 1H), 2.86-2.81 (m, 1H), 2.71-2.66 (m, 1H). m/z (LC-MS): Calculated for C₃₄H₃₀F₄N₂O₇ is 654.60; found, 655.6 (M+H)⁺.

Bottom spot: ¹H-NMR (DMSO-d₆, 400 MHz): δ 8.73 (d, J=7.60 Hz, 1H), 7.35-7.18 (m, 18H), 6.95 (d, J=8.8 Hz, 1H), 6.02 (d, J=6.0 Hz, 1H), 5.71 (d, J=7.6 Hz, 1H), 5.2-5.10 (m, 2H), 4.96 (d, J=12.8 Hz, 1H), 4.74 (d, J=12.8 Hz, 1H), 3.98-3.93 (m, 2H), 2.85-2.80 (m, 1H), 2.66-2.61 (m, 1H). m/z (LC-MS): Calculated for C₃₄H₃₀F₄N₂O₇ is 654.60; found, 655.6 (M+H)⁺.

### Synthesis of (R)-2-((2S,3R)-3-amino-2-hydroxy-4-phenylbutanamido)-2-(3-fluoro-5-(trifluoromethoxy)phenyl)acetic acid (Top spot; Compound 102)

Title compound was synthesized by using general procedure-H and obtained as a grey solid (24.5 mg, 27%). ¹H-NMR (DMSO-d₆, 400 MHz): δ 8.30 (brs, 2H), 8.15 (d, J=6.0 Hz, 1H), 7.36-7.32 (m, 2H), 7.29-7.25 (m, 3H), 7.21-7.17 (m, 3H), 7.02 (brs, 1H), 4.87 (d, J=6.0 Hz, 1H), 3.83 (brs, 1H), 3.58-3.50 (m, 1H), 2.90 (d, J=7.2 Hz, 2H). m/z (LC-MS): Calculated for C₁₉H₁₈F₄N₂O₅ is 430.35; found, 431.7 (M+H)⁺. Chiral purity 99.7% (AUC), tᵣ=3.31 min.

### Synthesis of (S)-2-((2S,3R)-3-amino-2-hydroxy-4-phenylbutanamido)-2-(3-fluoro-5-(trifluoromethoxy)phenyl)acetic acid (Bottom spot; Compound 103)

Title compound was synthesized by using general procedure-H and obtained as a grey solid (40.2 mg, 47%). ¹H-NMR (DMSO-d₆, 400 MHz): δ 8.61 (d, J=5.2 Hz, 1H), 8.30 (brs, 2H), 7.33-7.17 (m, 8H), 6.60 (brs, 1H), 4.95 (d, J=4.4 Hz, 1H), 4.02 (d, J=2.4 Hz, 1H), 3.57-3.50 (m, 1H), 2.98-2.93 (m, 1H), 2.80-2.75 (m, 1H). m/z (LC-MS): Calculated for C₁₉H₁₈F₄N₂O₅ is 430.35; found, 431.7 (M+H)⁺. Chiral purity 99.7% (AUC), tᵣ=3.46 min.

### Synthesis of (R)-2-((2S,3R)-3-amino-2-hydroxy-4-phenylbutanamido)-2-(4-fluoro-3-(trifluoromethoxy)phenyl)acetic acid (Compound 104) and (S)-2-((2S,3R)-3-amino-2-hydroxy-4-phenylbutanamido)-2-(4-fluoro-3-(trifluoromethoxy)phenyl)acetic acid (Compound 105)

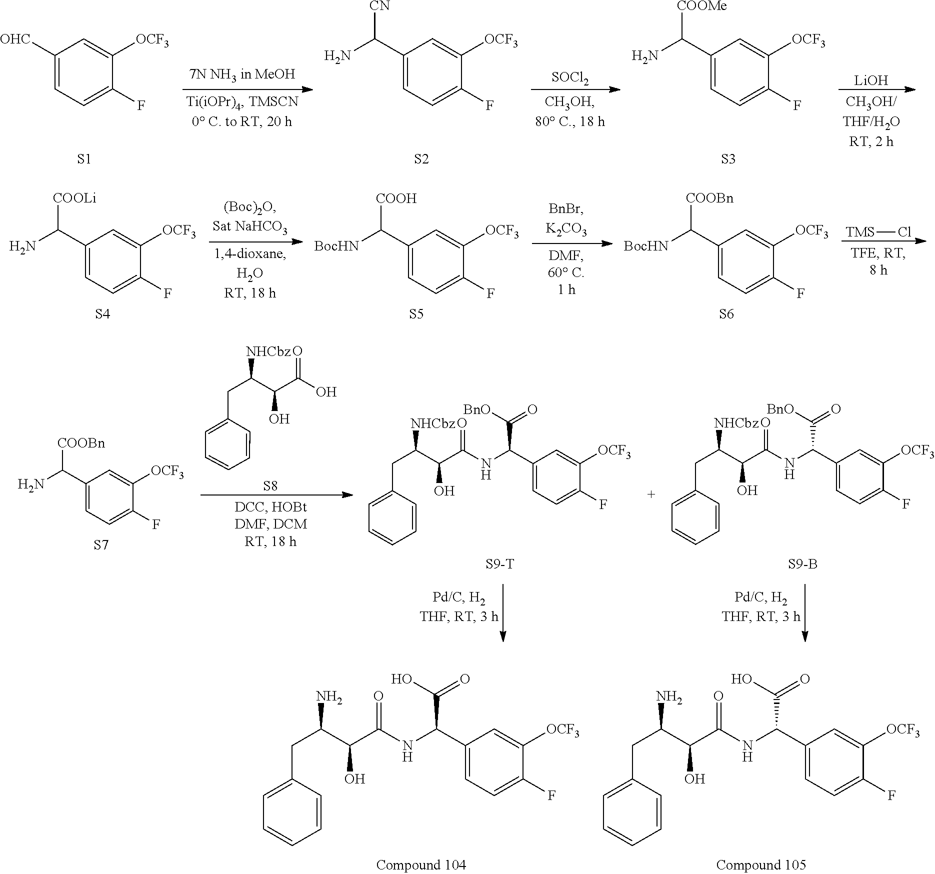

### Synthesis of 2-amino-2-(4-fluoro-3-(trifluoromethoxy)phenyl)acetonitrile

Title compound was synthesized by using general procedure-A and obtained as a gummy solid (2.00 g, 71%). m/z (LC-MS): Calculated for C₉H₆F₄N20 is 234.15; found, 235.20 (M+H)⁺.

### Synthesis of methyl 2-amino-2-(4-fluoro-3-(trifluoromethoxy)phenyl)acetate

Title compound was synthesized by using general procedure-B and obtained as an off white solid (1.70 g, 75%). m/z (LC-MS): Calculated for C₁₀H₉F₄NO is 267.18; found, 268.20 (M+H)⁺.

### Synthesis of lithium 2-amino-2-(4-fluoro-3-(trifluoromethoxy)phenyl)acetate

Title compound was synthesized by using general procedure-C and obtained as a color less gummy solid (1.60 g, 95%). ¹H-NMR (DMSO-d₆, 400 MHz): δ 7.55-7.53 (m, 1H), 7.50-7.46 (m, 1H), 7.33-7.28 (m, 1H), 4.10-4.07 (m, 1H), 2.08 (brs, 2H). m/z (LC-MS): Calculated for C₉H₆F₄LiNO₃ is 259.08; found, 254.20 (M-Li+H)⁺.

### Synthesis of 2-((tert-butoxycarbonyl)amino)-2-(4-fluoro-3-(trifluoromethoxy)phenyl)acetic acid

Title compound was synthesized by using general procedure-D and obtained as a color less gummy solid (2.10 g, crude). ¹H-NMR (DMSO-d₆, 400 MHz): δ 7.36-7.34 (m, 3H), 6.52 (d, J=5.2 Hz, 1H), 4.50 (d, J=5.2 Hz, 1H), 1.37 (s, 9H). m/z (LC-MS): Calculated for C₁₄H₁₅F₄NO₅ is 353.27; found, 376.30 (M+Na)⁺.

### Synthesis of benzyl 2-((tert-butoxycarbonyl)amino)-2-(4-fluoro-3-(trifluoromethoxy)phenyl)acetate

Title compound was synthesized by using general procedure-E and obtained as a white solid (2.00 g, 77% over two steps). ¹H-NMR (DMSO-d₆, 400 MHz): δ 7.99 (d, J=8.0 Hz, 1H), 7.64 (d, J=7.2 Hz, 1H), 7.51-7.47 (m, 2H), 7.31-7.29 (m, 3H), 7.23-7.21 (m, 2H), 5.41 (d, J=8.0 Hz, 1H), 5.18-5.08, 1.34 (s, 9H). m/z (LC-MS): Calculated for C₂₁H₂₁F₄NO₅ is 443.39; found, 466.1 (M+Na)⁺.

### Synthesis of benzyl 2-amino-2-(4-fluoro-3-(trifluoromethoxy)phenyl)acetate

Title compound was synthesized by using general procedure-F and obtained as a white solid (1.10 g, 71%). ¹H-NMR (DMSO-d₆, 400 MHz): δ 7.60 (d, J=7.6 Hz, 1H), 7.52-7.44 (m, 2H), 7.33-7.29 (m, 3H), 7.23-7.21 (m, 2H), 5.15-5.07 (m, 2H), 4.71 (s, 1H), 2.47 (brs, 1H). m/z (LC-MS): Calculated for C₁₆H₁₃F₄NO₃ is 343.27; found, 344.60 (M+H)⁺.

### Synthesis of (R)-benzyl 2-((2S,3R)-3-(((benzyloxy)carbonyl)amino)-2-hydroxy-4-phenylbutanamido)-2-(4-fluoro-3-(trifluoromethoxy)phenyl)acetate (Top spot and (S)-benzyl 2-((2S,3R)-3-(((benzyloxy)carbonyl)amino)-2-hydroxy-4-phenylbutanamido)-2-(4-fluoro-3-(trifluoromethoxy)phenyl)acetate (Bottom spot)

Title compounds were synthesized by using general procedure-G and obtained top spot as a white solid (200 mg, 18%) and Bottom spot as a white solid (210 mg, 18%).

Top spot: ¹H-NMR (DMSO-d₄, 400 MHz): δ 8.73 (d, J=7.60 Hz, 1H), 7.60 (d, J=7.2 Hz, 1H), 7.46-7.32 (m, 2H), 7.33-7.16 (m, 15H), 6.89 (d, J=9.6 Hz, 1H), 5.80 (d, J=6.4 Hz, 1H), 5.59-5.56 (m, 1H), 5.18-5.09 (m, 2H), 4.94-4.83 (m, 2H), 4.06-4.04 (m, 1H), 3.96-3.94 (m, 1H), 2.85-2.80 (m, 1H), 2.71-2.65 (m, 1H). m/z (LC-MS): Calculated for C₃₄H₃₀F₄N₂O₇ is 654.60; found, 655.9 (M+H)⁺.

Bottom spot: ¹H-NMR (DMSO-d₆, 400 MHz): 8.65 (d, J=7.20 Hz, 1H), 7.64 (d, J=7.2 Hz, 1H), 7.50-7.46 (m, 1H), 7.38-7.18 (m, 16H), 6.95 (d, J=9.2 Hz, 1H), 6.01 (d, J=6.4 Hz, 1H), 5.66 (d, J=7.2 Hz, 1H), 5.20-5.09 (m, 2H), 4.94 (d, J=12.8 Hz, 1H), 4.80 (d, J=12.8 Hz, 1H), 3.97-3.95 (m, 2H), 2.84-2.80 (m, 1H), 2.65-2.60 (m, 1H). m/z (LC-MS): Calculated for C₃₄H₃₀F₄N₂O₇ is 654.60; found, 655.8 (M+H)⁺.

### Synthesis of (R)-2-((2S,3R)-3-amino-2-hydroxy-4-phenylbutanamido)-2-(4-fluoro-3-(trifluoromethoxy)phenyl)acetic acid (Compound 104)

Title compound was synthesized by using general procedure-H and obtained as a grey solid (22.0 mg, 17%). ¹H-NMR (DMSO-d₆, 400 MHz): δ 8.30 (brs, 1H), 7.47 (d, J=6.8 Hz, 1H), 7.41-7.25 (m, 6H), 6.95 (brs, 1H), 4.90 (brs, 1H), 3.81 (brs, 1H), 3.56-3.50 (m, 1H), 2.90 (d, J=7.6 Hz, 2H). m/z (LC-MS): Calculated for C₁₉H₁₈F₄N₂O₅ is 430.35; found, 431.5 (M+H)⁺. Chiral purity 99.2% (AUC), tᵣ=3.40 min.

### Synthesis of (S)-2-((2S,3R)-3-amino-2-hydroxy-4-phenylbutanamido)-2-(4-fluoro-3-(trifluoromethoxy)phenyl)acetic acid (Compound 105)

Title compound was synthesized by using general procedure-H and obtained as a grey solid (65.0 mg, 49%). ¹H-NMR (DMSO-d₆, 400 MHz): δ 8.56 (d, J=6.0 Hz, 1H), 8.30 (brs, 2H), 7.80 (brs, 1H), 7.50 (d, J=7.2 Hz, 1H), 7.42-7.23 (m, 7H), 4.87 (d, J=5.6 Hz, 1H), 3.98 (d, J=2.8 Hz, 1H), 3.54 (brs, 1H), 2.97-2.91 (m, 1H), 2.78-2.73 (m, 1H). m/z (LC-MS): Calculated for C₁₉H₁₈F₄N₂O₅ is 430.35; found, 431.2 (M+H)⁺. Chiral purity 99.4% (AUC), tᵣ=3.51 min.

### Synthesis of (R)-2-((2S,3R)-3-amino-2-hydroxy-4-phenylbutanamido)-2-(4-chloro-3-(trifluoromethoxy)phenyl)acetic acid hydrochloride (Compound 106) and (S)-2-((2S,3R)-3-amino-2-hydroxy-4-phenylbutanamido)-2-(4-chloro-3-(trifluoromethoxy)phenyl)acetic acid hydrochloride (Compound 107)

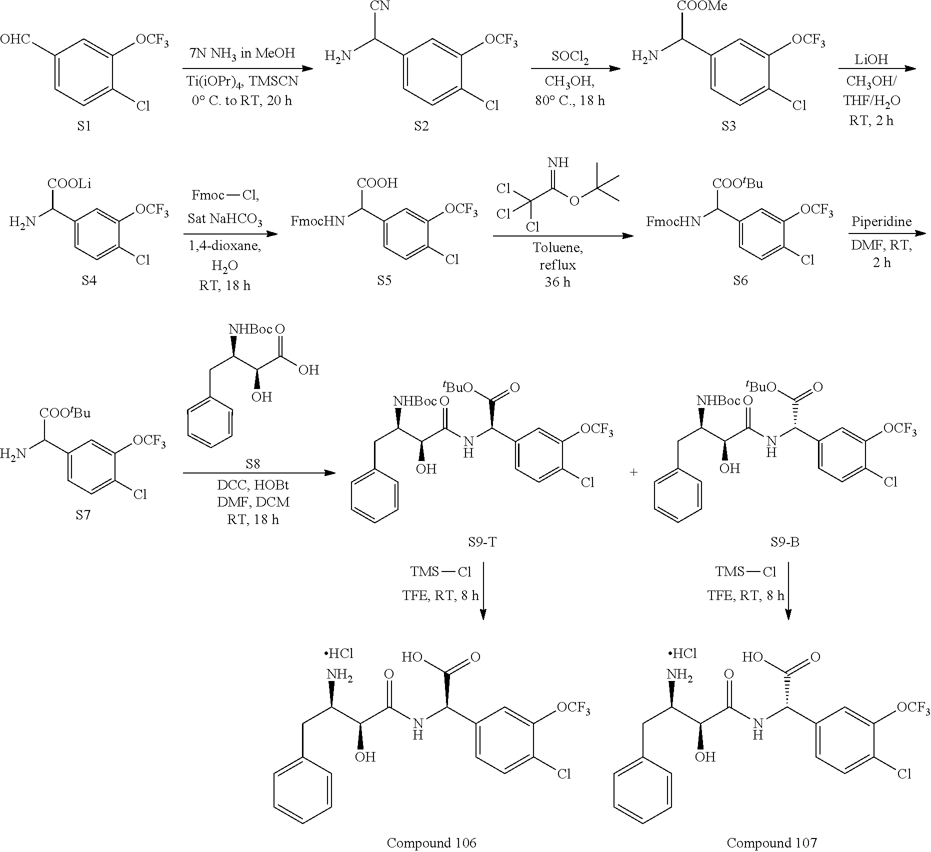

### Synthesis of 2-amino-2-(4-chloro-3-(trifluoromethoxy)phenyl)acetonitrile

Title compound was synthesized by using general procedure-A and obtained as a gummy solid (210 mg, 92%). ¹H-NMR (DMSO-d₆, 400 MHz): δ 7.77 (d, J=8.4 Hz, 1H), 7.71 (brs, 1H), 7.59 (dd, J=8.0, 11.4 Hz, 1H), 5.16 (brs, 1H), 3.01 (brs, 2H).

### Synthesis of methyl 2-amino-2-(4-chloro-3-(trifluoromethoxy)phenyl)acetate

Title compound was synthesized by using general procedure-B and obtained as an off white solid (1.40 g, 67%). m/z (LC-MS): Calculated for C₁₀H₉ClF₃NO₃ is 283.63; found, 284.20 (M+H)⁺.

### Synthesis of lithium 2-amino-2-(4-chloro-3-(trifluoromethoxy)phenyl)acetate

Title compound was synthesized by using general procedure-C and obtained as a color less gummy solid (1.30 g, crude). m/z (LC-MS): Calculated for C₉H₆ClF₃LiNO₃ is 275.54; found, 270.3 (M-Li+H)⁺.

### Synthesis of 2-((((9H-fluoren-9-yl)methoxy)carbonyl)amino)-2-(4-chloro-3-(trifluoromethoxy)phenyl)acetic acid

Title compound was synthesized by using general procedure-I and obtained as an off white solid (1.80 g, 85%). ¹H-NMR (DMSO-d₆, 400 MHz): δ 7.89 (d, J=7.2 Hz, 2H), 7.71 (d, J=7.2 Hz, 2H), 7.61 (d, J=8.0 Hz, 2H), 7.53 (s, 1H), 7.43-7.39 (m, 3H), 7.34-7.28 (m, 2H), 4.91 (d, J=6.40 Hz, 1H), 4.28-4.20 (m, 3H).

### Synthesis of tert-butyl 2-((((9H-fluoren-9-yl)methoxy)carbonyl)amino)-2-(4-chloro-3-(trifluoromethoxy)phenyl)acetate

Title compound was synthesized by using general procedure-J and obtained as a yellow solid (0.85 g, 48%). ¹H-NMR (DMSO-d₆, 400 MHz): δ 7.76 (d, J=7.6 Hz, 2H), 7.58 (d, J=7.2 Hz, 2H), 7.56-7.23 (m, 7H), 5.97 (d, J=6.40 Hz, 1H), 5.24 (d, J=6.80 Hz, 1H), 4.45-4.39 (m, 2H), 4.20 (t, J=6.4 Hz, 1H), 1.39 (s, 9H).

### Synthesis of tert-butyl 2-amino-2-(4-chloro-3-(trifluoromethoxy)phenyl)acetate

Title compound was synthesized by using general procedure-K and obtained as a yellow syrup (0.45 g, 96%). ¹H-NMR (DMSO-d₆, 400 MHz): δ 7.67 (d, J=8.0 Hz, 1H), 7.56 (s, 1H), 7.47 (dd, J=2.0, 8.4 Hz, 1H), 4.52 (s, 1H), 2.37 (s, 2H), 1.33 (s, 9H).

### Synthesis of (R)-tert-butyl 2-((2S,3R)-3-((tert-butoxycarbonyl)amino)-2-hydroxy-4-phenylbutanamido)-2-(4-chloro-3-(trifluoromethoxy)phenyl)acetate (Top spot) and (S)-tert-butyl 2-((2S,3R)-3-((tert-butoxycarbonyl)amino)-2-hydroxy-4-phenylbutanamido)-2-(4-chloro-3-(trifluoromethoxy)phenyl)acetate (Bottom spot)

Title compounds were synthesized by using general procedure-G and obtained top spot as a white solid (85 mg, 11%) and Bottom spot as a white solid (35 mg, 5%).

Top spot: ¹H-NMR (DMSO-d₆, 400 MHz): δ 8.53 (d, J=7.2 Hz, 1H), 7.67 (d, J=8.4 Hz, 1H), 7.61 (s, 1H), 7.42 (dd, J=2.0, 8.4 Hz, 1H), 7.30-7.15 (m, 5H), 6.27 (d, J=9.2 Hz, 1H), 5.83 (d, J=2.8 Hz, 1H), 5.38 (d, J=6.9 Hz, 1H), 3.99-3.94 (m, 1H), 3.90-3.88 (m, 1H), 2.82-2.77 (m, 1H), 2.68-2.63 (m, 1H), 1.33 (s, 9H), 1.23 (s, 9H). m/z (LC-MS): Calculated for C₂₈H₃₄ClF₃N₂O₇ is 603.03; found, 503.1 (M−100)*.

Bottom spot: ¹H-NMR (DMSO-d₄, 400 MHz): 8.53 (d, J=7.2 Hz, 1H), 7.65-7.58 (m, 2H), 7.45 (dd, J=1.6, 8.4 Hz, 1H), 7.24-7.28 (m, 2H), 7.19-7.15 (m, 3H), 6.31 (d, J=9.2 Hz, 1H), 5.98 (d, J=6.4 Hz, 1H), 5.43 (d, J=5.6 Hz, 1H), 3.92-3.85 (m, 2H), 2.81-2.76 (m, 1H), 2.64-2.58 (m, 1H), 1.31 (s, 9H), 1.26 (s, 9H). m/z (LC-MS): Calculated for C₂₈H₃₄ClF₃N₂O₇ is 603.03; found, 503.1 (M−100)*.

### Synthesis of (R)-2-((2S,3R)-3-amino-2-hydroxy-4-phenylbutanamido)-2-(4-chloro-3-(trifluoromethoxy)phenyl)acetic acid hydrochloride (Compound 106)

Title compound was synthesized by using general procedure-L and obtained as a white solid (28.0 mg, 41%). ¹H-NMR (DMSO-d₆, 400 MHz): δ 13.62 (brs, 1H), 8.54 (d, J=6.4 Hz, 1H), 7.95 (brs, 3H), 7.68 (d, J=8.4 Hz, 1H), 7.56 (s, 1H), 7.44 (dd, J=2.0, 8.4 Hz, 1H), 7.36-7.27 (m, 5H), 6.97 (d, J=6.0 Hz, 1H), 5.37 (d, J=6.8 Hz, 1H), 3.96-3.93 (m, 1H), 3.59 (brs, 1H), 2.90-2.88 (m, 2H). m/z (LC-MS): m/z (LC-MS): Calculated for C₁₉H₁₉Cl₂F₃N₂O₅ is 483.27; found, 447.1 (M-Cl)*. Chiral purity 98.5% (AUC), tᵣ=3.19 min.

### Synthesis of (S)-2-((2S,3R)-3-amino-2-hydroxy-4-phenylbutanamido)-2-(4-chloro-3-(trifluoromethoxy)phenyl)acetic acid hydrochloride (Compound 107)

Title compound was synthesized by using general procedure-L and obtained as a white solid (10.0 mg, 36%). ¹H-NMR (DMSO-d₆, 400 MHz): δ 13.39 (brs, 1H), 8.87 (d, J=7.2 Hz, 1H), 7.92 (brs, 3H), 7.71 (d, J=8.4 Hz, 1H), 7.70 (s, 1H), 7.50 (dd, J=2.0, 8.2 Hz, 1H), 7.36-7.26 (m, 5H), 6.75 (d, J=5.60 Hz, 1H), 5.49 (d, J=7.60 Hz, 1H), 4.09-4.07 (m, 1H), 3.53 (brs, 1H), 2.96-2.90 (m, 1H), 2.81-2.67 (m, 1H). m/z (LC-MS): Calculated for C₁₉H₁₉Cl₂F₃N₂O₅ is 483.27; found, 447.1 (M-Cl)*. Chiral purity 99.3% (AUC), tᵣ=3.27 min.

### Synthesis of (R)-2-((2S,3R)-3-amino-2-hydroxy-4-phenylbutanamido)-2-(3-chloro-5-(trifluoromethoxy)phenyl)acetic acid hydrochloride (Compound 108) and (S)-2-((2S,3R)-3-amino-2-hydroxy-4-phenylbutanamido)-2-(3-chloro-5-(trifluoromethoxy)phenyl)acetic acid hydrochloride (Compound 109)

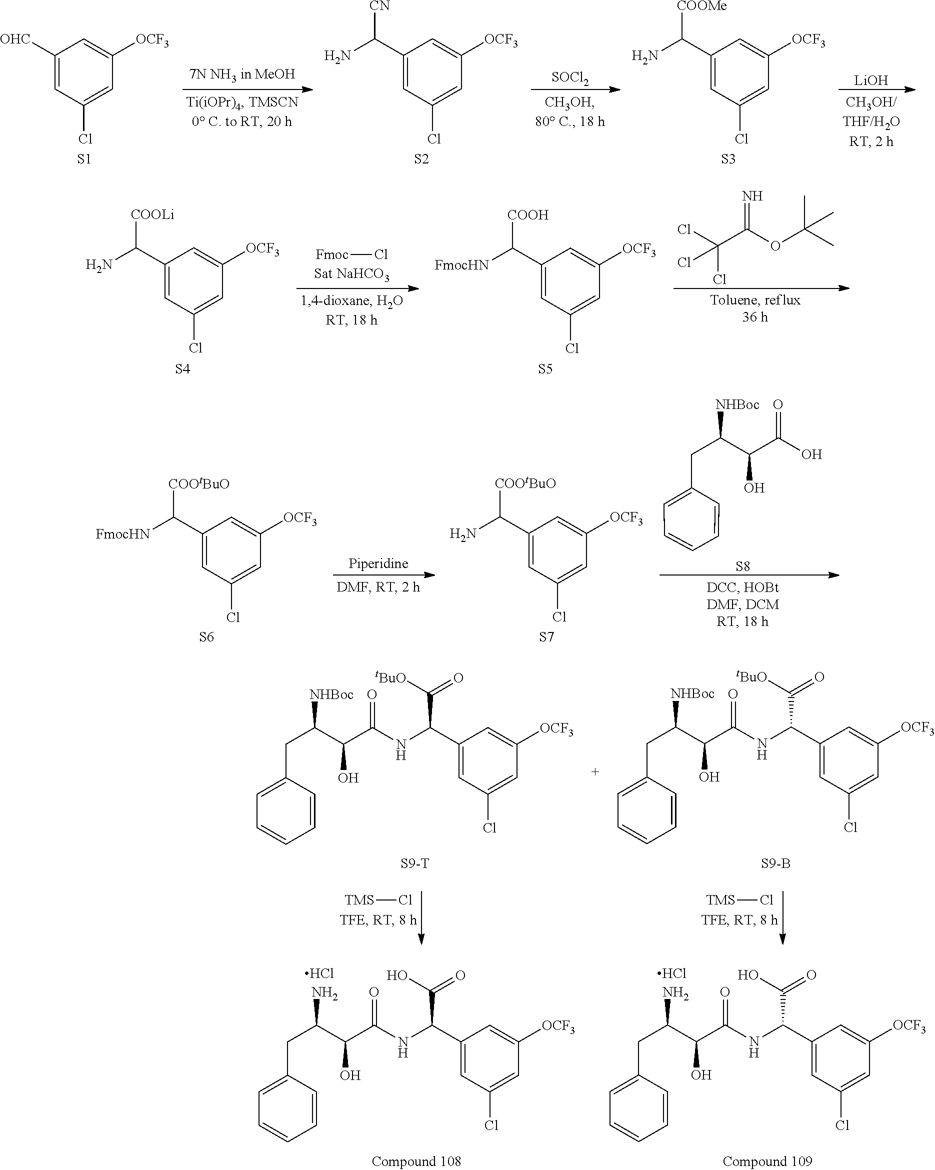

### Synthesis of 2-amino-2-(3-chloro-5-(trifluoromethoxy)phenyl)acetonitrile

Title compound was synthesized by using general procedure-A and obtained as a gummy solid (2.90 mg, 88%). ¹H-NMR (DMSO-d₆, 400 MHz): δ 7.67 (brs, 1H), 7.58 (brs, 1H), 7.53 (brs, 1H), 5.16 (brs, 1H), 3.03 (d, J=7.6 Hz, 2H).

### Synthesis of methyl 2-amino-2-(3-chloro-5-(trifluoromethoxy)phenyl)acetate

Title compound was synthesized by using general procedure-B and obtained as a yellow syrup (1.20 g, 37%%). ¹H-NMR (DMSO-d₆, 400 MHz): 7.56 (brs, 1H), 7.47 (brs, 1H), 7.42 (brs, 1H), 4.08 (s, 1H), 3.62 (s, 3H).

### Synthesis of lithium 2-amino-2-(3-chloro-5-(trifluoromethoxy)phenyl)acetate

Title compound was synthesized by using general procedure-C and obtained as a color less gummy solid (0.21 g, crude). ¹H-NMR (DMSO-d₄, 400 MHz): 7.52 (s, 1H), 7.38 (s, 1H), 7.22 (s, 1H), 4.06 (t, J=6.4 Hz, 1H), 2.08 (d, J=6.4 Hz, 1H).

### Synthesis of 2-((((9H-fluoren-9-yl)methoxy)carbonyl)amino)-2-(3-chloro-5-(trifluoromethoxy)phenyl)acetic acid

Title compound was synthesized by using general procedure-I and obtained as an off white solid (0.25 g, 73%). ¹H-NMR (DMSO-d₆, 400 MHz): δ 12.50 (brs, 1H), 8.26 (s, 1H), 7.89 (d, J=7.2 Hz, 2H), 7.85 (d, J=7.2 Hz, 2H), 7.73 (s, 1H), 7.71 (s, 1H), 7.59 (s, 1H), 7.52-7.32 (m, 2H), 7.30-7.22 (m, 2H), 5.30 (d, J=7.6 Hz, 1H), 4.35-4.21 (m, 3H).

### Synthesis of tert-butyl 2-((((9H-fluoren-9-yl)methoxy)carbonyl)amino)-2-(3-chloro-5-(trifluoromethoxy)phenyl)acetate

Title compound was synthesized by using general procedure-J and obtained as a yellow solid (1.50 g, 42%). m/z (LC-MS): Calculated for C₂₈H₂₅ClF₃NO₅ is 547.95; found, 549.1 (M+H)⁺.

### Synthesis of tert-butyl 2-amino-2-(3-chloro-5-(trifluoromethoxy)phenyl)acetate

Title compound was synthesized by using general procedure-K and obtained as a yellow solid (0.77 g, 93%). m/z (LC-MS): Calculated for C₁₃H₁₅ClF₃NO₃ is 325.71; found, 326.5 (M+H)⁺.

### Synthesis of (R)-tert-butyl 2-((2S,3R)-3-((tert-butoxycarbonyl)amino)-2-hydroxy-4-phenylbutanamido)-2-(3-chloro-5-(trifluoromethoxy)phenyl)acetate (Top spot) and (S)-tert-butyl 2-((2S,3R)-3-((tert-butoxycarbonyl)amino)-2-hydroxy-4-phenylbutanamido)-2-(3-chloro-5-(trifluoromethoxy)phenyl)acetate (Bottom spot)

Title compounds were synthesized by using general procedure-G and obtained top spot as a white solid (40 mg, 11%) and Bottom spot as a white solid (68 mg, 4%).

Top spot: ¹H-NMR (DMSO-d₆, 400 MHz): δ 7.90 (d, J=6.0 Hz, 1H), 7.32-7.28 (m, 3H), 7.26-7.22 (m, 3H), 7.15 (d, J=8.0 Hz, 2H), 5.76 (brs, 1H), 5.40 (d, J=7.2 Hz, 1H), 4.99 (d, J=7.6 Hz, 1H), 4.17-4.18 (m, 1H), 3.92-3.94 (m, 1H), 3.17-3.15 (m, 1H), 3.09-3.04 (m, 1H), 1.30 (s, 18H). m/z (LC-MS): Calculated for C₂₈H₃₄ClF₃N₂O₇ is 603.03; found, 503.1 (M−100)⁺.

Bottom spot: ¹H-NMR (DMSO-d₄, 400 MHz): 8.60 (d, J=7.6 Hz, 1H), 7.50-7.53 (m, 2H), 7.41 (s, 1H), 7.28-7.24 (m, 2H), 7.19-7.15 (m, 3H), 6.28 (d, J=9.2 Hz, 1H), 5.93 (d, J=6.4 Hz, 1H), 5.46 (d, J=7.6 Hz, 1H), 3.88-3.93 (m, 2H), 2.81-2.76 (m, 1H), 2.67-2.60 (m, 1H), 1.33 (s, 9H), 1.23 (s, 9H). m/z (LC-MS): Calculated for C₂₈H₃₄ClF₃N₂O₇ is 603.03; found, 503.1 (M−100)⁺.

### Synthesis of (R)-2-((2S,3R)-3-amino-2-hydroxy-4-phenylbutanamido)-2-(3-chloro-5-(trifluoromethoxy)phenyl)acetic acid hydrochloride (Compound 108)

Title compound was synthesized by using general procedure-L and obtained as a white solid (14.0 mg, 44%). ¹H-NMR (DMSO-d₆, 400 MHz): δ 13.81 (brs, 1H), 8.55 (d, J=6.0 Hz, 1H), 8.02 (brs, 3H), 7.52 (d, J=6.8 Hz, 2H), 7.39-7.28 (m, 6H), 7.00 (d, J=5.2 Hz, 1H), 5.37 (brs, 1H), 3.95 (brs, 1H), 3.59 (brs, 1H), 2.91 (d, J=7.2 Hz, 2H). m/z (LC-MS): Calculated for C₁₉H₁₉Cl₂F₃N₂O₅ is 483.27; found, 447.1 (M-Cl)⁺. Chiral purity 98.2% (AUC), tr=3.37 min.

### Synthesis of (S)-2-((2S,3R)-3-amino-2-hydroxy-4-phenylbutanamido)-2-(3-chloro-5-(trifluoromethoxy)phenyl)acetic acid hydrochloride (Compound 109)

Title compound was synthesized by using general procedure-L and obtained as a white solid (38.0 mg, 70%). ¹H-NMR (DMSO-d₆, 400 MHz): δ 13.45 (brs, 1H), 8.92 (d, J=7.2 Hz, 1H), 7.97 (brs, 3H), 7.59 (s, 1H), 7.53 (s, 1H), 7.46 (s, 1H), 7.36-7.32 (m, 2H), 7.29-7.25 (m, 3H), 6.78 (d, J=6.0 Hz, 1H), 5.51 (d, J=7.6 Hz, 1H), 4.11-4.08 (m, 1H), 3.54 (brs, 1H), 2.96-2.90 (m, 1H), 2.83-2.78 (m, 1H). m/z (LC-MS): Calculated for C₁₉H₁₉Cl₂F₃N₂O₅ is 483.27; found, 447.2 (M-Cl)⁺. Chiral purity 99.04% (AUC), tr=3.39 min.

### Synthesis of (R)-2-((2S,3R)-3-amino-2-hydroxy-4-phenylbutanamido)-2-(4-bromo-3-(trifluoromethoxy)phenyl)acetic acid hydrochloride (Compound 110) and (S)-2-((2S,3R)-3-amino-2-hydroxy-4-phenylbutanamido)-2-(4-bromo-3-(trifluoromethoxy)phenyl)acetic acid hydrochloride (Compound 111)

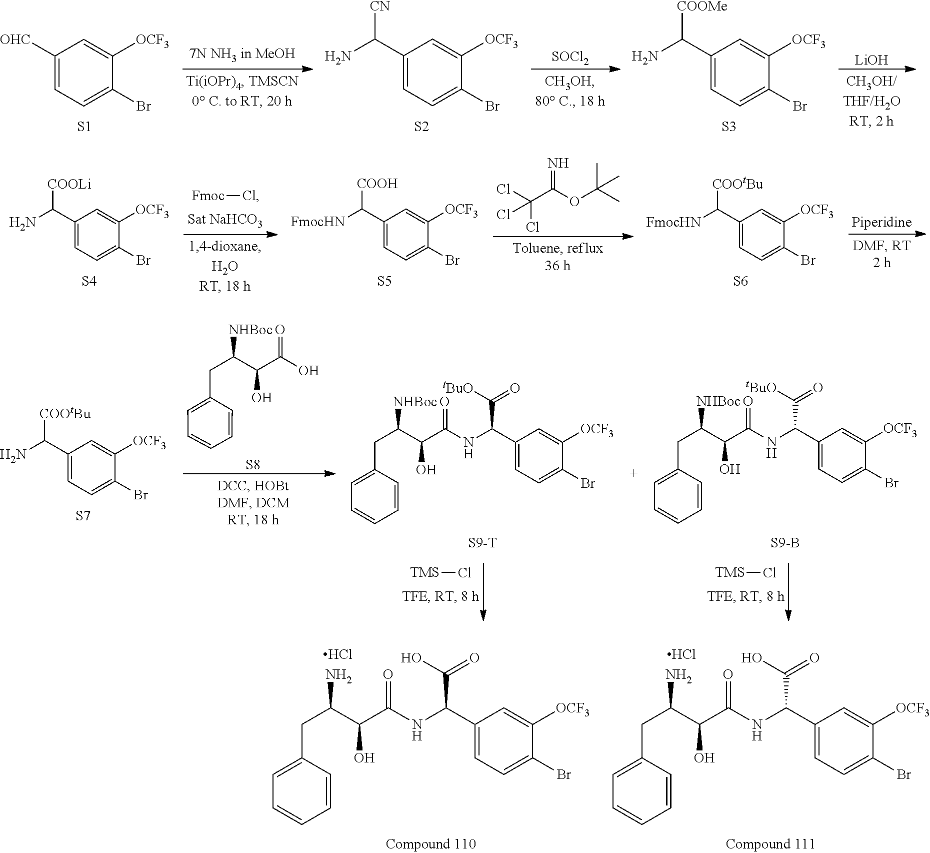

### Synthesis of 2-amino-2-(4-bromo-3-(trifluoromethoxy)phenyl)acetonitrile

Title compound was synthesized by using general procedure-A and obtained as a gummy solid (0.35 g, 63%). ¹H-NMR (CDCl₃, 400 MHz): δ 7.71 (d, J=8.4 Hz, 1H), 7.55 (s, 1H), 7.41 (dd, J=1.6, 8.2 Hz, 1H), 4.92 (s, 1H), 2.01 (s, 2H).

### Synthesis of methyl 2-amino-2-(4-bromo-3-(trifluoromethoxy)phenyl)acetate

Title compound was synthesized by using general procedure-B and obtained as an off white solid (1.80 g, 67%). m/z (LC-MS): Calculated for C₁₀H₉BrF₃NO₃ is 328.08; found, 328.1 (M)⁺.

### Synthesis of lithium 2-amino-2-(4-bromo-3-(trifluoromethoxy)phenyl)acetate

Title compound was synthesized by using general procedure-C and obtained as a color less gummy solid (1.70 g, crude). ¹H-NMR (DMSO-d₆, 400 MHz): 7.61 (d, J=8.0 Hz, 1H), 7.55 (s, 1H), 7.41 (dd, J=1.6, 8.4 Hz, 1H), 4.08 (t, J=6.4 Hz, 1H), 2.08 (d, J=6.4 Hz, 1H).

### Synthesis of 2-((((9H-fluoren-9-yl)methoxy)carbonyl)amino)-2-(4-bromo-3-(trifluoromethoxy)phenyl)acetic acid

Title compound was synthesized by using general procedure-I and obtained as an off white solid (2.00 g, 70%). ¹H-NMR (DMSO-d₆, 400 MHz): δ 7.89 (d, J=7.6 Hz, 2H), 7.76-7.71 (m, 4H), 7.53 (s, 1H), 7.43-7.39 (m, 2H), 7.36-7.28 (m, 3H), 4.99 (d, J=8.0 Hz, 1H), 4.28-4.20 (m, 3H).

### Synthesis of tert-butyl 2-((((9H-fluoren-9-yl)methoxy)carbonyl)amino)-2-(4-bromo-3-(trifluoromethoxy)phenyl)acetate

Title compound was synthesized by using general procedure-J and obtained as a white solid (1.00 g, 48%). ¹H-NMR (CDCl₃, 400 MHz): δ 7.77 (d, J=7.2 Hz, 2H), 7.62-7.56 (m, 3H), 7.42-7.38 (m, 2H), 7.34-7.26 (m, 3H), 7.18 (d, J=8.0 Hz, 1H), 5.97 (d, J=6.40 Hz, 1H), 5.23 (d, J=6.4 Hz, 1H), 4.45-4.38 (m, 2H), 4.21-4.19 (m, 1H), 1.39 (s, 9H).

### Synthesis of tert-butyl 2-amino-2-(4-bromo-3-(trifluoromethoxy)phenyl)acetate

Title compound was synthesized by using general procedure-K and obtained as a yellow syrup (0.93 g, 93%). ¹H-NMR (DMSO-d₆, 400 MHz): δ 7.61 (d, J=8.4 Hz, 1H), 7.38 (s, 1H), 7.25 (dd, J=2.0, 8.8 Hz, 1H), 4.49 (s, 1H), 1.39 (s, 9H).

### Synthesis of (R)-tert-butyl 2-(4-bromo-3-(trifluoromethoxy)phenyl)-2-((2S,3R)-3-((tert-butoxycarbonyl)amino)-2-hydroxy-4-phenylbutanamido)acetate (Top spot) and (S)-tert-butyl 2-(4-bromo-3-(trifluoromethoxy)phenyl)-2-((2S,3R)-3-((tert-butoxycarbonyl)amino)-2-hydroxy-4-phenylbutanamido)acetate (Bottom spot)

Title compounds were synthesized by using general procedure-G and obtained top spot as a white solid (0.27 g, 19%) and Bottom spot as a white solid (0.25 g, 17%).

Top spot: ¹H-NMR (DMSO-d₆, 400 MHz): δ 8.52 (d, J=7.2 Hz, 1H), 7.79 (d, J=8.4 Hz, 1H), 7.55 (s, 1H), 7.34 (d, J=8.4 Hz, 1H), 7.28-7.24 (m, 2H), 7.21-7.17 (m, 3H), 6.27 (d, J=9.2 Hz, 1H), 5.79 (d, J=6.8 Hz, 1H), 5.37 (d, J=7.2 Hz, 1H), 3.99-3.96 (m, 1H), 3.91-3.88 (m, 1H), 2.82-2.77 (m, 1H), 2.68-2.63 (m, 1H), 1.33 (s, 9H), 1.25 (s, 9H). m/z (LC-MS): Calculated for C₂₈H₃₄BrF₃N₂O₇ is 647.48; found, 547.20 (M−100)⁺.

Bottom spot: ¹H-NMR (DMSO-d₄, 400 MHz): 8.51 (d, J=7.6 Hz, 1H), 7.76 (d, J=8.4 Hz, 1H), 7.56 (s, 1H), 7.37 (dd, J=1.6, 8.2 Hz, 1H), 7.33-7.28 (m, 2H), 7.26-7.15 (m, 3H), 6.31 (d, J=9.2 Hz, 1H), 5.98 (d, J=6.4 Hz, 1H), 5.42 (d, J=7.6 Hz, 1H), 3.91-3.85 (m, 2H), 2.81-2.76 (m, 1H), 2.67-2.61 (m, 1H), 1.32 (s, 9H), 1.23 (s, 9H). m/z (LC-MS): Calculated for C₂₈H₃₄BrF₃N₂O₇ is 647.48; found, 547.1 (M−100)⁺.

### Synthesis of (R)-2-((2S,3R)-3-amino-2-hydroxy-4-phenylbutanamido)-2-(4-bromo-3-(trifluoromethoxy)phenyl)acetic acid hydrochloride (Compound 110)

Title compound was synthesized by using general procedure-L and obtained as a white solid (70 mg, 15%). ¹H-NMR (DMSO-d₆, 400 MHz): δ 13.51 (brs, 1H), 8.53 (d, J=6.4 Hz, 1H), 8.03 (brs, 3H), 7.80 (d, J=8.4 Hz, 1H), 7.53 (s, 1H), 7.37-7.24 (m, 6H), 7.04 (d, J=6.0 Hz, 1H), 5.35 (d, J=6.8 Hz, 1H), 3.95-3.94 (m, 1H), 3.59-3.56 (m, 1H), 2.91-2.89 (m, 2H). m/z (LC-MS): Calculated for C₁₉H₁₉BrClF₃N₂O₅ is 527.72; found, 491.2 (M−HCl)⁺. Chiral purity 99.4% (AUC), tᵣ=3.70 min.

### Synthesis of (S)-2-((2S,3R)-3-amino-2-hydroxy-4-phenylbutanamido)-2-(4-bromo-3-(trifluoromethoxy)phenyl)acetic acid hydrochloride (Compound 111)

Title compound was synthesized by using general procedure-L and obtained as a white solid (33.0 mg, 85%). ¹H-NMR (DMSO-d₆, 400 MHz): δ 13.41 (brs, 1H), 8.83 (d, J=7.6 Hz, 1H), 7.91 (brs, 3H), 7.80 (d, J=8.4 Hz, 1H), 7.57 (s, 1H), 7.41-7.37 (m, 1H), 7.32-7.29 (m, 2H), 7.25-7.23 (m, 3H), 6.72 (d, J=6.0 Hz, 1H), 5.43 (d, J=7.2 Hz, 1H), 4.05-4.03 (m, 1H), 3.49 (brs, 1H), 2.92-2.87 (m, 1H), 2.78-2.73 (m, 1H). m/z (LC-MS): Calculated for C₁₉H₁₉BrClF₃N₂O₅ is 527.72; found, 491.4 (M−HCl)⁺. Chiral purity 99.5% (AUC), tr=3.53 min.

### Synthesis of (R)-2-((2S,3R)-3-amino-2-hydroxy-4-phenylbutanamido)-2-(2-fluoro-3-(trifluoromethoxy)phenyl)acetic acid hydrochloride (Compound 112) and (S)-2-((2S,3R)-3-amino-2-hydroxy-4-phenylbutanamido)-2-(2-fluoro-3-(trifluoromethoxy)phenyl)acetic acid hydrochloride (Compound 113)

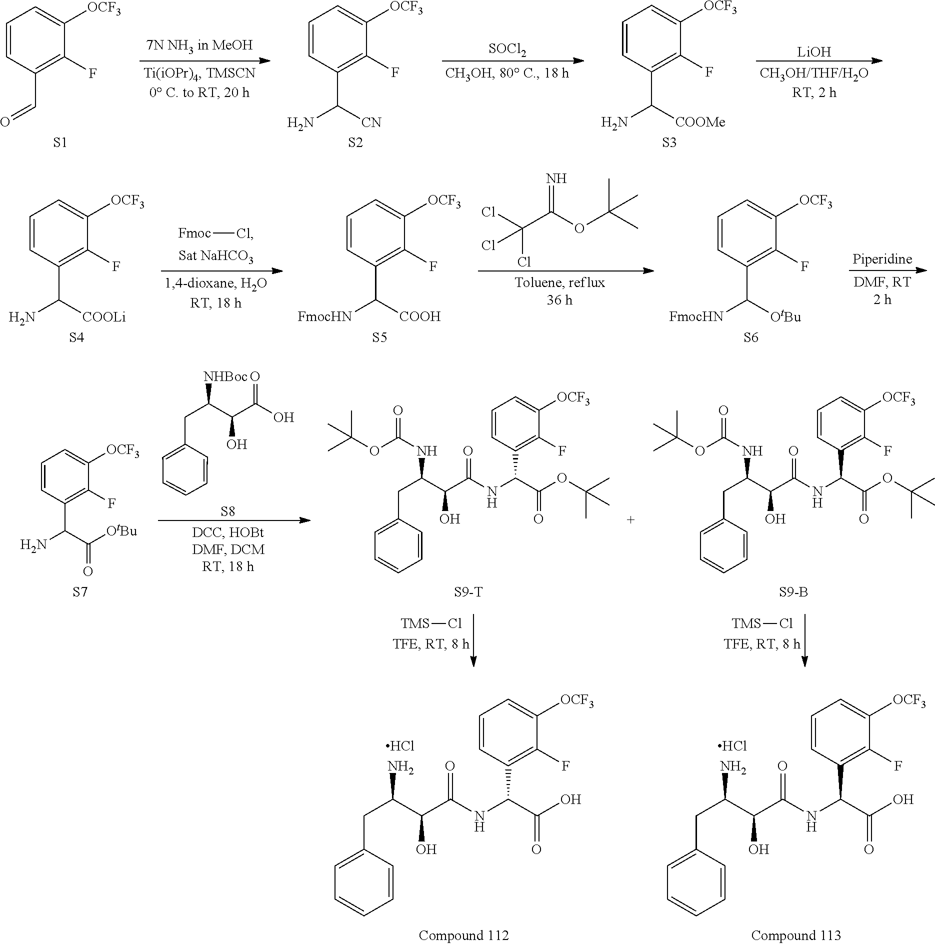

### Synthesis of 2-amino-2-(2-fluoro-3-(trifluoromethoxy)phenyl)acetonitrile

Title compound was synthesized by using general procedure-A and obtained as a yellow solid (1.70 g, 51%). ¹H-NMR (CDCl₃, 400 MHz): δ 7.56-7.52 (m, 1H), 7.40-7.36 (m, 1H), 7.28-7.24 (m, 1H), 5.16 (s, 1H), 2.04 (s, 2H).

### Synthesis of methyl 2-amino-2-(2-fluoro-3-(trifluoromethoxy)phenyl)acetate

Title compound was synthesized by using general procedure-B and obtained as an off white solid (1.50 g, 78%). m/z (LC-MS): Calculated for C₁₀H₉F₄NO₃ is 267.18; found, 368.2 (M)⁺.

### Synthesis of lithium 2-amino-2-(2-fluoro-3-(trifluoromethoxy)phenyl)acetate

Title compound was synthesized by using general procedure-C and obtained as a color less gummy solid (1.40 g, crude). m/z (LC-MS): Calculated for C₉H₆F₄LiNO₃ is 259.08; found, 254.2 (M-Li+H)⁺.

### Synthesis of 2-((((9H-fluoren-9-yl)methoxy)carbonyl)amino)-2-(2-fluoro-3-(trifluoromethoxy)phenyl)acetic acid

Title compound was synthesized by using general procedure-I and obtained as an off white solid (1.80 g, 69%). ¹H-NMR (DMSO-d₄, 400 MHz): δ 7.89-7.87 (m, 2H), 7.71-7.69 (m, 1H), 7.45-7.38 (m, 5H), 7.34-7.26 (m, 4H), 5.22-5.24 (m, 1H), 4.29-4.21 (m, 3H).

### Synthesis of tert-butyl 2-((((9H-fluoren-9-yl)methoxy)carbonyl)amino)-2-(2-fluoro-3-(trifluoromethoxy)phenyl)acetate

Title compound was synthesized by using general procedure-J and obtained as a white solid (0.56 g, 28%). ¹H-NMR (CDCl₃, 400 MHz): δ 7.76 (d, J=7.6 Hz, 2H), 7.57 (d, J=7.2 Hz, 2H), 7.41-7.37 (m, 2H), 7.31-7.26 (m, 4H), 7.17-7.13 (m, 1H), 5.99 (d, J=6.8 Hz, 1H), 5.50 (d, J=6.8 Hz, 1H), 4.39-4.36 (m, 2H), 4.22-4.18 (m, 1H), 1.38 (s, 9H).

### Synthesis of tert-butyl 2-amino-2-(2-fluoro-3-(trifluoromethoxy)phenyl)acetate

Title compound was synthesized by using general procedure-K and obtained as a yellow syrup (0.30 g, 75%). ¹H-NMR (CDCl₃, 400 MHz): δ 7.33-7.29 (m, 1H), 7.26-7.22 (m, 1H), 7.16-7.12 (m, 1H), 4.78 (s, 1H), 1.36 (s, 9H).

### Synthesis of (R)-tert-butyl 2-((2S,3R)-3-((tert-butoxycarbonyl)amino)-2-hydroxy-4-phenylbutanamido)-2-(2-fluoro-3-(trifluoromethoxy)phenyl)acetate (Top spot) and (S)-tert-butyl 2-((2S,3R)-3-((tert-butoxycarbonyl)amino)-2-hydroxy-4-phenylbutanamido)-2-(2-fluoro-3-(trifluoromethoxy)phenyl)acetate (Bottom spot)

Title compounds were synthesized by using general procedure-G and obtained top spot as a white solid (60 mg, 13%) and Bottom spot as a white solid (55 mg, 12%).

Top spot: ¹H-NMR (CDCl₃, 400 MHz): δ 7.83 (brs, 1H), 7.31-7.21 (m, 7H), 7.13-7.08 (m, 1H), 5.68 (d, J=7.2 Hz, 1H), 5.54 (brs, 1H), 5.01 (d, J=7.2 Hz, 1H), 4.13-4.11 (m, 1H), 3.98-3.91 (m, 1H), 3.17-3.03 (m, 2H), 1.36 (s, 18H). m/z (LC-MS): Calculated for C₂₈H₃₄F₄N₂O₇ is 586.57; found, 609.1 (M+Na)⁺.

Bottom spot: ¹H-NMR (DMSO-d₄, 400 MHz): 8.50 (d, J=7.2 Hz, 1H), 7.55-7.46 (m, 2H), 7.31-7.28 (m, 3H), 7.25-7.17 (m, 3H), 6.28 (d, J=9.2 Hz, 1H), 6.03 (d, J=6.4 Hz, 1H), 5.62 (d, J=7.2 Hz, 1H), 3.93-3.91 (m, 1H), 3.88-3.84 (m, 1H), 2.81-2.75 (m, 1H), 2.61-2.58 (m, 1H), 1.31 (s, 9H), 1.22 (s, 9H). m/z (LC-MS): Calculated for C₂₈H₃₄F₄N₂O₇ is 586.57; found, 609.1 (M+Na)⁺.

### Synthesis of (R)-2-((2S,3R)-3-amino-2-hydroxy-4-phenylbutanamido)-2-(2-fluoro-3-(trifluoromethoxy)phenyl)acetic acid hydrochloride (Compound 112)

Title compound was synthesized by using general procedure-L and obtained as a white solid (16.1 mg, 12%). ¹H-NMR (DMSO-d₆, 400 MHz): δ 13.91 (brs, 1H), 8.54 (d, J=6.0 Hz, 1H), 8.02 (brs, 3H), 7.53-7.43 (m, 2H), 7.34-7.25 (m, 6H), 7.08 (d, J=6.0 Hz, 1H), 5.59 (d, J=6.4 Hz, 1H), 3.94 (d, J=4.0 Hz, 1H), 3.58 (brs, 1H), 2.91-2.89 (m, 2H). m/z (LC-MS): Calculated for C₁₉H₁₉ClF₄N₂O₅ is 466.81; found, 431.2 (M-Cl)⁺. Chiral purity 97.8% (AUC), tᵣ=3.20 min.

### Synthesis of (S)-2-((2S,3R)-3-amino-2-hydroxy-4-phenylbutanamido)-2-(2-fluoro-3-(trifluoromethoxy)phenyl)acetic acid hydrochloride (Compound 113)

Title compound was synthesized by using general procedure-L and obtained as a white solid (12.9 mg, 30%). ¹H-NMR (DMSO-d₆, 400 MHz): δ 13.51 (s, 1H), 8.88 (d, J=7.6 Hz, 1H), 7.88 (s, 3H), 7.57-7.52 (m, 2H), 7.36-7.25 (m, 6H), 6.70 (d, J=4.8 Hz, 1H), 5.68 (d, J=7.2 Hz, 1H), 4.09-4.07 (m, 1H), 3.53 (s, 1H), 2.95-2.91 (m, 1H), 2.78-2.66 (m, 1H). m/z (LC-MS): Calculated for C₁₉H₁₉ClF₄N205 is 466.81; found, 431.3 (M-Cl)⁺. Chiral purity 98.1% (AUC), tᵣ=3.19 min.

### Synthesis of (S)-2-((2S,3R)-3-amino-2-hydroxy-4-phenylbutanamido)-2-(2,4-difluoro-3-(trifluoromethoxy)phenyl)acetic acid hydrochloride (Compound 114)

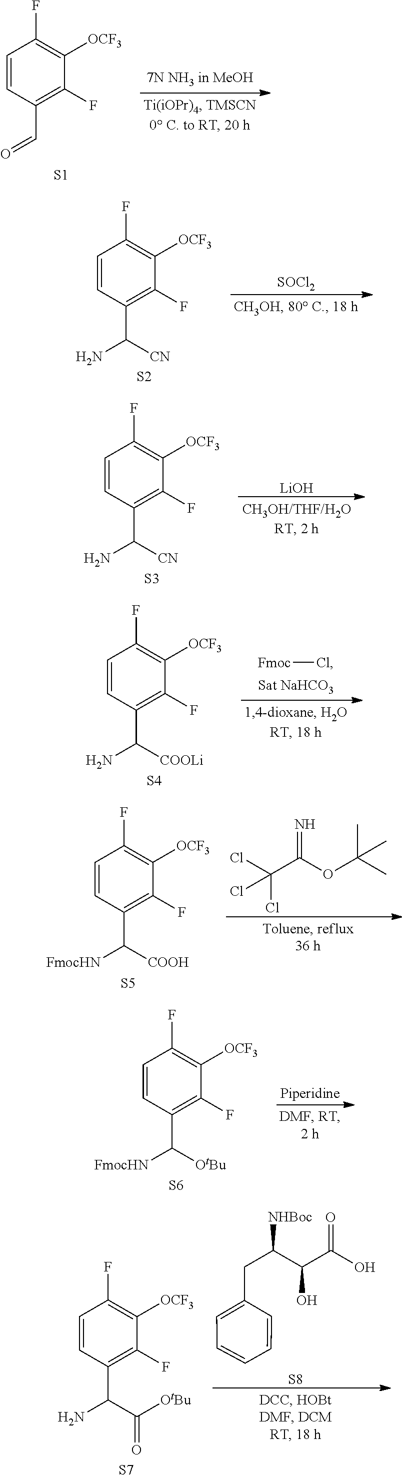

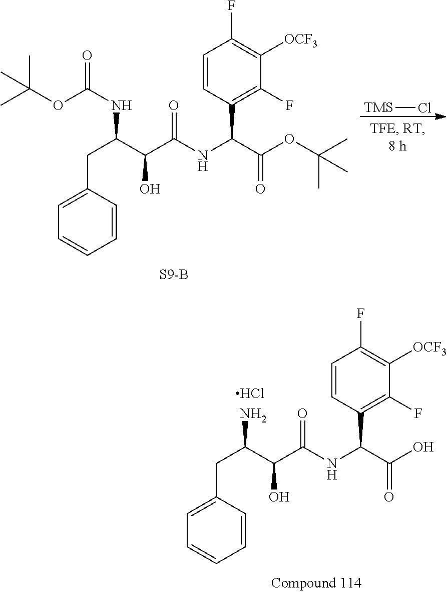 [SMILES: CC(C)(C)OC(=O)N[C@H](Cc1ccccc1)[C@H](O)C(=O)N[C@H](C(=O)OC(C)(C)C)c1ccc(F)c(OC(F)(F)F)c1F ; C[Si](C)(C)Cl ; N[C@H](Cc1ccccc1)[C@H](O)C(=O)N[C@H](C(=O)O)c1ccc(F)c(OC(F)(F)F)c1F ; Cl]

### Synthesis of 2-amino-2-(2,4-difluoro-3-(trifluoromethoxy)phenyl)acetonitrile

Title compound was synthesized by using general procedure-A and obtained as a yellow solid (0.10 g, 45%). ¹H-NMR (DMSO-d₆, 400 MHz): δ 7.77-7.71 (m, 1H), 7.54-7.49 (m, 1H), 5.27 (s, 1H), 3.01 (brs, 2H).

### Synthesis of methyl 2-amino-2-(2,4-difluoro-3-(trifluoromethoxy)phenyl)acetate

Title compound was synthesized by using general procedure-B and obtained as an off white solid (0.38 g, 73%). ¹H-NMR (DMSO-d₆, 400 MHz): 7.66-7.61 (m, 1H), 7.44-7.38 (m, 1H), 4.81 (s, 1H), 3.62 (s, 3H), 1.91 (s, 2H).

### Synthesis of lithium 2-amino-2-(2,4-difluoro-3-(trifluoromethoxy)phenyl)acetate

Title compound was synthesized by using general procedure-C and obtained as a color less gummy solid (1.80 g, crude). m/z (LC-MS): Calculated for C₉H₅F₅LiNO₃ is 277.07; found, 272.1 (M-Li+H)⁺.

### Synthesis of 2-((((9H-fluoren-9-yl)methoxy)carbonyl)amino)-2-(2,4-difluoro-3-(trifluoromethoxy)phenyl)acetic acid

Title compound was synthesized by using general procedure-I and obtained as an off white solid (3.00 g, 93%). m/z (LC-MS): Calculated for C₂₄H₁₆F₅NO₅ is 493.38; found, 494.2 (M+H)⁺.

### Synthesis of tert-butyl 2-((((9H-fluoren-9-yl)methoxy)carbonyl)amino)-2-(2,4-difluoro-3-(trifluoromethoxy)phenyl)acetate

Title compound was synthesized by using general procedure-J and obtained as a white solid (1.60 g, 89%). ¹H-NMR (CDCl₃, 400 MHz): δ 7.76 (d, J=7.6 Hz, 2H), 7.57 (d, J=7.2 Hz, 2H), 7.41-7.37 (m, 2H), 7.33-7.25 (m, 3H), 7.03-6.98 (m, 1H), 6.00 (d, J=6.4 Hz, 1H), 5.43 (d, J=6.4 Hz, 1H), 4.39-4.35 (m, 2H), 4.21-4.17 (m, 1H), 1.33 (s, 9H).

### Synthesis of tert-butyl 2-amino-2-(2,4-difluoro-3-(trifluoromethoxy)phenyl)acetate

Title compound was synthesized by using general procedure-K and obtained as a yellow syrup (0.70 g, 73%). ¹H-NMR (CDCl₃, 400 MHz): δ 7.38-7.24 (m, 1H), 7.03-6.97 (m, 1H), 4.74 (s, 1H), 1.48 (s, 9H).

### Synthesis of (S)-tert-butyl 2-((2S,3R)-3-((tert-butoxycarbonyl)amino)-2-hydroxy-4-phenylbutanamido)-2-(2,4-difluoro-3-(trifluoromethoxy)phenyl)acetate

Title compounds were synthesized by using general procedure-G and obtained bottom spot as a white solid (45 mg, 6%). ¹H-NMR (DMSO-d₆, 400 MHz): 8.55 (d, J=7.6 Hz, 1H), 7.59-7.55 (m, 1H), 7.41-7.35 (m, 1H), 7.27-7.24 (m, 2H), 7.18-7.17 (m, 3H), 6.26 (d, J=9.2 Hz, 1H), 6.04 (d, J=6.4 Hz, 1H), 5.61 (d, J=7.2 Hz, 1H), 3.93-3.84 (m, 2H), 2.81-2.75 (m, 1H), 2.61-2.51 (m, 1H), 1.32 (s, 9H), 1.21 (s, 9H). m/z (LC-MS): Calculated for C₂₈H₃₃F₅N₂O₇ is 604.56; found, 627.1 (M+Na)⁺.

### Synthesis of (S)-2-((2S,3R)-3-amino-2-hydroxy-4-phenylbutanamido)-2-(2,4-difluoro-3-(trifluoromethoxy)phenyl)acetic acid hydrochloride (Compound 114)

Title compound was synthesized by using general procedure-L and obtained as a white solid (25.0 mg, 70%). ¹H-NMR (DMSO-d₆, 400 MHz): δ 13.51 (brs, 1H), 8.92 (d, J=7.6 Hz, 1H), 7.91 (brs, 3H), 7.64-7.59 (m, 1H), 7.47-7.42 (m, 1H), 7.36-7.26 (m, 5H), 6.76 (d, J=6.0 Hz, 1H), 5.67 (d, J=7.2 Hz, 1H), 4.09-4.06 (m, 1H), 3.53 (s, 1H), 3.02-2.85 (m, 1H), 2.80-2.61 (m, 1H). m/z (LC-MS): Calculated for C₁₉H₁₈ClF₅N₂O₅ is 484.80; found, 449.1 (M-Cl)⁺. Chiral purity 99.6% (AUC), tᵣ=3.25 min.

### Synthesis of (S)-2-((2S,3R)-3-amino-2-hydroxy-4-phenylbutanamido)-2-(2,2-difluorobenzo[d][1,3]dioxol-4-yl)acetic acid hydrochloride (Compound 115)

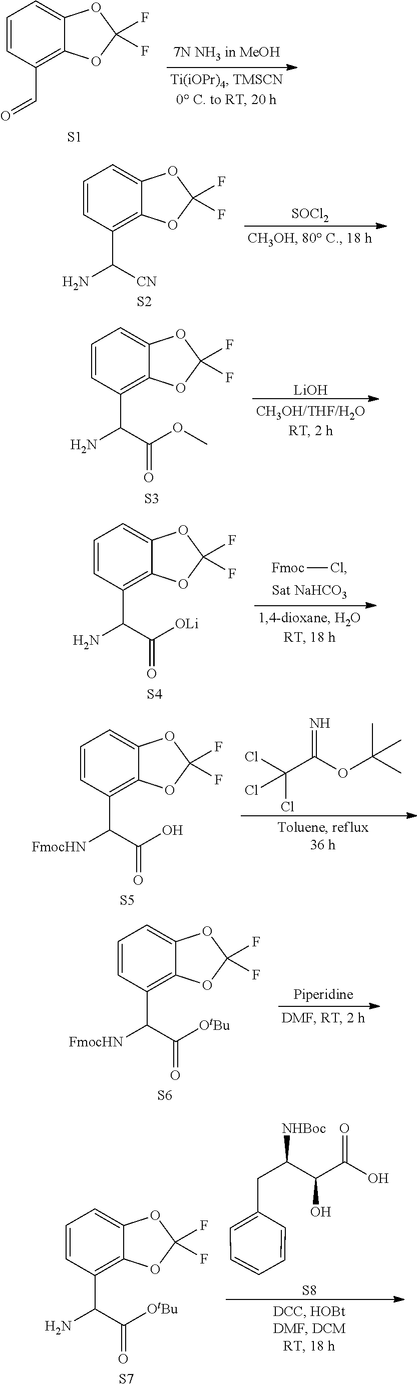

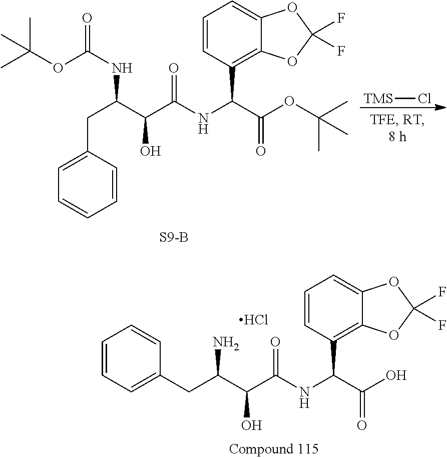

### Synthesis of 2-amino-2-(2,2-difluorobenzo[d][1,3]dioxol-4-yl)acetonitrile

Title compound was synthesized by using general procedure-A and obtained as a yellow solid (0.10 g, 45%). ¹H-NMR (CDCl₃, 400 MHz): δ 7.27-7.25 (m, 1H), 7.18-7.10 (m, 2H), 5.07 (s, 1H), 2.05 (s, 2H).

### Synthesis of methyl 2-amino-2-(2,2-difluorobenzo[d][1,3]dioxol-4-yl)acetate

Title compound was synthesized by using general procedure-B and obtained as a yellow syrup (1.70 g, 79%). m/z (LC-MS): Calculated for C₁₀H₉F₂NO₄ is 245.18; found, 246.2 (M+H)⁺.

### Synthesis of lithium 2-amino-2-(2,2-difluorobenzo[d][1,3]dioxol-4-yl)acetate

Title compound was synthesized by using general procedure-C and obtained as a color less gummy solid (1.40 g, crude). m/z (LC-MS): Calculated for C₉H₆F₂LiNO₄ is 237.09; found, 232.2 (M-Li+H)⁺.

### Synthesis of 2-((((9H-fluoren-9-yl)methoxy)carbonyl)amino)-2-(2,2-difluorobenzo[d][1,3]dioxol-4-yl)acetic acid

Title compound was synthesized by using general procedure-I and obtained as an off white solid (2.20 g, 81%). ¹H-NMR (DMSO-d₆, 400 MHz): 8.12 (brs, 1H), 7.89 (d, J=8.0 Hz, 2H), 7.72-7.71 (m, 2H), 7.42-7.39 (m, 2H), 7.39-7.28 (m, 3H), 7.22-7.18 (m, 2H), 5.21 (brs, 1H), 4.30-4.11 (m, 3H), 4.10 (brs, 1H).

### Synthesis of tert-butyl 2-((((9H-fluoren-9-yl)methoxy)carbonyl)amino)-2-(2,2-difluorobenzo[d][1,3]dioxol-4-yl)acetate

Title compound was synthesized by using general procedure-J and obtained as a white solid (1.90 g, 79%). ¹H-NMR (CDCl₃, 400 MHz): δ 7.76 (d, J=8.0 Hz, 2H), 7.58 (d, J=7.6 Hz, 2H), 7.41-7.37 (m, 2H), 7.31-7.25 (m, 2H), 7.06-7.01 (m, 3H), 6.01 (d, J=6.4 Hz, 1H), 5.44 (d, J=7.2 Hz, 1H), 4.38 (d, J=6.8 Hz, 2H), 4.21-4.14 (m, 1H), 1.40 (s, 9H).

### Synthesis of tert-butyl 2-amino-2-(2,2-difluorobenzo[d][1,3]dioxol-4-yl)acetate

Title compound was synthesized by using general procedure-K and obtained as a yellow gummy solid (0.83 g, 83%). ¹H-NMR (CDCl₃, 400 MHz): δ 7.09-7.04 (m, 2H), 7.01-6.98 (m, 1H), 4.68 (s, 1H), 1.41 (s, 9H).

### Synthesis of (S)-tert-butyl 2-((2S,3R)-3-((tert-butoxycarbonyl)amino)-2-hydroxy-4-phenylbutanamido)-2-(2,2-difluorobenzo[d][1,3]dioxol-4-yl)acetate

Title compound was synthesized by using general procedure-G and obtained bottom spot as a white solid (30 mg, 2%). ¹H-NMR (CDCl₃, 400 MHz): 7.93 (s, 1H), 7.33-7.30 (m, 2H), 7.26-7.19 (m, 3H), 7.00-6.98 (m, 3H), 5.62-5.58 (m, 2H), 4.89 (d, J=8.4 Hz, 1H), 4.20-4.18 (m, 1H), 3.93 (s, 1H), 3.12-3.04 (m, 2H), 1.33 (s, 9H), 1.27 (s, 9H). m/z (LC-MS): Calculated for C₂₈H₃₄F₂N₂O₈ is 564.57; found, 587.3 (M+Na)⁺.

### Synthesis of (S)-2-((2S,3R)-3-amino-2-hydroxy-4-phenylbutanamido)-2-(2,2-difluorobenzo[d][1,3]dioxol-4-yl)acetic acid hydrochloride (Compound 115)

Title compound was synthesized by using general procedure-L and obtained as a white solid (18.0 mg, 78%). ¹H-NMR (DMSO-d₆, 400 MHz): δ 13.50 (brs, 1H), 8.82 (d, J=7.2 Hz, 1H), 7.92 (brs, 3H), 7.40-7.32 (m, 3H), 7.29-7.20 (m, 5H), 6.76 (d, J=5.6 Hz, 1H), 5.53 (d, J=7.2 Hz, 1H), 4.10-4.08 (m, 1H), 3.53 (brs, 1H), 2.96-2.91 (m, 1H), 2.80-2.67 (m, 1H). m/z (LC-MS): Calculated for C₁₉H₁₉ClF₂N₂O₆ is 444.81; found, 409.3 (M-Cl)⁺. Chiral purity 99.1% (AUC), tᵣ=3.45 min.

### Synthesis of (S)-2-((2S,3R)-3-amino-2-hydroxy-4-phenylbutanamido)-3-(4-(trifluoromethoxy)phenyl)propanoic acid (Compound 117)

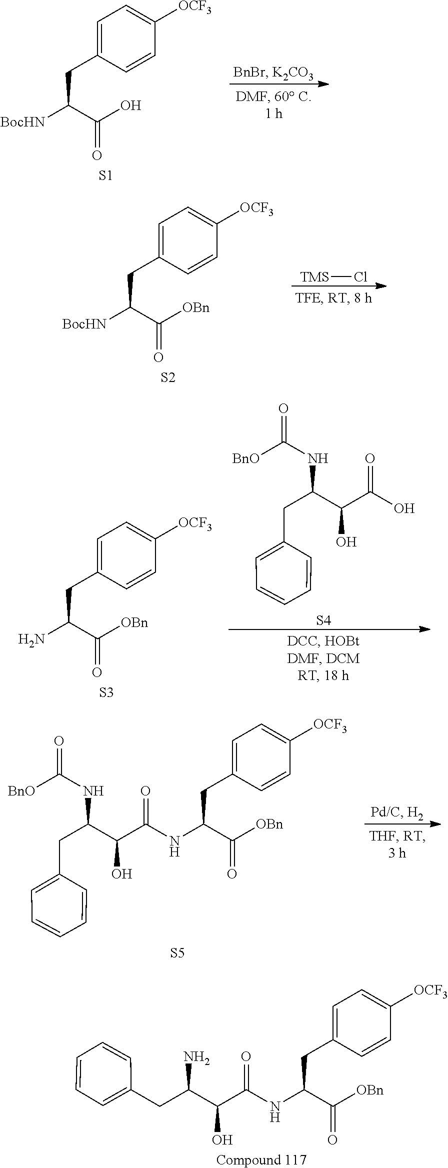

### Synthesis of (S)-benzyl 2-((tert-butoxycarbonyl)amino)-3-(4-(trifluoromethoxy)phenyl)propanoate

Title compound was synthesized by using general procedure-E and obtained as a white solid (0.11 g, 85%). ¹H-NMR (CDCl₃, 400 MHz): δ 7.37-7.35 (m, 3H), 7.30-7.26 (m, 2H), 7.08-7.01 (m, 4H), 5.19-5.08 (m, 2H), 5.00 (d, J=8.0 Hz, 1H), 4.63-4.61 (m, 1H), 3.10-3.04 (m, 2H), 1.41 (s, 9H).

### Synthesis of (S)-benzyl 2-amino-3-(4-(trifluoromethoxy)phenyl)propanoate

Title compound was synthesized by using general procedure-F and obtained as a white solid (0.07 g, 90%). ¹H-NMR (CDCl₃, 400 MHz): δ 7.37-7.34 (m, 3H), 7.30-7.28 (m, 2H), 7.16-7.13 (m, 2H), 7.10-7.08 (m, 2H), 5.16-5.09 (m, 2H), 3.77-3.74 (m, 1H), 3.08-3.03 (m, 1H), 2.93-2.88 (m, 1H).

### Synthesis of (S)-benzyl 2-((2S,3R)-3-(((benzyloxy)carbonyl)amino)-2-hydroxy-4-phenylbutanamido)-3-(4-(trifluoromethoxy)phenyl)propanoate

Title compounds were synthesized by using general procedure-G and obtained as a white solid (0.32 g, 29%). ¹H-NMR (CDCl₃, 400 MHz): δ7.35-7.34 (m, 3H), 7.32-7.21 (m, 11H), 7.16-7.14 (m, 2H), 6.99-6.95 (m, 4H), 5.17-5.13 (m, 2H), 5.08-4.99 (m, 3H), 4.86-4.81 (m, 2H), 4.14-4.09 (m, 2H), 3.04-2.95 (m, 4H). m/z (LC-MS): Calculated for C₃₅H₃₃F₃N₂O₇ is 650.64; found, 651.3 (M+H₁)⁺.

### Synthesis of (S)-2-((2S,3R)-3-amino-2-hydroxy-4-phenylbutanamido)-3-(4-(trifluoromethoxy)phenyl)propanoic acid (Compound 117)

Title compound was synthesized by using general procedure-H and obtained as a white solid (30.0 mg, 46%). ¹H-NMR (DMSO-d₆, 400 MHz): δ 7.98 (d, J=7.60 Hz, 1H), 7.34-7.24 (m, 6H), 7.22-7.19 (m, 2H), 5.81 (s, 1H), 4.05 (s, 1H), 3.77-3.76 (m, 1H), 3.50-3.49 (m, 1H), 3.29-3.26 (m, 1H), 3.16-3.01 (m, 1H), 2.87-2.79 (m, 2H). m/z (LC-MS): Calculated for C₂₀H₂₁F₃N₂O₅ is 426.39; found, 427.2 (M+H)⁺. Chiral purity 97.3% (AUC), tr=3.32 min.

### Synthesis of (S)-2-((2S,3R)-3-amino-2-hydroxy-4-phenylbutanamido)-3-(3-(trifluoromethoxy)phenyl)propanoic acid (Compound 118)

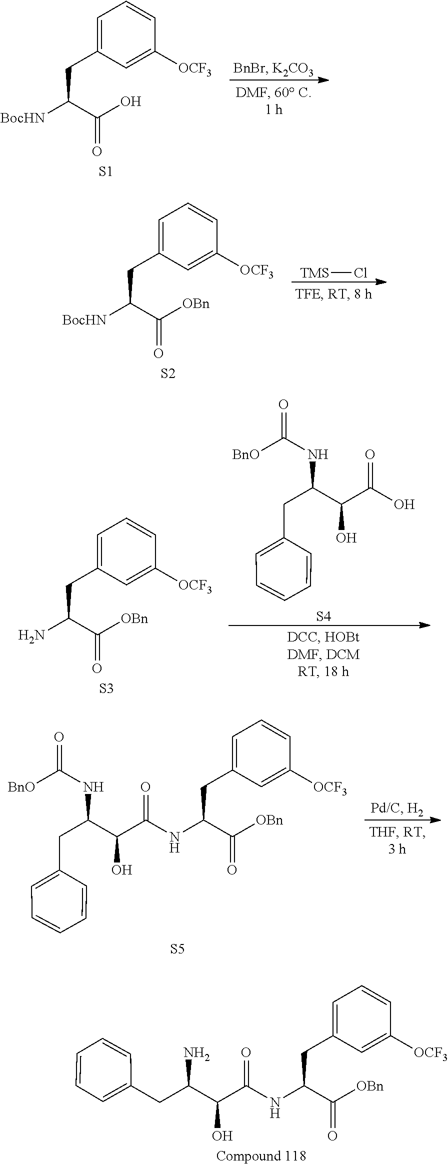

### Synthesis of (S)-benzyl 2-((tert-butoxycarbonyl)amino)-3-(3-(trifluoromethoxy)phenyl)propanoate

Title compound was synthesized by using general procedure-E and obtained as a white solid (0.09 g, 72%). ¹H-NMR (CDCl₃, 400 MHz): δ 7.38-7.35 (m, 2H), 7.31-7.26 (m, 2H), 7.23-7.21 (m, 1H), 7.08-7.06 (m, 1H), 6.96-6.92 (m, 2H), 5.18-5.09 (m, 2H), 5.01 (d, J=8.0 Hz, 1H), 3.14-3.12 (m, 1H), 3.09-3.07 (m, 1H), 1.41 (s, 9H).

### Synthesis of (S)-benzyl 2-amino-3-(3-(trifluoromethoxy)phenyl)propanoate

Title compound was synthesized by using general procedure-F and obtained as a white solid (0.09 g, crude). ¹H-NMR (CDCl₃, 400 MHz): δ 7.39-7.37 (m, 3H), 7.35-7.28 (m, 2H), 7.08-7.06 (m, 2H), 7.03 (s, 1H), 5.13 (s, 2H), 3.79-3.75 (m, 1H), 3.10-3.06 (m, 1H), 2.95-2.89 (m, 1H).

### Synthesis of (S)-benzyl 2-((2S,3R)-3-(((benzyloxy)carbonyl)amino)-2-hydroxy-4-phenylbutanamido)-3-(3-(trifluoromethoxy)phenyl)propanoate

Title compound was synthesized by using general procedure-G and obtained as a white solid (0.12 g, 21%). ¹H-NMR (DMSO-d₆, 400 MHz): δ 8.15 (d, J=8.0 Hz, 1H), 7.35-7.12 (m, 20H), 6.75 (d, J=8.0 Hz, 1H), 6.02 (d, J=5.2 Hz, 1H), 5.07 (s, 2H), 5.05-4.91 (m, 2H), 4.69-4.63 (m, 1H), 3.91-3.88 (m, 1H), 3.17-3.04 (m, 2H), 2.73-2.67 (m, 1H), 2.49-2.32 (m, 1H). m/z (LC-MS): Calculated for C₃₅H₃₃F₃N₂O₇ is 650.64; found, 651.3 (M+H)⁺.

### Synthesis of (S)-2-((2S,3R)-3-amino-2-hydroxy-4-phenylbutanamido)-3-(3-(trifluoromethoxy)phenyl)propanoic acid (Compound 118)

Title compound was synthesized by using general procedure-H and obtained as a white solid (25.0 mg, 14%). ¹H-NMR (DMSO-d₆, 400 MHz): δ 7.99 (d, J=8.0 Hz, 1H), 7.37-7.22 (m, 6H), 7.16-7.11 (m, 2H), 4.07 (brs, 1H), 3.76 (d, J=2.4 Hz, 1H), 3.50-3.48 (m, 1H), 3.32 (d, J=4.0 Hz, 1H), 3.29 (d, J=3.6 Hz, 1H), 3.09-3.03 (m, 1H), 2.90-2.78 (m, 2H). m/z (LC-MS): Calculated for C₂₀H₂₁F₃N₂O₅ is 426.39; found, 427.2 (M+H)⁺. Chiral purity >99% (AUC), tᵣ=3.32 min.

### Synthesis of (S)-2-((2S,3R)-3-amino-2-hydroxy-4-phenylbutanamido)-3-(3-fluoro-4-(trifluoromethoxy phenyl propanoic acid (Compound 119)

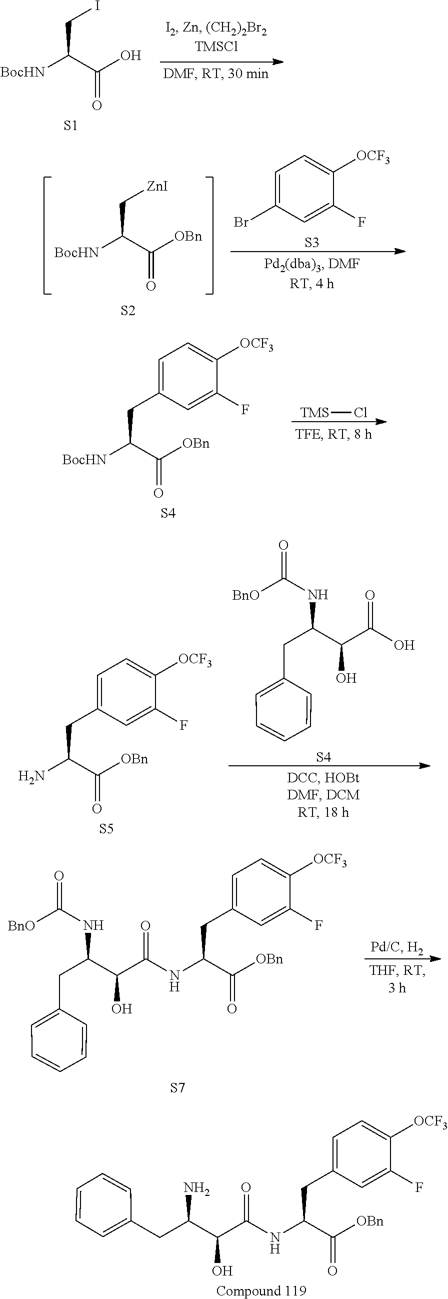

### Synthesis of (R)-(3-(benzyloxy)-2-((tert-butoxycarbonyl)amino)-3-oxopropyl)zinc(II) iodide

Title compound was synthesized by using general procedure-M and supernatant layer was used in next step without further purification.

### Synthesis of (S)-benzyl 2-((tert-butoxycarbonyl)amino)-3-(3-fluoro-4-(trifluoromethoxy)phenyl)propanoate

Title compound was synthesized by using general procedure-N and obtained as a white solid (0.61 g, 55%). ¹H-NMR (CDCl₃, 400 MHz): δ 7.38-7.36 (m, 3H), 7.32-7.26 (m, 2H), 7.13-7.09 (m, 1H), 6.87 (d, J=10.0 Hz, 1H), 6.79 (d, J=8.0 Hz, 1H), 5.21-5.18 (m, 1H), 5.12-5.09 (m, 1H), 5.04 (d, J=8.0 Hz, 1H), 4.62-4.60 (m, 1H), 3.12-3.10 (m, 1H), 3.03-3.01 (m, 1H), 1.45 (s, 9H).

### Synthesis of (S)-benzyl 2-amino-3-(3-fluoro-4-(trifluoromethoxy)phenyl)propanoate

Title compound was synthesized by using general procedure-F and obtained as a yellow syrup (0.50 g, crude). ¹H-NMR (CDCl₃, 400 MHz): δ 7.38-7.35 (m, 3H), 7.33-7.29 (m, 2H), 7.16 (t, J=8.4 Hz, 1H), 7.00 (dd, J=2.0, 10.8 Hz, 1H), 6.91 (d, J=8.4 Hz, 1H), 5.14 (q, J=12.0 Hz, 2H), 3.76-3.73 (m, 1H), 3.06-3.01 (m, 1H), 2.91-2.85 (m, 1H). m/z (LC-MS): Calculated for C₁₇H₁₅F₄NO₃ is 357.30; found, 358.2 (M+H)⁺.

### Synthesis of (S)-benzyl 2-((2S,3R)-3-(((benzyloxy)carbonyl)amino)-2-hydroxy-4-phenylbutanamido)-3-(3-fluoro-4-(trifluoromethoxy)phenyl)propanoate

Title compound was synthesized by using general procedure-G and obtained as a white solid (0.30 g, 27%). ¹H-NMR (DMSO-d₆, 400 MHz): δ 8.21 (d, J=8.0 Hz, 1H), 7.36-7.10 (m, 18H), 6.81 (d, J=9.2 Hz, 1H), 6.03 (d, J=5.6 Hz, 1H), 5.10 (q, J=12.8 Hz, 2H), 4.95-4.91 (m, 1H), 4.86-4.82 (m, 1H), 4.68 (q, J=7.6 Hz, 1H), 3.91-3.90 (m, 2H), 3.08 (d, J=6.8 Hz, 2H), 2.71 (dd, J=4.8, 13.6 Hz, 1H), 2.46-2.41 (m, 1H). m/z (LC-MS): Calculated for C₃₅H₃₂F₄N₂O₇ is 668.63; found, 667.1 (M−H)⁺.

### Synthesis of (S)-2-((2S,3R)-3-amino-2-hydroxy-4-phenylbutanamido)-3-(3-fluoro-4-(trifluoromethoxy)phenyl)propanoic acid (Compound 119)

Title compound was synthesized by using general procedure-H and obtained as a white solid (58.0 mg, 20%). ¹H-NMR (DMSO-d₆+D₂O, 400 MHz): δ 7.40-7.25 (m, 7H), 7.14 (d, J=8.4 Hz, 1H), 4.04 (dd, J=4.0, 10.0 Hz, 1H), 3.82 (d, J=2.4 Hz, 1H), 3.56-3.52 (m, 1H), 3.29 (dd, J=4.0, 14.0 Hz, 1H), 3.17-3.07 (m, 1H), 2.93-2.82 (m, 2H). m/z (LC-MS): Calculated for C₂₀H₂₀F₄N₂O₅ is 444.38; found, 445.3 (M+H)⁺. Chiral purity 99.26% (AUC), tᵣ=3.28 min.

### Synthesis of (S)-2-((2S,3R)-3-amino-2-hydroxy-4-phenylbutanamido)-3-(3,5-difluoro-4-(trifluoromethyl)phenyl)propanoic acid (Compound 120)

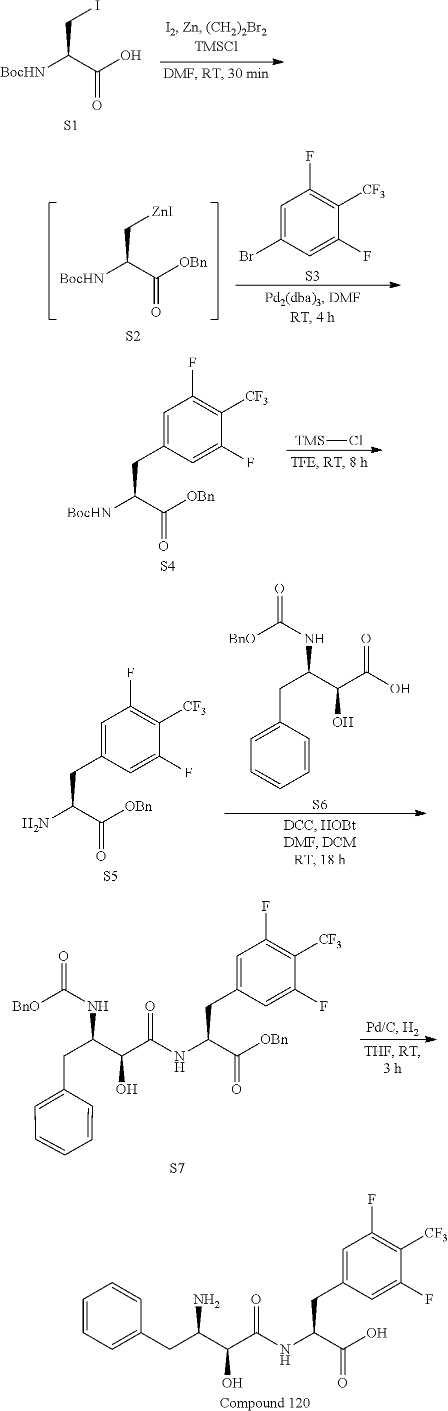

### Synthesis of (R)-(3-(benzyloxy)-2-((tert-butoxycarbonyl)amino)-3-oxopropyl)zinc(II) iodide

Title compound was synthesized by using general procedure-M and supernatant layer was used in next step without further purification.

### Synthesis of (S)-benzyl 2-((tert-butoxycarbonyl)amino)-3-(3,5-difluoro-4-(trifluoromethyl)phenyl)propanoate

Title compound was synthesized by using general procedure-N and obtained as a white solid (0.80 g, 72%). ¹H-NMR (CDCl₃, 400 MHz): δ 7.38-7.36 (m, 3H), 7.32-7.30 (m, 2H), 6.66 (d, J=10.4 Hz, 2H), 5.24 (d, J=12.0 Hz, 1H), 5.11-5.08 (m, 2H), 4.61 (q, J=6.8 Hz, 1H), 3.16 (dd, J=6.0, 13.8 Hz, 1H), 3.02 (dd, J=5.6, 13.6 Hz, 1H), 1.42 (s, 9H).

### Synthesis of (S)-benzyl 2-amino-3-(3,5-difluoro-4-(trifluoromethyl)phenyl)propanoate

Title compound was synthesized by using general procedure-F and obtained as a yellow syrup (0.46 g, 98%). ¹H-NMR (CDCl₃, 400 MHz): δ 7.38-7.50 (m, 3H), 7.33-7.29 (m, 2H), 6.81 (d, J=10.4 Hz, 2H), 5.15 (q, J=12.0 Hz, 2H), 3.77-3.74 (m, 1H), 3.05 (dd, J=5.6, 13.8 Hz, 1H), 2.88 (dd, J=7.6, 14.0 Hz, 1H).

### Synthesis of (S)-benzyl 2-((2S,3R)-3-(((benzyloxy)carbonyl)amino)-2-hydroxy-4-phenylbutanamido)-3-(3,5-difluoro-4-(trifluoromethyl)phenyl)propanoate

Title compounds were synthesized by using general procedure-G and obtained as a white solid (0.20 g, 23%). ¹H-NMR (DMSO-d₆, 400 MHz): δ 8.32 (d, J=8.4 Hz, 1H), 7.36-7.06 (m, 17H), 6.78 (d, J=9.2 Hz, 1H), 6.04 (d, J=6.0 Hz, 1H), 5.12 (q, J=12.4 Hz, 2H), 4.93-4.90 (m, 1H), 4.84-4.74 (m, 2H), 3.93-3.85 (m, 2H), 3.17-3.14 (m, 2H), 2.67 (dd, J=5.2, 13.4 Hz, 1H), 2.34-2.29 (m, 1H), m/z (LC-MS): Calculated for C₃₅H₃₁F₅N₂O₆ is 670.62; found, 671.2 (M−H)⁺.

### Synthesis of (S)-2-((2S,3R)-3-amino-2-hydroxy-4-phenylbutanamido)-3-(3,5-difluoro-4-(trifluoromethyl)phenyl)propanoic acid (Compound 120)

Title compound was synthesized by using general procedure-H and obtained as a white solid (38.0 mg, 10%). ¹H-NMR (DMSO-d₆+D₂O, 400 MHz): δ 7.37-7.35 (m, 2H), 7.29-7.26 (m, 3H), 7.19 (d, J=11.2 Hz, 2H), 4.06 (dd, J=4.4, 9.2 Hz, 1H), 3.83-3.77 (m, 2H), 3.51-3.49 (m, 1H), 3.31 (dd, J=4.0, 13.8 Hz, 1H), 3.15-3.14 (m, 1H), 2.91-2.86 (m, 1H), 2.77-2.73 (m, 1H). m/z (LC-MS): Calculated for C₂₀H₁₉F₅N₂O₄ is 446.37; found, 447.2 (M+H)⁺. Chiral purity 99.52% (AUC), tᵣ=3.25 min.

### Chiral HPLC Method Details:

Column: Amylose-SA (250×4.6 mm, 5 μm)
Mobile phase: Hexane:EtOH (50:50)
Flow rate: 1.0 mL/min
Injection Volume: 20 μL
Wavelength: 210 nm
Mode: Isocratic
Diluent: Methanol
Sample concentration: 1 mg/mL

Compounds of Formula (I) in Table C3 can be prepared analogously to the Examples above. As the skilled person will appreciate, the compounds described herein can be prepared using variations of any one or more of the procedures described herein, e.g., substituting one or more starting materials and/or reagents for those described in one or more of the working examples.

TABLE C3

| Compound | |
|---|---|
| Compound 64 | 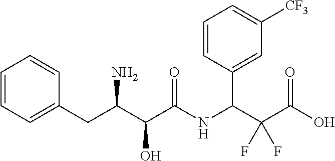 [SMILES: N[C@H](Cc1ccccc1)[C@H](O)C(=O)NC(c1cccc(C(F)(F)F)c1)C(F)(F)C(=O)O] |
| Compound 65 | 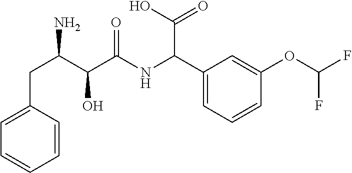 [SMILES: N[C@H](Cc1ccccc1)[C@H](O)C(=O)NC(C(=O)O)c1cccc(OC(F)F)c1] |
| Compound 66 | 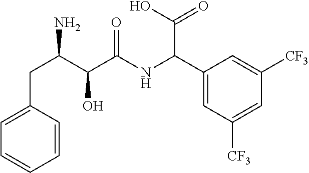 [SMILES: N[C@H](Cc1ccccc1)[C@H](O)C(=O)NC(C(=O)O)c1cc(C(F)(F)F)cc(C(F)(F)F)c1] |
| Compound 67 | 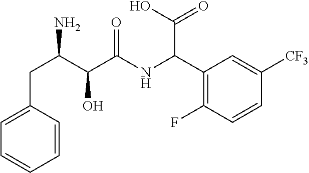 [SMILES: N[C@H](Cc1ccccc1)[C@H](O)C(=O)NC(C(=O)O)c1cc(C(F)(F)F)ccc1F] |
| Compound 68 | 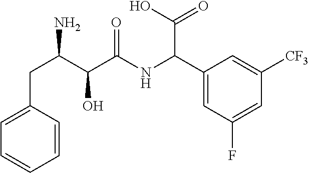 [SMILES: N[C@H](Cc1ccccc1)[C@H](O)C(=O)NC(C(=O)O)c1cc(F)cc(C(F)(F)F)c1] |
| Compound 69 | 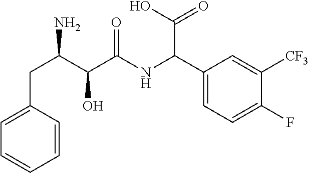 [SMILES: N[C@H](Cc1ccccc1)[C@H](O)C(=O)NC(C(=O)O)c1ccc(F)c(C(F)(F)F)c1] |
| Compound 70 | 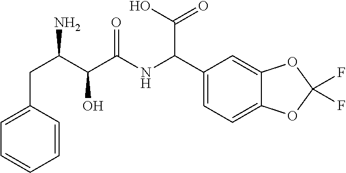 [SMILES: N[C@H](Cc1ccccc1)[C@H](O)C(=O)NC(C(=O)O)c1ccc2c(c1)OC(F)(F)O2] |
| Compound 71 | 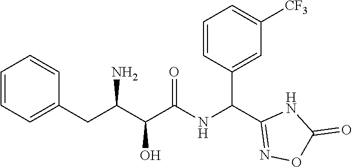 [SMILES: N[C@H](Cc1ccccc1)[C@H](O)C(=O)NC(c1cccc(C(F)(F)F)c1)c1noc(=O)[nH]1] |
| Compound 72 | 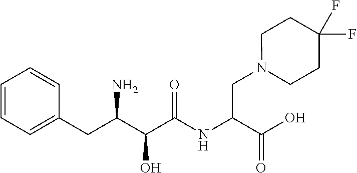 [SMILES: N[C@H](Cc1ccccc1)[C@H](O)C(=O)NC(CN1CCC(F)(F)CC1)C(=O)O] |
| Compound 73 | 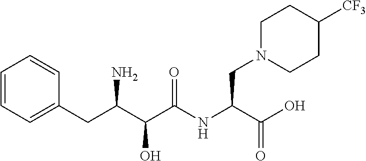 [SMILES: N[C@H](Cc1ccccc1)[C@H](O)C(=O)N[C@@H](CN1CCC(C(F)(F)F)CC1)C(=O)O] |
| Compound 74 | 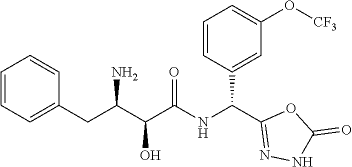 [SMILES: N[C@H](Cc1ccccc1)[C@H](O)C(=O)N[C@H](c1cccc(OC(F)(F)F)c1)c1n[nH]c(=O)o1] |
| Compound 75 | 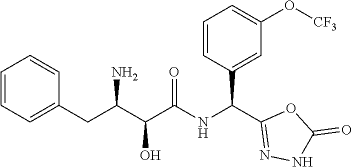 [SMILES: N[C@H](Cc1ccccc1)[C@H](O)C(=O)N[C@@H](c1cccc(OC(F)(F)F)c1)c1n[nH]c(=O)o1] |
| Compound 76 | 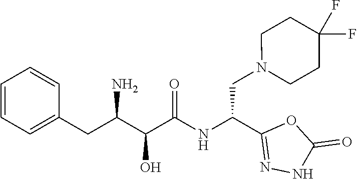 [SMILES: N[C@H](Cc1ccccc1)[C@H](O)C(=O)N[C@H](CN1CCC(F)(F)CC1)c1n[nH]c(=O)o1] |
| Compound 77 | 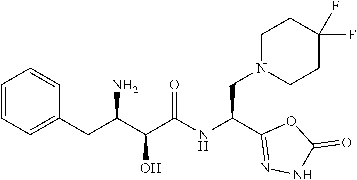 [SMILES: N[C@H](Cc1ccccc1)[C@H](O)C(=O)N[C@@H](CN1CCC(F)(F)CC1)c1n[nH]c(=O)o1] |
| Compound 78 | 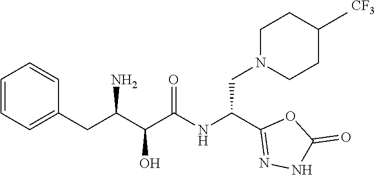 |
| Compound 79 | 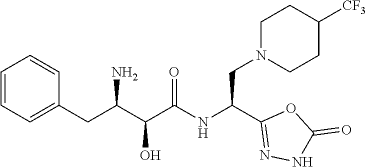 [SMILES: N[C@H](Cc1ccccc1)[C@H](O)C(=O)N[C@@H](CN1CCC(C(F)(F)F)CC1)c1n[nH]c(=O)o1] |
| Compound 80 | 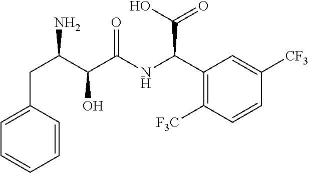 [SMILES: N[C@H](Cc1ccccc1)[C@H](O)C(=O)N[C@@H](C(=O)O)c1cc(C(F)(F)F)ccc1C(F)(F)F] |
| Compound 81 | 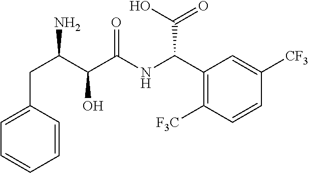 [SMILES: N[C@H](Cc1ccccc1)[C@H](O)C(=O)N[C@H](C(=O)O)c1cc(C(F)(F)F)ccc1C(F)(F)F] |
| Compound 82 | 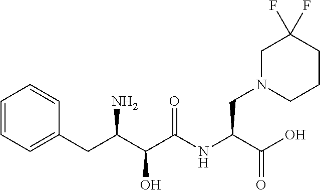 [SMILES: N[C@H](Cc1ccccc1)[C@H](O)C(=O)N[C@@H](CN1CCCC(F)(F)C1)C(=O)O] |
| Compound 83 | 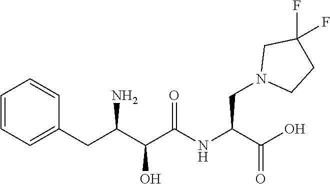 [SMILES: N[C@H](Cc1ccccc1)[C@H](O)C(=O)N[C@@H](CN1CCC(F)(F)C1)C(=O)O] |
| Compound 98 | 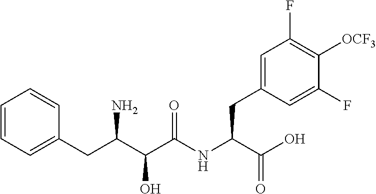 [SMILES: N[C@H](Cc1ccccc1)[C@H](O)C(=O)N[C@@H](Cc1cc(F)c(OC(F)(F)F)c(F)c1)C(=O)O] |
| Compound 99 | 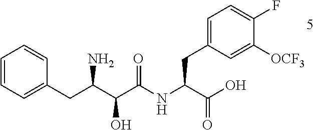 [SMILES: N[C@H](Cc1ccccc1)[C@H](O)C(=O)N[C@@H](Cc1ccc(F)c(OC(F)(F)F)c1)C(=O)O] |
| Compound 100 | 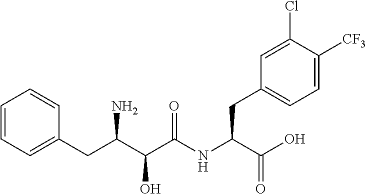 [SMILES: N[C@H](Cc1ccccc1)[C@H](O)C(=O)N[C@@H](Cc1ccc(C(F)(F)F)c(Cl)c1)C(=O)O] |
| Compound 101 | 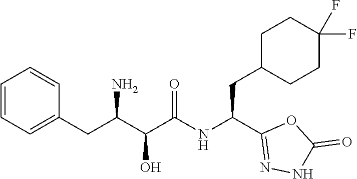 [SMILES: N[C@H](Cc1ccccc1)[C@H](O)C(=O)N[C@@H](CC1CCC(F)(F)CC1)c1n[nH]c(=O)o1] |
| Compound 116 | 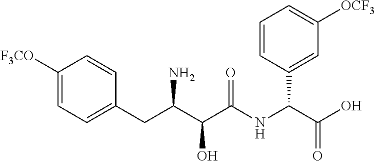 [SMILES: N[C@H](Cc1ccc(OC(F)(F)F)cc1)[C@H](O)C(=O)N[C@@H](C(=O)O)c1cccc(OC(F)(F)F)c1] |
| Compound 135 | 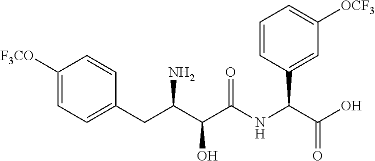 [SMILES: N[C@H](Cc1ccc(OC(F)(F)F)cc1)[C@H](O)C(=O)N[C@H](C(=O)O)c1cccc(OC(F)(F)F)c1] |

### Biological Assays

To identify optimal conditions for identifying novel inhibitors of CNDP2, enzyme assays were performed with varied sources of purified enzyme, concentrations of enzyme, peptide substrates, concentrations of peptide substrates and metal cofactors. Empirically-determined optimal conditions included 50 nM purified CNDP2 (Sino Biological) in Assay Buffer that contained 100 mM Tris pH 7.4, 100 mM NaCl, 100 microM MnCl₂, 1 mM DTT, 1% DMSO and Met-His peptide substrate added at 1 mM (near the enzyme Km for this substrate). The reaction mixture was incubated for 30 minutes at 37° C. before it was quenched with an equal volume of 1% TCA. Free histidine was quantified after derivatization with a fluorophore (o-phthaldialdehyde from Sigma-Aldrich, excitation 340 nm, emission 460 nm). The reaction was conducted in an automation-friendly format to meet future screening throughput demands. A histidine titration standard curve was also included to enable conversion of fluorescence signal to molar concentration of histidine released by the hydrolysis reaction and to ensure the linearity of the fluorescence signal vs. free histidine.

Dose Response inhibition by bestatin and Compound of Formula (I) disclosed herein: To confirm that the assay was suitable for measuring potency, the assay was run with a range of concentrations of a known CNDP2 inhibitor (bestatin from Caymen Chemical) ranging from 10 microM down to 5 pM. The IC₅₀ determined for bestatin had suitable intra- and inter-day precision, as evidenced by the small SEM between replicates in an individual run day and the consistent IC₅₀ near 2 micromolar across run days (See bestatin results in and Table 1).

Compounds of Formula (I) disclosed herein were screened for potency in inhibiting CNDP2 enzyme activity in a dose-response experiment. Bestatin was included with each batch of test molecules as a benchmark for assay quality control. An example set of dose-response curves for a batch of novel molecules is shown in FIG. 1. The IC₅₀ values determined from the dose-response experiments are reported for bestatin and compounds of Formula (I) disclosed herein in Table 1 and Table 2.

### Table 1 and Table 2

“+++” indicates that IC₅ (100 nM; “++” indicates that 100 nM<=C₅₀<1000 nM;
“+” indicates that IC₅₀>=1000 nM; and “ND” indicates that the IC₅₀ data is not available for this compound.

TABLE 1

| Compound | IC₅₀ |
|---|---|
| Bestatin | (+) |
| Compound 1 | (+) |
| Compound 2 | ND |
| Compound 3 | (+) |
| Compound 4 | (++) |
| Compound 5 | (++) |
| Compound 6 | ND |
| Compound 7 | ND |
| Compound 8 | (+) |
| Compound 9 | (+) |
| Compound 10 | (+++) |
| Compound 11 | (+) |
| Compound 12 | ND |
| Compound 13 | ND |
| Compound 14 | (+) |
| Compound 15 | (+) |
| Compound 16 | (++) |
| Compound 17 | (+++) |
| Compound 18 | (+) |
| Compound 19 | (+) |
| Compound 20 | (+) |
| Compound 21 | (+) |
| Compound 22 | (+) |
| Compound 23 | (+++) |
| Compound 24 | (+) |
| Compound 25 | (+++) |
| Compound 26 | (+) |
| Compound 27 | (+) |
| Compound 28 | (+) |
| Compound 29 | (+) |
| Compound 30 | (++) |
| Compound 31 | (+++) |
| Compound 32 | (+) |
| Compound 33 | (+) |
| Compound 34 | (++) |
| Compound 35 | (+) |
| Compound 36 | (+++) |
| Compound 37 | ND |
| Compound 38 | (+) |
| Compound 39 | (+) |
| Compound 40 | (+) |
| Compound 41 | (+) |
| Compound 42 | (+) |
| Compound 43 | (+) |
| Compound 44 | (+) |
| Compound 45 | (++) |
| Compound 46 | ND |
| Compound 47 | ND |
| Compound 48 | ND |
| Compound 49 | (+++) |
| Compound 50 | ND |
| Compound 51 | ND |
| Compound 52 | (+) |
| Compound 53 | (++) |
| Compound 54 | (+++) |
| Compound 55 | (+) |
| Compound 56 | (+++) |
| Compound 57 | (+) |
| Compound 58 | ND |
| Compound 59 | ND |
| Compound 60 | ND |
| Compound 61 | (++) |

TABLE 2

| Compound No. | IC₅₀ |
|---|---|
| Compound 64 | (+) |
| Compound 65 | (+++) |
| Compound 66 | (+) |
| Compound 67 | (+) |
| Compound 68 | (+++) |
| Compound 69 | (+++) |
| Compound 71 | (++) |
| Compound 72 | (+) |
| Compound 73 | (+) |
| Compound 74 | (+) |
| Compound 75 | (+) |
| Compound 76 | ND |
| Compound 77 | ND |
| Compound 78 | ND |
| Compound 79 | ND |
| Compound 80 | (+) |
| Compound 81 | (+) |
| Compound 82 | (+) |
| Compound 83 | (+) |
| Compound 84 | (+) |
| Compound 85 | (+) |
| Compound 86 | (+) |
| Compound 87 | (++) |
| Compound 88 | (+) |
| Compound 89 | (+) |
| Compound 90 | (+++) |
| Compound 91 | (++) |
| Compound 92 | (+++) |
| Compound 93 | (+) |
| Compound 94 | (+++) |
| Compound 95 | (+) |
| Compound 96 | (+++) |
| Compound 97 | (++) |
| Compound 98 | (++) |
| Compound 99 | (+) |
| Compound 100 | (++) |
| Compound 101 | (+) |
| Compound 102 | (+) |
| Compound 103 | (+++) |
| Compound 104 | (+) |
| Compound 105 | (+++) |
| Compound 106 | (+) |
| Compound 107 | (+++) |
| Compound 108 | (+) |
| Compound 109 | (+++) |
| Compound 110 | (+) |
| Compound 111 | (+++) |
| Compound 112 | (++) |
| Compound 113 | (+++) |
| Compound 114 | (+++) |
| Compound 115 | (+) |
| Compound 116 | ND |
| Compound 117 | (+) |
| Compound 118 | (+++) |
| Compound 119 | (++) |
| Compound 120 | ND |
| Compound 121 | (+) |
| Compound 122 | (++) |
| Compound 123 | (+) |
| Compound 124 | (+) |
| Compound 125 | (++) |
| Compound 126 | (+) |
| Compound 127 | (+) |
| Compound 128 | (+) |
| Compound 129 | (+) |
| Compound 130 | (+) |
| Compound 131 | (+) |
| Compound 132 | (++) |
| Compound 133 | (+) |
| Compound 134 | (+) |
| Compound 135 | ND |
| Compound 115 | (++) |

### Numbered Clauses

The compounds, compositions, methods, and other subject matter described herein are further described in the following numbered clauses:

1. A compound having formula I:

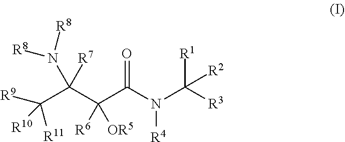

or a pharmaceutically acceptable thereof, wherein:
R¹, R², and R³ are defined according to (A) and (B) below:
   (A)
R¹ is selected from the group consisting of —CO₂H, —CO—NR¹²R¹³, and R^{1A};
wherein:
R^{1A} is a carboxylic acid isostere or bioisostere;
each occurrence of R¹² and R¹³ is independently selected from the group consisting of: Hand C₁₋₃ alkyl; R^{12a} and R^{13a}, together with the carbon atom to which each is attached, form a C₃-C₆ cycloalkyl;
R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
      (B)
R¹, R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
R⁴, R⁵, R⁶, R⁷, R⁸, R⁹, R¹⁰, and R¹¹ are defined according to (C) and (D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R^{b}; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R⁸ is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a};
each occurrence of R′ and R″ is independently selected from the group consisting of: H; —OH; and C₁₋₄ alkyl;
A) provided when:
R¹ is CO₂H,
each of R⁴, R⁵, R⁶, R⁷, R⁸, R⁹, and R¹⁰ is H,
R¹¹ is phenyl that is optionally substituted with one or more of NO₂, F, Cl, —OCH₃, —CH₃, phenyl, 2-benzothiazolyloxy, benzyloxy, iodo, —SCH₃, —SO₂CH₃, iso-propyl, iso-propoxy, iso-butyl, —OH, N₃, and NH₂; and
one of R² and R³ is H,
then the other of R² and R³ cannot be:
(i) the amino acid sidechain present in leucine, iso-leucine, threonine, tyrosine, O-benzyl-protected tyrosine, tryptophan, allothreonine, histidine, valine, alanine, arginine, glutamine, glutamic acid, aspartic acid, serine, lysine, N-benzyloxy-protected lysine, or methionine; or
(ii) n-hexyl, tert-butyl, n-butyl, or n-propyl;
B) further provided when:
R¹ is CO₂H,
each of R⁴, R⁵, R⁶, R⁷, R⁸, R⁹, and R¹⁰ is H,
R² and R³ together with the carbon atom to which each is attached form a 3, 4, 5, or 6 member ring; and
R¹ is phenyl
then the phenyl ring cannot be substituted at the para position with —SO₂CH₃; and C) further provided the compound is not

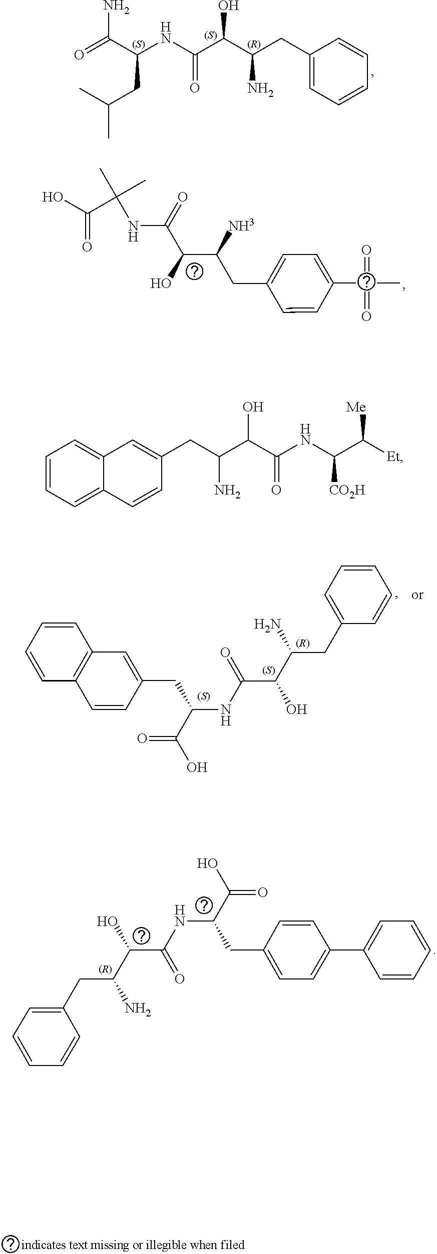

2. A compound having formula I:

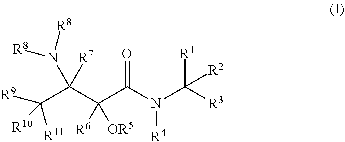

or a pharmaceutically acceptable thereof, wherein:
R¹, R², and R³ are defined according to (A) and (B) below:
   (A)
R¹ is selected from the group consisting of —CO₂H, —CR^{12a}R^{13a}CO₂H, —CO(NR¹²)—OH, —CO—NR¹²R¹³, —CO—NR¹²—SO₂R⁴, —CO—NR¹²—CN, —CO—NR¹²—CH₂—CF₃,

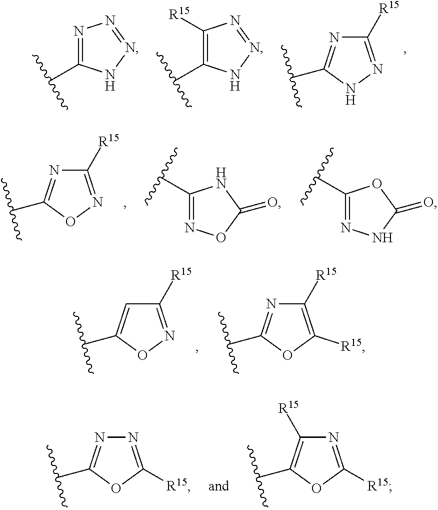

wherein:
each occurrence of R¹² and R¹³ is independently selected from the group consisting of: H, halogen, and C₁₋₃ alkyl; or R¹² and R¹³, together with the carbon atom to which each is attached, form a C₃-C₆ cycloalkyl;
R¹⁴ is:
   H;
   C₁₋₆ alkyl optionally substituted with 1-3 independently selected R^{a}; or
   C₃₋₆ cycloalkyl which is optionally substituted with 1-6 independently selected R^{b};
each occurrence of R¹¹ is independently selected from the group consisting of:
   H;
   halo;
   C₁₋₆ alkyl optionally substituted with 1-3 independently selected R^{a}; and
   C₃₋₆ cycloalkyl which is optionally substituted with 1-6 independently selected R^{b};
R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
      (B)
R¹, R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
R⁴, R⁵, R⁶, R⁷, R⁸, R⁹, R¹⁰, and R¹¹ are defined according to (C) and (D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R^{b}; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R^{g}) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R^{g} is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a};
each occurrence of R′ and R″ is independently selected from the group consisting of: H; —OH; and C₁₋₄ alkyl;
A) provided when:
R¹ is CO₂H,
each of R⁴, R⁵, R⁶, R⁷, R⁸, R⁹, and R¹⁰ is H,
R¹¹ is phenyl that is optionally substituted with one or more of NO₂, F, Cl, —OCH₃, —CH₃, phenyl, 2-benzothiazolyloxy, benzyloxy, iodo, —SCH₃, —SO₂CH₃, iso-propyl, iso-propoxy, iso-butyl, —OH, N₃, and NH₂; and
one of R² and R³ is H,
then the other of R² and R³ cannot be:
(i) the amino acid sidechain present in leucine, iso-leucine, threonine, tyrosine, O-benzyl-protected tyrosine, tryptophan, allothreonine, histidine, valine, alanine, arginine, glutamine, glutamic acid, aspartic acid, serine, lysine, N-benzyloxy-protected lysine, or methionine; or
(ii) n-hexyl, tert-butyl, n-butyl, or n-propyl;
B) further provided when:
R¹ is CO₂H,
each of R⁴, R⁵, R⁶, R⁷, R⁸, R⁹, and R¹⁰ is H,
R² and R³ together with the carbon atom to which each is attached form a 3, 4, 5, or 6 member ring; and
R¹ is phenyl
then the phenyl ring cannot be substituted at the para position with —SO₂CH₃; and
C) further provided the compound is not

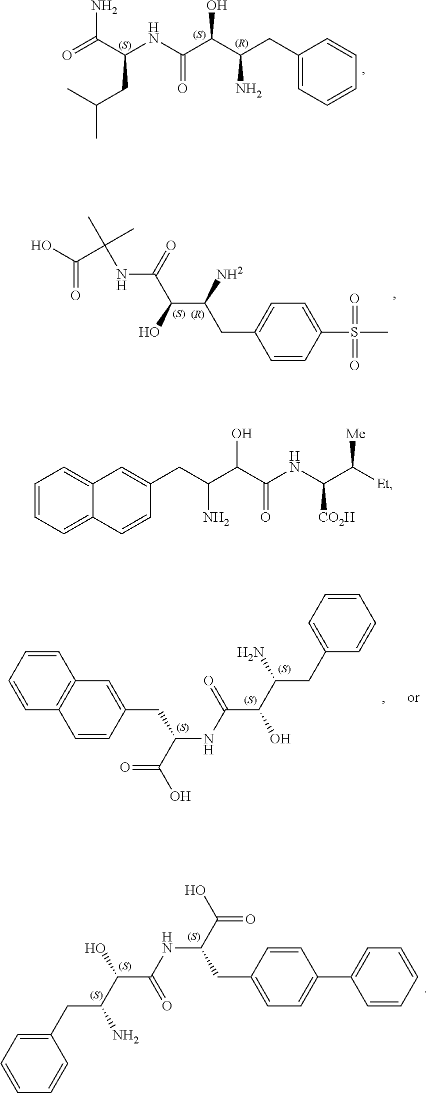

3. The compound of clause 1 or 2, wherein R¹, R², and R³ are defined according to (A).

4. The compound of any one of clauses 1-3, wherein R¹ is selected from the group consisting of —CO₂H, —CR^{12a}R^{13a}CO₂H, —CO(NR¹²)—OH, —CO—NR¹²R¹³, —CO—NR¹²—SO₂R¹⁴, —CO—NR¹²—CN, and —CO—NR¹²—CH₂—CF₃.

5. The compound of any one of clauses 1-4, wherein R¹ is —CO₂H.

6. The compound of any one of clauses 1-4, wherein R¹ is —CO—NR¹²—CN,

7. The compound of clause 5, wherein R¹² is H.

8. The compound of any one of clauses 1-4, wherein R¹ is —CO—NR¹²—SO₂R^{M}.

9. The compound of clause 8, wherein R¹² is H.

10. The compound of clause 8 or 9, wherein R¹⁴ is C₁₋₆ alkyl optionally substituted with 1-3 independently selected R^{a}.

11. The compound of any one of clauses 8-10, wherein R⁴ is CH₃.

12. The compound of any one of clauses 1-3, wherein R¹ is selected from the group consisting of:

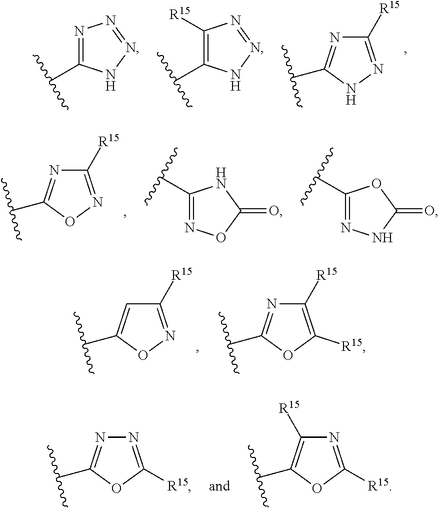

13. The compound of clause 1 or 12, wherein R¹ is

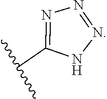 [SMILES: *c1nnn[nH]1]

14. The compound of any one of clauses 1-13, wherein R² and R³ are each independently selected from the group consisting of:
H;
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H.

15. The compound of any one of clauses 1-14, wherein one of R² and R³ is selected from the group consisting of:
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; and and the other of R² and R³ is H.

16. The compound of any one of clauses 1-15, wherein one of R² and R³ is L⁴-C₆₋₁₀ aryl optionally substituted with 1-4 substituents independently selected from the group consisting of oxo and R^{c}, wherein L⁴ is a bond or unsubstituted C₁₋₆ alkylene; and the other of R² and R³ is H.

17. The compound of clause 16, wherein one of R² and R³ is L⁴-phenyl optionally substituted with 1-4 substituents independently selected from the group consisting of oxo and R^{c}, wherein L⁴ is a bond or unsubstituted C₁₋₆ alkylene; and the other of R² and R³ is H.

18. The compound of clause 16 or 17, wherein L⁴ is C₁₋₆ alkylene.

19. The compound of any one of clauses 16-18, wherein L⁴ is CH₂.

20. The compound of clause 16 or 17, wherein L⁴ is a bond.

21. The compound of any one of clauses 1-15, wherein one of R² and R³ is L¹-C₃₋₁₀ cycloalkyl or L¹-C₃₋₁₀ cycloalkenyl, each of which is optionally substituted with 1-6 substituents independently selected from the group consisting of oxo and R^{b}, wherein Li is a bond or unsubstituted C₁₋₆ alkylene; and the other of R² and R³ is H.

22. The compound of clause 21, wherein one of R² and R³ is L¹-C₃₋₁₀ cycloalkyl, which is optionally substituted with 1-6 substituents independently selected from the group consisting of oxo and R^{b}, wherein L¹ is a bond or unsubstituted C₁₋₆ alkylene; and the other of R² and R³ is H.

23. The compound of clause 21 or 22, wherein one of R² and R³ is L¹-C₃₋₇ cycloalkyl, which is optionally substituted with 1-6 substituents independently selected from the group consisting of oxo and R^{b}, wherein L¹ is a bond or unsubstituted C₁₋₆ alkylene; and the other of R² and R³ is H.

24. The compound of any one of clauses 21-23, wherein L¹ is C₁₋₆ alkylene.

25. The compound of any one of clauses 21-24, wherein L¹ is CH₂.

26. The compound of any one of clauses 21-23, wherein L¹ is a bond.

27. The compound of any one of clauses 1-15, wherein one of R² and R³ is L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene; and the other of R² and R³ is H.

28. The compound of clause 27, wherein one of R² and R³ is L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

29. The compound of clause 27 or 28, wherein one of R² and R³ is L²-heterocyclyl of 3-7 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

30. The compound of any one of clauses 27-29, wherein L² is C₁₋₆ alkylene.

31. The compound of any one of clauses 27-30, wherein L² is CH₂.

32. The compound of any one of clauses 27-30, wherein L² is a bond.

33. The compound of any one of clauses 1-13, wherein R² and R³, together with the carbon atom to which each is attached, form a ring selected from the group consisting of.
C₃₋₁₀ cycloalkyl or C₃₋₁₀ cycloalkenyl, each of which is optionally substituted with 1-6 substituents independently selected from the group consisting of oxo and R^{b}; and
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}.

34. The compound of clause 1, wherein R¹, R², and R³ are defined according to (B).

35. The compound of any one of clauses 1-34, wherein R⁴, R⁵, R⁶, R⁷, R⁸, R⁹, R¹⁰, and R¹¹ are defined according to (C).

36. The compound of any one of clauses 1-35, wherein R⁴, R, and each R⁸ are H.

37. The compound of any one of clauses 1-36, wherein R⁶ and R⁷ are H.

38. The compound of any one of clauses 1-37, wherein R⁹ and R¹⁰ are H.

39. The compound of any one of clauses 1-38, wherein R⁴, R⁵, R⁶, R⁷, R⁸, R⁹, and R¹⁰ are H.

40. The compound of any one of clauses 1-39, wherein R¹¹ is C₆₋₁₀ aryl optionally substituted with 1-4 substituents independently selected from the group consisting of oxo and R^{c}.

41. The compound of any one of clauses 1-40, wherein R¹¹ is phenyl optionally substituted with 1-4 substituents independently selected from the group consisting of oxo and R^{c}.

42. The compound of any one of clauses 1-41, wherein R¹¹ is unsubstituted phenyl.

43. The compound of any one of clauses 1-42, wherein R⁴, R⁵, R⁶, R⁷, R⁸, R⁹, R¹⁰, and R¹¹ are defined according to (D).

44. The compound of any one of clauses 1-43, wherein the carbon atom attached to R¹, R², and R³ has the S-configuration.

45. The compound of any one of clauses 1-44, wherein the carbon atom attached to OR⁵ and R⁶ has the S-configuration.

46. The compound of any one of clauses 1-45, wherein the carbon atom attached to N(R⁸)₂ and R⁷ has the R-configuration.

47. The compound of any one of clauses 1-46, wherein the carbon atom attached to OR⁵ and R⁶ has the S-configuration, and the carbon atom attached to N(R⁸)₂ and R⁷ has the R-configuration.

48. The compound of any one of clauses 1-47, wherein the carbon atom attached to R¹, R², and R³ has the S-configuration, the carbon atom attached to OR⁵ and R⁶ has the S-configuration, and the carbon atom attached to N(R⁸)₂ and R⁷ has the R-configuration.

49. The compounds of any one of clauses 1-42 and 49-52, wherein the compound is a compound of Formula (I-i), (I-j), (I-k), or (I-l):

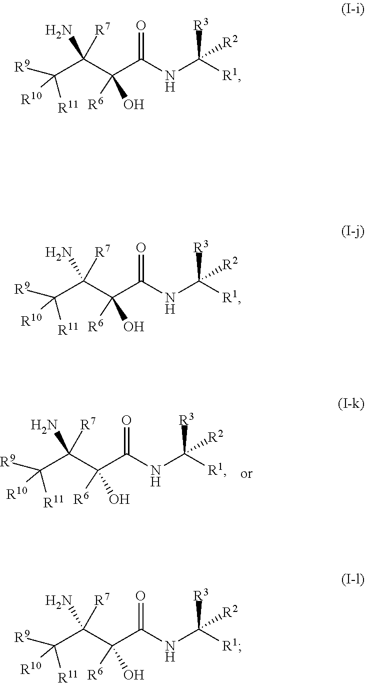

wherein:
R¹ is CO₂H, —CO—NR¹²R¹³, and R^{1A}.
wherein:
R^{1A} is a carboxylic acid isostere or bioisostere optionally substituted with 1-3 independently selected R¹⁵;
R² is H or CH₃;
R³ is selected from the group consisting of:
   H
   C₁₋₆ alkyl optionally substituted with 1-6 independently selected R^{a};
   L¹-C₃₋₁₀ cycloalkyl optionally substituted with 1-6 R^{b}, wherein L¹ is a bond or unsubstituted C₁₋₆ alkylene;
   L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
   L⁴-C₆₋₁₀ aryl optionally substituted with 1-4 R^{c}, wherein L⁴ is a bond or unsubstituted C₁₋₆ alkylene; provided that one or both of R² and R³ is other than H.

50. The compounds of clause 49, wherein the compound is a compound of Formula (I-i).

51. The compounds of any one of clauses 1-42 and 49-50, wherein the compound is a compound of Formula (I-ia), (I-ja), (I-ka), or (I-la):

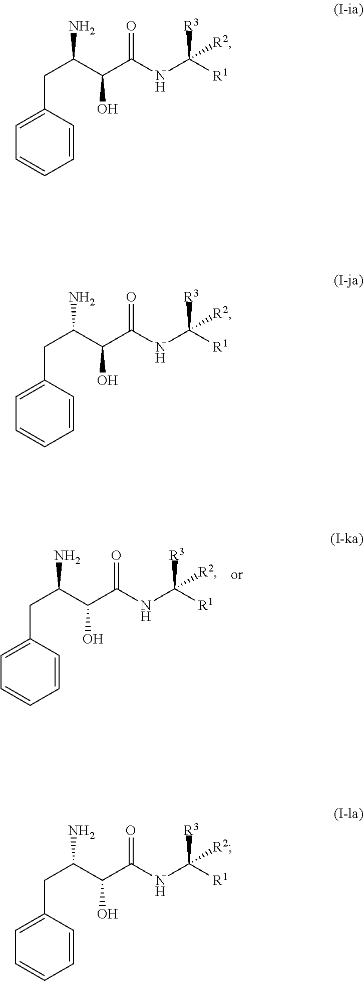

wherein:
R¹ is CO₂H, —CO—NR¹²R¹³, and R^{1A},
wherein:
R^{1A} is a carboxylic acid isostere or bioisostere optionally substituted with 1-3 independently selected R¹⁵;
R² is H or CH₃;
R³ is selected from the group consisting of:
   H
   C₁₋₆ alkyl optionally substituted with 1-6 independently selected R^{a};
   L¹-C₃₋₁₀ cycloalkyl optionally substituted with 1-6 R^{b}, wherein L¹ is a bond or unsubstituted C₁₋₆ alkylene;
   L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
   L⁴-C₆₋₁₀ aryl optionally substituted with 1-4 R^{c}, wherein L⁴ is a bond or unsubstituted C₁₋₆ alkylene; provided that one or both of R² and R³ is other than H.

52. The compounds of clause 51, wherein the compound is a compound of Formula (I-ia).

53. The compounds of any one of clauses 49-52, wherein R¹ is CO₂H.

54. The compounds of any one of clauses 49-52, wherein R¹ is R^{1A} a carboxylic acid isostere or bioisostere.

55. The compounds of any one of clauses 49-52 or 54, wherein R^{1A} is

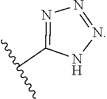 [SMILES: *c1nnn[nH]1]

56. A compound having Formula (I):

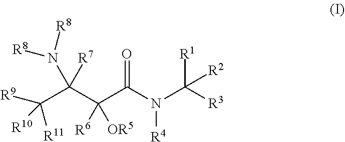

or a pharmaceutically acceptable thereof, wherein:
R¹ is —CO₂H;
R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
R⁴, R⁵, R⁶, R⁷, R⁸, R⁹, R¹⁰, and R¹¹ are defined according to_(Q and (D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R^{b}; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R^{g} is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a};
each occurrence of R′ and R″ is independently selected from the group consisting of: H; —OH; and C₁₋₄ alkyl;
A) provided when:
R¹ is CO₂H,
each of R⁴, R⁵, R⁶, R⁷, R⁸, R⁹, and R¹⁰ is H,
R¹¹ is phenyl that is optionally substituted with one or more of NO₂, F, Cl, —OCH₃, —CH₃, phenyl, 2-benzothiazolyloxy, benzyloxy, iodo, —SCH₃, —SO₂CH₃, iso-propyl, iso-propoxy, iso-butyl, —OH, N₃, and NH₂; and
one of R² and R³ is H,
then the other of R² and R³ cannot be:
(i) the amino acid sidechain present in leucine, iso-leucine, threonine, tyrosine, O-benzyl-protected tyrosine, tryptophan, allothreonine, histidine, valine, alanine, arginine, glutamine, glutamic acid, aspartic acid, serine, lysine, N-benzyloxy-protected lysine, or methionine; or
(ii) n-hexyl, tert-butyl, n-butyl, or n-propyl;
B) further provided when:
R¹ is CO₂H,
each of R⁴, R⁵, R⁶, R⁷, R⁸, R⁹, and R¹⁰ is H,
R² and R³ together with the carbon atom to which each is attached form a 3, 4, 5, or 6 member ring; and
R¹ is phenyl
then the phenyl ring cannot be substituted at the para position with —SO₂CH₃; and
C) further provided the compound is not

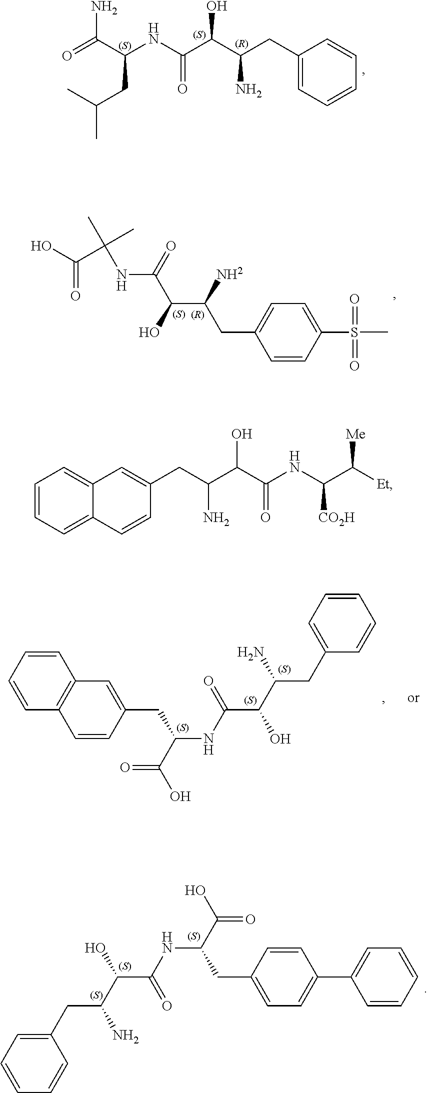

57. A compound having Formula (I):

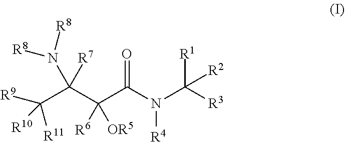

or a pharmaceutically acceptable thereof, wherein:
R¹ is R^{1A};
wherein:
R^{1A} is a carboxylic acid isostere or bioisostere optionally substituted with 1-3 independently selected R′⁵;
each occurrence of R¹⁵ is independently selected from the group consisting of:
   H;
   halo;
   C₁₋₆ alkyl optionally substituted with 1-3 independently selected R^{a}; and
   C₃₋₆ cycloalkyl which is optionally substituted with 1-6 independently selected R^{b};
R² and R³ are each independently selected from the group consisting of:
   H;
   halo;
   CN;
   C₁₋₂₀ alkyl optionally substituted with 1-6 independently selected R^{a};
   L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
R⁴, R⁵, R⁶, R⁷, R⁸, R⁹, R¹⁰, and R¹¹ are defined according to (C) and D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R^{b}; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R^{g}) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
   heteroaryl of 5-10 ring atoms, wherein 1-4 ring atoms (in addition to the ring N that is attached to R^{g}) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R^{g} is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a};
each occurrence of R′ and R″ is independently selected from the group consisting of: H; —OH; and C₁₋₄ alkyl.

58. A compound having Formula (I):

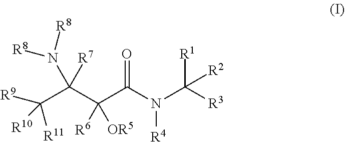

or a pharmaceutically acceptable thereof, wherein:
R¹ is

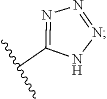 [SMILES: *c1nnn[nH]1]

R² and R³ are each independently selected from the group consisting of:
   H;
   halo;
   CN;
   C₁₋₂₀ alkyl optionally substituted with 1-6 independently selected R^{a};
   L-C₃₋₁₀ cycloalkyl or L-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
   L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
   L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
   C₃₋₁₀ cycloalkyl or C₃₋₁₀ cycloalkenyl, each of which is optionally substituted with 1-6 substituents independently selected from the group consisting of oxo and R^{b}; and
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
R⁴, R⁵, R⁶, R⁷, R⁸, R⁹, R¹⁰, and R¹¹ are defined according to (C) and (D) below:
   (C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
   C₃₋₆ cycloalkyl or C₃₋₆₀ cycloalkenyl, each of which is optionally substituted with 1-6 substituents independently selected from the group consisting of oxo and R^{b}; and
   heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
   C₃₋₁₀ cycloalkyl or C₃₋₁₀ cycloalkenyl, each of which is optionally substituted with 1-6 substituents independently selected from the group consisting of oxo and R^{b};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R;
   heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
   C₆₋₁₀ aryl optionally substituted with 1-4 substituents independently independently selected from the group consisting of oxo and R^{c};
      (D)
one of R⁸ and R¹¹, together with the atoms to which each is attached, form a ring selected from the group consisting of:
   heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R; and
   heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R⁸ is selected from the group consisting of: H and C₁₃ alkyl; and
each occurrence of R⁶, R⁷, R⁹, and R¹⁰ is independently selected from the group consisting of: H; F; C₁₋₆ alkyl optionally substituted with 1-4 independently selected R^{a}, and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently from the group consisting of:
   C₃₋₈ cycloalkyl or C₃₋₈ cycloalkenyl, each of which is optionally substituted with 1-6 substituents independently selected from the group consisting of C₁₋₆ alkyl and R^{a};
   heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
   heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
   C₆₋₁₀ aryl optionally substituted with 1-4 substituents independently independently selected from the group consisting of C₁₋₆ alkyl and R^{a};
each occurrence of R′ and R″ is independently selected from the group consisting of: H; —OH; and C₁₋₄ alkyl.

59. The compound of any one of clauses 56-58, wherein R² and R³ are each independently selected from the group consisting of:
H;
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; provided that one or both of R² and R³ is other than H.

60. The compound of any one of clauses 56-59, wherein one of R² and R³ is selected from the group consisting of:
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently independently selected from the group consisting of oxo and R^{c}, wherein L⁴ is a bond or unsubstituted C₁₋₆ alkylene; and
and the other of R² and R³ is H.

61. The compound of any one of clauses 56-60, wherein one of R² and R³ is L⁴-C₆₋₁₀ aryl optionally substituted with 1-4 substituents independently selected from the group consisting of oxo and R^{c}, wherein L⁴ is a bond or unsubstituted C₁₋₆ alkylene; and the other of R² and R³ is H.

62. The compound of clause 61, wherein one of R² and R³ is L⁴-phenyl optionally substituted with 1-4 substituents independently selected from the group consisting of oxo and R^{c}, wherein L⁴ is a bond or unsubstituted C₁₋₆ alkylene; and the other of R² and R³ is H.

63. The compound of clause 61 or 62, wherein L⁴ is C₁₋₆ alkylene.

64. The compound of any one of clauses 61-63, wherein L⁴ is CH₂.

65. The compound of clause 61 or 62, wherein L⁴ is a bond.

66. The compound of any one of clauses 56-60, wherein one of R² and R³ is L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene; and the other of R² and R³ is H.

67. The compound of clause 66, wherein one of R² and R³ is L¹-C₃₋₁₀ cycloalkyl, which is optionally substituted with 1-6 substituents independently selected from the group consisting of oxo and R^{b}, wherein L¹ is a bond or unsubstituted C₁₋₆ alkylene; and the other of R² and R³ is H.

68. The compound of clause 66 or 67, wherein one of R² and R³ is L¹-C₃₋₇ cycloalkyl, which is optionally substituted with 1-6 substituents independently selected from the group consisting of oxo and R^{b}, wherein L¹ is a bond or unsubstituted C₁₋₆ alkylene; and the other of R² and R³ is H.

69. The compound of any one of clauses 66-68, wherein L¹ is C₁₋₆ alkylene.

70. The compound of any one of clauses 66-69, wherein L¹ is CH₂.

71. The compound of any one of clauses 66-68, wherein L¹ is a bond.

72. The compound of any one of clauses 56-60, wherein one of R² and R³ is L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene; and the other of R² and R³ is H.

73. The compound of clause 72, wherein one of R² and R³ is L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

74. The compound of clause 72 or 73, wherein one of R² and R³ is L²-heterocyclyl of 3-7 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo and R^{b}, wherein L² is a bond or unsubstituted C₁₋₆ alkylene; and the other of R² and R³ is H.

75. The compound of any one of clauses 72-74, wherein L² is C₁₋₆ alkylene.

76. The compound of any one of clauses 72-75, wherein L² is CH₂. 77. The compound of any one of clauses 72-75, wherein L² is a bond.

78. The compound of any one of clauses 56-58, wherein R² and R³, together with the carbon atom to which each is attached, form a ring selected from the group consisting of:
C₃₋₁₀ cycloalkyl or C₃₋₁₀ cycloalkenyl, each of which is optionally substituted with 1-6 substituents independently selected from the group consisting of oxo and R^{b}; and
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}.

80. The compound of any one of clauses 56-79, wherein R⁴, R⁵, R⁶, R⁷, R⁸, R⁹, R¹⁰, and R¹¹ are defined according to (Q.

81. The compound of any one of clauses 56-80, wherein R⁴, R⁵, and each R⁸ are H.

82. The compound of any one of clauses 56-81, wherein R⁶ and R⁷ are H.

83. The compound of any one of clauses 56-82, wherein R⁹ and R¹⁰ are H.

84. The compound of any one of clauses 56-83, wherein R⁴, R⁵, R⁶, R⁷, R⁸, R⁹, and R¹⁰ are H.

85. The compound of any one of clauses 56-84, wherein R¹¹ is C₆₋₁₀ aryl optionally substituted with 1-4 substituents independently selected from the group consisting of oxo and R^{c}.

86. The compound of any one of clauses 56-85, wherein R¹¹ is phenyl optionally substituted with 1-4 substituents independently selected from the group consisting of oxo and R^{c}.

87. The compound of any one of clauses 56-86, wherein R¹¹ is unsubstituted phenyl.

88. The compound of any one of clauses 56-87, wherein R⁴, R⁵, R⁶, R⁷, R⁸, R⁹, R¹⁰, and R¹¹ are defined according to (D).

89. The compound of any one of clauses 56-88, wherein the carbon atom attached to R¹, R², and R³ has the S-configuration.

90. The compound of any one of clauses 56-89, wherein the carbon atom attached to OR⁵ and R⁶ has the S-configuration.

91. The compound of any one of clauses 56-90, wherein the carbon atom attached to N(R⁸)₂ and R⁷ has the R-configuration.

92. The compound of any one of clauses 56-91, wherein the carbon atom attached to OR⁵ and R⁶ has the S-configuration, and the carbon atom attached to N(R⁸)₂ and R⁷ has the R-configuration.

93. The compound of any one of clauses 56-92, wherein the carbon atom attached to R¹, R², and R³ has the S-configuration, the carbon atom attached to OR⁵ and R⁶ has the S-configuration, and the carbon atom attached to N(R⁸)₂ and R⁷ has the R-configuration.

94. The compounds of any one of clauses 56-87, wherein the compound is a compound of Formula (I-i), (I-j), (I-k), or (I-1):

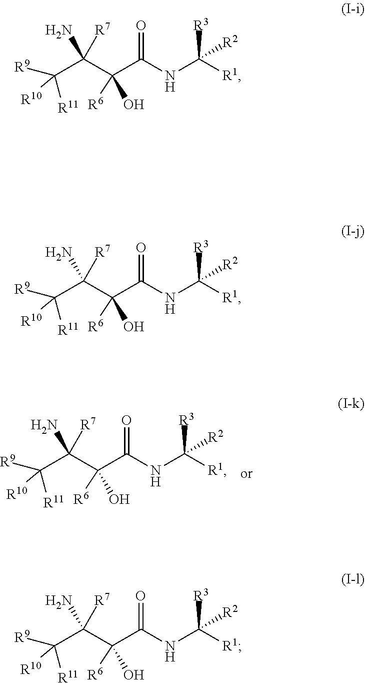

wherein:
R is CO₂H, —CO—NR¹²R¹³, and R^{1A}.
wherein:
R^{1A} is a carboxylic acid isostere or bioisostere;
R² is H or CH₃;
R³ is selected from the group consisting of:
   H
   C₁₋₆ alkyl optionally substituted with 1-6 independently selected R^{a};
   L-C₃₋₁₀ cycloalkyl optionally substituted with 1-6 R^{b}, wherein L¹ is a bond or unsubstituted C₁₋₆ alkylene;
   L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
   L⁴-C₆₋₁₀ aryl optionally substituted with 1-4 R^{c}, wherein L⁴ is a bond or unsubstituted C₁₋₆ alkylene; provided that one or both of R² and R³ is other than H.

95. The compounds of clause 94, wherein the compound is a compound of Formula (I-i).

96. The compounds of any one of clauses 56-87, wherein the compound is a compound of Formula (I-ia), (I-j a), (I-ka), or (I-la):

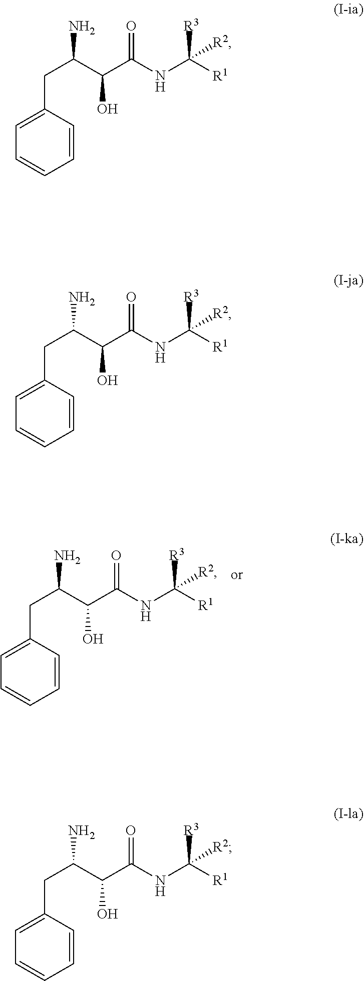

wherein:
R¹ is CO₂H, —CO—NR¹²R¹³, and R^{1A},
wherein:
R^{1A} is a carboxylic acid isostere or bioisostere;
R² is H or CH₃;
R³ is selected from the group consisting of:
   H
   C₁₋₆ alkyl optionally substituted with 1-6 independently selected R^{a};
   L¹-C₃₋₁₀ cycloalkyl optionally substituted with 1-6 R^{b}, wherein L¹ is a bond or unsubstituted C₁₋₆ alkylene;
   L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 R^{b}, wherein L² is a bond or unsubstituted C₁₋₆ alkylene;
   L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
   L⁴-C₆₋₁₀ aryl optionally substituted with 1-4 R^{c}, wherein L⁴ is a bond or unsubstituted C₁₋₆ alkylene; provided that one or both of R² and R³ is other than H.

97. The compounds of clause 96, wherein the compound is a compound of Formula (I-ia).

98. The compound of clause 1, wherein the compound is selected from the group of compounds delineated in Table C1.

99. A pharmaceutical composition comprising a compound of any one of clauses 1-97 and one or more pharmaceutically acceptable excipients.

1. A compound having formula I: 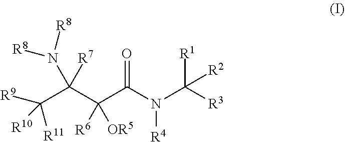
or a pharmaceutically acceptable thereof, wherein:
R¹, R², and R³ are defined according to (A) and (B) below:
(A)
R¹ is selected from the group consisting of —CO₂H, —CO—NR¹²R¹³, and R^{1A};
wherein:
R^{1A} is a carboxylic acid isostere or bioisostere;
each occurrence of R¹² and R¹³ is independently selected from the group consisting of H and C₁₋₃ alkyl;
R² and R³ are each independently selected from the group consisting of:
H;
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently selected from the group consisting of oxo and R^{c}, wherein L⁴ is a bond or unsubstituted C₁₋₆ alkylene; provided that one or both of R² and R³ is other than H; or
R² and R³, together with the carbon atom to which each is attached, form a ring selected from the group consisting of:
C₃₋₁₀ cycloalkyl or C₃₋₁₀ cycloalkenyl, each of which is optionally substituted with 1-6 substituents independently selected from the group consisting of oxo and R^{b}; and
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
R¹, R² and R³, together with the carbon atom to which each is attached, form a ring selected from the group consisting of:
C₃₋₁₀ cycloalkyl or C₃₋₁₀ cycloalkenyl, each of which is optionally substituted with 1-6 substituents independently selected from the group consisting of oxo and R^{b};
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
C₆₋₁₀ aryl optionally substituted with 1-4 substituents independently selected from the group consisting of oxo and R^{c};
R⁴, R⁵, R⁶, R⁷, R⁸, R⁹, R¹⁰, and R¹¹ are defined according to (C) and (D) below:
(C)
each occurrence of R⁴, R⁵, R⁸ is independently selected from the group consisting of: H and C₁₋₃ alkyl;
each occurrence of R⁶ and R⁷ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R⁹ and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b}; or
R⁹ and R¹⁰, together with the carbon atom to which each is attached, form a ring selected from the group consisting of:
C₃₋₆ cycloalkyl or C₃₋₆₀ cycloalkenyl, each of which is optionally substituted with 1-6 substituents independently selected from the group consisting of oxo and R^{b}; and
heterocyclyl or heterocycloalkenyl of 3-6 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}; and
R¹¹ is selected from the group consisting of:
C₃₋₁₀ cycloalkyl or C₃₋₁₀ cycloalkenyl, each of which is optionally substituted with 1-6 substituents independently selected from the group consisting of oxo and R^{b};
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b};
heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}; and
C₆₋₁₀ aryl optionally substituted with 1-4 substituents independently selected from the group consisting of oxo and R^{c};
(D)
one of R⁸ and R¹¹, together with the atoms to which each is attached, form a ring selected from the group consisting of:
heterocyclyl or heterocycloalkenyl of 3-10 ring atoms, wherein 1-3 ring atoms (in addition to the ring N that is attached to R⁸) are heteroatoms, each independently selected from the group consisting of N, N(H), N(R), O, and S(O)₀₋₂, and wherein the heterocyclyl or heterocycloalkenyl is optionally substituted with 1-4 substituents independently selected from the group consisting of oxo and R^{b}; and
heteroaryl of 5-10 ring atoms, wherein 1-4 ring atoms (in addition to the ring N that is attached to R⁸) are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c};
the other of R⁸ is selected from the group consisting of: H and C₁₋₃ alkyl; and
each occurrence of R⁶, R⁷, R⁹, and R¹⁰ is independently selected from the group consisting of: H; F; C₁₋₆ alkyl optionally substituted with 1-4 independently selected R^{a}; and C₃₋₆ cycloalkyl or C₃₋₆ cycloalkenyl, each of which is optionally substituted with 1-6 substituents independently selected from the group consisting of oxo and R^{b};
each occurrence of R^{a} is independently selected from the group consisting of: —OH; -halo;
—NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(=NH)(C₁₋₄ alkyl); —NR^{e}R^{f}; N₃; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; —NR′C(═O)(C₁₋₄ alkyl); Si(C₁₋₄ aklyl)₃, —SF₅, and R^{g};
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently from the group consisting of:
C₃₋₈ cycloalkyl or C₃₋₈ cycloalkenyl, each of which is optionally substituted with 1-6 substituents independently selected from the group consisting of C₁₋₆ alkyl and R^{a};
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of C₁₋₆ alkyl and R^{a};
heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of C₁₋₆ alkyl and R^{a}; and
C₆₋₁₀ aryl optionally substituted with 1-4 substituents independently selected from the group consisting of C₁₋₆ alkyl and R^{a};
each occurrence of R′ and R¹¹ is independently selected from the group consisting of: H; —OH; and C₁₋₄ alkyl;
A) provided when:
R¹ is CO₂H,
each of R⁴, R⁵, R⁶, R⁷, R⁸, R⁹, and R¹⁰ is H,
R¹¹ is phenyl that is optionally substituted with one or more of NO₂, F, Cl, —OCH₃, —CH₃, phenyl, 2-benzothiazolyloxy, benzyloxy, iodo, —SCH₃, —SO₂CH₃, iso-propyl, iso-propoxy, iso-butyl, —OH, N₃, and NH₂; and
one of R² and R³ is H,
then the other of R² and R³ cannot be:
(i) the amino acid sidechain present in leucine, iso-leucine, threonine, tyrosine, O-benzyl-protected tyrosine, tryptophan, allothreonine, histidine, valine, alanine, arginine, glutamine, glutamic acid, aspartic acid, serine, lysine, N-benzyloxy-protected lysine, or methionine; or
(ii) n-hexyl, tert-butyl, n-butyl, or n-propyl;
B) further provided when:
R¹ is CO₂H,
each of R⁴, R⁵, R⁶, R⁷, R⁸, R⁹, and R¹⁰ is H,
R² and R³ together with the carbon atom to which each is attached form a 3, 4, 5, or 6 member ring; and
R¹ is phenyl
then the phenyl ring cannot be substituted at the para position with —SO₂CH₃; and
C) further provided the compound is not

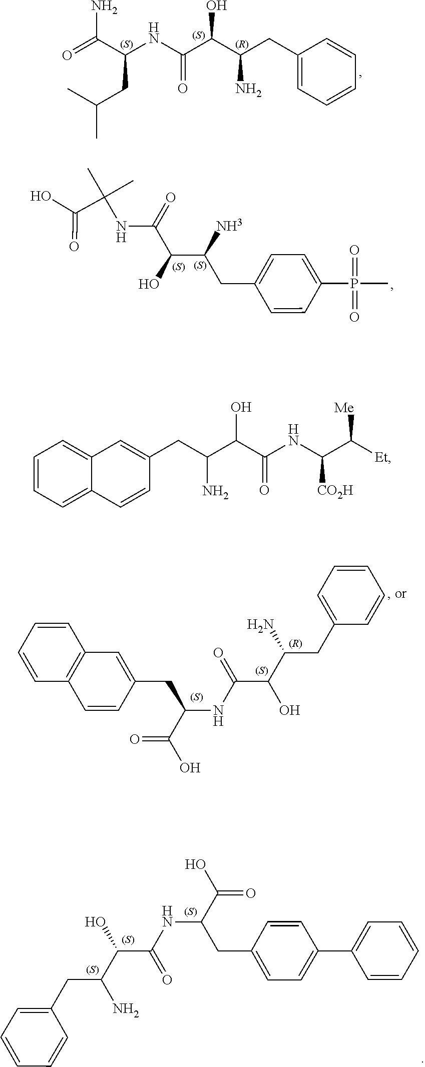

2. (canceled)

3. The compound of claim 1, wherein R¹, R², and R³ are defined according to (A).

4. The compound of claim 1, wherein R¹ is selected from the group consisting of —CO₂H, —CR^{12a}R^{13a}CO₂H, —CO(NR¹²)—OH, —CO—NR¹²R¹³, —CO—NR¹²—SO₂R⁴, —CO—NR¹²—CN, and —CO—NR¹²—CH₂—CF₃.

5. The compound of claim 1, wherein R¹ is —CO₂H.

6. (canceled)

7. (canceled)

8. (canceled)

9. (canceled)

10. (canceled)

11. (canceled)

12. The compound of claim 1, wherein R¹ is selected from the group consisting of:

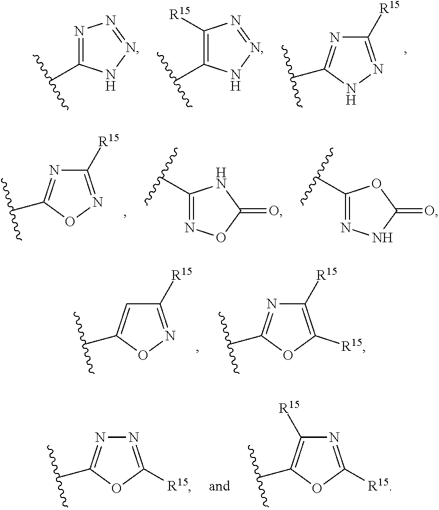

13. The compound of claim 1, wherein R¹ is

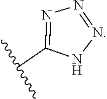 [SMILES: *c1nnn[nH]1]

14. The compound of claim 1, wherein R² and R³ are each independently selected from the group consisting of:
H;
halo;
CN;
C₁₋₂₀ alkyl optionally substituted with 1-6 independently selected R^{a};
L¹-C₃₋₁₀ cycloalkyl or L¹-C₃₋₁₀ cycloalkenyl, each of which is optionally substituted with 1-6 substituents independently selected from the group consisting of oxo and R^{b}, wherein L¹ is a bond or unsubstituted C₁₋₆ alkylene;
L²-heterocyclyl or L²-heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo and R^{c}, wherein L³ is a bond or unsubstituted C₁₋₆ alkylene; and
L⁴-C₆₋₁₀ aryl optionally substituted with 1-4 substituents independently selected from the group consisting of oxo and R^{c}, wherein L⁴ is a bond or unsubstituted C₁₋₆ alkylene; provided that one or both of R² and R³ is other than H.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The compound of claim 1, wherein R² and R³, together with the carbon atom to which each is attached, form a ring selected from the group consisting of:
C₃₋₁₀ cycloalkyl or C₃₋₁₀ cycloalkenyl, each of which is optionally substituted with 1-6 substituents independently selected from the group consisting of oxo and R^{b}; and
heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo and R^{b}.

34. The compound of claim 1, wherein R¹, R², and R³ are defined according to (B).

35. The compound of claim 1, wherein R⁴, R⁵, R⁶, R⁷, R⁸, R⁹, R¹⁰, and R¹¹ are defined according to (C).

36. (canceled)

37. (canceled)

38. (canceled)

39. The compound of claim 1, wherein R⁴, R⁵, R⁶, R⁷, R⁸, R⁹, and R¹⁰ are H.

40. The compound of claim 1, wherein R¹¹ is C₆₋₁₀ aryl optionally substituted with 1-4 substituents independently selected from the group consisting of oxo and R^{c}.

41. (canceled)

42. (canceled)

43. The compound of claim 1, wherein R⁴, R⁵, R⁶, R⁷, R⁸, R⁹, R¹⁰, and R¹¹ are defined according to (D).

44. (canceled)

45. (canceled)

46. (canceled)

47. (canceled)

48. (canceled)

49. The compound of claim 1, wherein the compound is a compound of Formula (I-i), (I-j), (I-k), or (I-l): 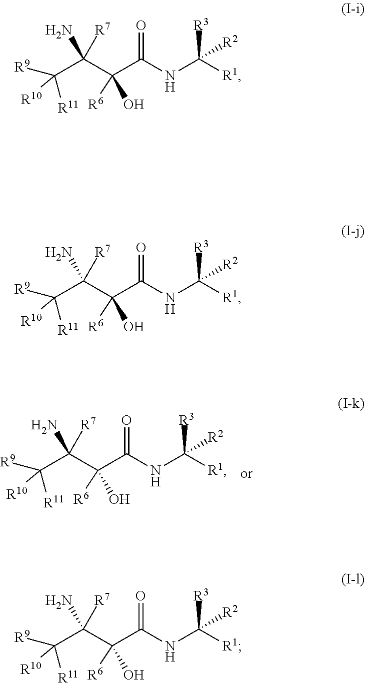
wherein:
R¹ is selected from the group consisting of CO₂H, —CO—NR¹²R¹³, and R^{1A};
wherein:
R^{1A} is a carboxylic acid isostere or bioisostere optionally substituted with 1-3 independently selected R's;
R² is H or CH₃;
R³ is selected from the group consisting of:
H
C₁₋₆ alkyl optionally substituted with 1-6 independently selected R^{a};
L¹-C₃₋₁₀ cycloalkyl optionally substituted with 1-6 R^{b}, wherein L¹ is a bond or unsubstituted C₁₋₆ alkylene;
L²-heterocyclyl of 3-10 ring atoms, wherein 1-3 ring atoms are heteroatoms, each independently selected from the group consisting of N, N(H), N(R), O, and S(O)₀₋₂, and wherein the heterocyclyl is optionally substituted with 1-4 R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
L⁴-C₆₋₁₀ aryl optionally substituted with 1-4 R^{c}, wherein L⁴ is a bond or unsubstituted C₁₋₆ alkylene; provided that one or both of R² and R³ is other than H.

50. (canceled)

51. The compound of claim 1, wherein the compound is a compound of Formula (I-ia), (I-ja), (I-ka), or (I-la): 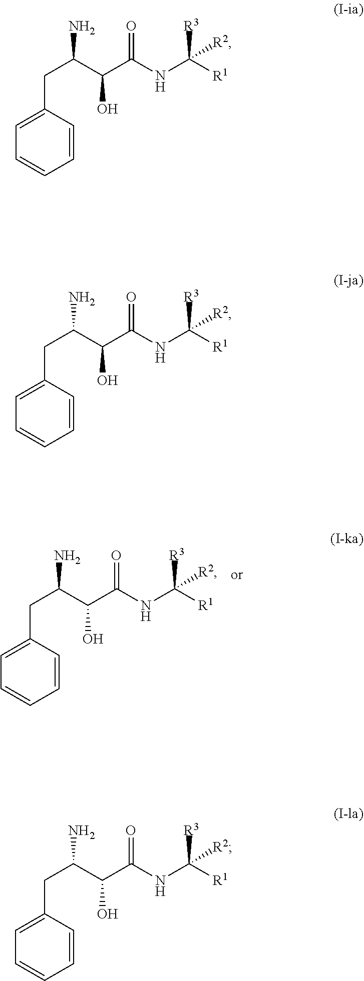
wherein:
R¹ is CO₂H, —CO—NR¹²R¹³, and R^{1A},
wherein:
R^{1A} is a carboxylic acid isostere or bioisostere optionally substituted with 1-3 independently selected R¹⁵;
R² is H or CH₃;
R³ is selected from the group consisting of:
H
C₁₋₆ alkyl optionally substituted with 1-6 independently selected R^{a};
L¹-C₃₋₁₀ cycloalkyl optionally substituted with 1-6 R^{b}, wherein L¹ is a bond or unsubstituted C₁₋₆ alkylene;
L²-heterocyclyl of 3-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 R^{b}, wherein L² is a bond or unsubstituted C₁₋₆ alkylene;
L³-heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 R^{c}, wherein L³ is a bond or unsubstituted C₁₋₆ alkylene; and
L⁴-C₆₋₁₀ aryl optionally substituted with 1-4 R^{c}, wherein L⁴ is a bond or unsubstituted C₁₋₆ alkylene; provided that one or both of R² and R³ is other than H.

52. (canceled)

53. (canceled)

54. (canceled)

55. (canceled)

56. (canceled)

57. (canceled)

58. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. The compound of claim 1, wherein the compound is selected from the group of compounds delineated in Table C₁.

99. A pharmaceutical composition comprising a compound of claim 1 and one or more pharmaceutically acceptable excipients.

100. A method for modulating CNDP2 in a mammalian cell, the method comprising contacting the mammalian cell with an effective amount of a compound of claim 1, or a pharmaceutically acceptable salt thereof.

101. (canceled)

102. (canceled)

103. (canceled)

104. A method of treating a condition, disease or disorder ameliorated by modulating (e.g., inhibiting) CNDP2 (e.g., a condition, disease or disorder in which altered (e.g., increased) CNDP2 expression or activity contributes to the pathology and/or symptoms and/or progression of the condition, disease or disorder in a subject comprising administering to a subject in need of such treatment an effective amount of a compound as claimed in claim 1.

105. A method of treating cancer, comprising administering to a patient in need thereof a therapeutically effective amount of a compound as claimed in claim 1.

106. (canceled)

107. (canceled)

108. (canceled)

109. (canceled)

110. The method of claim 104, wherein the condition, disease or disorder is frailty or one or more frailty conditions.

111. The method of claim 104, wherein the condition, disease or disorder is mitochondrial dysfunction.

112. (canceled)

113. The method of claim 104, wherein the condition, disease or disorder is muscle loss.

114. The method of claim 104, wherein the condition, disease or disorder is a metabolic disorder, obesity, insulin resistance, or insulin sensitivity.

115. (canceled)

116. (canceled)

117. (canceled)
